(19) United States
(12) Patent Application Publication
(10) Pub. No.: US 2007/0135803 A1
(43) Pub. Date: Jun. 14, 2007

(54) Methods and apparatus for performing transluminal and other procedures

(76) Inventor: Amir Belson, Sunnyvale, CA (US)

Correspondence Address: SHAY LAW GROUP, LLP, 2755 CAMPUS DRIVE, SUITE 210, SAN MATEO, CA, 94403, US

(21) Appl. No.: 11/522,305

(22) Filed: Sep. 14, 2006

Related U.S. Application Data
(60) Provisional application No. 60/717,230, filed on Sep. 14, 2005.

(51) Int. Cl. A61B 17/00; A61B 17/32

(52) U.S. Cl. 606/1; 606/167; 606/108

(57) ABSTRACT

The invention is directed to an apparatus for use in a transluminal procedure. The apparatus, comprising, for example, a housing having a guide lumen and a seal proximal to a distal end of the housing that extends across and completely seals the guide lumen; a fixation element in the housing and adapted to secure the distal end of the housing to tissue; and a channel extending through the side wall of the housing having an outlet in communication with the lumen distal of the seal. Methods are also provided. For example, a method includes, performing a transluminal procedure by: securing a datum and position indicator to a wall of a target lumen; forming an opening in the wall; advancing an instrument through the opening; and tracking the advancement of the instrument using the datum and position indicator.

### CROSS-REFERENCE TO RELATED APPLICATIONS

This application claims the benefits of priority to U.S. Provisional Patent Application Ser. No. 60/717,230 filed Sep. 14, 2005, the entirety of which is incorporated herein by reference.

### FIELD OF THE INVENTION

The present invention is directed to minimally invasive surgical procedures. In particular, the invention relates to improved methods, systems and devices for use in transluminal procedures.

### BACKGROUND OF THE INVENTION

There has been a steady progression in surgical procedures to reduce the difficulty for the surgeon and the recovery time required for the patent. Open surgical procedures have given way to laparoscopic surgery. Laparoscopic procedures are evolving towards minimally invasive surgical procedures.

While these advances are reducing the exterior incisions needed to access the internal organs, other procedures seek to remove external access and instead rely on the naturally occurring openings in the body to provide surgical access. Such procedures enter the body through a natural orifice and then create the surgical access within the body at the desired location.

While intra-abdominal and trans-luminal procedures have been suggested for several years, many problems remain unsolved or with sub-optimum solutions. Specifically, shortcomings exist in methods and instruments to create precise openings in the lumen wall or to close the lumen opening once created. Difficulties remain with creating a sterile surgical environment within the body, particularly in those procedures desiring access via the colon.

In view of the ongoing challenges confronting the advancement of trans-luminal procedures, improvements are still needed. In particular, improvements are needed in the manner by which instruments are controlled, trans-luminal openings are created and sterility is maintained.

### SUMMARY OF THE INVENTION

In keeping with the foregoing discussion, the present invention takes the form of methods and apparatus for performing endoscopic colectomy that combine the advantages of the laparoscopic and endolumenal approaches. The diseased portion of the colon to be resected is identified using either laparoscopic and/or colonoscopic techniques or using another imaging modality. A colectomy device mounted on a colonoscope grasps the colon wall at two sites adjacent to a diseased portion of the colon. Using laparoscopic techniques, the diseased portion of the colon is separated from the omentum and the blood vessels supplying it are ligated or cauterized. The colon wall is transected to remove the diseased portion and the excised tissue is removed using the laparoscope or drawn into the colectomy device for later removal upon withdrawal of the colonoscope. The colectomy device approximates the two ends of the colon and performs an end-to-end anastomosis. If the part to be resected is a tumor, prior to the resection, the edges of the segment to be resected will be stapled to seal it and prevent spillage of malignant cells to the healthy tissue.

The methods and apparatus of the present invention provide a number of benefits not realized by the prior art approaches to colectomy. As stated above, the purely endolumenal approach does not provide for separation of the colon from the omentum, which is necessary when resecting more than just a small portion of the colon wall. By combining laparoscopic techniques with a colonoscope-mounted colectomy device, the present invention overcomes this deficiency in the prior art allowing a more comprehensive approach to colectomy. Unlike prior art laparoscopic techniques, however, the colon does not need to be exteriorized for excision of the diseased portion or anastomosis of the remaining colon. The colonoscope-mounted colectomy device approximates the ends of the colon and performs an anastomosis from the interior of the lumen of the colon. The excised tissue can be drawn into the colectomy device for removal through the lumen of the colon along with the colonoscope or can be taken out by the laparoscope, which can be done through a very small incision in the patient's skin. The prior art approach also does not protect from leaking of malignant cells to the periphery. This idea will enable sealing of the tissue with staples at its ends to prevent such leakage. Optionally, it will be done with the help of a laparoscopic device that will serve as an anvil. Unlike the prior art procedure, the present invention will optionally use a balloon inflated in the lumen of the colon or any other resected organ before stapling, and by this assure the anastomosis will be ideal with the best possible approximation of the edges.

The use of colonoscopic techniques in the present invention provides another benefit not realized by a purely laparoscopic approach. Since colonoscopic examination is at present the most definitive diagnostic method for identifying diseases of the colon, locating the lesions through the exterior of the colon by laparoscopy or even by direct visualization can be somewhat problematic. Using the colonoscope to identify and isolate the diseased portion of the colon from within the lumen helps assure that the correct portions of the colon wall are excised and makes clean surgical margins without residual disease more assured as well.

In a preferred embodiment, the present invention utilizes a steerable colonoscope as described in U.S. patent application Ser. Nos. 09/790,204 (now U.S. Pat. No. 6,468,203); 09/969,927; and 10/229,577, which have been incorporated by reference. The steerable colonoscope described therein provides a number of additional benefits for performing endoscopic colectomy according to the present invention. The steerable colonoscope uses serpentine motion to facilitate rapid and safe insertion of the colonoscope into the patient's colon, which allows the endoscopic colectomy method to be performed more quickly and more safely. Beyond this however, the steerable colonoscope has the capability to create a three-dimensional mathematical model or map of the patient's colon and the location of any lesions identified during the initial examination. Lesions found during a previous examination by CT, MRI or any other imaging technology can also be mapped onto the three dimensional map of the colon. By generating a three dimensional map of the colon, the system knows where each part of the endoscope is in the colon and will be able to localize the two parts of the dissecting and stapling system exactly in the desired location. During surgery, this information can be used to quickly and accurately return the colonoscope to the location of the identified lesions where the colonoscope-mounted colectomy device will be used to complete the endoscopic colectomy procedure.

An aspect of the invention includes a method for performing a transluminal procedure. The method comprises: securing a datum and position indicator to a wall of a target lumen; forming an opening in the wall; advancing an instrument through the opening; and tracking the advancement of the instrument using the datum and position indicator. Additional steps include forming the opening with an instrument coupled to the datum and position indicator, or advancing the instrument through a guide lumen in the datum and position indicator. Additionally, a piercing step can be provided that includes piercing a sheath extending across the guide lumen while advancing the instrument through the lumen in the datum and position indicator. Additionally, a sheath contained in the datum and position indicator can be unrolled while advancing the instrument through the guide lumen. In some embodiments, the method can include the step of rigidizing a guide tube coupled to the datum and position indicator before tracking the advancement of the instrument. An additional step can include sterilizing the wall of the target lumen after securing the datum and position indicator for the wall of the target lumen. In some embodiments, the tracking step comprises providing instrument tracking information to a system used to monitor the progress of the instrument. Additionally, articulation of the instrument can be controlled using information from the tracking step.

Another aspect of the invention is directed to an apparatus for performing a transluminal procedure. The apparatus comprises: a cutting tool; and a datum and position indicator comprising a luminal wall attachment mechanism and an instrument tracking mechanism adapted to monitor passage of an instrument through a luminal wall opening formed by the cutting tool. In some aspects of the invention, the cutting tool is coupled to the datum and position indicator. Additionally, a guide lumen is provided which enables the instrument tracking mechanism to detect passage of an instrument through the guide lumen and through the luminal wall opening formed by the cutting tool. The guide lumen can comprise a rigidizable guide tube. Additionally, in some embodiments, a luminal wall sterilizing mechanism can be provided. In still other embodiments, an instrument tracking monitor in communication with the tracking mechanism to receive instrument tracking information.

Yet another aspect of the invention is directed to an apparatus for performing a transluminal procedure comprising: a cutting tool; a transluminal instrument; and a datum and position indicator comprising a guide lumen, a luminal wall attachment mechanism and an instrument tracking mechanism adapted to detect passage of the instrument through a luminal wall opening formed by the cutting tool. In some embodiments, the guide lumen comprises a sheath and the transluminal instrument comprises a sheath piercing mechanism adapted to pierce the sheath. In still other embodiments, the guide lumen comprises a rolled sheath adapted to unroll as the instrument advances through the guide lumen. The apparatus can also further comprise an instrument control in communication with the instrument tracking mechanism to control articulation of the instrument.

Still another method of the invention comprises a method for providing a sterile field during a transluminal procedure which includes: securing an elongated body to a wall of a lumen; advancing a sterilization device through the elongated body to a position adjacent the lumen wall; and sterilizing a target portion of the lumen wall with the sterilization device. In some embodiments of the method, the sterilizing step comprises spraying a sterile sealant onto the lumen wall. In other embodiments of the method, the sterilizing step comprises securing a patch against and completely covering the target portion of the lumen wall. In still other embodiments of the method, an opening is created through the lumen wall after the sterilizing step.

Yet another apparatus of the invention provides for performing a transluminal procedure comprising: an elongated body comprising a luminal wall attachment mechanism at a distal portion of the elongated body; and a luminal wall sterilization device extending from a proximal portion of the elongated body to the distal portion of the elongated body. In some embodiments of the claims, the sterilization device comprises a sprayer and a sterile sealant source. In other embodiments, the sterilization device comprises a patch, the patch comprising a luminal wall attachment mechanism. Other embodiments provide for a cutting tool extending from a proximal portion of the elongated body to the distal portion of the elongated body.

Still another aspect of the invention provides an apparatus for use in a transluminal procedure, comprising: a housing having a guide lumen and a seal proximal to a distal end of the housing that extends across and completely seals the guide lumen; a fixation element in the housing and adapted to secure the distal end of the housing to tissue; and a channel extending through the side wall of the housing having an outlet in communication with the lumen distal of the seal. A fixation element can also be provided in some embodiment, that comprises a plurality of tines, a shaft and a plurality of wires extending from the shaft, and/or a fixation element adapted to engage with tissue by rotating less than one half of one revolution. In still other embodiment, at least one cutting blade distal to the seal is provided. Where a cutting blade is provided, in some embodiments, it may be disposed entirely within the sidewall of the housing. Additionally, the housing can be a guide tube. In still other embodiments, the guide tube is a semi-rigidizable guide tube.

Yet another aspect of a method of the invention provides a method for performing a transluminal procedure comprising: securing a distal end of a housing to tissue, the housing comprising a guide lumen and a seal proximal to a distal end of the housing that extends across and completely seals the guide lumen; and sterilizing a region within the guide lumen distal to the seal. In some embodiments of the invention, an opening is formed in tissue distal to the seal after the sterilizing step. In still other embodiments, an instrument is advanced through the seal after the sterilizing step.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a segmented guide tube.

FIG. 1B is a partially segmented, controllable instrument.

FIG. 1C is an exemplary control system to articulated a controllable, segmented instrument.

FIGS. 2A-2F illustrate various alternative configurations for the guide tube of FIG. 1A so that the guide tube may manipulate tissue in support of transluminal procedures.

FIG. 3 is an introducer having a datum and position indicator placed in the mouth.

FIG. 4 illustrates a guide tube entering the esophagus and manipulated into a position inside the stomach.

FIGS. 5A,and 5B illustrate a controllable segmented instrument inside the guide tube and positioned near the stomach wall.

FIGS. 6A and 6B illustrate a needle being used to create a transluminal opening in the stomach wall.

FIGS. 7A and 7B illustrate a balloon placed in the stomach wall to dilate a transluminal opening.

FIGS. 8A and 8B illustrate a controllable instrument passing through a transluminal opening and into the abdominal cavity.

FIG. 9 illustrates an instrument accessing the gallbladder via a transluminal opening.

FIGS. 10-16 illustrate the removal of the gallbladder and closure of the transluminal opening.

FIG. 17 shows a first embodiment of the steerable endoscope of the present invention.

FIG. 18 shows a second embodiment of the steerable endoscope of the present invention.

FIG. 19 shows a wire frame model of a section of the body of the endoscope in a neutral or straight position.

FIG. 20 shows the wire frame model of the endoscope body shown in FIG. 8 passing through a curve in a patient's colon.

FIG. 21 shows a representative portion of an alternative endoscopic body embodiment having multiple segments interconnected by joints.

FIG. 22 shows a partial schematic representation of the embodiment of FIG. 10 showing two segments being pivotable about two independent axes.

FIG.23 shows a preferable endoscope embodiment having motorized segmented joints.

FIGS. 24A-24B show exploded isometric assembly views of two adjacent segments and an individual segment, respectively, from the embodiment shown in FIG. 12.

FIG. 25 shows a variation of the tendon driven endoscope of the present invention.

FIG. 26A shows the range of motion of a controllable segment of the present invention actuated by three tendons.

FIGS. 26B to 26F show the use of three tendons to actuate a controllable segment used in the endoscope of the present invention.

FIGS. 27A and 27B show the use of two tendons to actuate a controllable segment in the endoscope of the present invention.

FIGS. 27C and 27D show the use of four tendons to actuate a controllable segment in the endoscope of the present invention.

FIG. 28 shows a partial schematic representation of a single tendon bending a segment.

FIGS. 29A and 29B show an end view and a side view, respectively, of a vertebra-type control ring which may be used to form the controllable segments of the endoscope of the present invention.

FIG. 29C shows a side view of interconnected vertebra-type control rings used to form the controllable segments of the endoscope of the present invention.

FIGS. 29D and 29E show a side view and a perspective view, respectively, of another embodiment of a vertebra-type control ring.

FIG. 30A shows a perspective view of an endoscope device variation with the outer layers removed to reveal the control rings and backbone.

FIG. 30B shows an end view of a variation of the control ring for an endoscope of the present invention.

FIGS. 31A to 31C illustrate advancing the tendon driven endoscope of the present invention through a tortuous path.

FIG. 32 shows a variation of the tendon driven endoscope of the present invention that has segments of differing diameters.

FIG. 33 shows a variation of the tendon-driven endoscope of the present invention that has segments of different length.

FIGS. 34 (a) to 34 (c) show articulation of a portion of an endoscope using electro-polymeric materials when the material is contracted and/or expanded.

FIGS. 35 (a) and 35 (b) show perspective and end views, respectively, of a segment capable of bending along at least two axes.

FIGS. 35 (c) and 35 (d) show perspective and end views, respectively, of the segment bending in at least two directions.

FIGS. 35 (e) and 35 (f) illustrate an embodiment of an articulating instrument having a pre-set bias.

FIGS. 36 (a) to 36 (c) show end views of various possible configurations for positioning the electro-polymeric materials about a segment.

FIGS. 37 (a) to 37 (c) show articulation of a portion of an endoscope using electro-polymeric materials positioned between two adjacent segments.

FIG. 38 (a) shows a perspective view of segments having electro-polymeric materials formed in a continuous band about the segments.

FIGS. 38 (b) and 38 (c) show end views of different configurations for positioning regions of electro-polymeric material about the segment circumference.

FIGS. 39 (a) and 39 (b) show side and cross-sectional end views, respectively, of a continuous band of electro-polymeric material extending over several segments or joints.

FIGS. 40 (a) to 40 (c) show articulation of a portion of an endoscope using electro-polymeric materials positioned over a length of flexible material.

FIG. 41 (a) shows a perspective view of a flexible material having electro-polymeric materials formed in a continuous band about the material.

FIGS. 41 (b) and 41 (c) show end views of different configurations for positioning regions of electro-polymeric material about the circumference.

FIGS. 42 (a) and 42 (b) show side and cross-sectional end views, respectively, of-a continuous band of electro-polymeric material extending over a length of the endoscope.

FIGS. 43 (a) and 43 (b) show side and end views, respectively, of a plurality of links connected together via hinges, joints, or universal joints.

FIGS. 43 (c) and 43 (d) show electro-polymeric material formed in individual lengths and in a continuous band, respectively, about a portion of the endoscope.

FIG. 43 (e) shows a continuous sleeve of electro-polymeric material placed around the circumference of a number of segments.

FIG. 44 shows a length of electro-polymeric material having electrodes on either side to create a voltage potential through the electro-polymeric material.

FIG. 45 shows patterns for conductive ink that may be placed onto the electro-polymeric material that would allow for large degrees of stretching and contracting.

FIG. 46 shows a schematic illustration of individual conductors for connection to a controller using a separate wire or pair of wires.

FIG. 47 shows a schematic illustration of a network of small controllers that are each capable of switching and controlling a smaller number of electrodes for the electro-polymeric material.

FIGS. 48 A-F illustrate alternative segment embodiments.

FIGS. 49A and 49B illustrate additional embodiments of activated polymer segments.

FIGS. 50 A-C illustrate articulating instrument embodiments actuated or manipulated using embodiments of rolled and compound rolled (nested) polymer actuators.

FIG. 51 shows a schematic view of a system for articulating a controllable article.

FIG. 52 is a perspective view of one embodiment of the connector assembly.

FIG. 53 shows a detailed perspective view of one variation for a carriage assembly.

FIG. 54 shows a variation of a connector portion having a slack area.

FIGS. 55A and 55B illustrate a manner of engagement between the first and second connector portions.

FIG. 56 illustrates carriage assemblies having positioning elements.

FIGS. 57A and 57B illustrate alternative carriage assembly embodiments.

FIGS. 58A-58D illustrate alternative carriage assembly and guideway embodiments.

FIG. 59A shows a variation of a quick-release mechanism for attaching and detaching the tendon driven endoscope from the actuators that relies on pins to actuate the tendons.

FIGS. 59B and 59C shows a second variation of a quick-release mechanism for attaching and detaching the tendon driven endoscope from the actuators that relies on a nail-head configuration to actuate the tendons.

FIG. 60 is a flow chart showing the interaction of several components to provide a method of positioning a steerable endoscope system to facilitate treatment.

FIG. 61 is a cutaway drawing illustrating a steerable colonoscope with a colectomy device mounted thereon being inserted through the lumen of a patient's colon.

FIG. 62 is a cutaway drawing showing the gripping mechanism of the colonoscope-mounted colectomy device expanded within the lumen of the colon.

FIG. 63 illustrates the colon after the diseased portion has been excised and removed with the colonoscope-mounted colectomy device in position to approximate the transected ends of the colon.

FIG. 64 illustrates the colonoscope-mounted colectomy device performing an end-to-end anastomosis to complete the endoscopic colectomy procedure.

FIG. 65 shows a first embodiment of an endoscopic spectroscopy system according to the present invention that combines a fiberoptic spectroscopy device with a steerable colonoscope.

FIG. 66 shows a second embodiment of an endoscopic spectroscopy system with a spectroscopy device integrated directly into a steerable colonoscope.

FIGS. 67-75C illustrate alternative aspects and further details of the rigidizable elements that may be used in conjunction with a working channel.

FIGS. 76-77B illustrate alternative structures to rigidize an external working channel.

FIG. 78 illustrates an alternative nested element embodiment.

FIGS. 79-82 illustrate alternative nested element embodiments.

FIGS. 83A-84 illustrate working channel embodiments that utilize electro-active polymers.

FIGS. 85A and 85B illustrate a working channel having a multiplicity of nestable hourglass embodiments.

FIG. 86 shows a variation of the guide tube assembly in which an endoscope is pushed through and supported by a guide tube.

FIG. 87 shows a cross-sectional view of the guide tube assembly of FIG. 86.

FIG. 88 shows the guide tube variation of FIG. 86 with a portion of the tube partially removed for clarity.

FIG. 89A shows a variation in which the distal end of the endoscope remains unattached to the flexible covering.

FIG. 89B shows another variation in which the distal end of the endoscope is attached to the flexible covering.

FIG. 90 shows the distal end of the endoscope extending past the distal end of the guide tube and the flexible covering extending distally along with the endoscope.

FIG. 91A shows another variation in which the covering is configured as an elastic tubular structure.

FIG. 91B shows another variation in which the covering is configured as an elastic diaphragm structure.

FIG. 92 shows the variations of FIGS. 91A and 91B in which the endoscope is extended distally.

FIG. 93 shows yet another variation in which a plastic covering is used to cover the endoscope and guide tube.

FIGS. 94 and 95 illustrate semi-rigidizable guide tubes.

FIGS. 96A, 96B illustrate the use of primary and secondary rigidizable guide tubes.

FIG. 97 illustrates a controllable instrument having a plurality of controllable segments and a proximal flexible tube.

FIGS. 98A-E a illustrate a variety of curves achieved by using two rigidizable guide tubes.

FIGS. 99-100B illustrate the use of sealing rings with guide tubes.

FIG. 101A shows an example of an endoscope having an electrical circuit throughout the length of the instrument.

FIG. 101B shows an example of the device of FIG. 1A prior to being inserted into a patient.

FIG. 101C shows a device sensing its position as it is advanced through the anus of the patient.

FIG. 101D shows a cross-sectional view of one variation of the endoscope of FIG. 101A.

FIGS. 102A and 102B show an endoscopic device having a series of individual sensors or switches for sensing its insertion depth or position.

FIG. 103A shows another example of an endoscope which may have a number of sensors positioned along the length at discrete locations.

FIG. 103B shows the device of FIG. 103A with individual sensor wires leading to each of the sensors along the length.

FIG. 104 shows another example in which pairs of sensor wires may be placed along the length of the endoscope terminating at discrete locations.

FIGS. 105A to 105D show another example of an endoscope in which the endoscope position may be determined in part by the resistance measured between adjacent sensor rings.

FIG. 106 shows an example of an algorithm which may be utilized for determining and recording insertion depth of an endoscope.

FIGS. 107A and 107B show an example of an endoscope which may utilize an external device for determining endoscope position.

FIG. 107C shows another example of an endoscope having a non-uniform diameter utilizing an external device for determining endoscope position.

FIG. 108 shows another example of an external device which may be used to determine endoscope position.

FIG. 109 shows another example of an external device which may be used to detect sensors positioned on the endoscope.

FIG. 110 shows one example of determining endoscope insertion and/or withdrawal using at least two sensors.

FIGS. 111A and 111B show examples of plots indicating sensor readings from the two sensors of FIG. 110 which may be used to determine whether the endoscope is being advanced or withdrawn.

FIGS. 112A to 112D show at least four situations, respectively, on how the direction of travel for the endoscope may be determined using the two sensors of FIG. 110.

FIG. 113 shows an example of an algorithm which may be utilized for determining the endoscope direction of travel.

FIG. 114 shows a simplified example for determining endoscope position with an external device.

FIG. 115 shows an example illustrating the positioning which may be utilized for an external device with an endoscope.

FIG. 116 shows a schematic variation utilizing a single magnetic device and multiple sensors.

FIGS. 117A and 117B illustrate one example for sensing individual segments of an endoscopic device as it passes the sensor.

FIG. 118 shows another example for sensing individual segments of an endoscopic device having discrete permanent magnets or electromagnets positioned along the endoscope.

FIGS. 119A and 119B illustrate another example for sensing individual segments of an endoscopic device using multiple permanent magnets or electromagnets.

FIG. 120 shows only the vertebrae of an endoscopic device, for clarity, with discrete permanent magnets or electromagnets positioned along the endoscope.

FIGS. 121A and 121B show side and cross-sectional views, respectively, of another example for magnet positioning along the endoscope.

FIGS. 122A and 122B show another example for applying ferrous material, other materials that may alter or affect a magnetic field, permanent magnets, or electromagnets along the endoscope.

FIG. 123 shows another example in which magnets or ferrous material, or other materials that may alter or affect a magnetic field, may be positioned along an elongate support or tool which may then be positioned within the working lumen of a conventional endoscope.

FIGS. 124A to 124C show various examples for attaching ferrous materials or other materials that may alter or affect a magnetic field to individual vertebrae of an endoscope.

FIGS. 125A and 125B show examples of alternative sensing mechanisms using, e.g., force measurement.

FIGS. 126A and 126B show another example of alternative sensing mechanisms using, e.g., a rotatable wheel having discrete permanent magnets or electromagnets integrated within or upon the wheel.

FIGS. 127A and 127 B illustrate a flexible coding strip adapted for used with a datum and position indicator.

FIG. 128 is a diagram of an exemplary surgical instrument navigation system.

FIG. 129 is a diagram of an image capture and registration process.

FIG. 130 illustrates another type of secondary image that may be displayed in conjunction with the primary perspective image.

FIGS. 131 and 132 are method diagrams for displaying images.

FIG. 133 illustrates a registration device in the form of a guide tube.

FIG. 134 illustrates an exemplary registration process.

FIGS. 135A and 135B illustrate block diagrams of point of departure instrument control and tracking system embodiments.

FIGS. 136-139 illustrate various alternative datum and position indicators.

FIGS. 140-141G illustrate alterative fixation devices and techniques.

FIGS. 142 and 142A-C illustrate an embodiment of a rotating engagement member.

FIGS. 143A-143C illustrate the engagement member in FIG. 142 in use.

FIG. 144 is a perspective view of one embodiment of a tissue anchor including a manipulation and locking device for use with a guide tube or datum position indicator.

FIG. 145 is an enlarged view of the region encircled by line 2 of FIG. 144.

FIG. 146 is a perspective view of the embodiment of FIG. 144 configured to articulate.

FIG. 147 represents an enlarged view of the distal end of the shaft of the embodiment of FIG. 146 in a retracted position.

FIG. 148 which represents an enlarged view of the distal end of the shaft of FIG. 146 in an extended position.

FIG. 149 shows one embodiment of a handle of the tissue anchor of FIG. 146.

FIG. 150 illustrates another embodiment of the tissue anchor handle.

FIGS. 151 and 152 show an alternative embodiment of the tissue anchor.

FIGS. 153 and 154 illustrate additional tine engagement member embodiments.

FIGS. 155-159 illustrate alternative rotational engagement rings having pre-designed fracture points.

FIGS. 160-161 illustrate yet another embodiment of rotational engagement rings.

FIGS. 162-166 illustrate still another embodiment of rotational engagement rings.

FIGS. 167 through 168B illustrate another distal end attachment embodiment.

FIGS. 169A-C show an embodiment in which fixation elements are stowed in the guide tube.

FIGS. 170A-B shown an alternative fixation element having a plurality of retractable wires.

FIG. 171 shows yet another guide tube embodiment.

FIGS. 172A-D illustrate alternative tissue fixation devices.

FIGS. 173 and 174 illustrate alternative embodiments of the guide tube applicators.

FIG. 175 illustrates a guide tube engaging a lumen wall.

FIG. 176 illustrates a primary guide tube secured to tissue using a suction ring.

FIG. 177 is a flow chart showing a method for reducing the likelihood of inadvertent organ or tissue damage while piercing a wall in the body.

FIGS. 178-196 illustrate the use of a guide lumen with distal fixation in use to remove a tumor.

FIGS. 197A-214 illustrate the use of a guide lumen with distal fixation and an ultrasound transducer in use to remove a tumor.

FIGS. 215A-D illustrate an procedure intended to manipulate an empty stomach as an alternative to sealing and insufflating the stomach.

FIGS. 217A-217C illustrate an atraumatic element that is an expandable and/or inflatable sleeve.

FIG. 218-218C illustrate an open procedure that employs suture attachments S that are positioned about the projected open target.

FIGS. 219A-C illustrate different views of a cutter assembly.

FIGS. 221A-221E illustrate various views of a stowed and deployed pneumatic muscle 2210 used to create an opening in a lumen wall.

FIGS. 222A-222D illustrate the use of a split screw to create an opening in a lumen wall.

FIGS. 223A and 223B show how a stent may be used to create an opening.

FIGS. 224A-224C illustrate a flex point opener in operation.

FIGS. 225A-226B illustrate two alternative bladder seal configurations.

FIGS. 227A-D illustrate the operation of an integrated fixation and opening guide tube.

FIGS. 228-229 illustrate a guide tube having a sheath stowed in the distal end that is deployed as an instrument is advanced through the guide tube lumen.

FIGS. 230 A-C also illustrate the use of sheaths that are used initially within the guide tube.

FIGS. 231 and 232 illustrate a procedure approaching the thoracic cavity by landing the rigidizable overtube onto the stomach.

FIGS. 233 and 234 illustrate how multiple rigidizable guides may be used for trans-esophageal and trans-diaphragm access to the heart and/or other organs of the thoracic cavity.

FIG. 235 shows instruments of this invention advanced through the colon, attached to the colon wall and thence into the body cavity in a trans-colonic accessway.

FIG. 236 shows how embodiments of the present invention may be used transvaginally, transuterully, transcervixally.

### DETAILED DESCRIPTION OF THE INVENTION

Various procedures and techniques have has been proposed for performing the surgery within the body using a natural body orifice to access the internal portion of the body. Procedures that access through a natural body opening to create an artificial opening are often referred to by the bodily orifice used for access such as peroral for entering through the mouth or trans-vaginal for entering through the vagina. Additionally, procedures may be named for the body part in which the access is created such as transgastric for access through the gastric system such as the stomach, trans-colonic for access through the colon, trans-diaphragm for access created through the diaphragm. These procedures may be called out specifically in this application. The term transluminal refers generally to any procedures performed in the body where an access is created into the body to perform a procedure and includes both natural and artificial access into the body. Other procedures that would benefit from the improvements described herein are described in U.S. Pat. No. 5,458,131, U.S. Pat. No. 5,297,536 and U.S. Pat. No. 3,643,653, U.S. Patent Application Publication 2005/0107664, U.S. Patent Application Publication 2006/0025654, and U.S. Patent Application Publication 2005/0148818 each of which are incorporated herein by reference in their entirety.

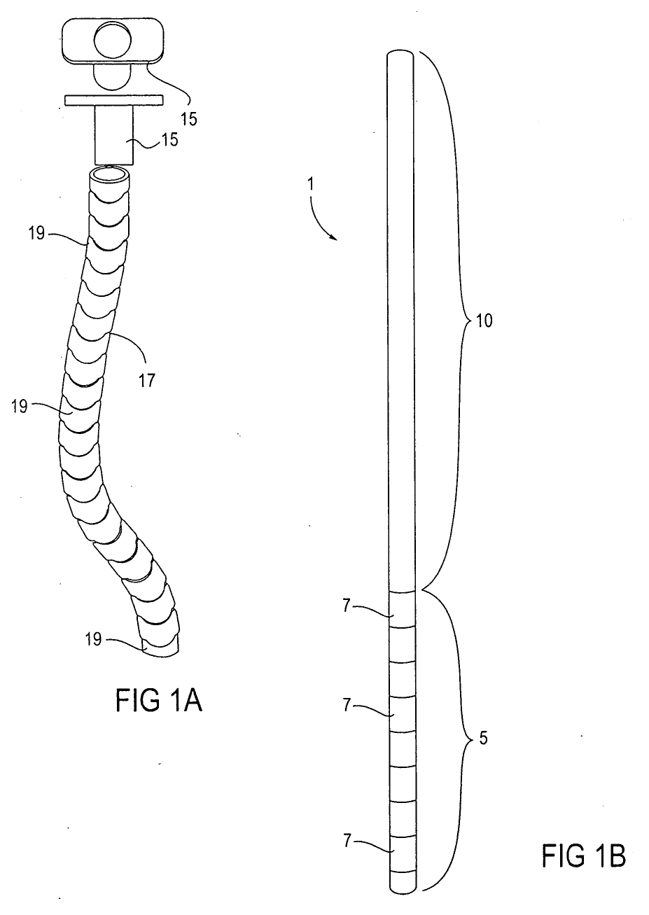

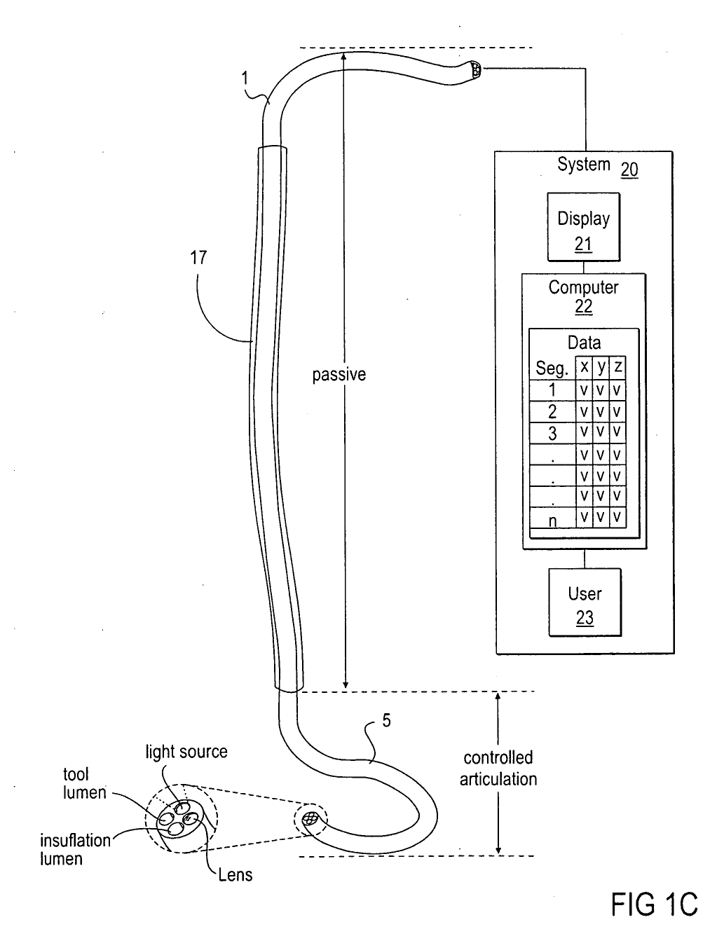

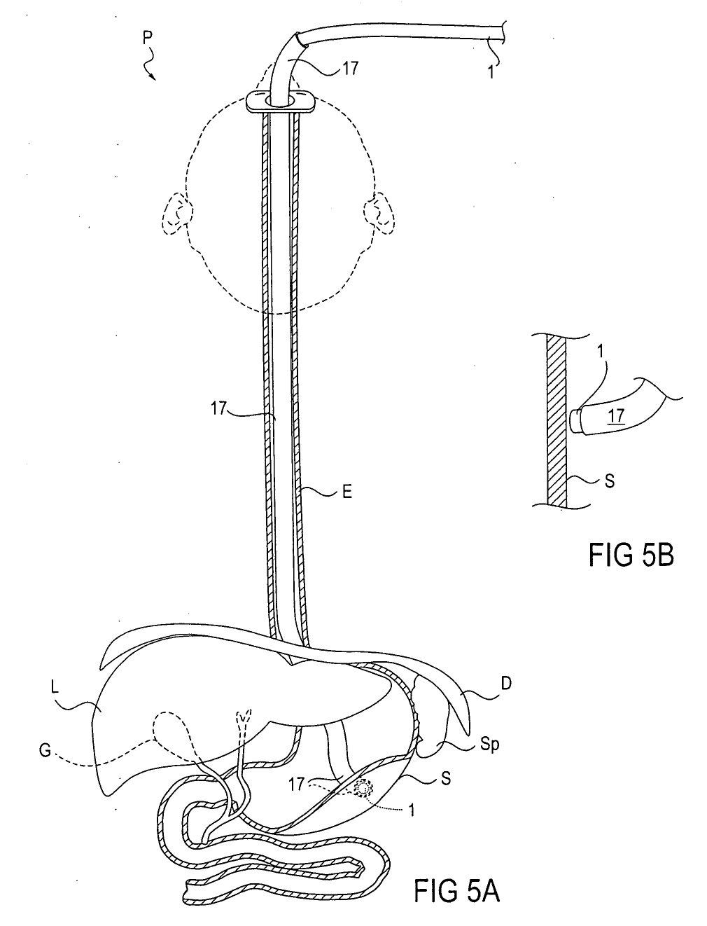

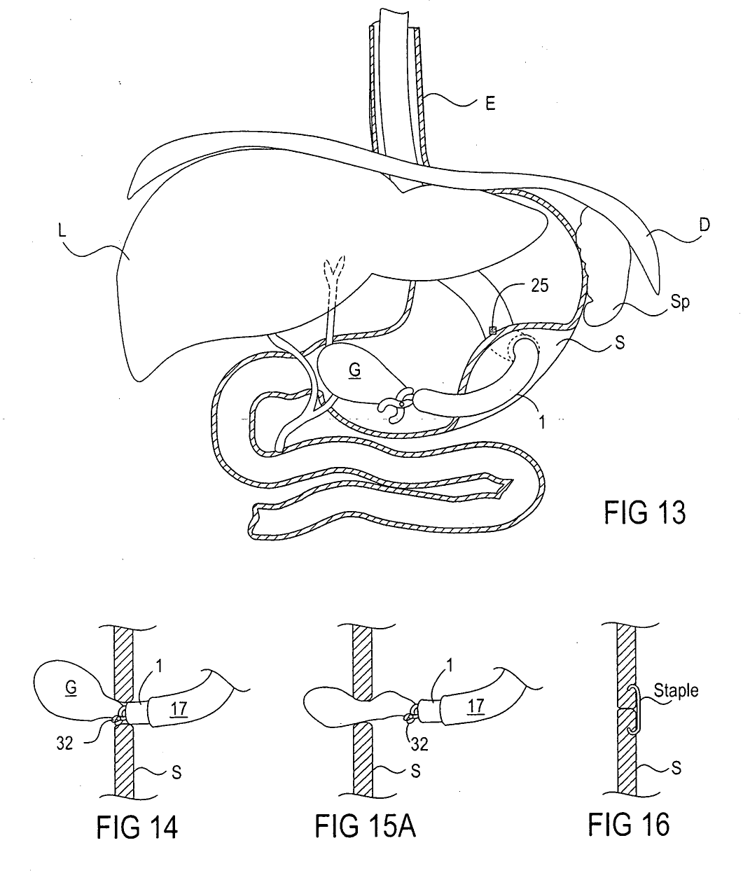

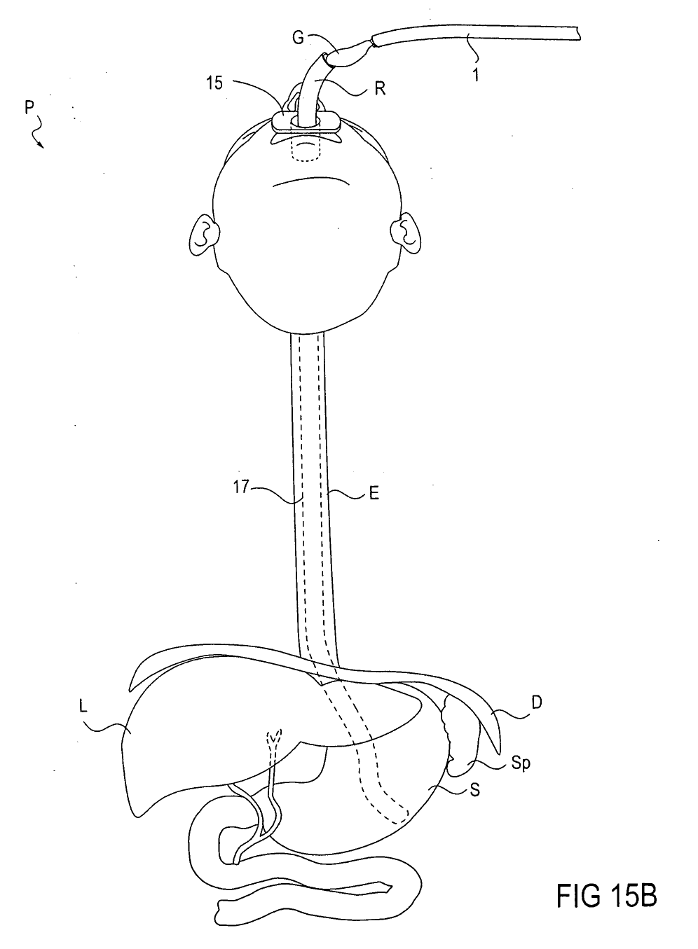

Embodiments of the present invention provide improved point of departure instrument position and control for transluminal access, as well as improved techniques for forming and closing openings made in support of such procedures. FIGS. 1A-16 will be used for general discussion of one method of peroral access to remove the gall bladder according to one embodiment of the present invention. This general discussion will assist in understanding the details of the numerous alternatives that follow.

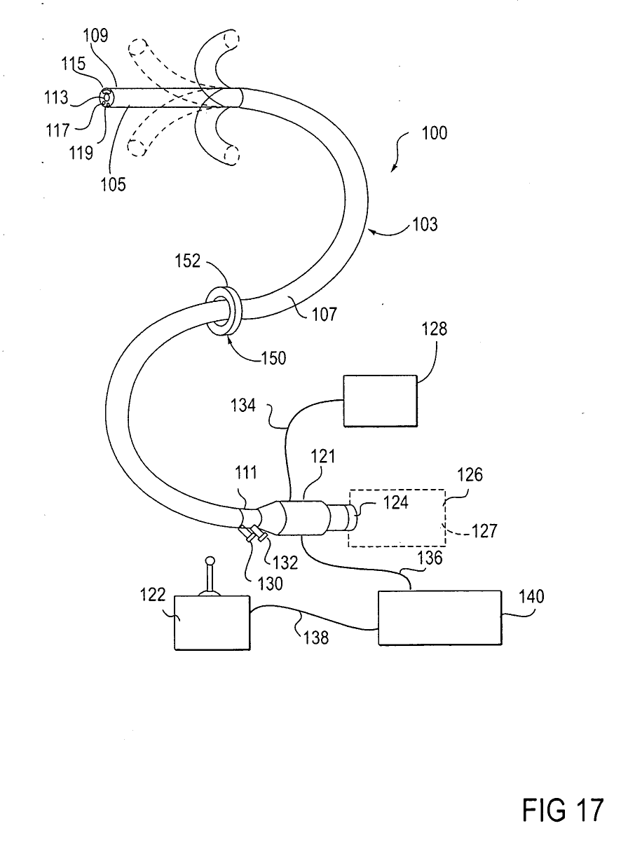

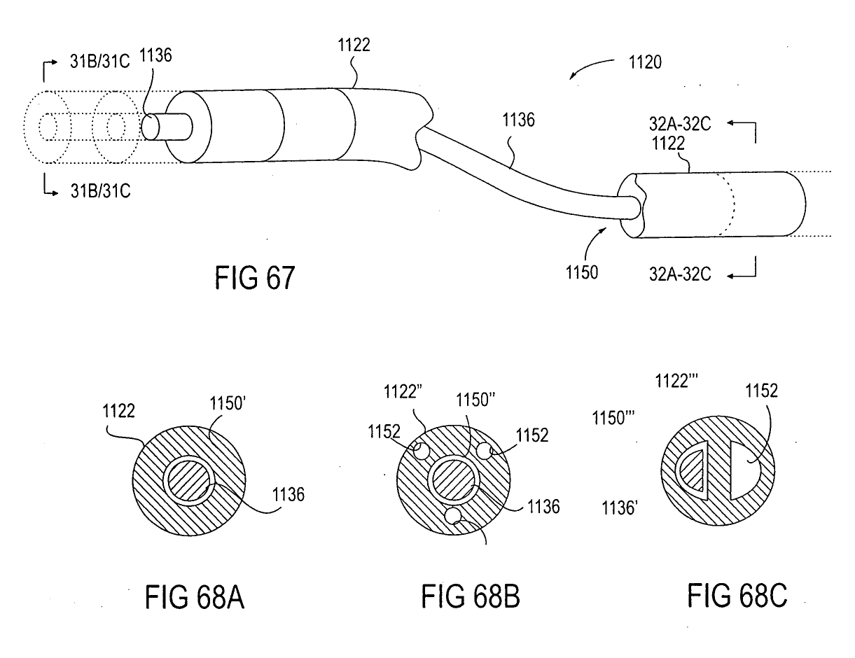

FIG. 1B illustrates an embodiment of a selectively steerable instrument 1 representative of the instruments described below in FIGS. 17 through 50. The selectively steerable instrument 1 has a controllable distal end 5 made up of several segments 7. In the illustrated embodiment, the proximal portion 10 of the selectively steerable instrument 1 is configured as a flexible tube or sheath. It is to be appreciated that the proximal portion 10 could also be segmented and controllable as the distal end 5. FIG. 1A illustrates a segmented guide tube 17 having a variety of articulating and locking segments 19 along its length. Guide tube 17 is representative of the various guide tubes described below with regard to FIGS. 67 through 95. The introducer 15 is provided to help align the guide 17 and the instrument 1 through the patient's mouth and into the alimentary canal to the stomach as will be described below. The introducer 15 may be integrally formed into the guide 17 or provided as a separate component.

FIG. 1C illustrates a basic control system 20 used to communicate control signals from a user 23 to the articulating controllable distal end 5 of the controllable instrument 1. The control system 20 includes a computer 22 and a display 21. Additional details of control systems are provided below with regard to FIGS. 17 through 33, 51 through 60 and 128 through 135. FIG. 1C also illustrates the arrangement of the controllable instrument 1 extending through the guide tube 17.

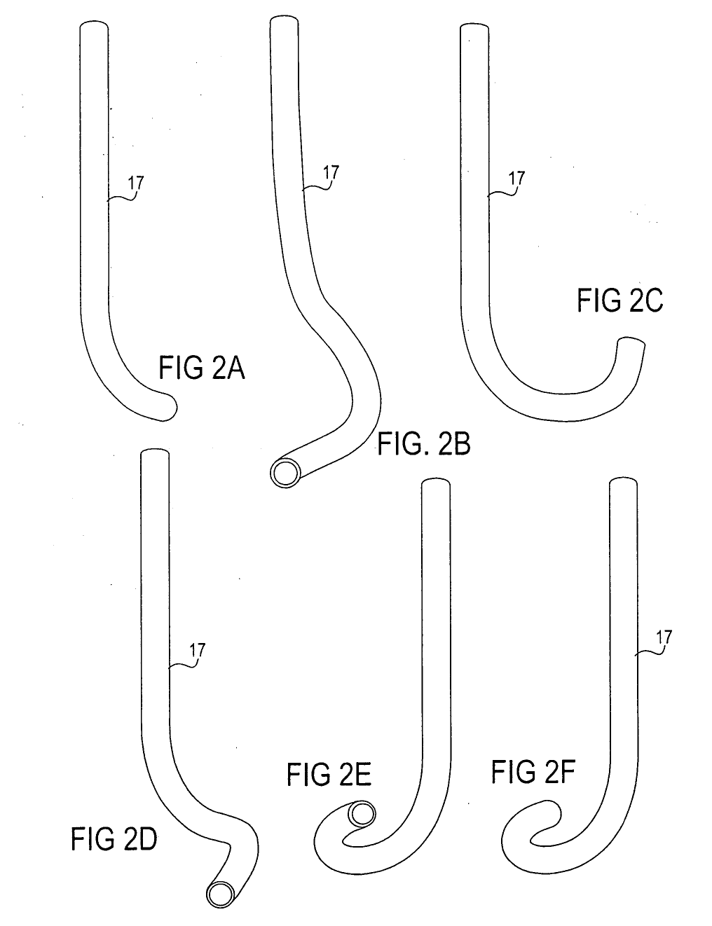

The guide to 17 may be manipulated into a wide variety of different shapes. FIGS. 2A-2F illustrate some possible guide configurations. As will be explained in greater detail below, the guide tube 17 may be locked in to any or different shapes to provide a user with leverage to manipulate the tissue attached to the distal end or along the guide to 17. The ability to manipulate tissue using a guide tube provides additional safety features and increases the atraumatic nature of a procedure as described below.

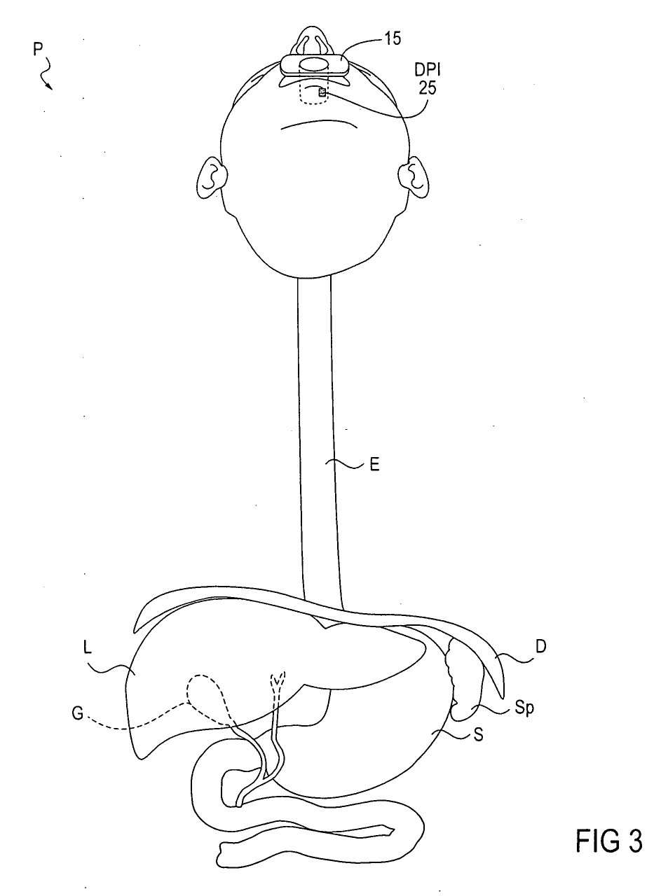

FIG. 3 illustrates a partial view of the anatomy of a patient P. The esophagus (E), the diaphragm (D), the liver (L), the gallbladder (G), the stomach (S) and the spleen (Sp) are shown. If needed, acid secretion into the stomach can be inhibited using any of the conventional techniques such as acid blockage medication, cutting vagus nerve or implanting a structure for blockage of nerve impulses. The introducer 15 is placed in the mouth of the patient.

In the illustrated embodiment, the introducer 15 includes a datum and position indicator 25. A datum and position indicator is any device used to measure, track or otherwise indicate the length of an instrument or the portion of an instrument passing by, in proximity to or detected by the datum and position indicator. A datum and position indicator is a convenient reference point that allows the synchronization of internally generated imaging, externally generated imaging or other forms of data to enable a procedure. One or more datum and position indicator could be used in the procedures described herein. Datum and position indicator is used generally to indicate the position of a transmitter, receiver, sensor detector or other component used to measure, track or otherwise indicate the length of an instrument or the portion of an instrument passing by, in proximity to or detected. Additional details of the datum and position indicator are provided below.

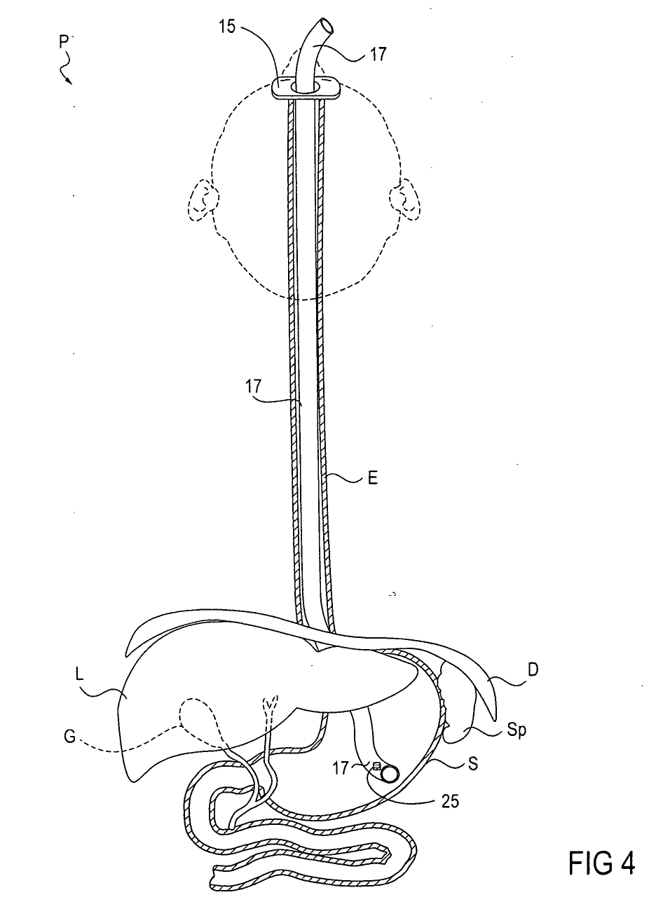

FIG. 4 illustrates the guide tube 17 passing through the introducer 15 to a position near the stomach wall. In the illustrated embodiment, the guide tube 17 is also provided with a position datum indicator 25. Just as the position and datum indicator 25 is used to detect and monitor the passage of instruments by the introducer 15, the position and datum indicator 25 on the distal end of guide tube 17 is used to detect and to monitor the passage of instruments by the distal end of the guide tube 17. Because the distal end of the guide tube 17 is adjacent the opening used in this transluminal procedure, the datum and position indicator on the distal end of the guide tube provides accurate information about the location of instruments as they passed through the transluminal opening into the abdominal cavity.

As illustrated in FIG. 4, the selectively rigidizable guide tube 17 enters the alimentary canal via the mouth, via gastrostomy tube (i.e., introducer 15) or enters the body through a natural or artificial opening. The guide tube (with or without an endoscope or steerable segmented instrument inside of it) is advanced along the esophagus E to the desired guide tube landing site to allow access for a segmented, steerable instrument 1. The guide tube landing site is selected based on a number of factors, such as for example, the procedure to be conducted using the guide tube, the region of the body to be accessed, and the specific physiology of the individual receiving treatment. One exemplary guide tube landing site is the wall of the stomach. Once positioned at the desired landing site, the rigidizable guide is fixed to the landing site, if desired. Numerous fixation alternatives are described below. After securing the distal end of the rigidizable tube to the tissue of interest and forming an opening in that tissue the segmented instrument may be advanced along and through the rigidizable guide and into the body cavity.

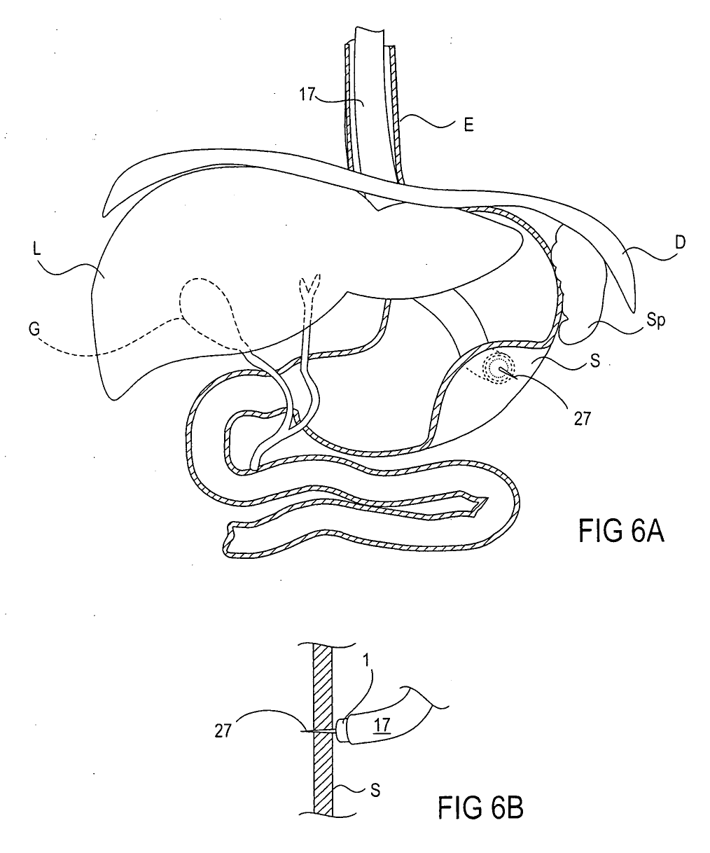

Next, form an opening in the tissue at the landing site. FIGS. 6A and 6B illustrate an alternative procedure where the distal end of the guide tube 17 is not secured to the stomach wall. As is best seen in FIG. 6B, a needle 27 is used to form an opening in the stomach wall near the region of interest. Thereafter the segmented instrument may be used to inspect the anatomy tissue or other structures to determine the proper course of treatment. Thereafter suitable endoscopic tools, instruments or other devices may be provided using one or both of the guide tube or segmented instrument to perform a procedure to treat a condition.

In the illustrative embodiment where the rigidizable guide lands against the stomach wall, an opening needs to be formed in the stomach wall. The opening could be formed using the needle 27, a knife, needle, laser, or any other surgical cutting tool. Additionally, one or more of the opening techniques detailed below could be used.

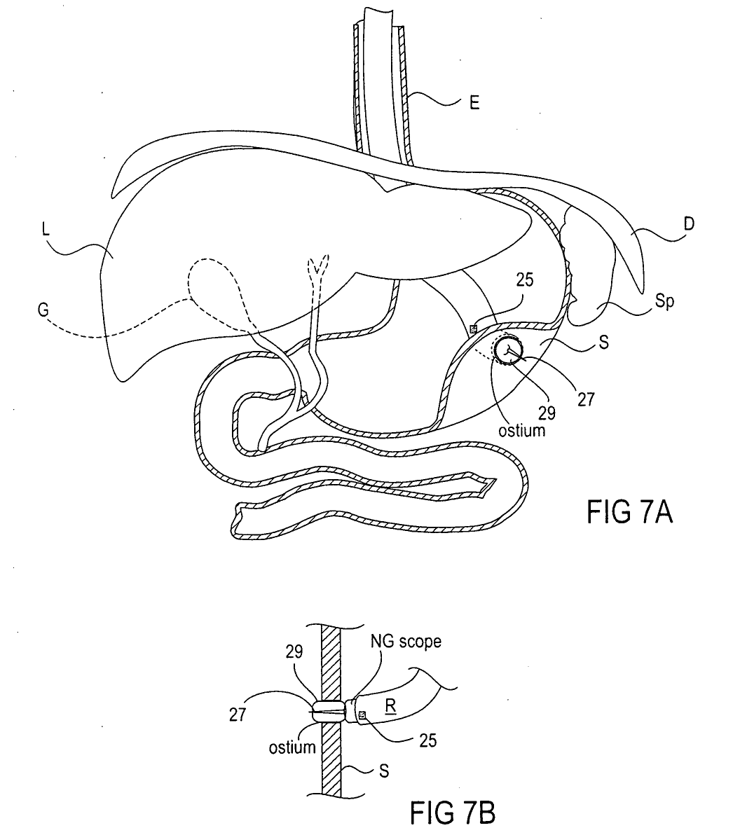

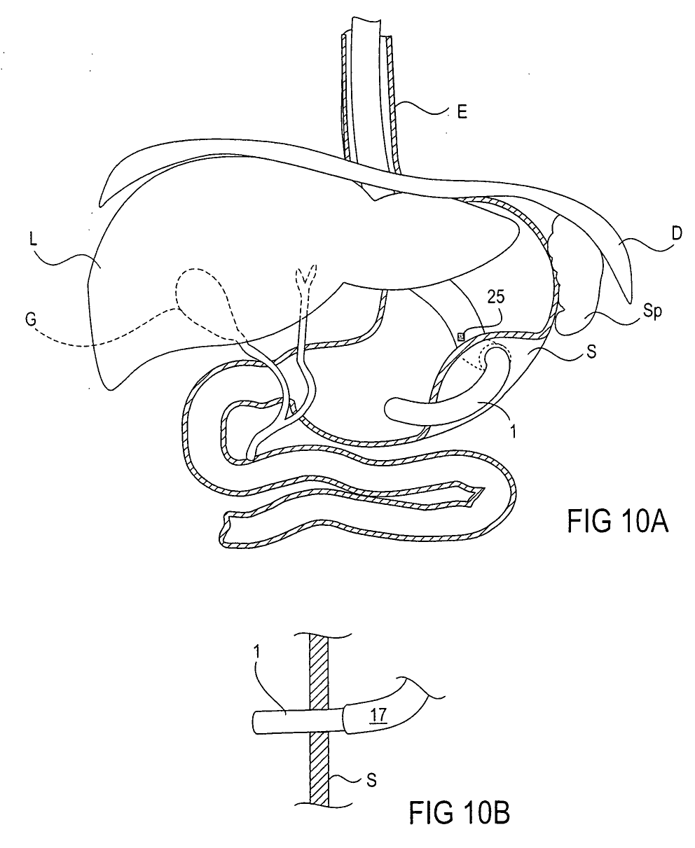

In some cases, the formed opening is large enough to provide access to other instruments needed to conduct a procedure. In some alternative tissue opening techniques, the tissue may be opened and subsequently dilated or by using an inventive opening device form and dilate an open in an integrated procedure. One exemplary embodiment is balloon dilation to open the hole in the side of the stomach. Balloon dilation may be available using some of the techniques described in US Patent publication 2005/0107664, incorporated herein by reference. Use of a balloon for opening the lumen perforation is shown in FIGS. 7A and 7B as balloon 29 is used to enlarge the opening in the stomach wall.

In some cases, it is desirable to provide insufflation as part of the procedure. If so desired, one or more sealing devices or techniques may be used to provide a gas tight seal to the opening to allow the use of positive pressure to the tissue that is the subject of the procedure. Once the hole is appropriately sealed, one can inflate the periodontal cavity or other cavity to be accessed using the techniques described herein. After positioning the guide tube against the stomach lining and seals are in place, insufflation from the working channel of the scope or small needle may be used locally to inject CO₂ or other gases to provide insufflation of the periodontal cavity.

As shown in FIGS. 8A and 8B, the segmented instrument I may be used to inspect the anatomy tissue or other structures to determine the proper course of treatment. Thereafter suitable endoscopic tools, instruments or other devices may be provided using one or both of the guide tube 17 or segmented instrument 1 to perform a procedure to treat a condition.

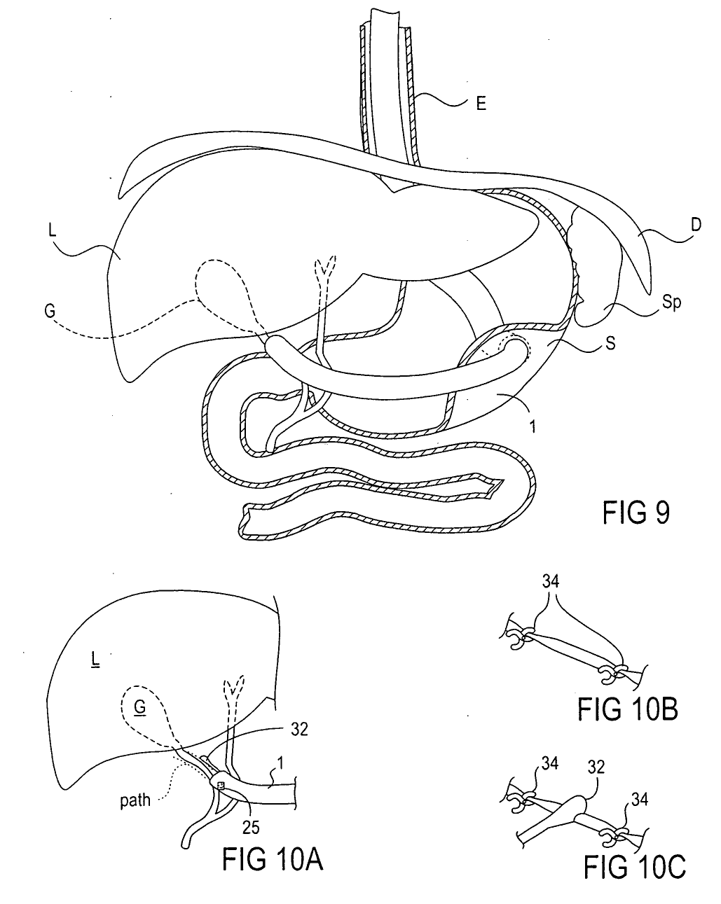

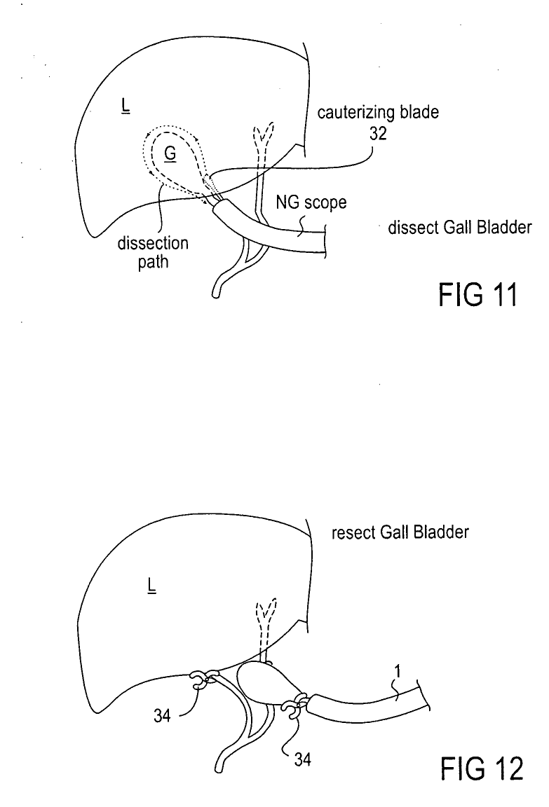

FIGS. 9-16 illustrate the removal of the gall bladder using a transluminal procedure. The rigidizable guide tube 17 stabilizes the steerable instrument I to the opening in the stomach wall. As shown in FIG. 9, this allows the steerable instrument 1 to be used for the removal of or treatment of the gall bladder G. The tip of the steerable instrument is steered by a user and the more proximal segments “follow the leader” of the leading tip as described below. FIG. 10A illustrates a cauterizing blade 32 provided via a working channel in the steerable instrument 1 to dissect the duct. Clips 34 are attached to the duct as shown in FIG. 10B and the duct is cut as shown in FIG. 10C. Next, the gallbladder is dissected and removed as shown in FIGS. 11, 12, 13, 14, 15A and 15B. A staple is then used to close the opening in the stomach wall using a stable or other conventional tissue closing techniques.

In the opened lumen position of FIG. 8A, access is also provided for the steerable instrument to be used for treatment of the liver L, spleen Sp, diaphragm D or small intestine or other areas within the abdominal cavity.

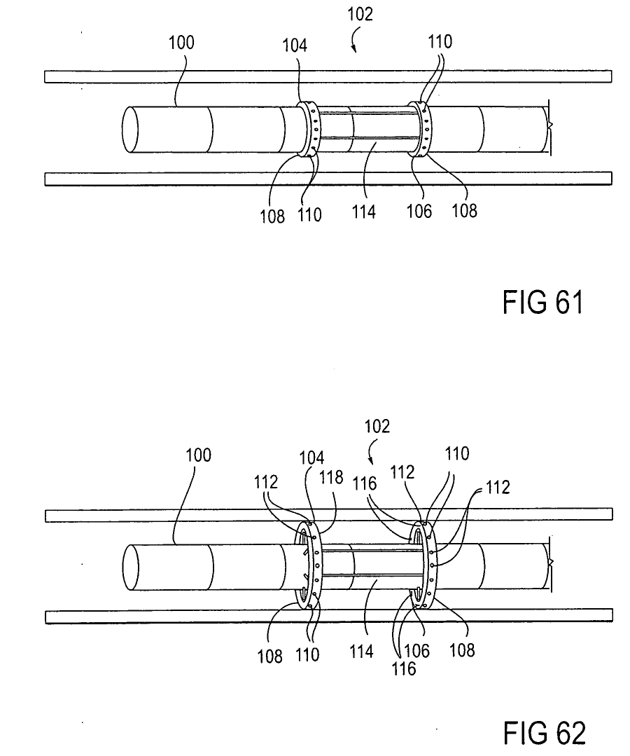

In another illustrative procedures using the instruments described herein, magnets may be appropriately placed within the gut to pull a portion of the small intestine up against the stomach wall as an alternative for gastric by-pass treatments in the small intestine. The rigidized guide may be used to advance the scope into the stomach and place a magnet against the stomach wall. Next, deploy a magnetic element into the small intestine. This may be accomplished using an embodiment of a rigidizable guide described herein equipped with a circumferential tissue grabber as described below in FIGS. 61-64. The tissue grabber may be used to pleat portions of the intestine and assist in advancing the magnet to the desired position. Next, advance the magnetic element to a desired point within the intestine to be adjoined to the stomach. Next, activate the magnets so that the magnetic force is used to draw the magnets-and the tissue joined to them-together, thereby adjoining the small intestine to stomach wall form an anastomosis. Alternatively, from the stomach use a balloon on a magnet and blow the magnet to the desired position within the intestine.

These and other illustrative advantages techniques are described in greater detail below such as, for example, perforation of tissue using a screw, an RF knife, or needle or other surgical implement; valves, seals or other restrictions to support the insufflation pressure; combinations of various overtube configurations with various degrees of controllable scopes; and the use of a hybrid scopes where only a few of the segments are controllable, in particular only those that segments extending beyond the rigidizable guide tube.

Numerous other details and specifics of the steerable instruments, guide tubes, sheaths, datum and position indicator techniques and devices and other details are described in the following patents and applications, commonly owned by the assignee of this application and each of which is incorporated herein by reference in their entirety: U.S. Pat. Nos. 6,468,203; 6,610,007; 6,858,005; 6,837,846; 6,800,056; and U.S. Patent Application Publications: 2003/167007; 2003/171775; 2006/052664; 2005/020901; 2005/165276; 2005/085693; and 2004/176683 (collectively, the “Neoguide applications”).

Steerable Instrument Variations

The steerable segmented controllable instruments described in the Neoguide applications could be used in a wide variety of endoluminal applications. In the first embodiment, the steerable segmented instrument is fully segmented. A fully segmented instrument is articulating and controllable throughout its length or throughout the entirety of the instrument that is implanted into any portions of the body. In a second alternative, the controllable, segmented instrument is only partially segmented and is used in conjunction with a guide tube. In this alternative, the controllable segmented portion of the steerable instrument is only that portion of the instrument that extends beyond the guide tube when the guide tube is fastened or secured within the body to provide a rigidized access port. In yet another alternative, the segmented portion of the controllable instrument has segments whose dimensions and articulation are adapted and depend upon the specifics of the anatomy with which the scope will be utilized. For example, a steerable segmented instrument for use via an esophageal delivery may have more lengthy sections that represent fractional portions of the esophagus. In contrast an instrument adapted for use in the colon may have more segments with smaller dimensions to allow for a greater flexibility given the more tortuous nature of the colon as compared with to the esophagus. It is to be appreciated that the segment and the various configurations may be fully articulating, controllable, passive, under manual control, manipulated by individually applied motors, under the control of a computer, using any of the variety of mechanical actuators, or other combinations of articulation, manipulation and control.

Steerable Instrument

FIG. 17 shows a first embodiment of the steerable endoscope 100 of the present invention. The endoscope 100 has an elongate body 103 with a manually or selectively steerable distal portion 105 and an automatically controlled proximal portion 107. The selectively steerable distal portion 105 can be selectively steered or bent up to a full 180 degree bend in any direction. A fiberoptic imaging bundle 113 and one or more illumination fibers 115 extend through the body 103 from the proximal end 111 to the distal end 109. Alternatively, the endoscope 100 can be configured as a video endoscope with a miniaturized video camera, such as a CCD camera, positioned at the distal end 109 of the endoscope body 103. The images from the video camera can be transmitted to a video monitor by a transmission cable or by wireless transmission. Optionally, the body 103 of the endoscope 100 may include one or two instrument channels 117, 119 that may also be used for insulation or irrigation. The body 103 of the endoscope 100 is highly flexible so that it is able to bend around small diameter curves without buckling or kinking. When configured for use as a colonoscope, the body 103 of the endoscope 100 is typically from 135 to 185 cm in length and approximately 12-13 mm in diameter. The endoscope 100 can be made in a variety of other sizes and configurations for other medical and industrial applications.

A proximal handle 121 is attached to the proximal end 111 of the elongate body 103. The handle 121 includes an ocular 124 connected to the fiberoptic imaging bundle 113 for direct viewing and/or for connection to a video camera 126. The handle 121 is connected to an illumination source 128 by an illumination cable 134 that is connected to or continuous with the illumination fibers 115. A first luer lock fitting 130 and a second luer lock fitting 132 on the handle 121 are connected to the instrument channels 117, 119.

The handle 121 is connected to an electronic motion controller 140 by way of a controller cable 136. A steering control 122 is connected to the electronic motion controller 140 by way of a second cable 138. The steering control 122 allows the user to selectively steer or bend the selectively steerable distal portion 105 of the body 103 in the desired direction. The steering control 122 may be a joystick controller as shown, or other known steering control mechanism. The electronic motion controller 140 controls the motion of the automatically controlled proximal portion 107 of the body 103. The electronic motion controller 140 may be implemented using a motion control program running on a microcomputer or using an application-specific motion controller. Alternatively, the electronic motion controller 140 may be implemented using a neural network controller.

An axial motion transducer 150 is provided to measure the axial motion of the endoscope body 103 as it is advanced and withdrawn. The axial motion transducer 150 can be made in many possible configurations. By way of example, the axial motion transducer 150 in FIG. 17 is configured as a ring 152 that surrounds the body 103 of the endoscope 100. The axial motion transducer 150 is attached to a fixed point of reference, such as the surgical table or the insertion point for the endoscope 100 on the patient's body. As the body 103 of the endoscope 100 slides through the axial motion transducer 150, it produces a signal indicative of the axial position of the endoscope body 103 with respect to the fixed point of reference and sends a signal to the electronic motion controller 140 by telemetry or by a cable (not shown). The axial motion transducer 150 may use optical, electronic or mechanical means to measure the axial position of the endoscope body 103. Other possible configurations for the axial motion transducer 150 are described below.

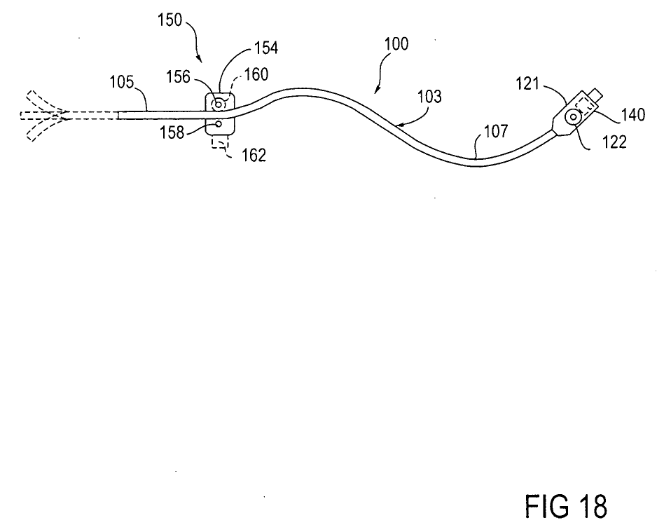

FIG. 18 shows a second embodiment of the endoscope 100 of the present invention. As in the embodiment of FIG. 17, the endoscope 100 has an elongate body 103 with a selectively steerable distal portion 105 and an automatically controlled proximal portion 107. The steering control 122 is integrated into proximal handle 121 in the form of one or two dials for selectively steering the selectively steerable distal portion 105 of the endoscope 100. Optionally, the electronic motion controller 140 may be miniaturized and integrated into proximal handle 121, as well. In this embodiment, the axial motion transducer 150 is configured with a base 154 that is attachable to a fixed point of reference, such as the surgical table. A first roller 156 and a second roller 158 contact the exterior of the endoscope body 103. A multi-turn potentiometer 160 or other motion transducer is connected to the first roller 156 to measure the axial motion of the endoscope body 103 and to produce a signal indicative of the axial position.

The endoscope 100 may be manually advanced or withdrawn by the user by grasping the body 103 distal to the axial motion transducer 150. Alternatively, the first roller 156 and/or second roller 158 may be connected to a motor 162 for automatically advancing and withdrawing the body 103 of the endoscope 100.

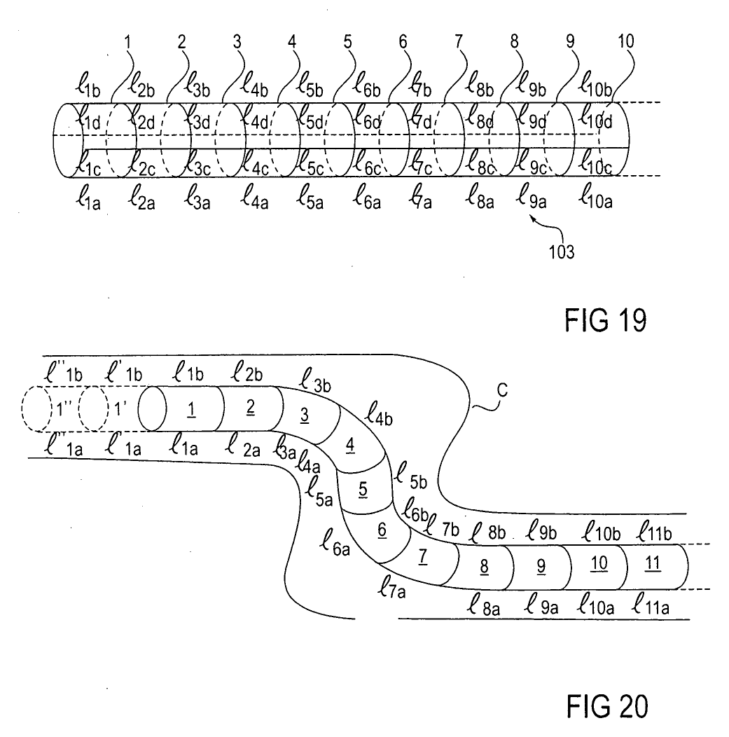

FIG. 19 shows a wire frame model of a section of the body 103 of the endoscope 100 in a neutral or straight position. Most of the internal structure of the endoscope body 103 has been eliminated in this drawing for the sake of clarity. The endoscope body 103 is divided up into sections 1, 2, 3 . . . 10, etc. The geometry of each section is defined by four length measurements along the a, b, c and d axes. For example, the geometry of section 1 is defined by the four length measurements 1.sub.1a, 1.sub.1b, 1.sub.1c, 1.sub.1d, and the geometry of section 2 is defined by the four length measurements 1.sub.2a, 1.sub.2b, 1.sub.2c, 1.sub.2d, etc. Preferably, each of the length measurements is individually controlled by a linear actuator (not shown). The linear actuators may utilize one of several different operating principles. For example, each of the linear actuators may be a self-heating NiTi alloy linear actuator or an electrorheological plastic actuator, or other known mechanical, pneumatic, hydraulic or electromechanical actuator. The geometry of each section may be altered using the linear actuators to change the four length measurements along the a, b, c and d axes. Preferably, the length measurements are changed in complementary pairs to selectively bend the endoscope body 103 in a desired direction. For example, to bend the endoscope body 103 in the direction of the a axis, the measurements 1.sub.1a, 1.sub.2a, 1.sub.3a . . . 1.sub.10a would be shortened and the measurements 1.sub.1b, 1.sub.2b, 1.sub.3b . . . 1.sub.10b would be lengthened an equal amount. The amount by which these measurements are changed determines the radius of the resultant curve.

In the selectively steerable distal portion 105 of the endoscope body 103, the linear actuators that control the a, b, c and d axis measurements of each section are selectively controlled by the user through the steering control 122. Thus, by appropriate control of the a, b, c and d axis measurements, the selectively steerable distal portion 105 of the endoscope body 103 can be selectively steered or bent up to a full 180 degrees in any direction.

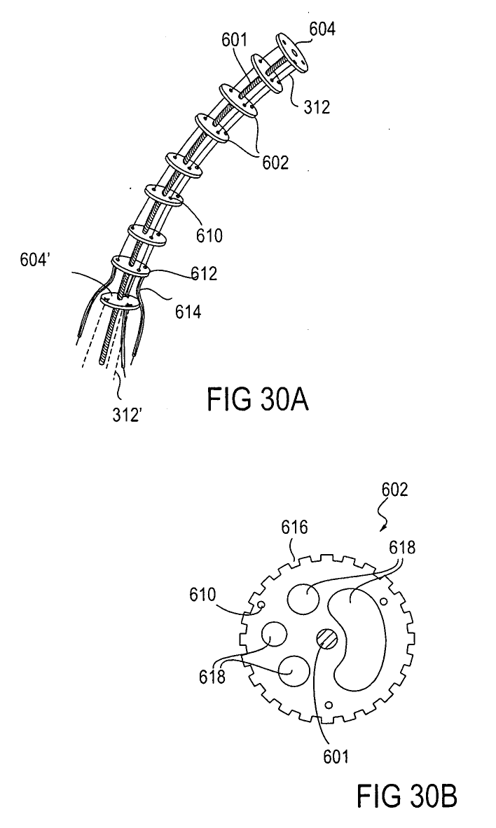

In the automatically controlled proximal portion 107, however, the a, b, c and d axis measurements of each section are automatically controlled by the electronic motion controller 140, which uses a curve propagation method to control the shape of the endoscope body 103. To explain how the curve propagation method operates, FIG. 20 shows the wire frame model of a part of the automatically controlled proximal portion 107 of the endoscope body 103 shown in FIG. 30 passing through a curve in a patient's colon C. For simplicity, an example of a two-dimensional curve is shown and only the a and b axes will be considered. In a three-dimensional curve all four of the a, b, c and d axes would be brought into play.

In FIG. 20, the endoscope body 103 has been maneuvered through the curve in the colon C with the benefit of the selectively steerable distal portion 105 (this part of the procedure is explained in more detail below) and now the automatically controlled proximal portion 107 resides in the curve. Sections 1 and 2 are in a relatively straight part of the colon C, therefore 1.sub.1a=1.sub.1b and 1.sub.2a=1.sub.2b. However, because sections 3-7 are in the S-shaped curved section, 1.sub.3a<1.sub.3b, 1.sub.4a<1.sub.4b and 1.sub.5a<1.sub.5b, but 1.sub.6a>1.sub.6b, 1.sub.7a>1.sub.7b and 1.sub.8a>1.sub.8b. When the endoscope body 103 is advanced distally by one unit, section 1 moves into the position marked 1′, section 2 moves into the position previously occupied by section 1, section 3 moves into the position previously occupied by section 2, etc. The axial motion transducer 150 produces a signal indicative of the axial position of the endoscope body 103 with respect to a fixed point of reference and sends the signal to the electronic motion controller 140. Under control of the electronic motion controller 140, each time the endoscope body 103 advances one unit, each section in the automatically controlled proximal portion 106 is signaled to assume the shape of the section that previously occupied the space that it is now in. Therefore, when the endoscope body 103 is advanced to the position marked 1′, 1.sub.1a=1.sub.1b, 1.sub.2a=1.sub.2b, 1.sub.3a=1.sub.3b, 1.sub.4a<1.sub.4b, 1.sub.5a<1.sub.5b, 1.sub.6a<1.sub.6b, 1.sub.7a>1.sub.7b, l.sub.8a>1.sub.8b, and 1.sub.9a>1.sub.9b, and, when the endoscope body 103 is advanced to the position marked 1″, 1.sub.1a=1.sub.1b, 1.sub.2a=1.sub.2b, 1.sub.3a=1.sub.3b, 1.sub.4a=1.sub.4b, 1.sub.5a<1.sub.5b, 1.sub.6a<1.sub.6b, 1.sub.7a<1.sub.7b, 1.sub.8a>1.sub.8b, 1.sub.9a>1.sub.9b, and 1.sub.10a>1.sub.10b. Thus, the S-shaped curve propagates proximally along the length of the automatically controlled proximal portion 107 of the endoscope body 103. The S-shaped curve appears to be fixed in space, as the endoscope body 103 advances distally.

Similarly, when the endoscope body 103 is withdrawn proximally, each time the endoscope body 103 is moved proximally by one unit, each section in the automatically controlled proximal portion 107 is signaled to assume the shape of the section that previously occupied the space that it is now in. The S-shaped curve propagates distally along the length of the automatically controlled proximal portion 107 of the endoscope body 103, and the S-shaped curve appears to be fixed in space, as the endoscope body 103 withdraws proximally.

Whenever the endoscope body 103 is advanced or withdrawn, the axial motion transducer 150 detects the change in position and the electronic motion controller 140 propagates the selected curves proximally or distally along the automatically controlled proximal portion 107 of the endoscope body 103 to maintain the curves in a spatially fixed position. This allows the endoscope body 103 to move through tortuous curves without putting unnecessary force on the wall of the colon C.

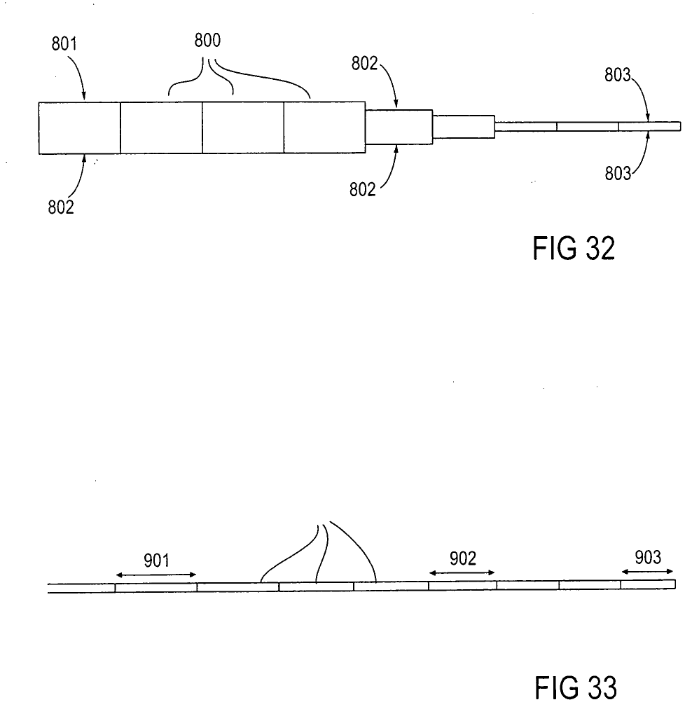

FIG. 32 shows a representative portion of an alternative endoscopic body embodiment 190 which has multiple segments 192 interconnected by joints 194. In this embodiment, adjacent segments 192 can be moved or angled relative to one another by a joint 194 having at least one degree-of-freedom, and preferably having multiple degrees-of-freedom, preferably about two axes as shown here. As seen further in FIG. 33, a partial schematic representation 196 of the embodiment 190 is shown where two segments 192 may be rotated about joint 194 about the two independent axes. The range of motion may be described in relation to spherical axes 198 by angles .alpha. and .beta..

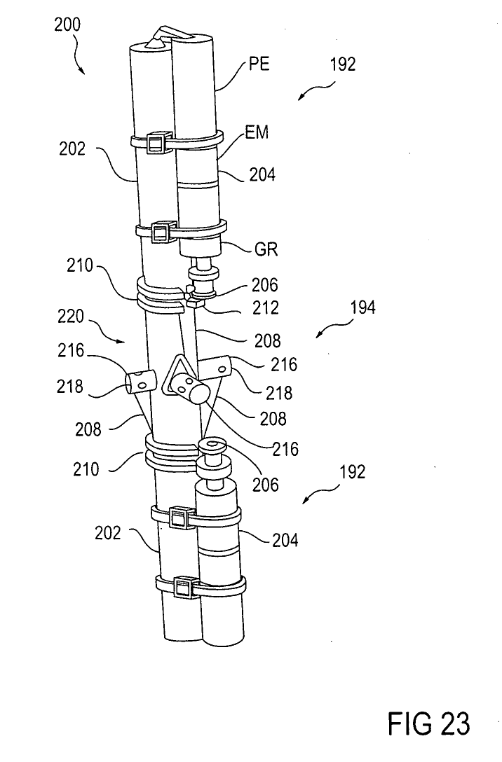

As mentioned above, such a segmented body may be actuated by a variety of methods. A preferable method involves the use of electromechanical motors individually mounted on each individual segment to move the segments relative to one another. FIG. 23 shows a preferable embodiment 200 having motorized segmented joints. Each segment 192 is preferably comprised of a backbone segment 202, which also preferably defines at least one lumen running through it to provide an access channel through which wires, optical fibers, air and/or water channels, various endoscopic tools, or any variety of devices and wires may be routed through. The backbone segment may be made of a variety of materials which are preferably biocompatible and which provide sufficient strength to support the various tools and other components, e.g., stainless steel. Although much of the description is to an individual segment 192, each of the segments 192 are preferably identical, except for the segment (or first few segments) located at the distal tip, and the following description readily applies to at least a majority of the segments 192.

A single motor, or multiple motors depending upon the desired result and application, may be attached to at least a majority of the segments. An embodiment having a single motor on a segment is illustrated in FIG. 23 where an individual motor 204 is preferably attached to backbone 202 and is sufficiently small and compact enough so as to present a relatively small diameter which is comfortable and small enough for insertion into a patient without trauma. Motor 204, which is shown here as being a small brushed DC motor, may be used for actuating adjacent segments 192 and may be controlled independently from other motors. Various motors, aside from small brushed DC motors, may also be used such as AC motors, linear motors, etc. Each motor 204 also preferably contains within the housing not only the electromechanical motor assembly EM itself, but also a gear reduction stage GR, and a position encoder PE. A gear reduction stage GR attached to the motor assembly EM will allow for the use of the motor 204 in its optimal speed and torque range by changing high-speed, low-torque operating conditions into a more useful low-speed, high-torque output. The position encoder PE may be a conventional encoder to allow the controlling computer to read the position of the segment's joint 194 by keeping track of the angular rotational movement of the output shaft of the motor 204.

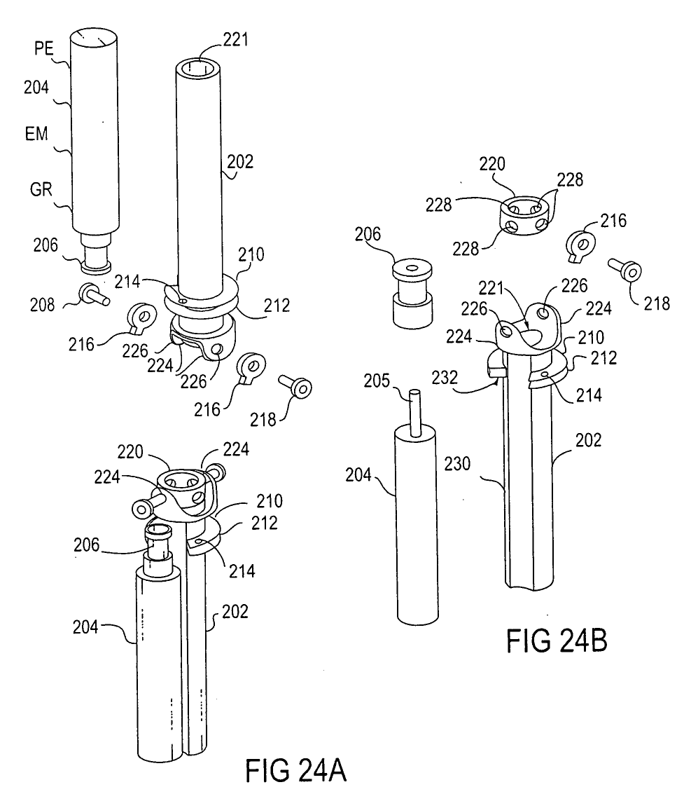

Each motor 204 has a rotatable shaft which extends from an end of the motor 204 to provide for the transmission of power to actuate the segments 192. Upon this shaft, a spool 206 may be rotatingly attached with a first end of the cable 208 further wound about the spool 206. The cable 208 may then be routed from spool 206 through a channel 212 which is defined in the cable guide 210 and out through opening 214 (as seen in greater detail in FIGS. 24A-24B) to cable anchor 216, to which the second end of the cable 208 is preferably attached, e.g., by crimping and/or soldering. The cable guide 210 serves to capture the cable 208 that is wound about the spool 206. The cable anchor 216 is attached across a universal joint pivot 220 to an adjacent segment 192 via a pin 218 and may be shaped like a conventional electronic ring connector having a round section defining a hole therethrough for mounting to the segment 192 and an extension protruding from the anchor 216 for attaching the second end of the cable 208. Cable 208 may comprise a wide variety of filaments, strands, wires, chains, braids, etc. any of which may be made of a wide variety of biocompatible materials, e.g., metals such as stainless steel, polymers such as plastics and Nylon, etc.

In operation, when the motor 204 is operated to spin the shaft in a first direction, e.g., clockwise, the spool 206 rotates accordingly and the cable 208 pulls in a corresponding direction on the adjacent segment 192 and transmits the torque to subsequently actuate it along a first axis. When the motor 204 is operated to spin the shaft in a second direction opposite to the first, e.g., counter-clockwise, the spool 206 again rotates accordingly and the cable 208 would then pull in the corresponding opposing direction on the adjacent segment 192 to subsequently transmit the torque and actuate it in the opposite direction.

FIGS. 24A and 24B show exploded isometric assembly views of two adjacent segments and an individual segment, respectively, from the embodiment shown in FIG. 23. As seen in FIG. 24A, backbone 202 is seen with the lumen 221, which may be used to provide a working channel, as described above. Also seen are channel 212 defined in cable guide 210 as well as opening 214 for the cable 208 to run through. In interconnecting adjacent segments and to provide the requisite degree-of-freedom between segments, a preferable method of joining involves using the universal joint pivot 220. However, other embodiments, rather than using a universal joint pivot 220, may use a variety of joining methods, e.g., a flexible tube used to join two segments at their respective centers, a series of single degree-of-freedom joints that may be closely spaced, etc. This particular embodiment describes the use of the universal joint pivot 220. At the ends of backbone 202 adjacent to other segments, a pair of universal yoke members 224 may be formed with a pair of corresponding pin openings 226. As the universal joint pivot 220 is connected to a first pair of yoke members 224 on one segment, a corresponding pair of yoke members 224 from the adjacent segment may also be attached to the joint pivot 220.

As seen further in FIG. 24B, the universal joint pivot 220 is shown in this embodiment as a cylindrical ring having two sets of opposing receiving holes 228 for pivotally receiving corresponding yoke members 224. The receiving holes 228 are shown as being spaced apart at 90 degree intervals, however, in other variations, receiving holes may be spaced apart at other angles depending upon the desired degree-of-freedom and application. Also seen is an exploded assembly of spool 206 removed from motor 204 exposing drive shaft 205. With motor 204 displaced from backbone 202, the groove 230 is revealed as formed in the backbone 202. This groove 230 may be depressed in backbone 202 to preferably match the radius of the motor 204 housing not only to help locate the motor 204 adjacent to backbone 202, but also to help in reducing the overall diameter of the assembled segment. The motor 204 may be attached to the backbone 202 by various methods, e.g., adhesives, clamps, bands, mechanical fasteners, etc. A notched portion 232 may also be formed in the cable guide 210 as shown to help in further reducing segment diameter.

Prior to insertion into a patient, the endoscope 200 may optionally be configured to have a diagnostic check performed automatically. When the endoscope 200 is wound onto a drum, adjacent segments 192 will have a predetermined angle relative to one another, as determined initially by the diameter of the drum and the initial configuration of the storage unit in which the endoscope 200 may be positioned. During a diagnostic check before insertion, a computer may be configured to automatically sense or measure the angles between each adjacent segments 192. If any of the adjacent segments 192 indicate a relative measured angle out of a predetermined acceptable range of angles, this may indicate a segment 192 being out of position and may indicate a potential point of problems during endoscope 200 use. Accordingly, the computer may subsequently sound an audible or visual alarm and may also place each of the segments 192 into a neutral position to automatically prevent further use or to prevent any trauma to the patient.

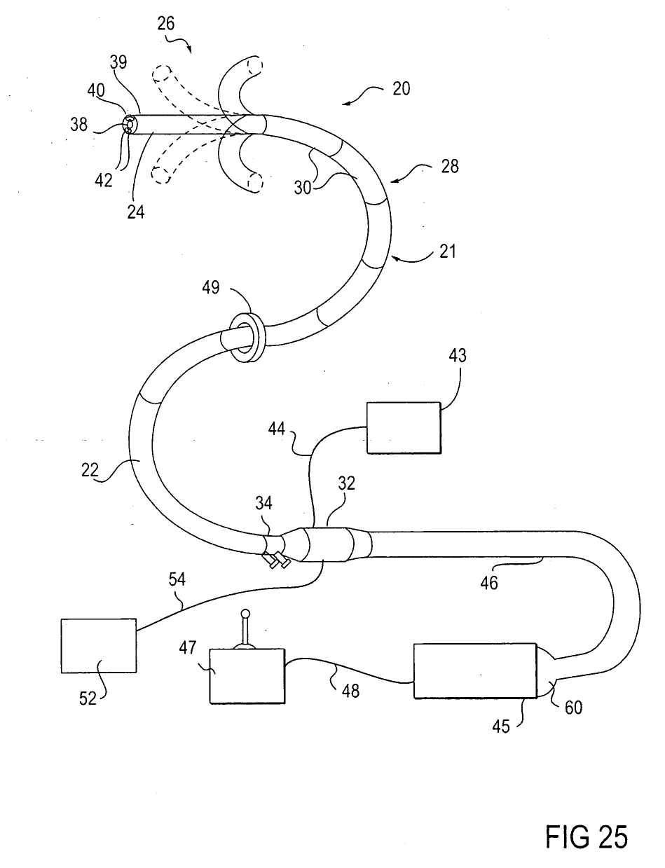

FIG. 25 shows a variation of the tendon driven endoscope 20 of the present invention. The endoscope 20 has an elongate body 21 with a manually or selectively steerable distal portion 24, an automatically controlled portion 28, and a flexible and passively manipulated proximal portion 22, which may be optionally omitted from the device. The steerable distal portion 24 can be articulated by hand or with mechanical assistance from actuators. The automatically controlled portion 28 is segmented, and each segment is capable of bending through a full range of steerable motion. The distal portion 24 is also a controllable segment.

The selectively steerable distal portion 24 can be selectively steered or bent up to, e.g., a full 180 degree bend in any direction 26, as shown. A fiberoptic imaging bundle 40 and one or more illumination fibers 42 may extend through the body 21 from the proximal portion 22 to the distal portion 24. Alternatively, the endoscope 20 may be configured as a video endoscope with a miniaturized video camera, such as a CCD or CMOS camera, positioned at the distal portion 24 of the endoscope body 21. The images from the video camera can be transmitted to a video monitor by a transmission cable or by wireless transmission where images may be viewed in real-time and/or recorded by a recording device onto analog recording medium, e.g., magnetic tape, or digital recording medium, e.g., compact disc, digital tape, etc. LEDs or other light sources could also be used for illumination at the distal tip of the end6scope.

The body 21 of the endoscope 20 may also include one or more access lumens 38 that may optionally be used for illumination fibers for providing a light source, insufflation or irrigation, air and water channels, and vacuum channels. Generally, the body 21 of the endoscope 20 is highly flexible so that it is able to bend around small diameter curves without buckling or kinking while maintaining the various channels intact. When configured for use as a colonoscope, the body 21 of the endoscope 20 may range typically from 135 to 185 cm in length and about 13-19 mm in diameter. The endoscope 20 can be made in a variety of other sizes and configurations for other medical and industrial applications.

The controllable portion 28 is composed of at least one segment 30, and preferably several segments 30, which are controllable via a computer and/or electronic controller (controller) 45 located at a distance from the endoscope 20. Each of the segments 30 has tendons mechanically connected to actuators to allow for the controlled motion of the segments 30 in space. The actuators driving the tendons may include a variety of different types of mechanisms capable of applying a force to a tendon, e.g., electromechanical motors, pneumatic and hydraulic cylinders, pneumatic and hydraulic motors, solenoids, shape memory alloy wires, electronic rotary actuators or other devices or methods as known in the art. If shape memory alloy wires are used, they are preferably configured into several wire bundles attached at a proximal end of each of the tendons within the controller. Segment articulation may be accomplished by applying energy, e.g., electrical current, heat, etc., to each of the bundles to actuate a linear motion in the wire bundles which in turn actuate the tendon movement. The linear translation of the actuators within the controller may be configured to move over a relatively short distance, e.g., within a few inches or less such as +/−0.1 inch, to accomplish effective articulation depending upon the desired degree of segment movement and articulation.

It is preferable that the length of the insertable portion of the endoscope comprises controllable segments 30, although a passive proximal portion 22 can also be used. This proximal portion 22 is preferably a flexible tubing member that may conform to an infinite variety of shapes, and may be made from a variety of materials such as thermoset and thermoplastic polymers which are used for fabricating the tubing of conventional endoscopes.

Each segment 30 preferably defines at least one lumen running throughout to provide an access channel through which wires, optical fibers, air and/or water channels, various endoscopic tools, or any variety of devices and wires may be routed. A polymeric covering, or sheath, 39 may also extend over the body of the endoscope 21 including the controllable portion 28 and steerable distal portion 24. This sheath 39 can preferably provide a smooth transition between the controllable segments 30, the steerable distal portion 24, and the flexible tubing of proximal portion 22.

A handle 32 may be attached to the proximal end of the endoscope. The handle 32 may include an ocular connected to the fiberoptic imaging bundle 42 for direct viewing. The handle 32 may otherwise have a connector 54 for connection to a video monitor, camera, e.g., a CCD or CMOS camera, or a recording device 52. The handle 32 may be connected to an illumination source 43 by an illumination cable 44 that is connected to or continuous with the illumination fibers 42. Alternatively, some or all of these connections could be made at the controller 45. Luer lock fittings 34 may be located on the handle 32 and connected to the various instrument channels.

The handle 32 may be connected to a motion controller 45 by way of a controller cable 46. A steering controller 47 may be connected to the motion controller 45 by way of a second cable 48 or it may optionally be connected directly to the handle 32. Alternatively, the handle may have the steering control mechanism integrated directly into the handle, e.g., in the form of a joystick, conventional disk controllers such as dials, pulleys or wheels, etc. The steering controller 47 allows the user to selectively steer or bend the selectively steerable distal portion 24 of the body 21 in the desired direction 26. The steering controller 47 may be a joystick controller as shown, or other steering control mechanism, e.g., dual dials or rotary knobs as in conventional endoscopes, track balls, touch pads, mouse, or sensory gloves. The motion controller 45 controls the movement of the segmented automatically controlled proximal portion 28 of the body 21. This controller 45 may be implemented using a motion control program running on a microcomputer or using an application-specific motion controller. Alternatively, the controller 45 may be implemented using, e.g., a neural network controller.

The actuators applying force to the tendons may be included in the motion controller unit 45, as shown, or may be located separately and connected by a control cable. The tendons controlling the steerable distal portion 24 and the controllable segments 30 extend down the length of the endoscope body 21 and connect to the actuators. FIG. 25 shows a variation in which the tendons pass through the handle 32 and connect directly to the motion controller 45 via a quick-release connector 60. In this variation, the tendons are part of the control cable 46, although they could independently connect to the actuators, so long as the actuators are in communication with the controller 45.

An axial motion transducer (also called a depth referencing device or datum) 49 may be provided for measuring the axial motion, i.e., the depth change, of the endoscope body 21 as it is advanced and withdrawn. The depth referencing device 49 can be made in many possible configurations. For example, the axial motion transducer 49 in FIG. 25 is configured as a ring 49 that may surround the body 21 of the endoscope 20. The axial motion transducer 49 is preferably attached to a fixed point of reference, such as the surgical table or the insertion point for the endoscope 20 on the patient's body. As the body 21 of the endoscope 20 slides through the axial motion transducer 49, it indicates the axial position of the endoscope body 21 with respect to the fixed point of reference and sends a signal to the electronic controller 45 by telemetry or by a cable. The axial motion transducer 49 may use optical, electronic, magnetic, radio frequency or mechanical methods to measure the axial position of the endoscope body 21.

When the endoscope body 21 is advanced or withdrawn, the axial motion transducer 49 detects the change in position and signals the motion controller 45. The controller can use this information to propagate the selected curves proximally or distally along the controllable portion 28 of the endoscope body 21 to keep the endoscope actively following the pathway selected by the user steering the distal portion 24. The axial motion transducer 49 also allows for the incrementing of a current depth within the colon C by the measured change in depth. This allows the endoscope body 21 to be guided through tortuous curves without putting unnecessary force on the wall of the colon C.

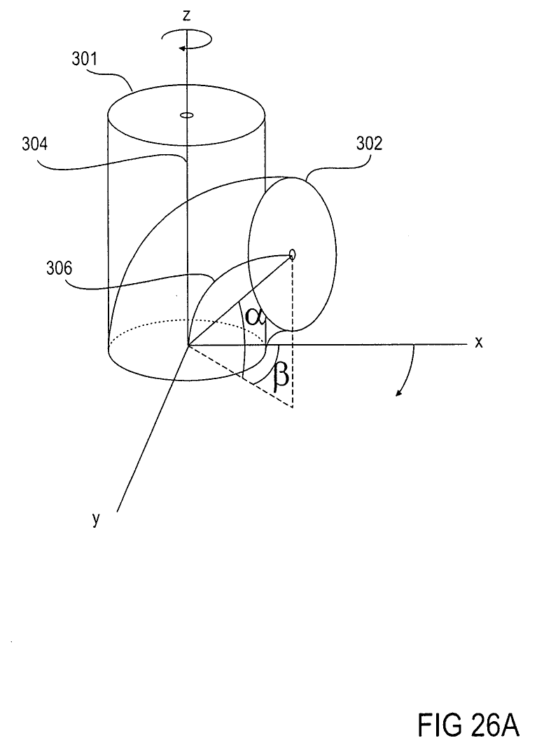

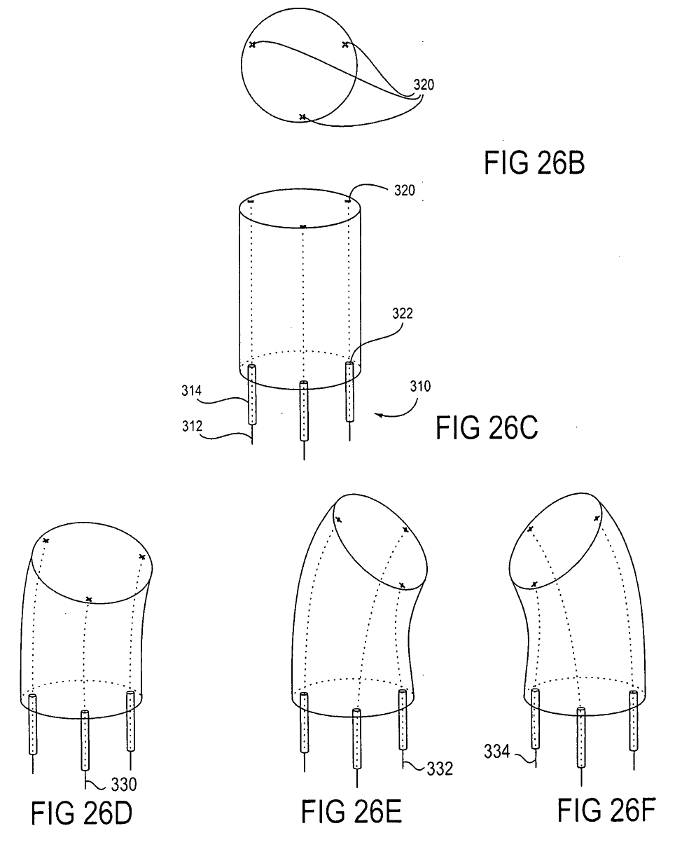

FIG. 26A shows an example of the resulting segment articulation which may be possible through the use of two or three tendons to articulate the controllable segments, including the steerable distal section. FIG. 26A shows one example of a possible range of motion of a controllable segment of the present invention actuated, in this example, by three tendons. A segment in the relaxed, upright position 301 can be bent in virtually any direction relative to the x-y plane. The figure, as an illustrative example, shows a segment 302 that has been bent down and at an angle relative to its original position 301. The angles alpha. and beta. describe the bend assumed by the segment. Angle .beta. gives the angle in the x-y plane, while a is the angle describing the motion in the x-z plane. In one variation, the controllable segments of the endoscope can bend through all 360 degrees in the .beta. angle and up to 90 degrees in the .alpha. angle. An angle a greater than 90 degrees would result in looping of the endoscope. In FIG. 26A, the segment is shown bent approximately 45 degrees along angle .alpha. The freedom of movement of a segment is, in part, determined by the articulation method, the size of the segment, the materials from which it is constructed, and the manner in which it is constructed, among others. Some of these factors are discussed herein.

The steerable distal portion, as well as the endoscope and the controllable segments are bendable but preferably not compressible or expansible. Thus, in FIG. 26A, the centerline 304 of the relaxed segment 301 is approximately the same length as the centerline 306 of the segment after bending 302.

FIGS. 26B to 26F show the use of three tendons to actuate a controllable segment used in an endoscope of the present invention. The tendons shown in this example are all Bowden type cables 310 that have an internal cable 312 coaxially surrounded by a housing or sleeve 314 in which the cable is free to move. Bowden cables can be used to apply either tensile or compressive forces, i.e., they may be pushed or pulled, to articulate the endoscope and can be actuated remotely to deliver forces as desired at locations along the endoscope. Force from a tendon is exerted across or through the segment by attaching the tendon cable at the distal end of the segment 320 and the tendon housing 314 at the proximal end of the segment 322. FIG. 26B shows a view of the top of the segment with three attachment sites for the tendon cables indicated 320.

In one variation, three tendons are used to actuate each segment, including the steerable distal portion, although four or more tendons could be used. Three tendons can reliably articulate a segment in any direction without having to rotate the segment or endoscope about its longitudinal axis. The three cable tendons 312 are preferably attached at the distal end of the segment 320 close to the segment's edge, spaced equally apart. In FIG. 26B, tendons are attached at the two o'clock, six o'clock and 10 o'clock positions. It is desirable to use fewer tendons, because of space concerns, since the tendons controlling each segment project proximally to the actuators. Thus, two tendons could be used to control a segment. It may also be desirable to include one or more biasing element, e.g., a spring, to assist in articulating a segment in three dimensions. In another variation, two tendons may be used to articulate a segment in three dimensional space by controlling motion in two directions while rotating the segment about its longitudinal axis.

FIG. 26C shows a relaxed segment with three tendons attached. The tendon sleeves 314 are shown attached to the proximal end of the segment 322 directly below the corresponding cable attachment sites. FIGS. 26D to 26F show this segment bent by each of the controlling tendons 310 separately.

As shown in FIG. 26D, applying tension by pulling on the first tendon 330 results in a bending in the direction of the first tendon 330. That is, looking down on the top of the unbent segment (as in FIG. 26B), if the first tendon is attached at the six o'clock position, then pulling on just this tendon results in bending the segment towards the six o'clock position. Likewise, in FIG. 26E, putting tension only on a second tendon 332 attached at the two o'clock position results in bending the segment towards the two o'clock direction. Finally, pulling on the tendon in the ten o'clock position 334 bends the segment towards the ten o'clock direction. In all cases, the bending is continuous; the greater the tension applied, the further the bending (the alpha. angle, in the x-z plane of FIG. 26A). A segment can be bent in any direction by pulling on individual tendons or a combination of two tendons. Thus, to bend the segment in the twelve o'clock direction, both the second 332 and the third 334 tendon could be pulled with equal force. Alternatively, first tendon 330 in the six o'clock position may be pushed either alone or in combination with second 332 and third tendons 334 being pulled to result in the same configuration.

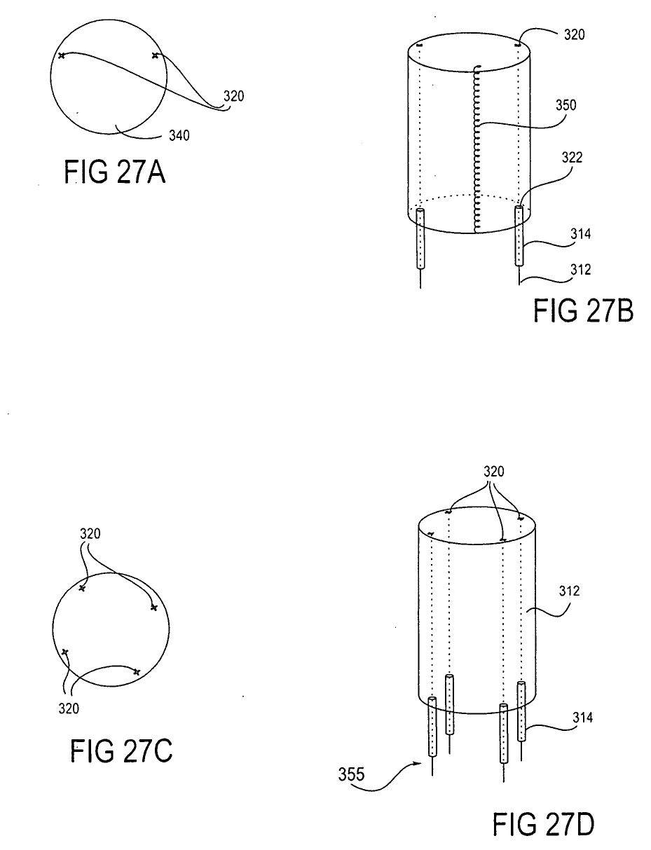

FIGS. 27A and 27B show a variation in which a segment is articulated by two tendons and one biasing element. FIG. 27A shows a planar top view of the segment. The attachment sites for the biasing element 340 and the two tendons 320 are spaced around the perimeter of the distal end of the segment as shown. The tendons 320 may be attached at the two o'clock and ten o'clock positions, looking down on the top of the section, and the biasing element 340 is attached at the six o'clock position. FIG. 27B shows a perspective view of the segment in the unbent configuration. In this variation, the biasing element is configured to apply tension to the side of the segment such that it will bend towards the six o'clock position. The biasing element can be any element that can apply compressive or tensile forces across the segment, e.g. a spring, elastic element, a piston, etc. The segment is held in the neutral or unbent position shown in FIG. 27B by applying tension from both tendons 312. Controlling the amount of tension applied by the tendons results in bending of the segment in three dimensional space. More than one biasing element could also be used with two or more tendons. For example, a biasing element could be located opposite each tendon.

Alternatively, if the tendon is a push-pull cable, and each tendon can apply compression as well as tension, then two tendons can control the motion of segment without any biasing element at all.

More than three tendons can also be used to control the bending of a segment. FIG. 27C shows a top planar view of a segment that is controlled by four tendons attached in the eleven o'clock, two o'clock, five o'clock and eight o'clock positions. As with the three-tendon embodiment, tension applied on one or a combination of the tendons results in shortening the side of the segment. Thus, if tension is applied only on the tendon attached distally at the eleven o'clock position 355, the corresponding side of the tendon will shorten, and the segment will bend in the eleven o'clock direction.

In all these variations, the circumferential locations of the tendons and/or biasing elements are illustrative and are not intended to be limited to the examples described herein. Rather, they may be varied according to the desired effects as understood by one of skill in the art.

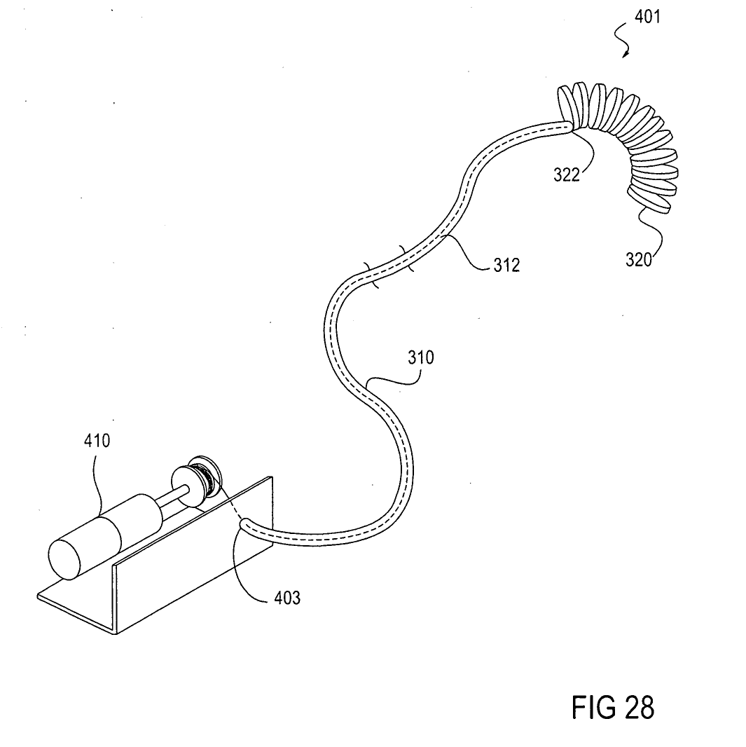

FIG. 28 shows a partial schematic representation of a single tendon bending a segment. For clarity, the other parts of a complete endoscope, including other tendons and segments, have been omitted from FIG. 28. Tension applied to a tendon cable is transferred across the entire segment, resulting in bending. By using a Bowden cable 310 whose sleeve 314 is attached to the base 322 of the segment and also fixed at the proximal actuator end 403, only the intended segment 401 is bent by applying tension to the tendon 312, and more proximal segments are unaffected. The tendon is placed in tension by the actuator 410, which is shown, in this variation, as a motor pulling on the tendon cable 312.

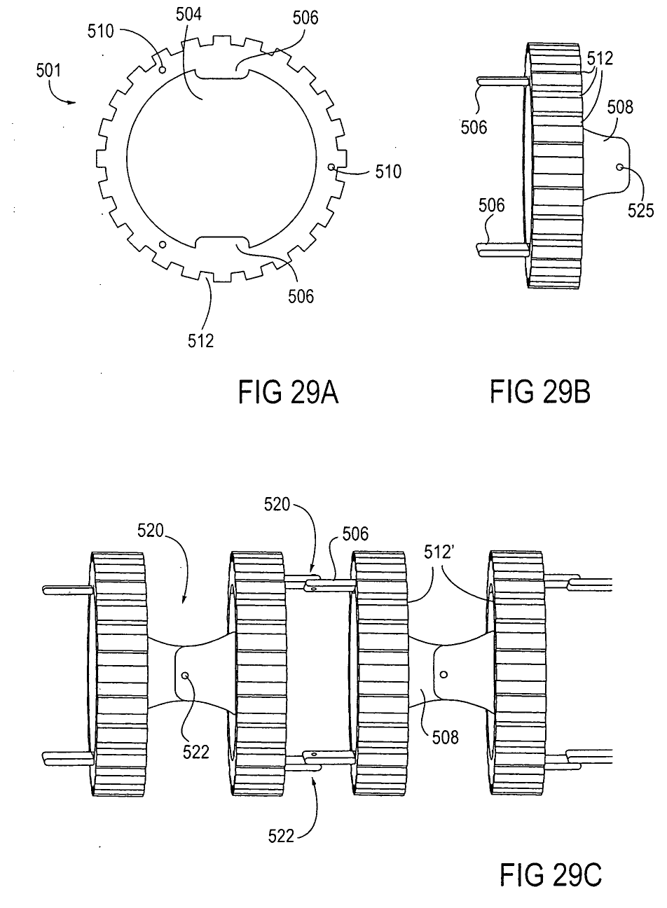

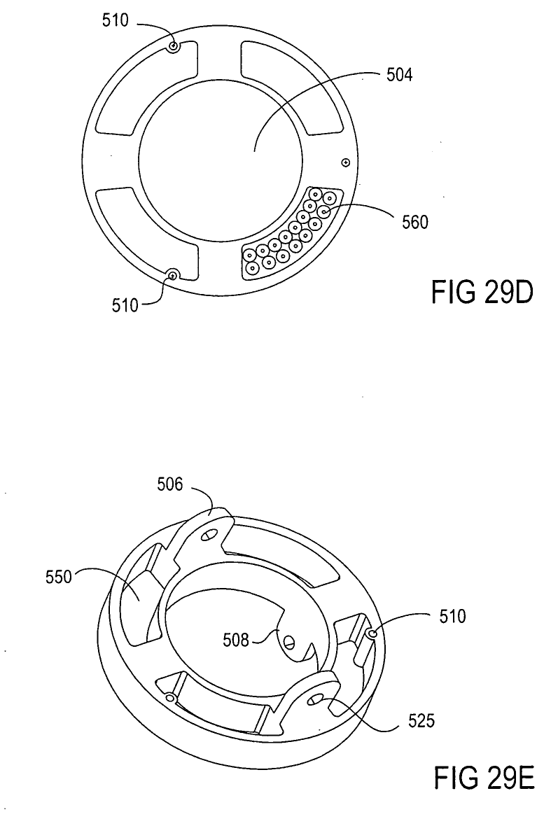

Linked control rings may provide the flexible structure needed to construct the steerable distal portion and the controllable segments. Two examples of the types of control rings that may be utilized are shown. The first is shown in FIG. 29A which shows a vertebra-type control ring that forms the controllable segments of the present invention. FIG. 29A shows an end view of a single vertebra. Each ring-shaped vertebra 501 can define a central channel or aperture 504 or apertures that can collectively form the internal lumen of the device as previously described. The vertebrae may have two pairs of hinges; the first pair 506 projecting perpendicularly from a first face of the vertebra and a second pair 508, located 90 degrees around the circumference from the first pair, projecting perpendicularly away from the face of the vertebra on a second face of the vertebra opposite to the first face. The hinges shown in FIGS. 29A and 29B are tab-shaped, however other shapes may also be used.

The vertebra control ring in FIG. 29A is shown with three holes 510 through the edge of the vertebra that may act, e.g., as attachment sites for the tendon cable 312 if the vertebra is the most distal vertebra in a segment, or as a throughway for a tendon cable that can actuate the segment in which the vertebra is used. These holes 510 can also be used to attach the sleeve of the Bowden-type tendon cable 314 when the vertebra is the most proximal control disk in a segment. Alternatively, rather than a hole 510, the attachment sites could be a recess or other specialized shape. Although FIG. 29A shows three holes 510, the number of holes may depend upon the number of tendons used to control the segment to which the vertebra belongs. Since the holes 510 may be used as attachment sites for the tendons, there are as many holes as there are tendons controlling the segment.

The outer edge of the vertebra in FIG. 29A may be scalloped to provide spaces 512 for tendon housings of tendons that control more distal segments and bypass the vertebra. These tendon bypass spaces preferably conform to the outer diameter of the tendons used. The number of tendon bypass spaces 512 may vary depending on the number of tendons. Also, the orientation of the tendon bypass spaces may be varied if it is desirable to vary the way in which the bypassing tendons are wound around the endoscope. For example, the spaces 512′ in FIG. 29C are oriented at an angle relative to the longitudinal axis of the vertebra, allowing the tendons to wind around the body of the endoscope as they project proximally. Furthermore, the tendon bypass spaces could be lubricated or composed of a lubricious material in order to facilitate free movement of the bypassing tendons across the segment, and prevent interference between the bending of the segment and the bypassing tendons.

FIGS. 29B and 29C show side views of the same vertebra as FIG. 29A. The two pairs of hinge joints 508, 506 are shown. Hinge joints 508, 506 are preferably located 90 degrees apart and extend axially so that the hinge joints can pivotally mate with hinge joints from adjacent vertebrae. This mating 520 with adjacent vertebrae is more clearly seen in FIG. 29C. These hinges can be joined, pinned, or connected through the holes 525 as shown 522. Alternatively, hinges may also be made from materials utilizing, e.g., thermoplastics, shape memory alloys, etc. Once hinged, each vertebra can rotate relative to an adjoining vertebra in one axis. However, because vertebrae are hinged to each other in directions alternating by 90 degrees, an assembly of multiple vertebrae is able to move in virtually any direction. The greater the number of vertebrae joined in this manner, the greater the range of motion. In one embodiment, two to ten vertebrae are used to comprise one segment, achieving a length of around 4 cm to 10 cm per segment. The dimensions of both the vertebrae and the hinge joints can be varied, e.g., longer hinge joints will have a greater bending radius when joined to another vertebra. Furthermore, the number of vertebrae per segment can vary, e.g. more than ten vertebrae could be used.

FIGS. 29D and 29E show another variation of a vertebra in sectional and perspective views, respectively. In FIGS. 29D and 29E, the tendons that bypass the segment may be contained within the body of the vertebra in a tendon bypassing space 550 rather than along the outer edge of the vertebra as shown in FIG. 29A. The vertebra of FIGS. 29D and 29E show four tendon bypassing spaces 550, and each space can hold approximately fifteen bypassing tendon sleeves. The number, shape and sizes of the tendon bypassing spaces can be varied. For example, a vertebra could have two tendon bypassing spaces that could hold more than thirty-five tendon sleeves. Moreover, the tendon bypassing space could also be located on the inside of the central aperture or lumen of the vertebra 504.

Although FIG. 29D shows tendon sleeves holding only a single tendon cable 560, more than one tendon cable could be contained in a tendon housing or sleeve. For example, if three tendons articulate a segment, all three tendons could be contained in a single tendon housing. Such a combined tendon housing could further utilize lubrication to accommodate independent movement by individual tendon cables and/or could be divided into compartments that isolate the tendons within the housing.

FIG. 29E also shows a perspective view of the hinge joints 506, 508 that can pivotally mate with pairs of hinge joints from adjacent vertebrae. Although FIGS. 29A and 29B shows two pairs of hinge joints projecting axially, a single hinge joint on each face of the vertebra could also be used. Moreover, as long as the hinge joints can pivotally mate with adjacent vertebrae, the hinge joints can be located at different radial locations from the center of the vertebra. For example, the pairs of hinge joints shown in FIGS. 29A to 29C are located closer to the center of the vertebra than the hinge joints in FIGS. 29D and 29E.

FIGS. 30A and 30B illustrate a second variation of control ring. The variation shown in the figure utilizes a flexible backbone 601 preferably made of a material that is relatively non-compressible and non-extensible, to which control rings 602 are attached at intervals. This structure allows bending in a continuous curve in any desired direction. FIG. 30A shows a side view of one controllable segment of this variation with the outer layers removed to show the control rings and backbone. Multiple control rings 602 may be attached to the flexible backbone at regular intervals. Fewer or more control rings could be used to comprise a single segment depending upon the desired degree of articulation. The tendon cable 312 attaches to the most distal control ring of the segment 604. As with the vertebra-type variation, this central backbone embodiment is shown actuated by three tendons 310 attached at sites equally spaced around the edge of the most distal control ring of the segment 604. The tendon cables controlling the segment 312 pass through spaces or holes 610 defined in the control rings 602 through which they are free to move. These holes 610 could be lubricated, lined with a lubricious material or the control rings 602 may be composed of some lubricious material to facilitate cable motion through the holes 610. The tendon sleeve preferably attaches at a location 614 to the most proximal control ring in the segment 612. When a tendon 312 is placed under tension, this force is distributed along the entire segment. Because the inner tendon cable 312 is freely slidable within the tendon sleeve 314, and the tendon sleeve is fixed at both ends of the tendon 614, pulling on the tendon cable causes bending only in the selected segment.

FIG. 30A also shows the first control ring of a more proximal segment 604′. The tendons controlling the more distal segment may pass over the outside of the more proximal segments as they project proximally to the actuators. The outer edge of the control rings for the flexible backbone embodiment are shown with channels or tendon bypassing spaces 616 for bypassing tendons, as seen in FIG. 30B. As with the vertebra-type control rings, these tendon bypassing spaces could also be located within the control ring, for example, in an enclosed tendon bypassing space.

FIG. 30B shows an end view of control ring 602 which may be used with the flexible backbone embodiment of the endoscope. The center of the control ring contains a channel through which the flexible backbone 601 can be attached. A number of additional channels through the control ring 618 are also shown. These channels can be aligned with channels in neighboring control rings to form an internal lumen or channel for a fiberoptic imaging bundle, illumination fibers, etc. as discussed above. Moreover, adjacent control rings may be spaced adjacently to one another at uniform or various distances depending upon the desired degree of bending or control. FIG. 30B shows three equally spaced holes 610 through which the tendon cable can pass; these holes 610 could also be used as attachment sites for the tendon cable, e.g., when the control ring is the most distal control ring in the segment 604, or for the tendon cable sleeve, e.g. when the control ring is the most proximal control ring in the segment 612. These holes 610 could be shaped specifically to receive either the tendon end or the tendon sleeve. Control rings of other designs could be used for different regions of the segment, or for different segments.

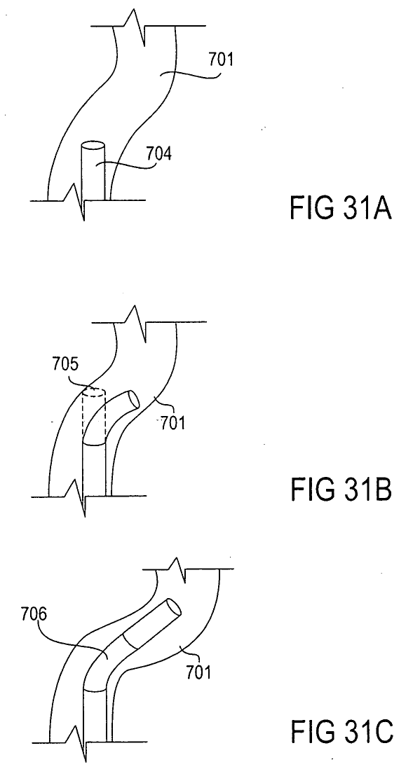

FIGS. 31A to 31C illustrate a variation of the tendon driven endoscope navigating a tortuous path. The path 701 is shown in FIG. 31A. This pathway may represent a portion of colon, for example. In FIG. 31A, the distal tip of the device 704 approaches the designated bend. FIG. 31B shows the distal tip being steered 705 to assume the appropriate curve. This steering could be performed manually by the user, e.g. a doctor, or automatically using an automatic detection method that could determine the proximity of the walls of the pathway. As described, the bending of the steerable tip is performed by placing tension on the tendon, or combination of tendons, that result in the appropriate bending.

The device is then advanced again in FIG. 31C; as it is advanced, the selected curve is propagated down the proximal length of the endoscope, so that the bend of the endoscope remains in relatively the same position with respect to the pathway 701. This prevents excessive contact with the walls, and allows the endoscope to move more easily along the tortuous pathway 701. The endoscope is in continuous communication with the motion controller, and the motion controller can monitor the location of the endoscope within the pathway, e.g., depth of insertion, as well as the selected bends or curves that define the pathway of the endoscope. Depth can be determined by, e.g., the axial motion transducer 49 previously described, or by more direct measurement techniques. Likewise, the shape of each segment could be determined by the tension applied to the tendons, or by direct measurement, such as direct measurement of displacement of the tendon cables. The motion controller can propagate the selected shape of a segment at a specified location, or depth, within the body, e.g., by setting the lengths of the sides of more proximal segments equal to the corresponding lengths of the sides of more distal segments as the device is moved distally. The controller can also use this information to automatically steer the body of the endoscope, or for other purposes, e.g. creating a virtual map of the endoscope pathway for analytic use.

In addition to measuring tendon displacement, the motion controller can also adjust for tendon stretch or compression. For example, the motion controller can control the “slack” in the tendons, particularly in tendons that are not actively under tension or compression. Allowing slack in inactive tendons reduces the amount of force that is required to articulate more proximal segments. In one variation, the umbilicus at the distal end of the endoscope may contain space to allow slack in individual tendons.

The bending and advancing process can be done in a stepwise or continuous manner. If stepwise, e.g., as the tendon is advanced by a segment length, the next proximal segment 706 is bent to the same shape as the previous segment or distal steerable portion. A more continuous process could also result by bending the segment incrementally as the tendon is advanced. This could be accomplished by the computer control, for example when the segments are smaller than the navigated curve.

Controllable segments, including the steerable distal portion, can be selected to have different dimensions, e.g., different diameters or lengths, even within the same endoscope. Segments of different dimensions may be desirable because of considerations of space, flexibility and method of bending. For example, the more segments in an endoscope, the further it can be steered within a body cavity; however, more segments require more tendons to control the segments. FIGS. 32 and 33 illustrate two variations on tendon driven endoscopes.

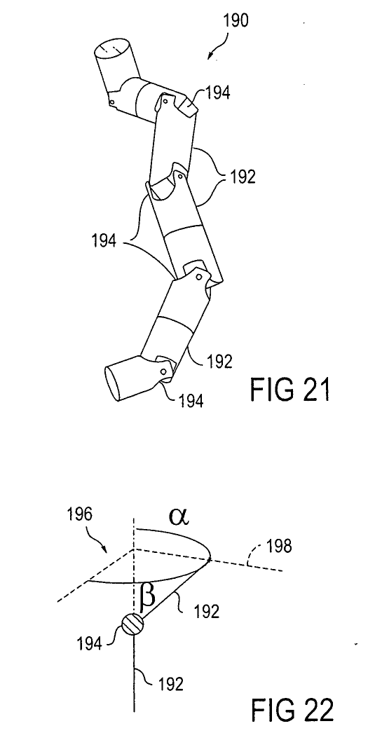

FIG. 32 shows a tendon driven endoscope variation that has segments 800 of differing diameters. More distal segments may have a smaller diameter 803 than more proximal segments, e.g., 802, 801. The diameter of a typical endoscope could decrease from, e.g., 20 mm, down to, e.g., 12.5 mm. The endoscope shown in FIG. 32 appears telescoped, as the diameter decreases distally in a stepwise manner. This design would be responsive, e.g., to internal body structures that become increasingly narrow. This design would also help accommodate bypassing tendons from more distal segments as they proceed towards the proximal actuators because of the larger diameter of the more proximal segments. FIG. 21 shows four differently sized segments; however, virtually any number of differently sized segments could be used. Moreover, although the segments appear stepped in this variation, the outer surface may be gently tapered to present a smooth outer surface decreasing in diameter towards the distal end.

FIG. 33 shows another variation of the tendon driven endoscope that has segments of different lengths. Using segments of different lengths may require fewer overall segments 900 to construct an equivalent length of articulatable endoscope. As shown in FIG. 33, more proximal segments 901 are increasingly longer than more distal, e.g., 902, 903, segments. For example, segment length could be decreased from 20 cm at a proximal segment down to 6 cm at a distal most segment. The lengths may be decreased incrementally segment to segment by a constant factor; alternatively, lengths may be decreased geometrically, exponentially, or arbitrarily depending upon the desired articulation. In practice this results in an “averaging” of curves by more distal segments as bends and turns are propagated proximally. In order to accomplish this, the motion controller may be configured to accommodate the differently sized segments accordingly. Alternatively, endoscopes could be comprised of a combination of segments of different length and thickness, depending upon the application.

The tendons that articulate the segments are in mechanical communication with the actuators. However, it may be desirable to have the insertable distal portion of the endoscope be removable from the actuators and controller, e.g., for cleaning or disinfecting. A quick-release mechanism between the proximal end of the endoscope and the actuators is an efficient way to achieve an endoscope that is easily removable, replaceable or interchangeable. For example, the proximal ends of the tendons can be organized to allow predictable attachment to corresponding actuators. The tendons may be organized into a bundle, array, or rack. This organization could also provide other advantages to the endoscope, such as allowing active or passive control of the tendon slack. Furthermore, the proximal ends of each tendon can be modified to allow attachment and manipulation, e.g., the ends of the tendons may be held in a specially configured sheath or casing.

In addition to the above described techniques for articulating instruments, including guide tubes and steerable instruments, activated polymer actuators may also be used as described in greater detail below.

A variety of electromechanical actuators based on the principal that certain types of polymers can change shape under certain conditions of stimulation have been under investigation for decades. During the 1990's, widespread international research was performed, numerous papers were published and several conferences held regarding activated polymer actuators. In January 2001, this research was organized by Yoseph Bar-Cohen in a book he edited entitled “Electroactive Polymer (EAP) Actuators as Artificial Muscles: Reality, Potential and Challenges” (SPIE Press, January 2001). As used herein, activated polymers refer generally to the families of polymers that exhibit change when subjected to an appropriate stimulus. See, for example, Bar-Cohen Topics 1, 3, and 7, Chapters 1 (pp. 1-38), 4 (pp. 89-117), 5 (pp. 123-134), 6 (pp. 139-184), 7 (pp. 193-214), 8 (223-243), and 16 (457-493) all of which are incorporated herein in their entirety.

One manner of categorizing activated polymers is by type of activation mechanism. Such categorization used by Bar-Cohen, and adopted herein, includes: non-electrically actuated polymers, ionically actuated polymers and electronically actuated polymers. There are numerous subcategories within each type of activation mechanism. Non-electrically activated polymers include chemically activated polymers, shape memory polymers, McKibben artificial muscles, light activated polymers, magnetically activated polymers, thermally activated polymer gels and polymers activated utilizing electrochemical action.

Ionically activated polymers include the groupings of electroactive polymer gels, ionomeric polymer-metal composites, conductive polymers, and carbon nanotubes. In one aspect, the invention provides an articulating instrument that is actuated or manipulated through the controlled use of an ionically activated polymer actuator activated without the use of an electrolyte. In a further aspect, the ionically activated polymer actuator comprises an electroactive polymer gel. In a further aspect, the ionically activated polymer gel actuator comprises a physical gel, a chemical gel, a chemically actuated gel, or an electrically actuated gel. In a further aspect, the ionically activated polymer actuator comprises an ionomeric polymer-metal composite. In a further aspect, the ionically activated polymer actuator comprises a carbon nanotube. In a further aspect, the ionically activated polymer actuator activates resulting in movement of the articulating instrument without the ionically activated polymer undergoing an oxidation/reduction process.

Electronically activated polymers include polymers activated using Coulomb forces, electrical forces, as well as electrostrictive, electrostatic, piezoelectric and/or ferroelectric forces. In a further aspect, the invention provides an articulating instrument that is actuated or manipulated through use of an electromechanical actuator from the category of an electronic electroactive polymer based actuator. In one aspect, an electronic electroactive polymer based actuator is used to articulate the controllable segments of an endoscope, including the distal steerable portion. In another aspect, embodiments of the electronic electroactive polymer based actuator include, but are not limited to, non-doped polymers, dielectric elastomers, electrostatically stricted polymers, electrostrictor polymer (i.e., polyvinylidene fluoride-triflouroethylene copolymer or P(VDF-TrFE)), polyurethane (such as manufactured by Deerfield: PT6100S), silicone (such as manufactured by Dow Corning: Sylgard 186), fluorosilicone (such as manufactured by Dow Corning: 730), fluoroelastomer (such as manufactured by LaurenL143HC), polybutadiene (such as manufactured by Aldrich: PBD), isoprene natural rubber latex, acrylic, acrylic elastomer, pre-strained dielectric elastomer, acrylic electroactive polymer artificial muscle, silicone (CF19-2186) electroactive polymer artificial muscle.

In another aspect, articulating instruments according to embodiments of the present invention employ a plastic actuator formed using a laminate polymer sheet structures including combinations of pre-strained polymers, unstrained polymers, compliant electrodes, active areas creating one planar direction of polymer deformation, active areas creating two planar directions of polymer deformation, compliant electrode patterning that produces multiple degrees of freedom and combinations of the above.

In some embodiments, an activated polymer is pre-strained. It is believed that the pre-strain improves conversion between electrical and mechanical energy. In one embodiment, pre-strain improves the dielectric strength of the polymer. The pre-strain allows the electroactive polymer to deflect more and provide greater mechanical work. Pre-strain of a polymer may be described in one or more directions as the change in dimension in that direction after pre-straining relative to the dimension in that direction before pre-straining. The pre-strain may comprise elastic deformation of a polymer and be formed, for example, by stretching the polymer in tension and fixing one or more of the edges while stretched. In one embodiment, the pre-strain is elastic. After actuation, an elastically pre-strained polymer could, in principle, be unfixed and return to its original state. The pre-strain may be imposed at the boundaries using a rigid frame or may be implemented locally for a portion of the polymer.

In one embodiment, pre-strain is applied uniformly over a portion of an active polymer to produce an isotropic pre-strained polymer. By way of example, an acrylic elastomeric polymer may be stretched by 200-400 percent in both planar directions. In another embodiment, pre-strain is applied unequally in different directions for a portion of the polymer to produce an anisotropic pre-strained polymer. In this case, the polymer may deflect greater in one direction than another when actuated. While not wishing to be bound by theory, it is believed that pre-straining a polymer in one direction may increase the stiffness of the polymer in the pre-strain direction. Correspondingly, the polymer is relatively stiffer in the high pre-strain direction and more compliant in the low pre-strain direction and, upon actuation, the majority of deflection occurs in the low pre-strain direction. By way of example, an acrylic elastomeric polymer used may be stretched by 100 percent in a first direction and by 500 percent in the direction perpendicular to the first direction. Additional details related to pre-straining activated polymers may be found in U.S. Pat. No. 6,664,718 to Pelrine et al. entitled “Monolithic Electroactive Polymers,” the entirety of which is incorporated herein by reference.

In other aspects of the invention, articulating instruments according to embodiments of the present invention utilize a plastic electromechanical actuator that relies on actuation from other materials, for example, infused mixtures of polymer gels with or without electrorheological fluid, electrorheological fluid, polydimethyl siloxane, polyacrylonitrile, carbon nanotubes and carbon single-wall nanotubes (SWNT).

Articulating instruments include a number of different types of articles including, for example, wireless endoscopes, robotic endoscopes, catheters, specific designed for use catheters such as, for example, thrombolysis catheters, electrophysiology catheters and guide catheters, cannulas, surgical instruments or introducer sheaths or other procedure specific articulating instruments.

Additionally, articulating instruments include steerable endoscopes, catheters and insertion devices for medical examination or treatment of internal body structures. Many such instruments are described in the following U.S. patents and U.S. patent applications, the disclosures of each are incorporated herein by reference in their entirety: U.S. Pat. Nos. 6,610,007; 6,468,203; 4,054,128; 4,543,090; 4,753,223; 4,873,965; 5,174,277; 5,337,732; 5,383,852; 5,487,757; 5,624,380; 5,662,587; 6,770,027; 6,679,836 and 6,835,173.

A steerable, multi-segmented, computer-controlled endoscopic device is one specific example useful for discussion purposes to describe some of the embodiments of the present invention. Examples of such endoscopes are described in U.S. Pat. Nos. 6,468,203 and 6,610,007 both assigned to the Applicant. These steerable segmented endoscopes may be utilized for insertion into a patient's body, e.g., through the anus for colonoscopy examinations. An example of such a device and a method for advancement within a patient utilizing a serpentine “follow-the-leader” type motion may be seen in U.S. Pat. No. 6,468,203, which is co-owned and has been incorporated herein by reference above. Each of the segments of the endoscope may be individually actuated and controlled to create arbitrary shapes. Using such a “follow-the-leader” type algorithm, the device may be advanced into tortuous lumens or paths without disturbing adjacent tissue or objects.

Another variation on segment actuation for realizing the “follow-the-leader” motion is described in U.S. Pat. App. Ser. No. 2002/0062062, filed Oct. 2, 2001. As described, one of the variations employs motors on board at least a majority of each individual segment. The motors described therein may be, in some embodiments of the present invention, replaced by electroactive polymer rotary clutch motors, such as those described in U.S. Pat. No. Application Publication US 2002/0175598 to Heim et al. entitled, “Electroactive Polymer Rotary Clutch Motors,” or electroactive polymer rotary motors, such as those described in U.S. Pat. No. Application Publication US 2002/0185937 to Heim et al. entitled, “Electroactive Polymer Rotary Motors,” both of which are incorporated herein by reference in their entirety. Adjacent segments may be pivoted relative to one another via hinges or joints. Another variation is described in U.S. Pat. App. Serial No. 2003/0045778, filed Aug. 27, 2002. As described, each of the segments of the multi-segmented endoscope may be actuated by push-pull cables or “tendons” (also known in the art as “Bowden cables”) connected to one or several actuators, e.g., motors, located remotely from the endoscopic device. Each of these publications is co-owned and incorporated herein by reference in its entirety.

As described herein, active polymer materials may be used in conjunction with multi-segmented articulating instruments to alter the relationship between, for example, two adjacent segments, a plurality of segments, a section of the articulating instrument or the entire length of the articulating instrument. Flexing of a portion of the instrument may result from inducing relative differences in size or length of material, e.g., active polymeric material, placed near, around or otherwise coupled to the instrument such that activation of the polymer results in controlled articulation of the instrument. For example, actuators utilizing an active polymer material may be located on opposing sides of a portion of an endoscope such that activation of the active polymer material results in the scope bending towards the side having the activated polymer actuator. In an alternative embodiment, another actuator utilizing an active polymer material may be located in opposition the earlier mentioned actuator so as to either not contract or to expand along the opposing side to facilitate bending or pivoting of that portion of the endoscope. The resulting shape will have the contracted portion of material along the inner radius, and the un-contracted or expanded length of material along the outer radius.

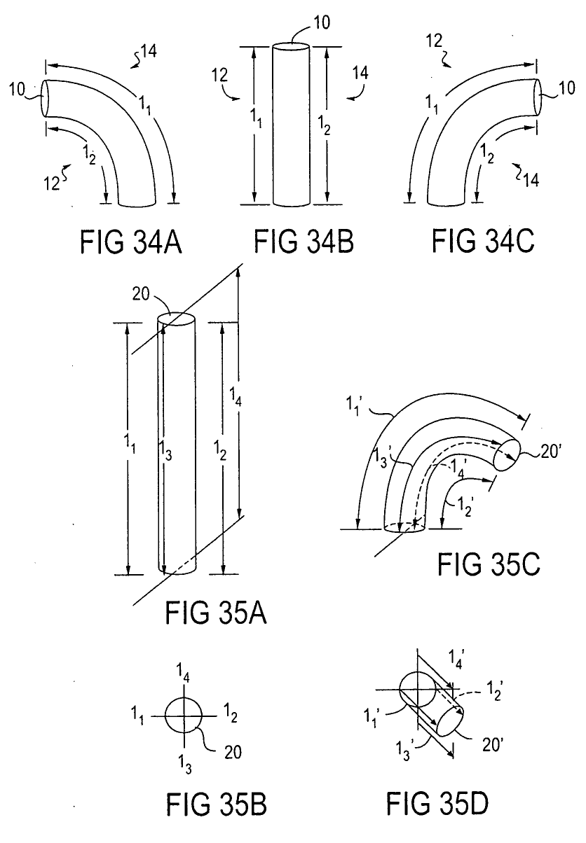

Consider a segment 10 having a first side 12 and a second side 14. Active polymer material or actuators are provided along the sides (not shown). When neither actuator or material is activated, the segment remains in a neutral position (FIG. 34B). On the other hand, FIG. 34 (a) shows the case where material located along the length of a first side 12 of the segment 10 shown, L.sub.1, is less than the length of material located along a second opposing side 14, L.sub.2, and the resulting bending of the segment towards the first side 12. FIG. 34 (b) shows the case where the length of the first side 12, L.sub.1, is equal to the length of the second side 14, L.sub.2, and the resulting straight, unbent, shape of the segment 10. FIG. 34 (c) shows the case where the length of the first side 12, L.sub.1, is greater than the length of the second side 14, L.sub.2, and the resulting bending of the segment 10 towards the second side 14.

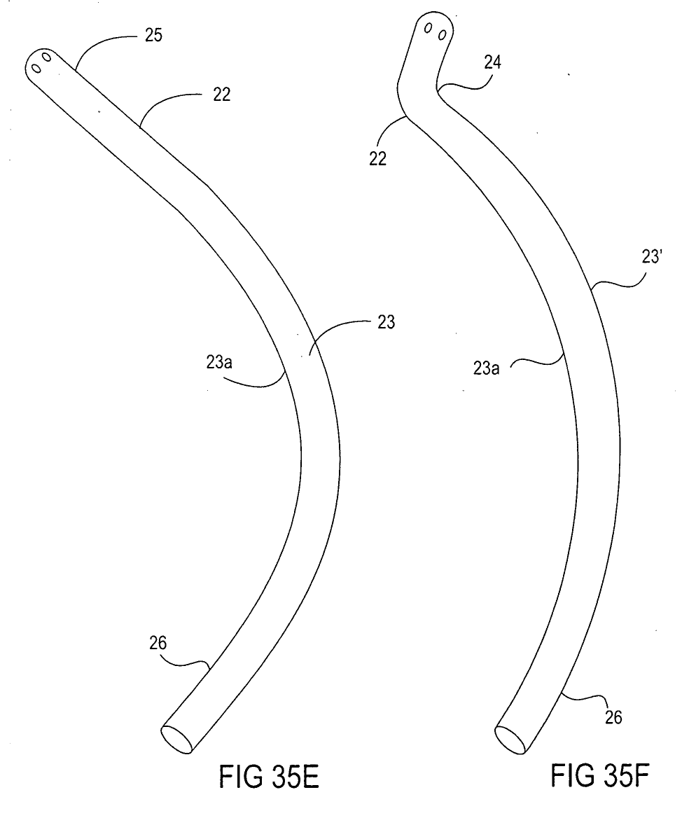

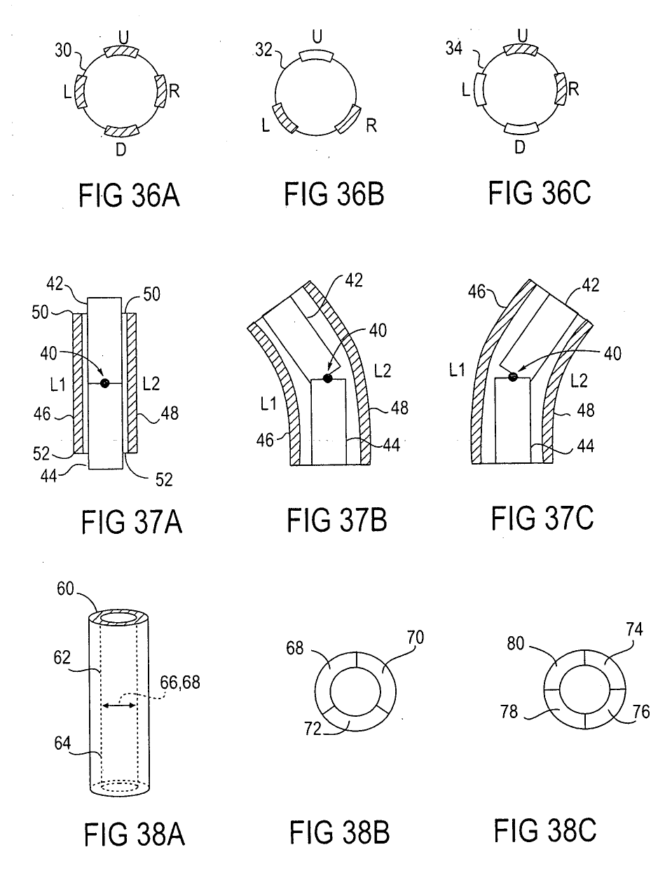

It is generally desirable to control the bending of the articulating instrument in all or as many directions as possible as suits the application. In one preferred embodiment, active polymer based actuators provide control rendering a segment capable of bending along at least two axes relative to a segment longitudinal axis. Segment 20 illustrates one configuration to achieve such control and articulation capable of bending along two axes (FIGS. 35a-35d). FIGS. 35 (a) and 35 (b) illustrate side and top views, respectively, of segment 20. The segment 20 is straight, and the lengths of the sides L.sub. 1, L.sub.2, L.sub.3 and L.sub.4 are all equal. FIGS. 35 (c) and 35 (d) illustrate side and top views, respectively, of an actuated or bent segment 20 or a segment 20′. As a result of the controlled actuation of activated polymer actuators coupled to the segment 20′, the segment 20′ has been articulated in two directions: towards the side denoted by L.sub.2, and also out of the plane of the page towards the side denoted by L.sub.3. In order to cause the depicted segment 20′ to bend as shown, length L.sub.2′ may be made shorter than length L.sub.1′, and length L.sub.3′ may be made shorter than length L.sub.4′, e.g. by causing the activated polymer materials or actuators located along L.sub.2′ and L.sub.3′ to contract. In this way, the segment 20′ may be caused to articulate, or bend, in two independent axes. Alternatively, the electro-polymeric materials along L.sub.2′ and L.sub.3′ may be remain un-actuated and the material along opposing sides L.sub.1′ and L.sub.4′ may be expanded to cause the resulting bending. In another alternative, all sides of the segment 20′ may be utilized in conjunction with another. For example, the material along sides L.sub.2′ and L.sub.3′ may be contracted while the material along sides L.sub.1′ and L.sub.4′ may be expanded simultaneously.

In yet another alternative, segment 20′ may represent an initial inactivated state for the segment that is pre-strained or has a bias condition with a predetermined and desired shape or curve. In this illustrative example, the segment 20′ is curved to the right in an inactivated state (FIGS. 35c and 35d). When the activated polymers or actuators coupled to the segment 20′ are activated, the segment is actuated into a straight condition. Pre-bias of a segment allows for actuation with fewer actuators. In this illustrative example, the actuator along side 12 may be removed since the pre-bias provides the curvature provided by the actuator in this position. During operation, the pre-bias is either reduced (i.e., less of a right turn), eliminated (i.e., straight up as in FIG. 35a) or articulated into another configuration as desired.

The use of pre-bias is also illustrated with articulating instrument 22 (FIGS. 35e, 35f). Articulating instrument 23 includes a plurality of segments (not shown for clarity) with selectively steerable distal portion 25 and an automatically controlled proximal portion 26. The articulating instrument 22 may be pre-biased into any desired curve. The curve may represent a typical pathway used, for example, in a surgical procedure such as an operation within the thoracic cavity, where the pre-bias shape is related to the likely shape of instrument when finally in position. The general pre-bias shape may be manipulated to fine tune the shape to patient specific anatomy. In another example, the pre-bias shape may relate to the pathway formed by vasculature or relate to the anatomy within an organ, such as the heart.

Articulating instrument 22 will now be described in relation to a use as a controllable, segmented colonoscope actuated through the use of active polymer layers or actuators. Once the articulating instrument 22 has been lubricated and inserted into the patient's colon through the anus A, the distal end is advanced through the rectum until the first turn in the colon is reached. This first turn is illustrated in FIG. 35F with bend 24. To negotiate the turn, the selectively steerable distal portion 25 is manually steered toward the sigmoid colon by the user through a steering control. The control signals from the steering control to the selectively steerable distal portion 25 are monitored by an electronic motion controller. Once the correct curve of the selectively steerable distal portion 25 for advancing the distal end of the instrument 22 into the sigmoid colon has been selected, the curve is logged into the memory of the electronic motion controller as a reference. Whether operated in manual mode or automatic mode, once the desired curve (24) has been selected with the selectively steerable distal portion 25, as the articulating instrument 22 advances distally, the selected curve 24 is propagated proximally along the automatically controlled proximal portion 26 using an electronic motion controller. As is common in “follow the leader” techniques (described below) the curve 24 remains fixed in space while the articulating instrument 22 advances distally through the sigmoid colon.

However, beyond the first turns to reach the sigmoid colon, traversing the colon may be thought of as a series of “left hand turns.” Consider, for example, that traversing the colon from the sigmoid colon into the descending colon, the descending colon into the transverse colon, and the transverse colon through the right (heptic) flexture into the ascending colon includes a series of left turns. As such, the pre-bias bend 23 is an example of a left hand pre-bias that may be used to approximate the general orientation of the articulating instrument once the colon has been traversed. In this way, in order for the instrument 22 to traverse the colon the pre-bias is selectively removed as it progresses. The pre-bias may also be removed selectively to more closely approximate the patient's anatomy. In alternative embodiments, the pre-bias may be shaped to any position other than the final position as described above.

FIG. 35F also illustrates how the instrument may be actuated in some portions while retaining the pre-bias condition in others. For example, the selectively steerable end 25 is articulated to form bend 24, the mid-region is actuated to diminish the pre-bias curvature while the proximal end retains the original pr-bias curvature. It is to be appreciated that the use of pre-bias may allow for fewer actuators to be needed to maintain the instrument in the final position or fewer actuators may be used overall. For example, in the left hand bias of instrument 22, actuators along the side 23a may be fewer or non-existent. Such an embodiment of the instrument 22 would thus be actuated through use of actuators to reduce, nullify or overcome and redirect the instrument out of the pre-bias shape.

There is provided a bendable instrument 22 having an elongate body with a distal end 25 and a proximal end 26. The elongate body is provided with a pre-bias shape. There is least one activated polymer actuator coupled to the elongate body such that when activated the at least one activated polymer actuator alters at least a portion of the elongate body out of the pre-bias shape. In one embodiment, the at least one activated polymer actuator comprises an electrically activated polymer actuator. In another embodiment, the at least one activated polymer actuator comprises an ionically activated polymer actuator. In yet another embodiment, the at least one activated polymer actuator comprises a non-electrically activated polymer actuator. In addition to or in combination with the pre-bias shapes described above, pre-bias shape embodiments also include: a pre-bias shape is related to: a typical pathway used in a surgical procedure, a portion of the vasculature; a portion of the skeleton, the shape of an organ, including both internal and external organ shapes. In some embodiments, the pre-bias shape is related to the internal shape of a portion of a heart, a colon, a gut, or a throat. In some embodiments, the pre-bias shape is related to the external shape of a portion of a heart, a liver, or a kidney.

In some embodiments, an articulating instrument is a restoring force that biases the entire assembly toward a substantially linear configuration in one embodiment, or into non-linear configurations or specialized configurations as described above. As discussed above, actuators may be used to deviate from this substantially linear configuration. It is to be appreciated that any of a number of conventional, known mechanisms can be provided to impart a suitable bias to the articulating instrument. For example, and as previously illustrated, an instrument may be disposed within an elastic sleeve, which tends to restore the system into a configuration determined by the strained, unstrained or otherwise configured shape of the sleeve. Alternatively, springs or other suitably elastic members can be disposed in relation to structural elements of a segment to restore the instrument to a desired configuration, linear, non-linear or other shape as discussed elsewhere. In yet another alternative, the structural elements of the instrument itself may, alone or in combination with other suitable elastic or restorative members to maintain or restore the instrument to a desired configuration.

In some embodiments of the articulating instruments of the present invention, at least two controllable lengths of the sides of an instrument segment are desirable. In some embodiments, at least two controllable segment lengths would be needed to provide two independent axes in order to allow the segment to bend in any number of directions. In some embodiments, each of the sides or controllable lengths are independently actuatable. Alternatively, a single controllable length may be utilized for each axis, along with a biased spring-type element positioned to oppose the controllable length or actuator. In one alternative embodiment, fixed the lengths on the sides of one axis and then vary the length of the opposing sides. With reference to FIG. 2(a), for example, if lengths L.sub.1 and L.sub.3 were fixed, then actuating the lengths L.sub.2 and L.sub.4 would enable the segment 20′ to bend in a number of directions.

In another alternative embodiment, three independently controllable actuators or activated polymer material may be coupled to the sides of an instrument to control the actuation of the instrument. Instead of being spaced at 90 degree intervals, as is shown in FIG. 35B, 35D, the independently controllable actuators or activated polymer material could be spaced at 120 degree intervals or form 60 degree arc segments about the circumference of the articulating instrument. By extension, any number of controllable actuators or activated polymer material formed into sections (including longitudinal, horizontal or lateral sections) may be coupled to the articulating instrument or it's segments, or groups of segments to provide bending and/or articulation of the instrument as desired.

In some embodiments, it is preferable to control at least one pair of activated polymer actuators coupled to opposing sides of an instrument. This may result in four independently controllable sides or portions of a segment which may be utilized to determine the bending of the segment. This may facilitate the simplicity of computation for determining the desired or necessary bending. This may further result in desirable controllability and responsiveness when causing a segment to bend. For example, FIG. 36(a) shows a top view of a segment 30 in a configuration utilizing four independently controllable actuators along the sides for determining the length of the sides or bending of the segment 30. In this embodiment, the actuators (U, D, L, and R) are arranged on opposing sides about a circumference of the segment 30 at 90 degree intervals. Alternatively, segment 32 in FIG. 36 (b) illustrates three independently controllable actuators along the sides (U, L, R) for determining the length of the sides. The three actuators U, L, R are spaced about the circumference of the segment 32 at 120 degree intervals. FIG. 36(c) shows yet another variation 34 showing two independently controllable sides U, R for determining the length of the sides of a segment 34 and two fixed-length sides D, L opposite with respect to sides U, R, arranged at 90 degree intervals.

Although the examples shown above are directed towards specific variations for placement of activated polymer materials and actuators circumferentially about a segment, these examples are intended to be illustrative and other variations and configurations for their placement are included within the scope of this disclosure.

In some embodiments, activated polymer materials and/or activated polymer based actuators may be configured for controlling the length of the sides of portions, or segments, of an articulated instrument to bend or otherwise manipulate the instrument into a desired direction, orientation or configuration. By positioning individually controllable pieces or regions of activated polymer material or actuators such that they may act on the segments of an instrument to modify, shorten, lengthen or otherwise alter the relative positions of segments or portions of the instrument and then controlling the contraction and/or activation of the activated polymers, the articulating instrument segments may be made to bend and flex as desired.

In one embodiment, pieces or lengths of activated polymer materials and/or activated polymer based actuators may be arranged around the periphery or circumference of a hinge or joint 40 between two adjacent segments 42, 44 (FIGS. 37 (a) to 37(c)). The ends of the pieces 50, 52 of activated polymer materials and/or activated polymer based actuators 46, 48 may be fixed to the adjacent segments 42, 44 around the hinge or joint 40. As such, activation of or changes of length of the activated polymer materials and/or activated polymer based actuators 46, 48 will exert forces on the hinge or joint 40 and bend it in its axis of motion. As shown in FIG. 37(a), constriction of the length of active polymer material 46 on a first side L.sub.1 is controlled so that it is the same length as that of the material 48 on a second side L.sub.2, the hinge 40 will not be caused to bend, and will configure into a straight configuration. In this case, the hinge 40 may optionally be under equal tension from both activated polymer materials and/or activated polymer based actuators 46, 48, or it may be under no tension from either length L.sub.1 or L.sub.2.

To bend the joint or hinge to a first side towards L.sub.1, as shown in FIG. 37(b), the length of polymeric material 46 may be caused to contract while the length L.sub.2 of polymeric material 48 may be caused to relax or expand. To bend the joint or hinge 40 to the opposing second side towards L.sub.2, as shown in FIG. 37(c), the length L.sub.2 of polymeric material 48 may be caused to contract while the length L.sub.1 of polymeric material 46 may be caused to relax or expand. The polymeric material may also be located inside an interstitial space or lumen defined within the adjacent segments 42, 44 and hinges 40. FIG. 37A is an exemplary embodiment where activated polymer materials and/or activated polymer based actuators are configured around the outside of the segments and hinges. Alternative configurations are also possible, such as a configuration where the activated polymer materials and/or activated polymer based actuators are disposed within or between the segments and/or hinges.

While the embodiment illustrated in FIG. 37A includes activated polymer actuators of equal lengths or sizes(i.e., L.sub.1 being equal in length to L.sub.2), other embodiments of the invention are not so limited. Other variations may utilize lengths, sizes and shapes of activated polymer actuators and/or material having different lengths about the same joint or hinge. In one embodiment, a first length L.sub.1 may be longer or shorter than a second length L.sub.2 when both lengths are in a neutral or non-activated configuration. When either or both lengths are stimulated to either contract or expand, the adjacent segments may be configured to bend at various angles about the joint or hinge relative to one another. Alternatively, activated polymer actuators and/or material of different lengths may be configured to effect a uniform bending of the segment about the longitudinal axis of the segment.

In another alternative embodiment, the design of the articulating instrument may be extended to two axes of bending by using a universal joint instead of a hinge. A universal joint allows for bending in any direction relative to the segment longitudinal axis. In this case, lengths of activated polymer material and/or activated polymer actuators may be arranged around the circumference of the segment across the universal joint such that adjacent segments may be caused to bend in any desired direction. This preferably utilizes at least two lengths of material arranged between the segments such that they are each able to effect motion of the joint in each of the two independent axes. In one embodiment, the minimum number of lengths of material or actuators is two. In other embodiments, any number may be used to cause the desired bending of the universal joint. In another specific embodiment, four lengths of activated polymer material or actuators are arranged in intervals around the periphery of the universal joint such that, when activated, they generate push and/or pull forces in each of the two independent axes of bending. In one embodiment, the interval is 90 degrees. In alternative embodiments, the interval is not a 90 degree interval but instead is in another arrangement suited to the particular geometry of the joint used.

Turning now to FIGS. 38A-C, there is illustrated another embodiment of an activated polymer actuated instrument of the present invention. In this embodiment, a continuous band of activated polymer material is formed into an annular ring 60 having a length and placed about two adjacent segments 62, 64. A hinge 66 is positioned between the segments 62, 64. The activated polymer ring 60 is disposed about the periphery of a hinge 66 that may bend in one or more axes. Alternatively, the segments 62, 64 may be coupled together using a universal joint 66′ that may bend in two or more axes, as shown in FIG. 38A. The annular ring 60 may be a single sheet of activated polymer material (FIG. 38A) having multiple active areas that deflect selected portions of the polymer to result in controllable movement of the segments 62, 64. In an alternative configuration, the annular ring may not be a single piece but instead a plurality of longitudinal activated polymer strips, such as polymer strips 68, 70 and 72 in FIG. 38B. In one embodiment, controllable activated polymer regions 68, 70, 72 individually (or alternatively, as a subset of the single piece, annular ring 60) are configured and controlled such that they may contract, relax, and/or expand as desired through the use of electrodes that may be energized, de-energized, and/or energized with polarities reversed to impart the desired shape or orientation of segments 62, 64. In one preferred embodiment, each of the controllable regions 68, 70, 72 or the single ring 60 are independently controlled. As such, a single piece or length of activated polymer material may be used to actuate either a hinge 66 or a universal joint 66′ in any desired direction.

While illustrated with three, any number of individually controllable regions of electro-polymeric material may be created. In some embodiments, the number of regions is greater than or equal to two. In one embodiment, the regions are arranged such that they act in the plane of the axis they control. For instance, three regions 68, 70, 72, as shown in FIG. 38B or four regions 74, 76, 78, 80, as shown in FIG. 38C, may be utilized to individually control regions as desired to create the push and/or pull forces.

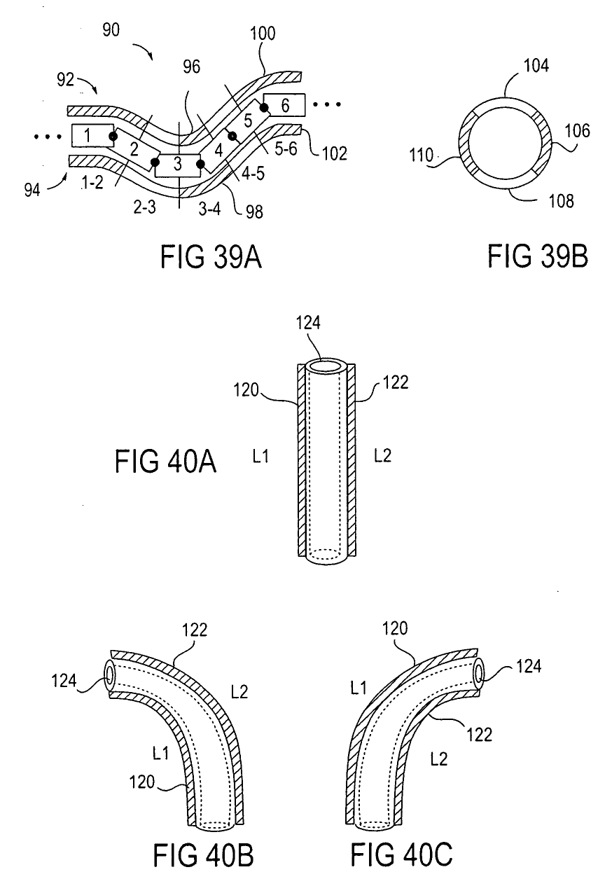

In yet another variation, a continuous band of electro-polymeric material that is formed in an annular ring and placed around the periphery of a segment may be made to be longer in length so that it extends over several, i.e., over at least two, hinges or universal joints, as shown in FIG. 39A. It may be made in a single continuous piece and may be made to cover a portion of the length or even the entire length of the flexible endoscope structure. In this configuration 90, independently controllable regions of the electro-polymeric material, e.g., regions 96, 98, 100, 102 and so on, may be created and located so that they are able to exert bending forces on each hinge, joint, or universal joint along the length of the endoscope, or as many hinges, joints or universal joints as are contained within the sleeve of electro-polymeric materials 92, 94. The electro-polymeric material may be fixed to the hinged or jointed structure at or near the midpoint of rigid sections between the hinges or joints in order to impart force to the hinges and joints to make them bend, or optionally the electro-polymeric material may be unattached to the structure, and either impart forces to the structure using frictional contact and elasticity or cause the structure to conform to the shape it is controlled to take on with the electrodes. Alternately, the length of electro-polymeric materials may be located inside the segments, hinges and/or universal joints, in any interstitial space defined within.

In another embodiment, a multi-segment articulating instrument 90 includes a plurality of individually controllable regions (FIG. 39A). In this embodiment, the articulating instrument 90 includes 6 hinged segments covered by activated polymer material 92, 94. In one embodiment, the activated polymer material is divided into a plurality of controllable segments that correspond to the hinged portions between segments. When activated, these activated polymer materials produce controlled movement between segments about the hinge (i.e., segment 5-6 may be altered by controllable segment 100 or controllable segment section 102. Articulating instrument 90 may bend each hinge or joint in the desired directions through activation of the activated polymers in the individually controllable regions 96, 98, 100, 102 of polymer material 92, 94. In one embodiment of the articulating instrument 90, a continuous band of active polymer material that runs the length, or a subset of the length, of the instrument and forms a sheath is provided. This sheath may be made of or coated by biocompatible materials, such as silicone, urethane, or any other biocompatible material as is commonly used in endoscopes or other medical devices, so that it may come in contact with living tissue without causing harm or damage. In one embodiment, the electrodes used to control the shape and length of the active polymer material or actuators are insulated or covered to prevent electric shock, which may also be accomplished with biocompatible materials. In another embodiment, the electrodes are compliant electrodes. In yet another embodiment, the sheath is part of a multi-layer laminate polymer actuator. In one embodiment, the sheath forms a disposable cover over a segmented structure comprising hinges and activated polymer materials coupled to the hinges. In another embodiment, the sheath is cleanable, washable and/or reusable.

FIG. 39B shows a cross-sectional view of an alternative embodiment of a controllable region. Rather than have the entire sleeve of activated polymer material, there may be provided sections of activated polymer material and non-activated polymer material. For example, sections 104, 110 may be the portions having activated polymers (for example, compliant electrodes distributed across a portion of their surface) while the sections 106, 108 would not have activated polymers or be formed from non-activated polymer material. Alternatively, each of the portions 104, 106, 108, 110-may be made of activated polymer materials and may each be controllable independently from one another. The sections need not be limited to the longitudinal sections illustrated. Other alternative embodiments include: more than four sections, a plurality of concentric longitudinal sections, annular sections, a plurality of concentric annular sections and combinations of longitudinal sections, annular sections and concentric sections.

In other alternative embodiments, a bendable instrument or articulating instrument does not use segments as in FIGS. 39B, C but rather a continuous flexible material. As illustrated in FIGS. 40A-C, a representative segment 124 is made of a flexible material, such as a hose, tube, spring or any other continuous material that may be bent or flexed. In the illustrated embodiment, sections, pieces or lengths of activated polymer material 120, 122 is arranged around the periphery of the segment 124. The pieces of activated polymer material are coupled to the segment 124 such that activation of the polymer resulting in the desired deflection, bending or other actuation of the segment 124. The activated polymer material may be coupled to the structure of the segment 124 in any number of positions, for example, along the outside of the segment, the inside of the segment, only at the segment ends, continuously along. the segment length, or in any other manner such that activation of the activated polymer material results in controlled changes in the shape, orientation, bending or overall geometry of the segment 124.

An exemplary actuation of segment 124 will now be described with reference to FIGS. 40A-C. As shown in FIG. 40A, when the length of electro-polymeric material 120 on the first side with length L.sub.1 is controlled so that it is the same length as that of the material 122 on the second side with length L.sub.2, segment 124 will not be caused to bend, and will be in a straight configuration. In this case, the segment 124 may optionally be under equal tension from both activated polymer materials 120, 122, or, alternatively, the segment 124 be under no tension from either activated polymer. To bend the segment 124 to a first side, as shown in FIG. 40B, the activated polymer material or actuator 120 on the left of segment 124 (L.sub.1) may be caused to contract while the activated polymer material or actuator 122 on the right (L.sub.2) is caused to relax or expand. To bend segment 124 to the right, as shown in FIG. 40C, the activated polymer material or actuator 122 to the right of segment 124 (L.sub.2) may be caused to contract while the activated polymer material or actuator 120 to the left (L.sub.1) is caused to relax or expand. FIG. 40 shows the hose, tube or spring bending in one axis (left-right) for illustrative purposes, and may be extended to two axes and three dimensions by adding additional, individually controllable lengths of electro-polymeric material to cause the hose, tube or spring to bend in a plane out of the page (up-down).

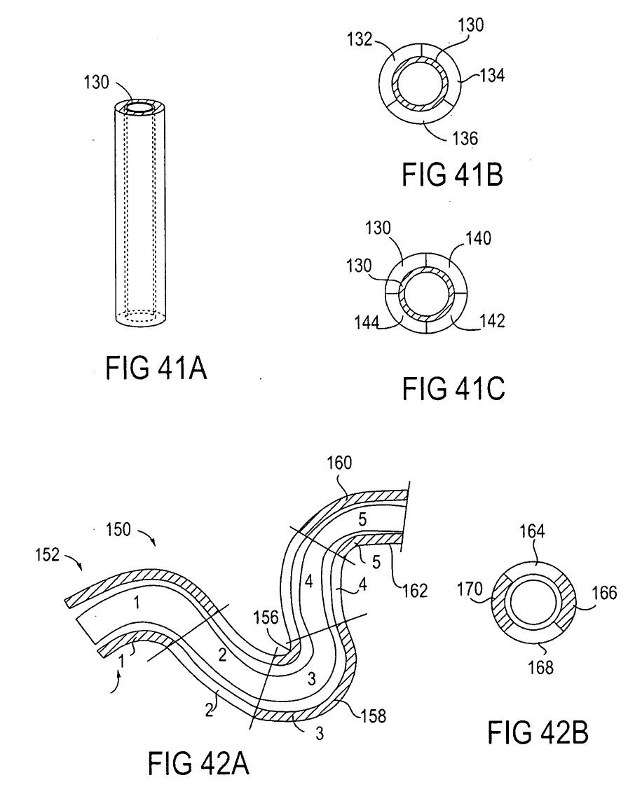

In yet another variation, a continuous band of activated polymer material may be formed in an annular ring and placed around the periphery of a segment 130, e.g., hose, tube, spring or any other continuous material that may be bent or flexed in any direction. In this configuration, as shown in FIG. 41A, independently controllable regions 132, 134, 136 of activated polymer material are created such that they may contract, relax, and expand as desired through the use of electrodes that may be energized, de-energized, or energized with polarities reversed. In this way, a single piece of activated polymer material may be used to actuate a length of segment 130. Any number of individually controllable regions 132, 134, 136 of activated polymer material may be created. In one embodiment, there are two controllable regions. In another embodiment, there are three controllable regions as in the three regions 132, 134, 136 shown in FIG. 41B. In yet another embodiment there are four or more controllable regions such as the four regions 138, 140, 142, 144 shown in FIG. 41C. In any of the above described regions, the regions may be arranged such that they expand and/or contract in the plane of the axis they control and/or may be used to individually control regions to create push and/or pull forces on the segment 130.

FIG. 42A illustrates alternative embodiment of an articulated instrument of the present invention. Articulating instrument 150 includes in a continuous band of activated polymer material 152, 154 that is formed, in this embodiment, as an annular ring and may be placed around the periphery of or along the inner diameter of the interstitial space defined by a length of hose, tube, spring or any other continuous material 153 that may be bent or flexed in a desired direction. In some embodiments, the activated polymer material is of sufficient length such that it extends over several “segments.” In FIG. 42A, five “segments” of the continuous structure are created because of the individual control over each of the controllable sections or regions 156, 158, 160, 162. These segments are defined as independently controllable sections that may be caused to bend in any direction. Segments may be chosen to be any desired length. In an exemplary embodiment where the articulating instrument is an endoscope the segments may, for example, range in length from, e.g., I cm to 10 cm. For other applications even smaller segment lengths may be used and will depend on the application. In some embodiments where the articulating instrument is intended to navigate the vasculature or other confined pathways, the segment length may be less than one cm, such as 50 mm or 25 mm.

The activated polymer material 152, 154 used may be made in a single continuous piece, and may be made to cover the entire length of the hose, tube, spring, or other flexible material making up the flexible endoscope structure 150. In this configuration, independently controllable regions 156, 158, 160, 162 of the activated polymer material are created and located so that they are able to exert bending forces on each segment along the length of the endoscope, or as many segments as are contained within the sleeve of the activated polymer material, which may be less than the entire length of the endoscope. The activated polymer material 152, 154 may be fixed to the hose, tube, spring, or other flexible material making up the endoscope at or near the endpoints of each of the segments in order to impart force to the segments to make them bend, or optionally the activated polymer material 152, 154 may be unattached to the structure, and either impart forces to the structure using frictional contact and elasticity or cause the structure to conform to the shape it is controlled to take on with the electrodes.

FIG. 42A illustrates an embodiment having individually controllable regions 156, 158, 160, 162 of activated polymer material configured to act such that they are able to bend each hinge or joint in the desired directions. In this structure, the continuous band of activated polymer material that runs the length, or a subset of the length, of the endoscope made of a series of segments forms a sheath. This sheath may be made of or coated by biocompatible materials, such as silicone, urethane, or any other biocompatible material as is commonly used in endoscopes or other medical devices, so that it may come in contact with living tissue without causing harm or damage. The electrodes used to control the shape and length of activated polymer material may be compliant electrodes and may also be insulated or covered to prevent electric shock, which may also be accomplished with biocompatible materials. In one embodiment, the sheath is disposable. In another embodiment, the sheath is cleanable and reusable.

FIG. 42B illustrates a cross-sectional view of one embodiment of one portion of the controllable region. Controllable region portions 166, 168 may be configured with the activated polymer material while portions 164, 170 may be made of non-activated polymer material. In another alternative embodiment, each of the controllable region portions 164, 166, 168, 170 may include activated polymer material and may each be controllable independently one from the others.

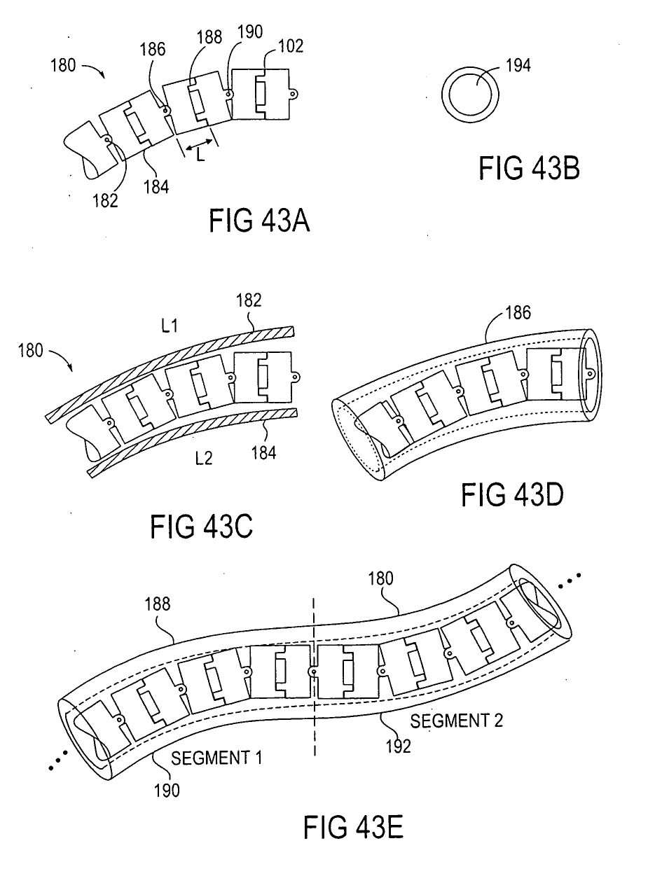

In yet another variation, a length 180 of hose, tube, spring, or alternate flexible material or structure may be comprised of a plurality of hinges, joints, or universal joints 182 to 192, as shown in FIG. 43A. The hinges, joints, or universal joints 182 to 192 may be connected together to form a segment 180, shown in FIG. 43A, which may then be caused to bend in two axes, e.g., via the use of activated polymer material. The hinges, joints, or universal joints 182 to 192 may define an inner lumen 194, or working channel, as shown in the end view of segment 180 in FIG. 43B, which is large enough so that components may be assembled or passed within the defined lumen 194. Tools and components such as cables, tubes, working channels, optical fibers, and other tools, illumination bundles, etc., may be passed through the lumen 194. For arrangements that make use of hinges or joints that are configured to bend only in one axis (as opposed to universal joints, which are able to bend in at least two axes), it is preferable to alternate the orientation of the hinges or joints so that every other hinge or joint bends in one axis (e.g., left-right) with intermediate hinges or joints bending in another axis (e.g., transverse or up-down).

The spacing between the joints 182 to 192 lengthwise down the segment 180 is preferably small relative to the diameter of each link (e.g., 1:1 or less), so that the lengths of straight, un-articulated material covering the joint between adjacent links is correspondingly small. In this way, the series of discrete hinges, joints, or universal joints 182 to 192 may approximate the continuous shape of a flexible material (e.g., a hose, tube, spring, etc.). In this variation, activated polymer material may be used in any of the variations described above.

In one embodiment, illustrated in FIG. 43C, individual pieces or lengths of activated polymer material 182, 184 may be used either outside the segments or inside to apply bending forces to the segments made of hinges or joints. Alternatively, as shown in FIG. 43D, a continuous band 186 may be placed around the circumference of a segment or within the inner diameter of the segment that is the length of the segment or at least a partial length of the segment and is attached to the segment at or near the endpoints. In another alternative, as shown in FIG. 43E, a continuous sleeve 188 may be placed around the circumference of a number of segments 190, 192 that may comprise the entire endoscope or a subset of the segments making up the endoscope. In the variations where a continuous band or sleeve is used, it may be preferable to configure the activated polymer material so that it has, in some embodiments, four individually controllable regions about the circumference per segment, and that these regions may exert push and/or pull forces in line with the axis of bending of the hinges or joints. Individually controllable pieces or lengths of activated polymer material, or individually controllable electrodes covering individual regions of activated polymer material, may be used to bend each of the segments individually in any desired direction. In addition, a sheath may be provided that is made of or coated by biocompatible materials, such as silicone, urethane, or any other biocompatible material as is commonly used in endoscopes or other medical devices. The sheath coating or material is selected so that it may come in contact with living tissue without causing harm or damage. The electrodes used to control the shape and length of the activated polymer material may, in some embodiments, be insulated or covered to prevent electric shock, which may also be accomplished with biocompatible materials. In other embodiments, the electrodes are compatible electrodes. In one embodiment, the sheath is disposable. In another embodiment, the sheath is cleanable and reusable.

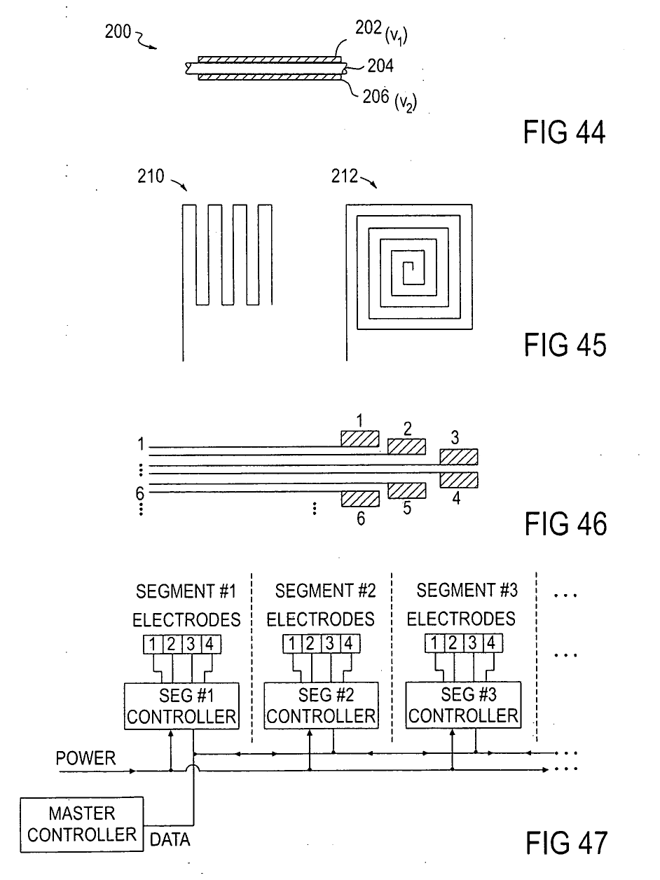

Actuation of the activated polymer material may occur in any of a number of ways depending upon the activation mechanism of that particular polymer. For example, the activation may occur for some polymers by placing them, or parts, or regions of them, in the presence of an electric field. In other cases, an activation mechanism may be related to placing an activated polymer in contact with substances that have varying levels of pH. In some embodiments, electrically activated polymer materials and actuators are actuated through use of electric fields order to create the electric fields, electrodes may be used, as shown in FIG. 44. These electrodes 202, 206 may be created by placing conductive materials on either side of a piece or region of electro-polymeric material 204, and causing the conductive material 202 on one side of the electro-polymeric material to be at one voltage potential (V.sub.1) while causing the conductive material 206 on the other side of the electro-polymeric material to be at another voltage potential (V.sub.2). In this way, an electric field is established across the electro-polymeric material. The voltage potential may be steady and constant, or may be time-varying.

In another variation, the electrodes may be separate materials in very close contact with the electro-polymeric material. The-arrangement of electrodes and electro-polymeric material may be created, e.g., in a sandwich configuration, with each component comprised of a separate piece. The layers may be either flat or tubular. A thin, conductive, flexible material such as Mylar may be used. In order to allow for the contraction, relaxation, and/or expansion of the electro-polymeric material, the layers of the sandwich arrangement may be able to slide relative to each other. For this reason, slippery or lubricious materials may be utilized.

In yet another variation, the electrodes may be bonded directly to the surface of the activated polymer material. In this case, the electrodes are preferably flexible and able to be compressed and expanded so that they may move along with the electro-polymeric material as it is caused to contract, relax and expand. Electrodes made out of flexible material, such as conductive rubber or compliant weaves of conductive material may be used to allow the activated polymer material the maximum range of motion. In some embodiments, flexible methods of attaching the electrodes to the surface of the electro-polymeric material are preferred, such as rubber cement, urethane bonding, or other flexible adhesives. Additional electrode embodiments and compliant electrode embodiments are described in U.S. Pat. No. 6,376,971 to Pelrine et al. entitled, “Electroactive Polymer Electrodes,” the entirety of which is incorporated herein by reference.

In yet another variation, the electrodes may be printed directly onto the surface of an activated polymer material, using a process such as silk-screening with conductive ink, or a reductive process such as is used in the production of printed circuit boards. In this variation, the conductive ink may need to expand and contract along with the movement of the activated polymer material. In order to achieve this, the electrode may be subdivided into regions to allow for gross motions, such as wavy lines or other geometric shapes. FIG. 45 shows patterns 210, 212 of conductive ink that would allow for large degrees of stretching and contracting. In this variation, it may also be desirable to print all connections needed to individually control any or all of the regions of electrodes, so that a large number of regions of activated polymer material may be controlled, thus reducing or eliminating the requirement for additional wiring, as shown in FIG. 46.

Controlling the voltage potential of each of the individually controllable electrodes effects the control of the shape of the pieces or regions of the electro-polymeric material used to control the shape of the articulating instrument. This may be done by use of a controller that switches each of the electrodes on or off, and controls the voltage at each of the electrodes individually to any desired voltage. This may be accomplished by use of a computer or other programmable controller. The controller will then be capable of actuating each individually controllable region, portion, or piece of electro-polymeric material of the endoscope. In this way, the shape of the entire length of the endoscope may be controlled in any way desired, including the “follow-the-leader” algorithm, as described above.

In yet another variation, a separate connection may be made between each of the individual electrodes and a controller. In this variation, a separate wire or pair of wires, or printed trace comprising a wire, may be used to connect each electrode to a controller, such as is shown in the schematic illustration in FIG. 46.

In yet another variation, a network of small controllers that are each capable of switching and controlling a smaller number of electrodes, such as would be required to actuate a single segment of an endoscope, are connected together to a main controller with a data network and a power network, as shown in FIG. 47. The main controller would then configure each of the segments individually by communicating the settings for each of the electrodes to each communications node on the network. This significantly reduces the number of connections that must be made from each electrode to the main controller of the endoscope. Additional controller are described in the incorporated Heim and Pelrine patents and applications as well as US Patent Application publication US 2003/0067245 to Pelrine et al. entitled “Master/Slave Electroactive Polymer Systems,” incorporated herein by reference.

In order to cause the segments, regardless of the variation of design selected, to actuate as quickly and responsively as possible, it may be beneficial to actively pull against regions of electro-polymeric material that have been caused to stop contracting and are in the process of relaxing. This has the benefit of decreasing the response time required for a segment to achieve a newly commanded position, as the time for a region or piece of electro-polymeric material to relax passively is longer than that required for the opposing piece or region of electro-polymeric material to pull the segment to the new required position. Using this algorithm, segments, joints or hinges are actively pulled into new positions, instead of allowing them to relax to achieve new positions.

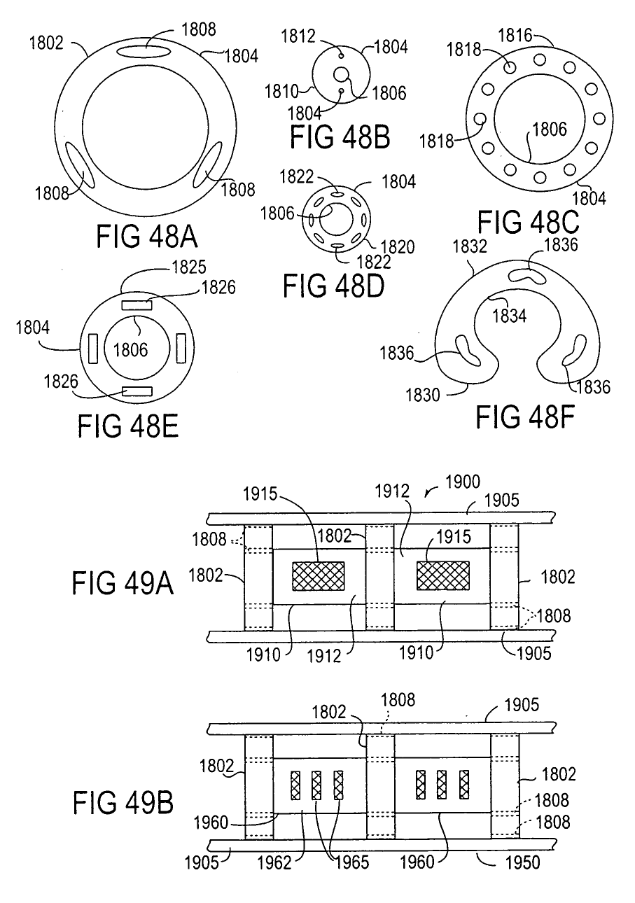

A number of alternative segment embodiments will now be described with regard to FIGS. 48A-48F. In some embodiments there is provided an articulating instrument having at least two segments, each segment having an outer surface and an inner surface and comprising at least two internal actuator access ports disposed between the outer surface and the inner surface. In addition, at least one electromechanical actuator extending through each of the internal actuator access ports and coupled to the at least two segments so that actuation of the at least one electromechanical actuator results in deflection between the at least two segments.

Segment 1802 is an example of an annular and continuous segment having an outer surface 1804 and an inner surface 1806 (FIG. 48A). Three internal actuator access ports 1808 are disposed between the outer surface 1804 and the inner surface 1806. The internal access ports 1808 have, in this embodiment, a generally oval or elliptical shape. Other shapes are possible. As will be described in greater detail below, embodiments of the internal access ports provide an attachment point between the segment and an activated polymer component such as an actuator, a rolled actuator, a sheet of activated polymer material having one or more active areas.

Segment 1810 is generally circular in shape and has an outer surface 1804 and an inner surface 1806 (FIG. 48B). Two internal actuator access ports 1812 are disposed between the outer surface 1804 and the inner surface 1806. The internal access ports 1812 have, in this embodiment, a generally circular shape.

Segment 1816 is generally circular in shape and has an outer surface 1804 and an inner surface 1806 (FIG. 48C). Twelve evenly spaced actuator access ports 1818 are disposed between the outer surface 1804 and the inner surface 1806 and about the circumference of the segment 1816. The internal access ports 1818 have, in this embodiment, a generally circular shape. The shape of each internal access port need not be the same for every port in a given segment and the ports need not be evenly arrayed about the segment. Some ports may be closer to the outer surface 1804 or the inner surface 1806 or two or more ports could be positioned along the same radius and distributed between the inner surface 1806 and the outer surface 1816. While these alternatives are described in relation to an embodiment of segment 1816, they apply as well to the other segment embodiments described herein.

Segment 1820 is generally circular in shape and has an outer surface 1804 and an inner surface 1806 (FIG. 48D). Eight actuator access ports 1822 are arrayed about the segment perimeter between the outer surface 1804 and the inner surface 1806. The internal access ports 1818 have, in this embodiment, a variety of generally oval shapes.

Segment 1825 is generally circular in shape and has an outer surface 1804 and an inner surface 1806 (FIG. 48E). Four actuator access ports 1826 are disposed between the outer surface 1804 and the inner surface 1806 about the circumference of the segment 1825. The internal access ports 1826 have, in this embodiment, a rectangular shape.

Segment 1830 is generally circular and, unlike the earlier segment embodiments, is non-continuous (FIG. 48F). Segment 1830 has an outer surface 1832 and an inner surface 1834. Three actuator access ports 1836 are disposed between the outer surface 1832 and the inner surface 1834 and about the segment 1830. The internal access ports 1836 have, in this embodiment, a compound geometric shape. In this embodiment, the compound geometric shape resembles the shape of a kidney bean. As described below, compound geometric shaped access ports may provide advantageous curvatures for sheets or sections or segments of activated polymer material. Segment 1832 also illustrates a non-annular or non-circular segment shape. Portions of the segment are flared to provide a more oval shape in some embodiments and in other embodiments the shape may resemble a flattened triangle or rounded conical shape.

It is to be appreciated from the above discussion of the various segments and access ports that at least one of the access ports in a segment has a regular geometric shape. In some embodiments, an access ports has a regular geometric shape selected from the group consisting of: circle, rectangle, oval, ellipse. In other embodiments, an access port may have a compound geometric shape. Additionally, the internal access ports could be of any shape, number, orientation and spatial arrangement with without uniform spacing. For example, in an embodiment where an embodiment of a segment is advantageously combined with a pre-bias shape instrument described above, the segment access ports may be distributed in a manner than recognizes the need for actuators to be positioned to counteract the pre-bias shape. In other embodiments, more than one activated polymer actuator or material is provided through, coupled to or terminated in an access port.

FIGS. 49A and 49B illustrate additional embodiment of activated polymer segments that may be used to articulate, bend or otherwise manipulate embodiments of the articulated instruments of the present invention. Articulating segment 1900 and 1950 share a similar construction. These are least two segments, each segment having an outer surface and an inner surface and comprising at least two internal actuator access ports disposed between the outer surface and the inner surface. The illustrated embodiments show segment 1802 with access ports 1808 it is to be appreciated that any of the other described segments or the like may also be used. The articulating segments also include at least one electromechanical actuator extending through each of the internal actuator access ports and coupled to the at least two segments so that actuation of the at least one electromechanical actuator results in deflection between the at least two segments. In one embodiment, the activated polymer actuator 1910 is attached to (i.e. terminates) the outer segments 1802 and passes through and is coupled sufficiently to the middle segment 1802 to allow deflection between each, any and/or all of the segments 1802. In the embodiment illustrated in FIG. 49A, the activated polymer actuator 1910 includes a polymer sheet 1910 and an active area 1915 including an electrode. The polymer sheet may be formed from an activated polymer that has only a portion used in the active area 1915. It is to be appreciated that rather than requiring an additional backing sheet of a different material, the activated polymer material could be used as the structural sheet 1912 used for the actuator.

In addition, a sheath 1905 is attached to the outer surface 1816 of the at least two segments. In an alternative embodiment, the sheath 1905 is attached to the inner surface 1806 of the at least two segments. In some embodiments, the sheath is formed from a suitable material known in the medical arts that is durable, flexible and washable so that it may be reused. In other embodiments, the sheath is removable from the segments and disposable. In yet another embodiment, the sheath material comprises a biocompatible material.

Articulating segment 1950 (FIG. 49B) differs from articulating segment 1900 in that multiple active areas 1965 are provided between segments 1802. Three active areas 1965 are shown in FIG. 49B. More are possible. Moreover, the active areas need not be evenly spaced nor aligned only along the longitudinal axis of the segments. In addition, for all embodiments of segments 1900, 1950, the structure of the active areas and the polymer sheets 1912, 1962 may include pre-strained and unstrained polymers, multi-laminated electrode structures, compliant electrodes, other structural elements to provide for the proper operation of an activated polymer actuator. For example, providing an electrolyte adjacent a conductive polymer type actuator.

While the segments depicted above are closed loops and open loops, the segments may also be used in combination with or replaced by tubes of various lengths if desired. For example, a series of short tubes constructed in a fashion similar to known vascular, biliary or esophageal stents can be used. Such a structure may include the placement of a plurality of actuators positioned between a series of short stent-like elements.

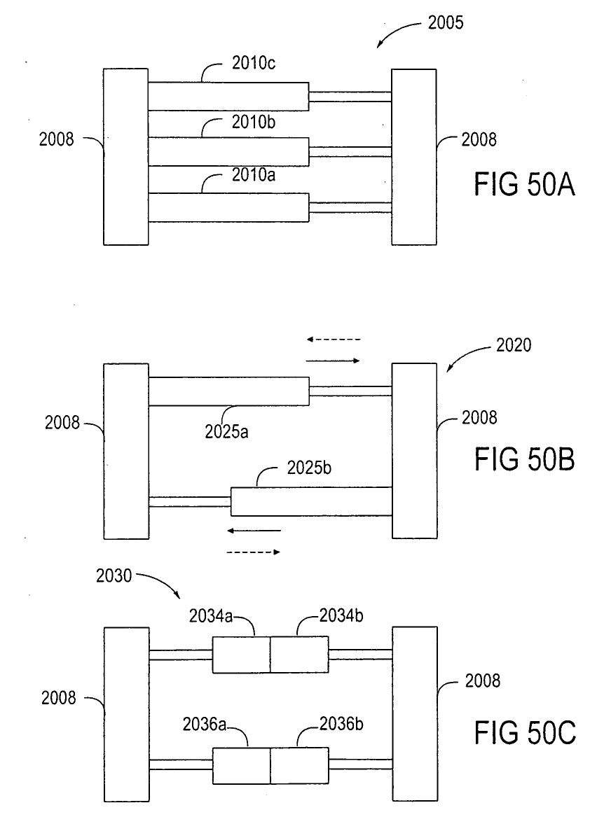

In some embodiments of the present invention, the articulating instrument is actuated, bent or otherwise manipulated using embodiments of the rolled polymer actuators described above. In general, the rolled polymer actuators are extended between a pair of segments 2008. In FIG. 50A, activated segment 2005 includes rolled polymer actuators 2010a, b, and c distributed between the segments 2008. Suitable electronic controls are provided allowing the actuators to be operated separately or in combination to produce the desired deflections between the segments 2008.

Activated segment 2020 includes a cooperative pair of rolled polymers actuators 2025a and 2025b (FIG. 50B). Rolled actuators 2025a, 2025b also illustrate how the potential applied to the actuator may be reversed to provide reversible operation. For example, the solid lines indicate application of positive potential and the dashed lines represent the application of negative potential. Suitable electronic controls are provided allowing the actuators to be operated using reversible actuation separately or in combination to produce the desired deflections between the segments 2008.

Activated segment 2030 includes an alternative embodiment of a cooperative rolled polymer actuator pair. Rolled actuator pairs 2034a, b and 2036a, b are disposed between segments 2008. In one embodiment, the segments 2008 may be manipulated or articulated by having the actuator 2034b push on its attached segment 2008 while the actuator 2034a pulls on its attached segment 2008. In another embodiment, both actuator pairs 2034a, b and 2036a, b are operating in the above described push-pull mode. In another embodiment, less than all the actuators are activated to deflect the segments 2008. Other alternative rolled activated polymer actuator configurations are possible. For example, the reversible aspect described in FIG. 50B may be applied to other embodiments, and combinations of actuator configurations 2010, 2025 and 2034 may be used between the same segment pair.

Further to the embodiments described in FIGS. 38, 39, 40, 41 and 42, a single elongated tube 2100 can be used as a structural element to form an embodiment of an articulating instrument of the present invention. In some embodiments, the design of the structure may also be in the form of a plurality of stent-like elements. In some embodiments, the elongate member 2100 is formed from a flexible or elastic material such that the member 2100 can be configured so that it will possess an inherent bias or memory such as discussed above in FIGS. 35E and 35F. The bias acts to restore the assembly to a substantially linear configuration as illustrated or into any desired bias shape as discussed above. Similarly, actuators coupled to the member 2100 can then be used to deflect it from an original or bias configuration as needed to reflect, for example, the shape of a lumen, organ or body cavity into which the articulating instrument is inserted. Of course, a source of bias such as an elastic sleeve (i.e., inserted within or about the structure as discussed above) may also be provided.

Connector Assemblies and Drive Systems for Segmented Controllable Instruments

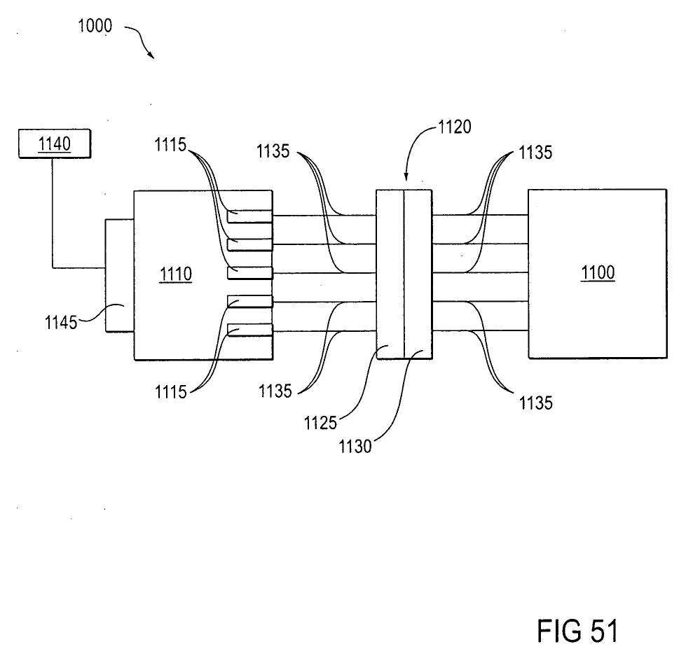

FIG. 51 illustrates a schematic view of a system 1000 for moving a controllable article 1100, a force generator under control of one or both of a user input device 1140 and a system controller 1145 generates forces that are used to move the controllable article 1100. The forces generated by the force generator are transmitted to the controllable article using force connecting elements 1135 and a connector assembly 1120. The controllable article may also be an articulating instrument

A connector assembly 1120 completes the transmission of power generated by the force generator 1110 and applied to the controllable article 1100. The two portions 1125, 1130 of the connector assembly 1120 are disengagably coupled. The connector portion 1125 is the first connector portion or the force generation side connector. The connector 1130 is the second connector portion or the controllable article side connector portion. When the connector portions 1125, 1130 are in a coupled condition, the force transmission elements 1135 are joined and force generated by the force generator 1110 is applied to the controllable article 1100. When the connector portions 1125, 1130 are not coupled, the connector portion 1130, force transmission elements. 1135 and the controllable article 1100 may be removed, in some embodiments as a single integrated unit, from the connector portion 1125, force transmission elements 1135 and the force generator 1110 or actuators 1115.

The connector assembly 1120 represents one advantage of the present invention. The ability to quickly connect and disconnect the two portions 1125, 1130 allows a single force transmission portion to be used with multiple controllable articles. Currently, articulating instruments such as, for example, endoscopes typically have only 4 cables to provide limited control at the tip of the endoscope. The present invention may be advantageously utilized by existing articulating instruments to allow endoscopes with only a few force transmission elements to be quickly and more readily connected to a force generator. Moreover, connector embodiments of the present invention provide compact organization and efficient coupling of numerous force transmission elements used by highly maneuverable controllable articles. As the degree of control exerted over controllable articles increases, the number of force transmission elements needed to exert that control also increases. Increasing numbers of force transmission elements drive the need for connector solutions such as those presented by embodiments of the present invention that afford a highly compact and organized coupling arrangement of the force transmission elements.

One advantage of the simplified connection/disconnection aspect of the present invention, is that in many instances it may be desirable to have the controllable article easily separable from the actuators, force generators or controllers for cleaning, disinfecting or maintenance. The quick-release characteristics of tee connectors of the present invention enable an efficient way to achieve a controllable article that is easily removable, replaceable or interchangeable. In this manner, a single controller and actuator system may be used to articulate multiple controllable instruments. After one instrument is released, another is quickly and easily connected and ready for service.

Another advantage of the connectors of the present invention is that the proximal ends of the force transmission elements attached to the controllable article can be organized to allow predictable attachment point to the corresponding force transmission elements coupled to the actuators. The plurality of force transmission elements may be organized into a bundle, array, or rack. Such organization provides a known attachment point between the force transmission elements of the actuators to the force transmission elements of the articulating instrument. Additionally, as will be seen in the examples that follow, dozens of force transmission elements will be utilized in advanced articulating instruments. Embodiments of the connectors of the present invention provides a scaleable solution that allows a user, in a single motion, to connect all the force transmission elements coupled to the actuators to those coupled to the controllable article. Moreover, the single action connection feature of some embodiments of the present invention also provides an important safely feature if an unsafe condition arises, the actuators or force generators may be quickly disconnected from the articulating instrument.

As will be detailed below, this organization could also provide other advantages to the controllable article such as allowing active or passive control of the tendon slack. Furthermore, the proximal ends of each tendon can be modified to allow attachment and manipulation, e.g., the ends of the tendons may be held in a specially configured sheath or casing.

Additionally, the connector 1120 may include sensors and/or safety features to help ensure proper operation and articulation of the controllable article. In the discussion that follows, the connector refers to embodiments of the connector 1120 as well as embodiments of the first and second connector portions 1125, 1130. One sensor or feature may indicate or detect translation or movement of the engaging elements (i.e., carriage assemblies 120 described below) or the force transmission elements 1135 themselves. Another sensor or feature may also detect and measure or otherwise quantify the amount of translation or movement of the engaging elements (i.e., carriage assemblies 120 described below) or the force transmission elements 1135 themselves. Another sensor may be utilized to indicate proper engagement of either the connector portions 1125, 1130 or each of the individual engaging elements (i.e., carriage assemblies 120). Another sensor or indicator may be used to generate a signal based on contacting a limit stop or the length of travel of a particular component. Yet another sensor may be used to detect component failure within the connector 1120.

Returning to FIG. 51, the system includes a force generator 1110. The force generator may be any conventional generator used to provide or generate sufficient force to the movement of the controllable article 1100. The force generator may provide, for example, mechanical force, hydraulic force, rotational force, or pneumatic force. Force generators may also utilize shape memory alloys (SMA) and/or electroactive polymers (EAP). Optionally, and illustrated in the embodiment of FIG. 51, the force generator 1110 may itself be an actuator or include a plurality of individual actuators 1115 acting as individually controllable force generators for each force transmission element 1135. Alternatively, an individual actuator 1115 may be connected to drive and control a subset of the total number of elements 1135 but more than one element. For example, an individual actuator may be connected to drive two, three, four or more individual force transmission elements. A plurality of first force transmission elements 1135 are illustrated having a first end connected to a force generator 1110, 1115 and a second end having a first connecting element within the connector portion 1125. Details of several illustrative connecting element embodiments are described in detail below.

The controllable article 1100 is connected to the connector portion 1130 by a plurality of force transmission elements 1135. The controllable article may be any of a number of commercial, industrial or medical devices. These force transmission elements have a first end connected to the controllable elements, modules or components within the controllable article. The controllable article may be, for example, a robotic handler having a number of articulating linkages. In this example, the force transmission elements 1135 attached to the connector 1130 are connected to transmit force to the articulating linkages. In another illustrative embodiment, the controllable article may be a segmented, articulating instrument. In this case, the force transmission elements 1135 attached to the connector 1130 will also be connected so as to transmit force to the individual segments to articulate the instrument. The ends of the force transmission elements 1135 within the connector 1120 are adapted to engage one another when the connector portions 1125, 1130 are coupled. In some embodiments, the first and the second elements are mechanically coupled. Other types of coupling configurations are possible and are described in greater detail below.

A controllable article 1100 includes at least one segment or module, and preferably several segments or modules, which are controllable via a computer and/or electronic controller (controller) 1140 located at a distance from the controllable article 1100. Each of the segments has force transmission elements 1135, tendons, mechanical linkages or elements connected to a force generator 1110 or an actuator 1115 to allow for the controlled motion of the segments or modules. The actuators driving the tendons (as a specific example of a force transmission element 1135) may include a variety of different types of mechanisms capable of applying a force to a tendon, e.g., electromechanical motors, pneumatic and hydraulic cylinders, pneumatic and hydraulic motors, solenoids, shape memory alloy wires, electroactive polymer actuated devices, electronic rotary actuators or other devices or methods as known in the art. If shape memory alloy wires are used, they are preferably configured into several wire bundles attached at a proximal end of each of the tendons within the controller. Segment articulation may be accomplished by applying energy, e.g., electrical current, heat, etc., to each of the bundles to actuate a linear motion in the wire bundles which in turn actuate the tendon movement. The linear translation of the actuators within the controller is configured and scaled in conformity with the desired movement of the controllable article and may vary depending upon application of the controllable article. Some commercial applications may include controllable articles articulating in large movements measured in feet Still other applications, such as for example, medical applications, may find that the controllable article is configured for tighter control to enable more precise movement over a relatively short distance, e.g., within a few inches or less such as .+-.inch, to accomplish effective articulation depending upon the desired degree of segment movement and articulation.

In one specific embodiment, the force generator is a motor. The motor is coupled to a leadscrew assembly, so that when the motor rotates, it transmits torque to the leadscrew. A modified nut on the leadscrew is constrained to prevent rotational motion, so that when the leadscrew is rotated, the nut is translated along the axis of the leadscrew. The torque from the motor is thereby translated into linear motion. In this specific embodiment, the force transmission element is a cable that is connected to the nut on one end and a carriage assembly 120 on the other end. The linear motion of the nut translates into force on the cable. As such, the leadscrew movement is translated into linear movement of a carriage assembly in one connector hence to another carriage assembly in another connector assembly connected to the controllable article. In one specific embodiment, 64 of the leadscrew assemblies are arranged in modules for easy organization and maintenance. The modules are supported in a chassis that also houses the first portion of the connector described above. More or fewer leadscrew assemblies may be used depending upon application.

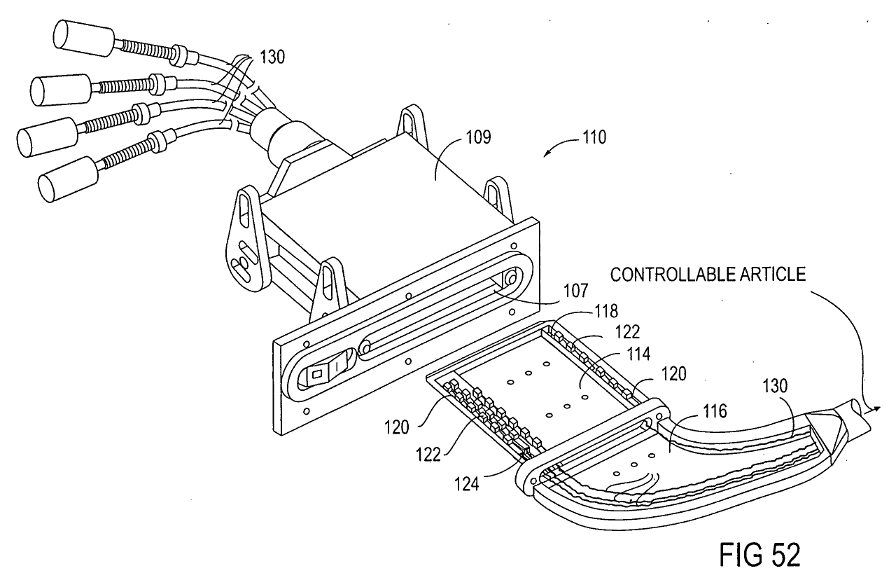

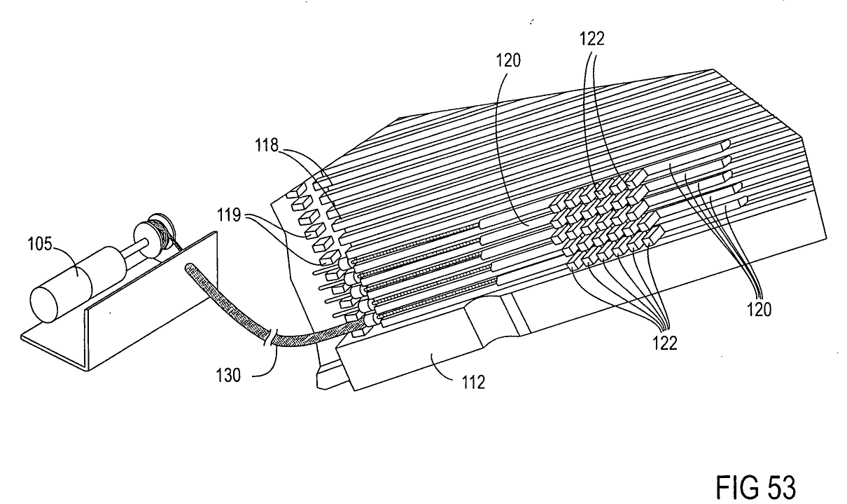

FIG. 52 illustrates a perspective view of a connector assembly 110 according to one embodiment of the present invention. The connector assembly 110 includes a first connector portion 112 (not shown but within housing 109) and a second connector portion 114. The first connector portion 112 is positioned within the housing 109. The second connector assembly 114 includes a plurality of guideways 118 each containing a carriage assembly 120. Each carriage assembly contains one or more than one engaging feature 122. Engaging features 122 on carriage assemblies 120 in the second connector portion 114 are adapted to engage with the engaging features 122 on carriage assemblies 120 of the first connector portion 112 (see FIG. 53). One end of the carriage assemblies is connected to force transmission elements or cables 130. In the illustrated embodiment, the cables are Bowden cables. The cables run through a slack area 116. The slack area 116 allows added space for cable slack that may build up during controllable article movement. Thereafter, the cables are connected as desired to the controllable article.

The housing 109 provides a structural base for supporting the connector assembly 110. In this embodiment, the first connector portion 112 (not shown) is secured within the housing 109. The first connector portion and its carriage assemblies are connected via force transmission elements 130 to actuators 105. While four actuators 105 are illustrated, it is to be appreciated that more actuators may be used to drive a corresponding number of carriage assemblies. The housing 109 also provides an opening 107 configured to receive the second connector portion 114. Optionally, either one or both of the opening 107 or a portion of the second connector portion 114 may be keyed to ensure correct orientation prior to connection. When the second connector portion 114 is placed within the opening 107, the first and second connector portions 112, 114 are brought into engagement using an appropriate quick release mechanism, such as for example a cam actuated lever or other engagement device as known to those of ordinary skill in the art. When the first and second connector portion 112, 114 are engaged, forces generated by actuators 105 are transmitted to the controllable article. In one embodiment, relative movement between the first connector portion and the second connector portion is used to couple the first connector portion to the second connector portion. In one embodiment, nearly vertical movement between the first connector portion and the second connector portion is used to engage the first and second connector portions. In another embodiment, the coupling force between the first and second connection portions acts nearly orthogonal to the direction of movement of the individual connection elements (i.e., carriage assemblies 120) within the first and second connection portions.

The connector 110 embodiment of FIG. 52 and other embodiments of the present invention may also provide a number of safety features. For example, the forces used to bring together and hold the carriage assemblies of the first and second connector portions in locking cooperation may be set to such a level that ensures these is positive engagement and no slip. The force holding the connector portions could be set to separate at a force below a threshold force that could damage a component in the force transmission pathway, such as for example, a force transmission element. Alternatively, carriage assemblies could be attached to their force transmission elements at some margin of safety whereby in the event an actuator loses control, the respective carriage assembly would separate from its force transmission element.

FIG. 53 shows an embodiment of the first connection portion 112 coupled to an actuator 105. The first connector portion 112 is constructed similar to the second connector portion 114 described above. As such, the first connector portion 112 includes a plurality of guideways 118 and carriage assemblies 120. Instead of connecting to the controllable article, the carriage assemblies 120 of the first connector portion 112 are appropriately connected to actuators 105.

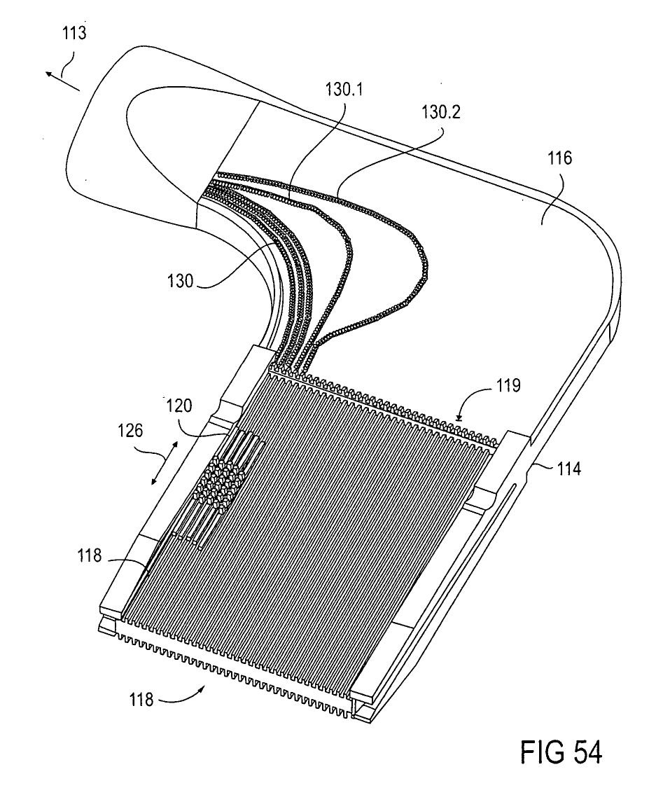

FIG. 54 shows a perspective view of one embodiment of the second connector portion 114. The second connector portion 114 organizes and houses the carriage assemblies 120 within guideways 118. By connecting the force transmission elements to the carriage assemblies, this organization is provided to the plurality of force transmission elements needed in highly controlled articulating instruments and controllable articles. In the illustrated embodiment, the second connector portion provides 64 guideways with 32 guideways in the upper face 114A and 32 guideways in the lower face 114B (the edge of the guideways 118 of the lower face 114B are visible). The embodiment of FIG. 54 illustrates the compact nature of connectors according to the present invention. Because of the highly efficient space utilization, a connector of the present invention may provide articulation force to 64 separate cables in a space only slightly larger than the width of 32 cables or one-half the total number of cables. Alternatively, the width of the connector is only slightly larger than the width of a single carriage assembly multiplied by one-half the number of force transmission elements.

It is to be appreciated that both double and single sided connector portions are possible. For example, the double-sided second connector portion may be coupled to two single sided first connector portions (i.e., one single sided first connector engages with the second connector upper face and the other engages with the lower face. Many different connector shapes and configurations are possible. For example, in another alternative configuration, two double-sided second connectors 114 may be engaged by one double sided first connector portion 122 between the double sided second connectors 114 and a it single sided first connector above one and a second single sided first connector below the other second connector portion 114. In each of these alternatives, the mechanical workings within the housing 109 provide proper alignment and quick disconnect between the various connector portions regardless of the numbers used

The connectors and housing 109 may be formed from any suitable material having sufficient strength to transmit the forces or energy used. Suitable materials include metals, plastics, extrusions, injection molded parts, forged, and/or metal injection molded parts. In addition, the bearing surfaces may be coated with suitable low friction coatings to reduce friction losses within the connectors such as between the carriage assemblies and the guideways. One or more surfaces within the connector assembly may be coated as desired Suitable coatings include, for example, Teflon, PTFE, and other low friction coatings. In addition, the bearing surfaces may include a viscous coating or include other bearing structure or surfaces such as, for example, ball bearings, linear bearings, or air bearings and the like.

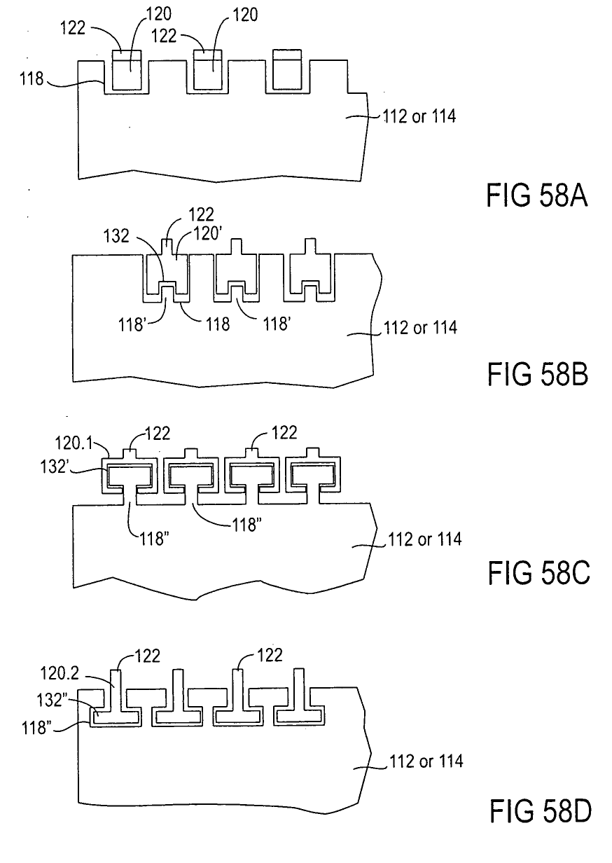

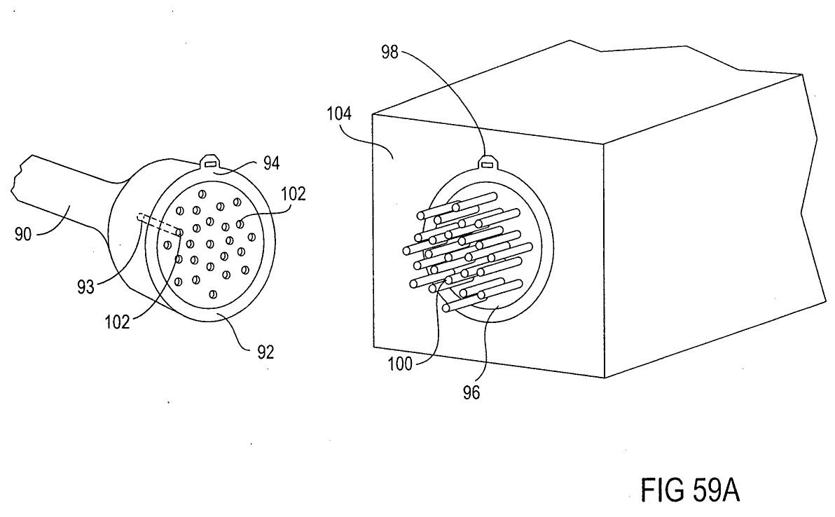

Connector assembly portion 114 has a plurality of guideways 118 for organizing the array of tensioning-members and/or cables 121 used to control a controllable article. Guideway 118 may be a U-shaped channel formed integrally within housing 114 as illustrated or it may be manufactured separately and attached onto housing 114. As described in greater detail below with regard to FIGS. 58A-58D, embodiments of the guideway 118 may comprise tracks or rails aligned adjacent to each other. In some embodiments, each of the rails may form a protrusion extending along the length of guideway 118 such that a rectangularly shaped-rail is formed. The rail or track may be of any shape such as, for example, rectangular, concave, convex, rounded or curvilinear. A complementary shape is formed in the engaging face of the carriage assembly for that guideway. The number of rails may correspond to the number of tensioning members utilized for a controllable device or more rails may be provided to accommodate for additional tensioning members. In some embodiments, the rails align parallel to one another although in other variations, the shape and alignment of the rails may be varied.

As illustrated in FIG. 53 and 54, connected to at least one of the tensioning members and/or cables 121 are cable carriage assemblies 120. There may be one or several carriage assemblies 120 configured to traverse along the guideway 118. As in these illusive examples, each carriage assembly 120 may be secured to a cable 121 extending from a tensioning member or force transmission element. As best seen in FIG. 53, the cable 121 passes through the cable stop 117, the coil tube 111 and is suitably coupled to the actuator 105. In the illustrative embodiment of FIG. 53, the cable 121 is wrapped about the end of the actuator 105. The cable stop 117 is anchored in the gap between the frame stop 119 and the end of the guideway 118. The ends of the coil tube 111 are anchored between the actuator fame or a support 115 and the cable stop 117. Similar to a conventional Bowden cable arrangement, the above described anchoring configuration retains the coil tube 111 in compression while the cable 121 remains in tension and transmits force from the actuator 105. As best illustrated in FIG. 54, the carriage assembly 120 is moved within the guideway 118 as indicated by the direction of travel 126. A force transmission element (i.e., 130, 130.1, 130.2) attached to a carriage assembly 120 and the controllable article 1100 transfers that motion 126 to move the controllable article 1100 accordingly. The number of carriage assemblies 120 utilized will vary depending upon the number of tensioning members utilized for articulation of the controllable article.

Guideway 118 may be configured to provide a limited range of travel for the translational movement of cable carriage assemblies 120. For instance, guideway 118 may have a frame stop 119 defined at one end of the guideway 118 so that carriage assemblies 120 may be securely seated and aligned with each rail. Frame stop 119 may define a portion of the guideway that is discontinuous such that a carriage assembly 120 may be seated within the discontinuity. Although the discontinuity is shown in FIG. 54 at one end of the guideway 118, it may alternatively be positioned at the other end of in another location along the guideway 118. Alternatively, frame stop 119 may define a crimp, clamp, adhesive, mechanical fastener, or some other method as known in the art for securing to prevent the excess movement of carriage assembly 120.

In the illustrated embodiment of the second connector portion 114, the second connector portion 114 includes a cable passageway or slack area 116. Slack area 116 is an area sufficiently spacious to allow for the inclusion of slack in the tendons and/or cables which may be routed through and/or bend within the passageway 116, as described in further detail below. The passageway 116 may be curved such that controllable article interface 113 and guideway 118 are angled relative to one another, such as the illustrated angle of about 90.degree. but may range between 0.degree. to 180.degree. The slack area angle is measured between a line representing the direction of movement of the carriage assembles—i.e., direction of travel 126—and a line directed towards the articulating instrument through interface 113. The size and exact configuration of the slack area, if included, will depend upon the number size, shape and flexibility of the force transmission elements used in a particular application. As such, the slack area may have any of a wide variety of shapes or curvature to provide an accommodation for the excess or slack cable length temporarily created during movement or manipulation of the controllable article.

In the illustrated embodiment of FIG. 54, the force transmission elements 130, 130.1 and 130.2 are Bowden cables (i.e., cable 121 within a flexible housing or coil tube 111). As the controllable article is manipulated by movement of the cables, the cable housings for the cables may be moved longitudinally proximally and/or distally as well. The slack area 116 is shaped and sized to accommodate a number of tensioning elements in the connector assembly. The slack area 116 may simply be a compartment sufficiently large enough to provide space for the cables 130, 130.1, and 130.2, for example, to extend in an expanded configuration, to allow for cable slack in the connector 110. The force transmission elements 130, 130.1, and 130.2 illustrate the relative amount of space required for coil tube and cable extension. Illustrated are various degrees of extension from low extension in force transmission element 130 to moderate and high degrees of extension in force transmission elements 130.1 and 130.2 respectively. Where a slack area 116 is utilized, the relationship between the connector assembly portion and the slack area need not be angled, as illustrated, but may instead provide for a collinear arrangement.

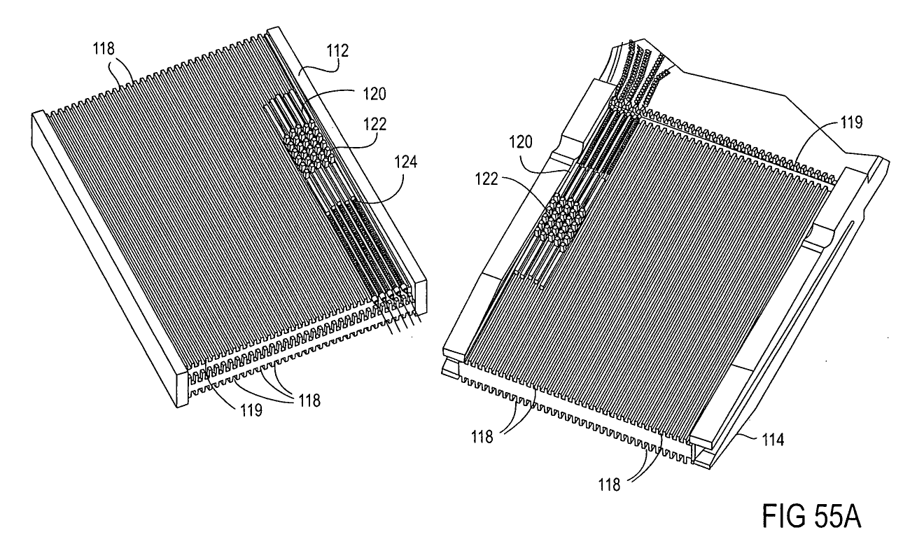

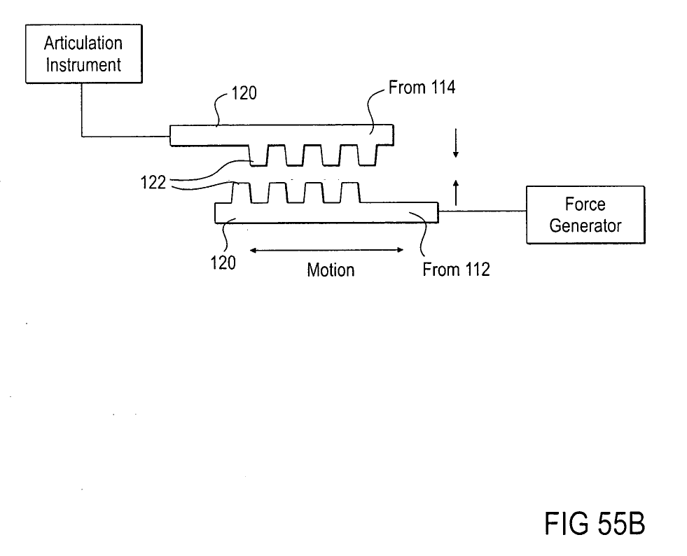

FIGS. 55A and 55B illustrate one manner of engagement between the carriage assemblies 120 of the connector portions 114, 112. When the two connector portions are brought together, the carriage assemblies 120 arranged on one side of a double sided first connector portion 112 align with and engage the carriage assemblies 120 arranged on one side of a double sided second connector portion 114 (FIG. 55A). One possible engagement between the features 122 of the carriage assemblies 120 is illustrated in FIG. 55B as the carriage assemblies are moved in the direction of the arrows. While FIG. 55B illustrates both connectors 112, 114 moving together to join one face, it is to be appreciated that the connectors may engage through relative movement in another direction. (i.e., lateral or circular movement, for example) and that one connector may remain fixed while the other moves to engage and that when engaged, the connectors engage on more than one face.

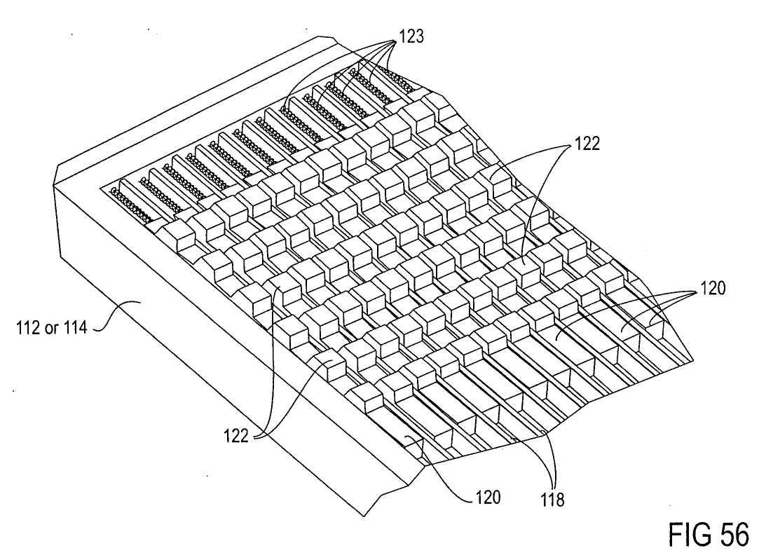

One potential problem when engaging connector portions 112, 114 is the proper alignment of the carriage assemblies prior to engagement. Any number of mechanical alignment features and techniques may be used to align the carriage assemblies into a zero or alignment position prior to engagement between a first and a second connector portion. FIG. 56 illustrates one embodiment where carriage assembly alignment is provided by placing an alignment element 123 adjacent a car e assembly. In this manner, each carriage assembly is urged into a similar position within a guideway 118. In the illustrated embodiment the alignment element may be a bias element such as spring. Alternatively, a small alignment feature may be provided in each guideway to temporarily engage a carriage assembly. The initial application of force applied to the connector after engagement is used to move the carriage assemblies off the alignment feature in preparation for articulating the controllable article. In one embodiment, each connector portion 112, 114 includes an alignment feature that urges the carriage assembly into an alignment position. An alignment position is a position of a carriage assembly in a guideway of one connector portion to further the engagement of that carriage assembly to a similarly positioned carriage assembly on the other connector portion.

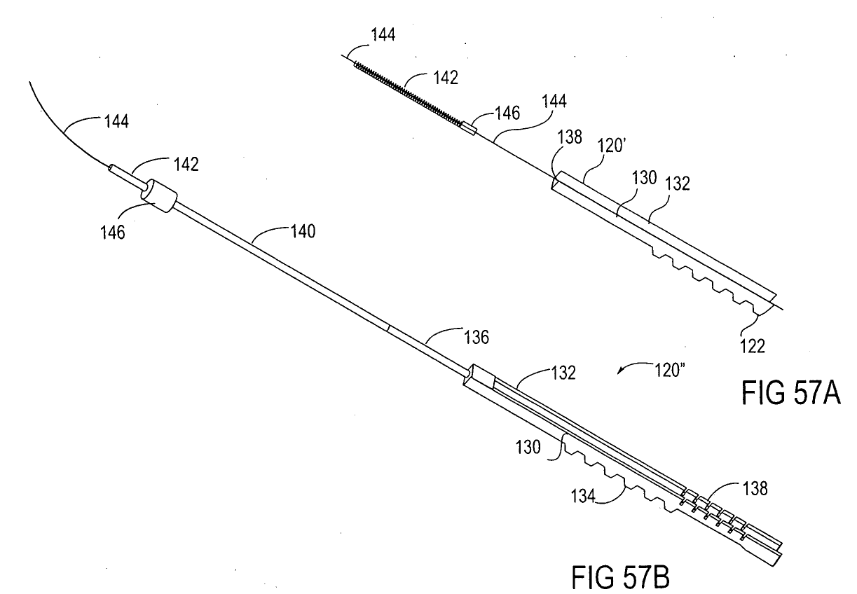

FIGS. 57A and 57B show detailed perspective views of two alternative carriage assembly embodiments 120′ and 120″. Carriage assemblies 120′, 120″ provide a rack 130 configured to correspondingly fit and slide along a rail disposed within or other feature configured within a guideway 118. In the illustrated embodiments, rack 130 defines a U-shaped or generally rectangular channel 132.

FIG. 57A illustrates an embodiment of a rack 130 having a channel 132. One end of a force transmission cable 144 is crimped 138 or otherwise fastened with adhesive, soldered, etc. to the rack 130. The cable 144 extends through stop 146 and through coil tube 142. Cable 144 may extend beyond stop 146 and the other end connected, for example, to a force generator or to an articulating segment of an articulating instrument. A coil tube 142 may optionally extend beyond assembly stop 146 to provide support between the cable 144 and stop 146 interface to aid in preventing cable 144 from kinking. Once the rack 130 is placed within an appropriately configured guideway 118 (i.e., one having a complementary shaped rail or feature to engage channel 132), the stop 146 is held within assembly stop 119 in connector assembly first portion 112 or second portion 114.

FIG. 57B illustrates an embodiment utilizing a telescoping tube 140, 136. Inner tube 136 extends within the interior of telescoping tube 140 in a slideable arrangement Inner tube 136 may be attached within channel 132 at crimps 138 or, alternatively, to the rack 130 with adhesive, solder or other fastening techniques known in the art. One end of telescoping tube 140 may terminate in stop 146, which may be positioned within or adjacent to frame stop 119. Extending from assembly stop 146 is cable 144, which may further extend and be connected to a force generator or a portion of an articulating instrument. Alternatively, cable 144 may further extend from assembly stop 146 directly into a segment of an articulating endoscope. A coil tube 142 may extend partially beyond assembly stop 146 to provide support between the cable 144 and stop 146 interface to aid in preventing cable 144 from kinking or to conventionally assist in the transmission of force. Optionally, one end of cable 144 may be secured to the distal end of inner tubing 136 or cable 144 may be disposed through inner tubing 136 and extend towards the proximal end of inner tubing 136 for direct attachment to rack 130 using a crimp 138. During operation as carriage assembly 120″ is translated, rack 13.0 rides along a rail in guideway 118 while inner tubing 136 slides through telescoping tube 140 relative to the stationary assembly stop 146. The distal and/or proximal movement of rack 130 will likewise urge cable 144 to move in accordance with rack 130, thereby transferring the longitudinal motion either directly or indirectly to the articulating instrument segment or portion attached to the cable 144.

Also shown in FIG. 57B is an interface portion 134 upon at least one of the outer surfaces of rack 130 to provide for a secure engagement interface with an actuator, e.g., electric motor, shape-memory alloy actuator, hydraulic or pneumatic actuator, etc. Interface portion 134 may be formed into a series of gear-shaped ridges, as shown, to provide engagement surfaces against a corresponding member attached to the actuator; alternatively, interface portion 134 may be configured to have a receiving clamp or slotted interface to provide for engagement or any other type of engagement interface as known in the art.

Although the embodiments of FIGS. 57A and 57B illustrate a rectangular or generally U-shaped channel 132, other configurations of the channel and corresponding rail are possible. The sliding channel 132 may also be formed in a variety of open shapes, such as semi-spherical, semi-elliptical, etc., provided the rail upon which rack 130 moves upon is formed in a corresponding shape. Examples of alternative rail and channel configurations are illustrated in FIGS. 58A-58D.

FIGS. 58A-58D illustrate alternative guideway and carriage assembly arrangements. Note that the carriage assembly/guideway arrangements described are applicable to either or both of the first and second connector assemblies 112, 114. FIG. 58A illustrates the carriage assembly 120 guideway 118 arrangement of FIGS. 53, 54 and 55A. The carriage assembly 120 shape is accommodated by the shape of guideway 118.

FIG. 58B illustrates one embodiment of a guideway 118 configured to accept a carriage assembly 120′ or 120″ as described above in FIG. 57A and 57B. The guideway 118 includes a feature or rail 118′ configured to cooperate in a sliding arrangement with the channel 132. FIG. 58C illustrates an alternative embodiment where the guideway is a raised feature 118″ adapted to engage with a complementary shaped channel 132′ in carriage assembly 120.1. FIG. 58D illustrates another alternative embodiment where the carriage assembly 120.2 includes a shaped feature 132″ adapted to slide along within the recessed, shaped guideway 118′″. In all these variations, the arrangement and shape of the complementary surfaces of the carriage assembly embodiment and the guideway embodiment are illusive and are not intended to be limited to the examples described herein. Rather, the specific shapes used to interface the carriage assembly to the guideway may be varied accordingly as understood by one of skill in the art.

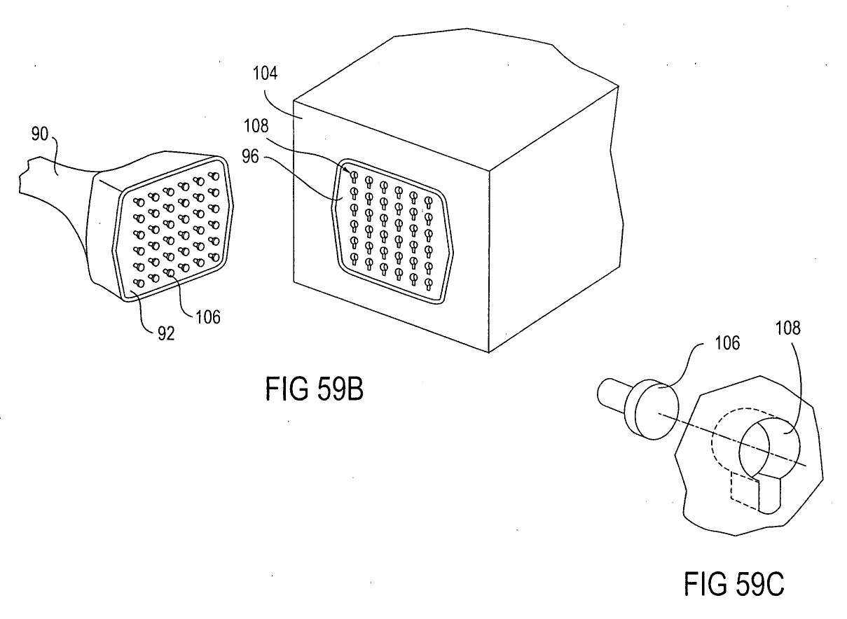

FIGS. 59A and 59B show two variations of quick-release mechanisms for attaching and detaching an articulating instrument from a set of actuators or force generators. FIG. 59A shows one variation of this quick-release, mechanism. The proximal end of the force transmission elements may be bundled in an umbilicus 90, and the individual elements may terminate in dimpled connectors 102 that are held in an organized array in a connector interface 92. For clarity only a single force transmission element 93 is shown (in phantom) within a connector 102. It is appreciated that each connector 102 could have a force transmission element 93 to mate with a corresponding pin 100. The connector interface 92 mates to a complementary receiving interface 96 on the structure that houses the actuators 104, e.g. as part of the controller box. The actuators may project “pins” 100 which can mate with the dimpled connectors and convey force from the actuators to the tendons. Thus, for example, an actuator may cause a pin 100 to apply pressure to a corresponding dimpled receiver 102. The dimpled receiver translates the pushing of the pin into a tensile or compressive force applied to the affiliated force transmission element. This could be achieved using levers to reverse the direction of the force, for example. Since every pin preferably mates to a corresponding receiver, it is desirable to maintain the register of the connectors from the endoscope and the actuators. An orientation notch 94 on the connector that fits into a receiving orientation mate 98 on the actuator could be used to align both interfaces. Alternatively, the arrangement of the pins and receptacles could be orientation specific.

This feature is not limited to pins and receptacles, since virtually any convenient mechanism for transferring force from the actuator to the force transmission elements would work. FIG. 59B shows another variation of a quick-release mechanism for attaching and detaching an articulating instrument from the actuators that relies on a nail-head configuration to actuate the force transmission elements. The force transmission elements may terminate in a flattened out protrusion resembling a nail-head 106. The array of nail-heads 106 project from the connector interface 92 at the end of the umbilicus 90, and can mate with slotted holes 108 on the interface 96 of actuator mechanism 104 (FIG. 59C). Thus the slotted holes 108 of the actuators can be individually retracted by the actuators to apply tension to individual force transmission elements. The quick-release mechanism could also be designed to allow the use of different controllable instruments, even of different configurations, from the same actuator set and/or controller unit.

As will be described in greater detail below, embodiments of the transluminal systems and methods described herein may be used to access numerous portions of the body and do so with a wade variety of mapping and imaging systems.

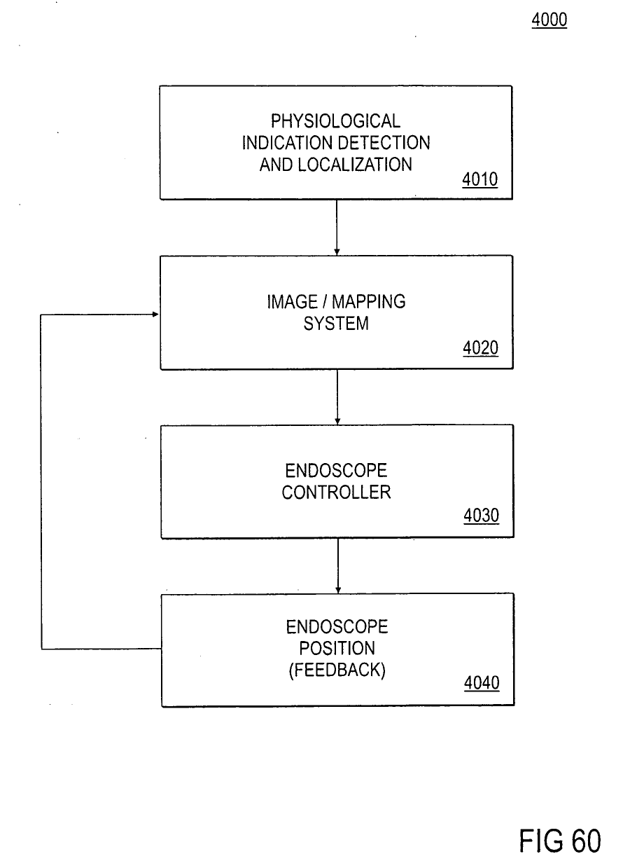

FIG. 60 shows the interaction of several components to provide a method of positioning a steerable endoscope system to facilitate treatment. As mentioned above, the movement, position, tracking and control of endoscopic devices according to the present invention is performed by a user alone or in cooperation with any or all of imaging systems, position and location systems, and surgical planning methods and techniques. The system schematic 4000 illustrates one embodiment of an integrated detection, mapping and control system for positioning and controlling a steerable, controllable endoscope of the present invention. First, a suitable device, element or system is used to detect and localize a physiological indication (4010). A physiological indication could be any perceptible indicia of a condition for which treatment may be facilitated. In a coronary example, physiological indicators include electrophysiology data or electrical signals from the heart. This system would be capable of identifying or performing analysis of monitored data to identify or determine the location of errant activity.

Next, information regarding the detected and localized physiological indication is passed to an image/mapping system (4020). An image/mapping system includes any imaging modality that may provide position, location, tissue type, disease state, or any other information that facilitates correlating the physiological activity to a identifiable and/or localizable position within the anatomy or within a frame of reference. Examples of image/mapping systems include any of the imaging technologies such as x-ray, fluoroscopy, computed tomography (CT), three dimensional CAT scan, magnetic resonance imaging (MRI), and magnetic field locating systems. Examples of image/mapping systems specifically suited for the treatment of cardiovascular disorders include electrocardiogram detection systems, cardiac electrophysiology mapping systems, endocardial mapping systems, or other systems and methods that provide the ability acquire, visualize, interpret and act on cardiac electrophysiological data. An example of such a system is described in U.S. Pat. No. 5,848,972 entitled, “Method for Endocardial Activation Mapping Using a Multi-Electrode Catheter” the entirety of which is incorporated herein by reference. Additional examples are described in U.S. Pat. No. 5,487,385; U.S. Pat. No. 5,848,972; and U.S. Pat. No. 5,645,064, the entirety of each of these patents is incorporated by reference. Integrated mapping, detection and/or ablation probes and devices may also be delivered using the steerable endoscope of the present invention. One such integrated system is described in US Patent Application Publication US 2003/0236455 to Swanson et al the entirety of which is incorporated herein by reference. Additional other systems may provide mapping, display or position information of a local isochronal activation map of the heart along with the relative position of the endoscope and direction information or movement commands to position the endoscope (or components, elements or systems onboard the endoscope) to provide treatment to the source of the arrhythmia.

Next, information provided, compiled and/or analyzed in the prior steps or other additional information provided by a user or other system used by the user is input into or utilized by the endoscope controller (4030). This step indicates the ability of the endoscope controller to respond to the indication, position, image, mapping and other data and utilize that data for altering the scope configuration, position, orientation or other relational information indicative of the scope controller responding to the information provided. The endoscope is configured to provide of facilitate providing components, elements or systems to facilitate a treatment of the physiological indication being monitored. The controller utilizes the data provided to position the steerable, controllable endoscope into a position related to the location or site that exhibits the errant activity. The proximity of the endoscope to the location or site of the errant activity will vary depending upon, for example, the treatment being implemented, the element, component or system being used to facilitate treatment.

Finally, the position of the endoscope is supplied back into the image or mapping system as a form of feedback to better assist in guiding the endoscope into the desired position to facilitate treatment (4040).

In another embodiment, the system 4000 may include an overall mapping system that provides medically significant data that facilitates a treatment. This overall mapping or imaging system may include mapping or imaging an area of monitored activity. The area of monitored activity includes not only the portion of the body important to the treatment but also imaging information of those other parts of the body not impacted by the treatment but are instead the likely pathway(s) of the steerable, controllable endoscope to reach the area where the treatment will be facilitated. In addition, some embodiments of the system may include the ability to detect, localize or otherwise indicate the position of the treatment area or area of errant activity or conditions subject to treatment. These indications may then be utilized to augment the guidance of the steerable, controllable endoscope into the desired position to facilitate treatment. In addition, other medical imaging and tracking systems may be utilized to provide tracking, guidance and position feedback information to the control of the steerable endoscope. An exemplary system is described by Dumoulin et al. in U.S. Pat. No. 5,377,678 which is incorporated herein by reference in its entirety.

The above steps are only representative of one embodiment of how physiological indications, and position information may be utilized to improve the guidance system and controls used by steerable endoscopes to ensure the placement of the endoscope to facilitate treatment. It is to be appreciated that the steps were utilized for clarity and ease of discussion. The methods of embodiments of the invention are not so limited. For example, a single system could be used as an integrated indication, imaging, endoscope controller that receives endoscope position feedback in real time. In an alternative example, the physiological indication and image/mapping functions may be combined into a single unit. As such, while the above steps have been described as happening only once or in a serial fashion, it is to be appreciated that the steps may be conducted in as different order or multiple times. Other physiological indication detection and localization systems may be used and will correspond to an appropriate system useful in the treatment being performed. In addition, alternative image and mapping systems may also be employed and may also be selected depending upon the treatment being facilitated through the use of a steerable controllable endoscope of the present invention. The system may also control the movement of the endoscope automatically based on inputs from the user, pre-surgical planning data, or other indications of desired pathways or pathways to avoid. Alternatively, or in addition, a user may input additional guidance or control information into the system for furthering the guidance or desired placement of the endoscope.

Other endoscopic devices may also be used to enhance transluminal systems and methods.

FIG. 61 is a cutaway drawing illustrating a steerable colonoscope 100 with a colectomy device 102 mounted thereon being inserted through the lumen of a patient's colon. As mentioned before, the same technique may apply for every other tubular shaped organ. Preferably, the steerable colonoscope 100 is constructed as described in U.S. patent application Ser. Nos. 09/790,204 (now U.S. Pat. No. 6,468,203); 09/969,927; and 10/229/577, with multiple articulating segments that are controlled to move with serpentine motion that facilitates insertion and withdrawal of the colonoscope with a minimum of contact and stress applied to the colon walls. Additional details and various embodiments of the steerable colonoscope 100 are described below with reference to FIGS. 28-33. In addition, the control system of the steerable colonoscope 100 has the capability to construct a three-dimensional mathematical model or map of the colon as it advances through lumen under control of the operator. The three-dimensional mathematical model of the colon and the location and nature of any lesions identified in the course of an initial colonoscopic examination can be stored and used in performance of the endoscopic colectomy procedure. In alternate embodiments, the colectomy device 102 of the present invention may be mounted on a colonoscope of a different design and construction.

The colectomy device 102 can be permanently or removably mounted on the steerable colonoscope 100. The colectomy device 102 has a distal component 104 and a proximal component 106. The distal component 104 and the proximal component 106 each have an expandable member 108 and a gripping mechanism 110 for gripping the wall of the colon. The expandable member 108 may be an inflatable balloon or a mechanically expandable mechanism. The gripping mechanism 110 may comprise a plurality of circumferentially located ports within which attachment points 112, e.g., needles, hooks, barbs, etc., may be retractably positioned about an exterior surface of the expandable member 108. Alternatively, the gripping mechanism 110 may utilize a vacuum gripper through a plurality of circumferentially located ports around the distal component 104 and/or the proximal component 106 or other known gripping mechanisms. In the case of the vacuum gripper, gripping mechanism 110 is in fluid communication through the ports and through the colonoscope 100 to the proximal end of the colonoscope 100 to a vacuum pump (not shown). At least one, and optionally both, of the distal component 104 and the proximal component 106 are movable longitudinally with respect to the body of the steerable colonoscope 100. Rails, grooves or the like 114 may be provided on the body of the steerable colonoscope 100 for guiding the longitudinal movement of the distal component 104 and the proximal component 106.

In addition, the colectomy device 102 includes a surgical stapler 116 or other anastomosis mechanism. The surgical stapler 116 is carried on either the distal component 104 or the proximal component 106 and a stapler anvil 118 is carried on the other of these components. The surgical stapler 116 may be configured similarly to any number of conventional stapling devices which are adapted to actuate staples into tissue. Another option is that there is a stapler and an anvil on both components for stapling and sealing the edges. Optionally, the colectomy device 102 may include a cutting device and/or electrocautery and/or a laser device for transecting the colon wall. Optionally, the colectomy device 102 may also include a vacuum mechanism or the like for drawing the excised tissue into the colectomy device 102 for later removal along with the steerable colonoscope 100.

FIG. 61 shows the steerable colonoscope 100 with the expandable members 108 of the distal component 104 and the proximal component 106 in a contracted or deflated condition for easy passage through the lumen of the patient's colon. The control system of the steerable colonoscope 100 monitors the position of each segment of the colonoscope 100 as it is advanced within the colon and can signal to the operator when the segments carrying the distal component 104 and the proximal component 106 of the colectomy device 102 are correctly positioned with respect to a previously detected lesion in the colon. Alternatively, the control system of the steerable colonoscope 100 can be programmed to advance the colonoscope 100 automatically through the lumen of the colon and to stop it when the distal component 104 and the proximal component 106 of the colectomy device 102 are correctly positioned with respect to the lesion in the colon. Alternatively, the control system will be able to automatically guide and deliver the two components to the desired location after the colonoscope has been inserted to the colon.

FIG. 62 is a cutaway drawing showing the expandable members 108 of the distal component 104 and the proximal component 106 of the colonoscope-mounted colectomy device 102 expanded within the lumen of the colon so that the gripping mechanism 110 grips the wall of the colon. The distal component 104 and the proximal component 106 may be expanded through any number of expansion devices. For instance, they may be radially expanded upon spoke-like support structures or they may be configured to radially expand in a rotational motion until the desired expansion diameter is attained. At this point, with the diseased portion of the colon identified and isolated by the colonoscope-mounted colectomy device 102, the diseased portion is separated from the omentum and the blood vessels supplying it are ligated and/or cauterized using laparoscopic techniques.

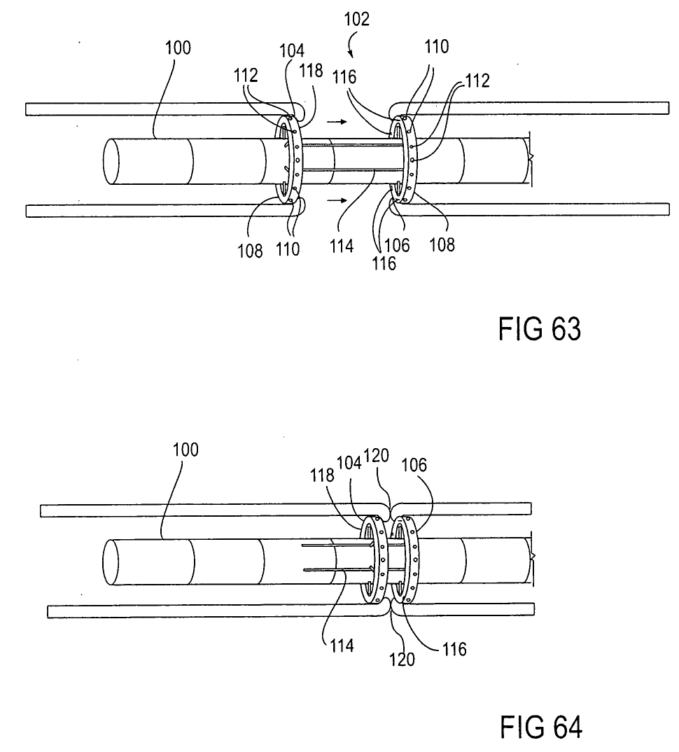

Next, the diseased portion of the colon is excised by transecting the colon at the proximal and distal end of the diseased portion. The colon may be transected using laparoscopic techniques or using a cutting mechanism and/or electrocautery device mounted on the colectomy device 102. The excised tissue is removed using the laparoscope or drawn into the colectomy device 102 for later removal upon withdrawal of the steerable colonoscope 100. FIG. 63 illustrates the colon after the diseased portion has been excised and removed with the colonoscope-mounted colectomy device 102 in position to approximate the transected ends of the colon.

The remaining ends of the colon are approximated one to the other by moving the distal component 104 and/or the proximal component 106 longitudinally with respect to the body of the steerable colonoscope 100, as shown by the arrows. Optionally, the proximal component 106 may be longitudinally translated towards the distal component 104 or both components 104, 106 may be approximated simultaneously towards one another. The ends of the colon are stapled to one another to create an end-to-end anastomosis 120 using the surgical stapler 116 and stapler anvil 118 of the colectomy device 102. Once the ends of the tissue have been approximated, staples or other fastening devices, e.g. clips, screws, adhesives, sutures, and combinations thereof etc., may be actuated through the surgical stapler 116 such that they pierce both ends of the tissue against the stapler anvil 118. FIG. 64 illustrates the colonoscope-mounted colectomy device performing an end-to-end anastomosis 120 to complete the endoscopic colectomy procedure. Once the anastomosis 120 is complete, the expandable members 108 of the distal component 104 and the proximal component 106 are deflated or contracted and the steerable colonoscope 100 and the colectomy device 102 are withdrawn from the patient's body. The expanded members will assure a very accurate end-to-end anastomosis and prevent stenosis that can happen as a result of inaccurate approximation of the two ends.

In an alternative method using the colonoscope-mounted colectomy device 102, the diseased portion of the colon may be excised using a cutting device within the colectomy device 102 after the ends of the diseased portion have been approximated and anastomosed. The excised tissue is drawn into the colectomy device 102 and removed when the steerable colonoscope 100 is withdrawn from the patient.

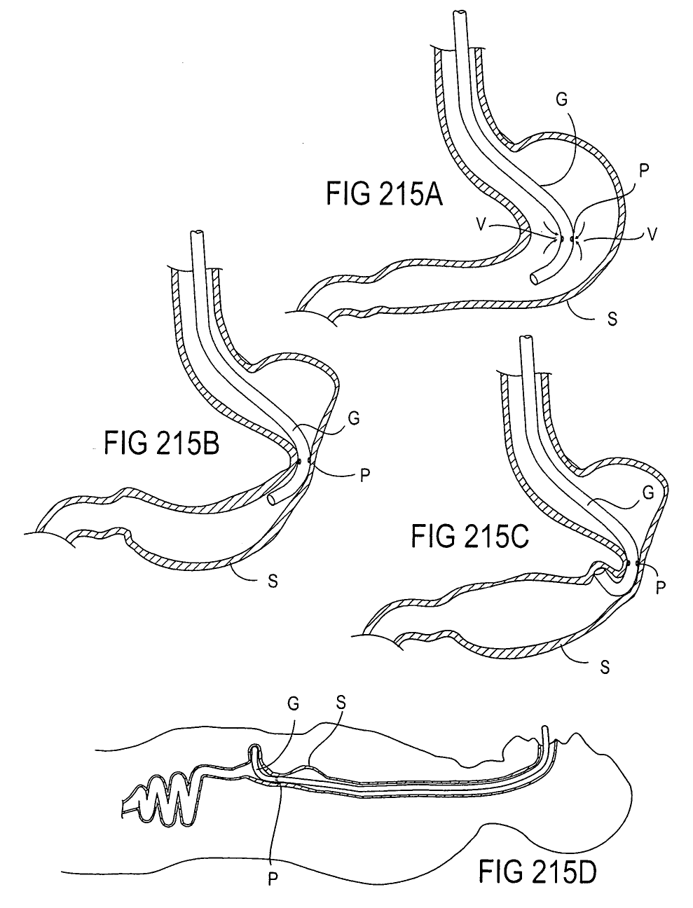

In another alternative method, the colectomy procedure may be performed entirely from the endolumenal approach using the colonoscope-mounted colectomy device 102 without laparoscopic assistance. This method would be particularly advantageous for resection of small portions of the colon where it may not be necessary to mobilize an extended portion of the colon from the omentum to achieve successful approximation and anastomosis. The three-dimensional mapping capability of the steerable colonoscope 102 would be used to locate previously identified lesions without laparoscopic assistance. While described for the colon, it is to be appreciated that attachment, movement and joining if tissue using the above described methods and devices may be applied to other portions of the body or to natural and artificial lumens. For example, this technique and the instrument mounted colectomy device 102 may be used to grasp and manipulate other portions of the gut such as the esophagus, the stomach and the small intestine. The device 102 may also be used in the empty stomach manipulation procedure described below with regard to FIGS. 215A-215D. Gripping mechanism 110 grips the wall of the stomach in addition to or in lieu of the instrument based tissue gripping device (i.e., instrument sidewall suction, instrument sidewall mechanical gripper/anchors or distal instrument end suction or anchors.

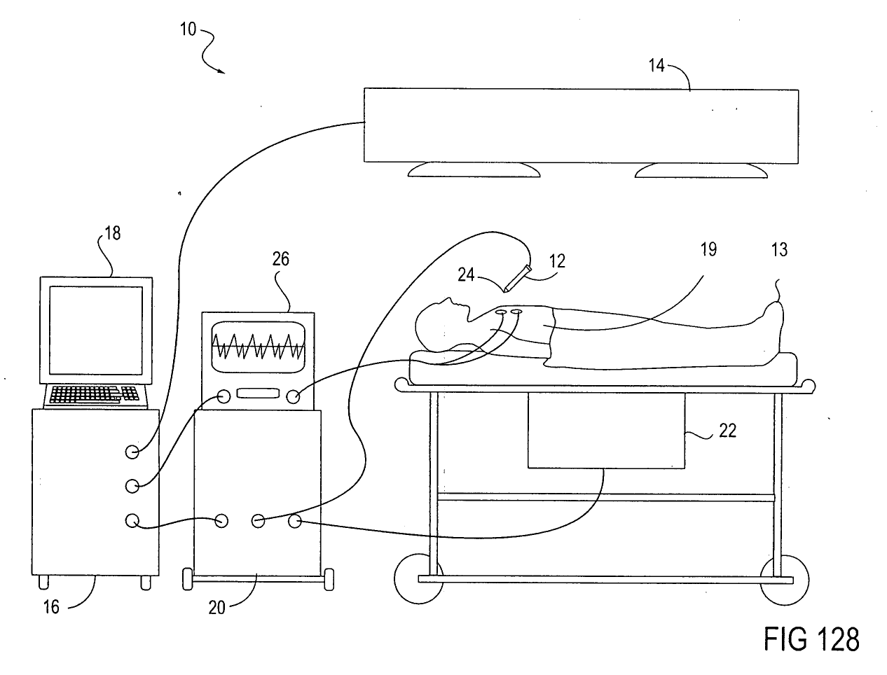

In another alternative embodiment, a steerable instrument, a guide tube, a datum and position indicator may be adapted to include a spectroscopic instrument. For example, the illumination device 112 and the image capture device 114 may be integrated into a datum and position indicator or a guide tube. As such, while the following description is directed towards an endoscope with spectroscopic capabilities, the spectroscopic qualities may be applied to other components in the system. Regardless of what component the spectroscopic devices and capabilities are provided, the spectroscopic information gathered may be used as another input into the mapping and tracking systems described below in FIGS. 128-135

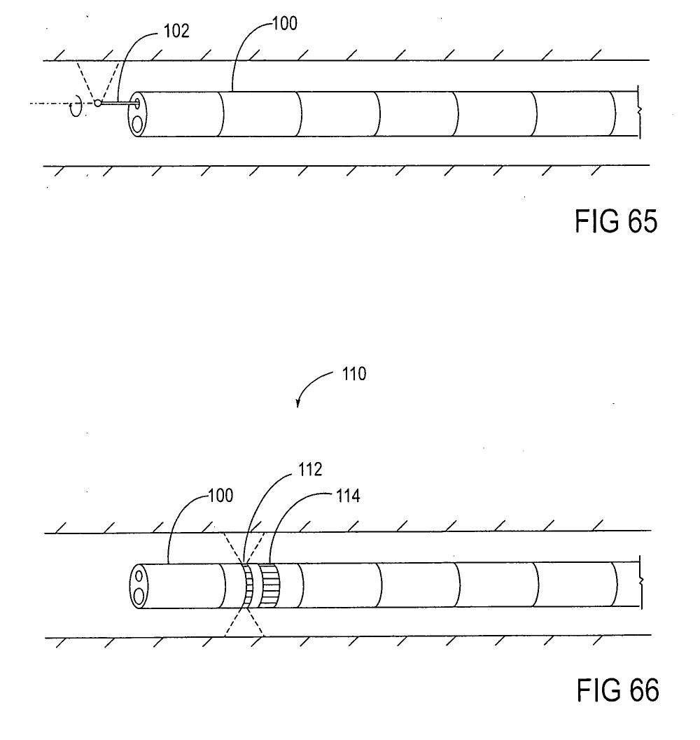

FIG. 65 shows a first embodiment of an endoscopic spectroscopy system according to the present invention that combines a fiberoptic spectroscopy device 102 with a steerable colonoscope 100. Preferably, the steerable colonoscope 100 is constructed as described in U.S. patent application Ser. Nos. 09/790,204 (U.S. Pat. No. 6,468,203); 09/969,927; and 10/229,577, with multiple articulating segments that are controlled to move with a serpentine motion that facilitates insertion and withdrawal of the colonoscope with a minimum of contact and stress applied to the colon walls. The steerable colonoscope 100 may be a fiberoptic endoscope or, more preferably, a video endoscope that uses a CCD camera or the like to capture images of the inside of the colon. In addition, the control system of the steerable colonoscope 100 has the capability to construct a three-dimensional mathematical model of the colon as it advances through lumen under control of the operator. The three-dimensional mathematical model of the colon and the location and nature of any lesions identified in the course of an initial colonoscopic examination can be stored and used for accurately navigating the colonoscope 100 back to the point of the suspected lesion for further diagnostic studies or surgical intervention. The fiberoptic spectroscopy device 102 can be integrated directly into the steerable colonoscope 100 or the fiberoptic spectroscopy device 102 and the steerable colonoscope 100 can be separate instruments that are functionally combined for performing endoscopic spectroscopy, for example by inserting the fiberoptic spectroscopy device 102 through the working channel of the steerable colonoscope 100.

The fiberoptic spectroscopy device 102 delivers a beam of light with one or more excitation frequencies to illuminate the patient's tissues. The excitation frequencies may comprise UV, IR, NIR, blue light and/or other visible or invisible frequencies of light. The fiberoptic spectroscopy device 102 rotates to scan the tissues as the steerable colonoscope 100 advances or retreats. The fiberoptic spectroscopy device 102 captures the light that returns from the surface of the tissue by reflection, by natural fluorescence and/or by dye-enhanced fluorescence or other known spectroscopic technique. The steerable colonoscope 100 provides position information and the fiberoptic spectroscopy device 102 provides rotational information, as well as spectroscopic imaging data, to create a three-dimensional map of the spectroscopic properties of the tissues. The spectroscopic image of the colon captured by the fiberoptic spectroscopy device 102 may be superimposed on the white light endoscopic image of the colon captured by the steerable colonoscope 100 to facilitate analysis of the tissues and any suspected lesions identified. The spectroscopic examination and the white light endoscopic examination may be performed simultaneously if the wavelengths used for each are compatible and/or if the two images can be separated by appropriate optical or electronic filtering. Alternatively, the spectroscopic examination and the white light endoscopic examination may be performed intermittently or in an alternating fashion so that the wavelengths used do not interfere with one another. The three-dimensional map that is generated will enable the operator to return to an area that had some pathology or was suspected as having one in a previous exam, and then perform spectroscopic analysis of the area, and compare it to the previous picture from the same area.

FIG. 66 shows a second embodiment of an endoscopic spectroscopy system with a spectroscopy device 110 integrated directly into a steerable colonoscope 100. Preferably, the steerable colonoscope 100 is constructed as described in U.S. patent application Ser. Nos. 09/790,204 (U.S. Pat. No. 6,468,203); 09/969,927; and 10/229,577, with multiple articulating segments that are controlled to move with a serpentine motion that facilitates insertion and withdrawal of the colonoscope with a minimum of contact and stress applied to the colon walls. The steerable colonoscope 100 maybe a fiberoptic endoscope or, more preferably, a video endoscope that uses a CCD camera, or the like, to capture images of the inside of the colon. In addition, the control system of the steerable colonoscope 100 has the capability to construct a three-dimensional mathematical model of the colon as it advances through lumen under control of the operator. The three-dimensional mathematical model of the colon and the location and nature of any lesions identified in the course of an initial colonoscopic examination can be stored and used for accurately navigating the colonoscope 100 back to the point of the suspected lesion for further diagnostic studies or surgical intervention.

Preferably, the spectroscopy device 110 is integrated directly into the steerable colonoscope 100, for example by integrating the spectroscopy device 110 into one of the articulating segments of the steerable colonoscope 100. In one particularly preferred embodiment, the spectroscopy device 110 extends around the circumference of the steerable colonoscope 100 and is capable of capturing spectroscopic data simultaneously from a 360-degree circle of tissue around the spectroscopy device 110. Alternatively, the spectroscopy device 110 can be configured to mechanically or electronically scan the tissues around the spectroscopy device 110 as the steerable colonoscope 100 advances or retreats.

The spectroscopy device 110 includes an illumination device 112 delivers a beam of light with one or more excitation frequencies to illuminate the patient's tissues. Preferably, the illumination device 112 delivers a ring of illumination in a 360-degree circle around the spectroscopy device 110. Preferably, the illumination device 112 includes one or more LED's or diode lasers or other known light source internal to the device to produce light at one or more excitation frequencies.

Alternatively, the illumination device 112 may use an external light source and a fiberoptic illumination cable to deliver the beam of light. The excitation frequencies may comprise UV, IR, NIR, blue light and/or other frequencies of light in a visible or invisible range. The spectroscopy device 110 includes an image capture device 114 to capture the light that returns from the surface of the tissue by reflection, by natural fluorescence and/or by dye-enhanced fluorescence or other known spectroscopic technique. Preferably, the image capture device 114 extends around the circumference of the steerable colonoscope 100 and is capable of capturing spectroscopic imaging data simultaneously from a 360-degree circle of tissue around the spectroscopy device 110. In a preferred embodiment, the image capture device 114 utilizes a CCD camera or the like internal to the device to capture the spectroscopic imaging data. The CCD camera may be configured to be sensitive only to the spectroscopic imaging frequencies of interest and/or appropriate optical or electronic filtering may be used. Alternatively, the image capture device may use a fiberoptic imaging cable and an external imaging device, such as a CCD camera, to capture the spectroscopic imaging data. The CCD camera may be configured to capture a wide-angle picture of the interior of the colon. Possible ways to capture a wide-angle picture include, but not limited to, using fish eye lens or spherical lens based camera.

The steerable colonoscope 100 provides position information and the spectroscopy device 110 provides spectroscopic imaging data to create a three-dimensional map of the spectroscopic properties of the tissues. The spectroscopic image of the colon captured by the spectroscopy device 110 may be superimposed on the white light endoscopic image of the colon captured by the steerable colonoscope 100 to facilitate analysis of the tissues and any suspected lesions identified. The spectroscopic examination and the white light endoscopic examination may be performed simultaneously if the wavelengths used for each are compatible and/or if the two images can be separated by appropriate optical or electronic filtering. Alternatively, the spectroscopic examination and the white light endoscopic examination may be performed intermittently or in an alternating fashion so that the wavelengths used do not interfere with one another. Another option is that the spectroscopic device will be located far enough from the tip so the light used for vision will not interfere with the spectroscopic exam.

The spectroscopic imaging data and the white light endoscopic imaging data may be viewed in real-time and/or recorded and stored for later analysis and diagnosis of any suspected lesions that are identified. In one preferred method of using the endoscopic spectroscopy system of the present invention, the spectroscopic examination takes place automatically as the steerable colonoscope 100 is advanced and retracted within the patient's colon. The operator is thus freed up to concentrate on manipulating the steerable colonoscope 100 to navigate the tortuous path of the colon and to perform the white light endoscopic examination. Both the spectroscopic imaging data and the white light endoscopic imaging data are recorded and stored together with the information of their exact location, for later analysis and diagnosis of any suspected lesions that are identified. The endoscopic spectroscopy system may also utilize pattern recognition software or the like to identify potential lesions from the spectroscopic imaging data and/or the white light endoscopic imaging data and to inform the operator that a particular portion of the colon warrants closer examination. This function will preferably be performed in real-time during the colonoscopic examination so that suspected lesions can be immediately investigated. In addition, this function may be performed on the recorded image data to enhance diagnostic accuracy.

In one preferred option the spectroscopic data that was recorded on the way in will be shown to the operator on the way out when the pictures shown are the pictures that were taken earlier from the location where the tip of the colonoscope is currently located. It will be achieved by using the three-dimensional mapping capability of the steerable colonoscope 100.

Another option is that the software that analyzes the spectroscopic data will identify suspected areas and when the colonoscope is withdrawn and arrives at the area of those suspected lesions (that were found on the way in), the system will signal to the operator about the suspected lesion and the operator will perform another spectroscopic exam or take a biopsy from the suspected area or lesion.

The stored imaging data from the endoscopic spectroscopy system and the three-dimensional mathematical model of the colon produced by the steerable colonoscope 100 can also be used for tracking progression of disease over time and/or for navigating the steerable colonoscope 100 to the identified lesions for subsequent surgical intervention.

Selectively Rigidizable Guide Variations

Embodiments of the guide tube and steerable controllable instruments described herein could be used anywhere within the gastrointestinal tract including the upper GI tract, the stomach, the intestines, the colon and the like. It is to be appreciated that the steerable controllable instruments described herein could be used in conjunction with a rigidizable guide tube. In some embodiments, the guide tube is anchored to the tissue of interest, thereafter an opening is formed to provide an access between the interior lumen of the guide tube and the now-open tissue. Thereafter the controllable segmented instrument is maneuvered through the interior lumen of the guide tube out to the opening and then to the body portion of interest. Alternatively, it is also to be appreciated that the controllable segmented instrument may be used without a guide tube. In this manner the controllable segmented instrument may be advanced to a position where an opening is desired in tissue. After forming the opening the controllable segmented instrument may be advanced through the opening to a desired position. Once in the desired position or any desired orientation within the body with respect to particular tissue, the controllable segmented instrument could be placed into a locked position. As a result, one or more working channels within the controllable segmented instrument provide a working pathway for instruments devices or other apparatus to be provided through a lumen on or in the controllable segmented instrument into the tissue now accessed.

Additional details of the rigidizable guide tubes are provided in the following descriptions.

FIGS. 62-75C illustrate alternative aspects and further details of the rigidizable elements that may be used in conjunction with rigidizable guide embodiments of the present invention. U.S. Pat. No. 6,800,056 is incorporated herein by reference in its entirely for all purposes. In some embodiments, these elements may be used to rigidizable a guide tube, or a steerable instrument.

FIG. 67 shows an isometric view of a length of the working channel 1120, in this example part of the proximal portion 1122, with a section of the working channel body 1120 removed for clarity. As seen, a representative illustration of the rigidizable element 1136 may be seen disposed within rigidizable element channel or lumen 1150 within the proximal portion 1122. Lumen 1150 may be an existing working channel, i.e., an access channel for other tools, or it may be a designated channel for rigidizable element 1136 depending upon the desired application. Rigidizable element 1136 may be inserted within rigidizable element channel 1150 through a working channel handle or proximal opening and pushed proximally or, alternatively, it may be pushed proximally or pulled distally as described in FIGS. 16-21. Although rigidizable element 36 is shown in this variation as being slidably disposed interiorly of working channel body 20, it may also be disposed exteriorly of the body 20 to slide along a rigidizable element rail or exterior channel in other variations.

FIGS. 68A to 68C show variations on possible cross-sections 32A-32A, 32B-32B, and 32C-32C, respectively, taken from FIG. 67. FIG. 68A shows a simplified cross-section 1122′ of a rigidizable element 1136 having a circular diameter slidably disposed within proximal portion 1122. As seen, rigidizable element 1136 may be slidably positioned within channel 1150′, which may also be used as a working channel upon removal of rigidizable element 1136 during, e.g., a transluminal procedure, for providing access for various instruments or tools to a treatment site. FIG. 68B shows another possible variation in cross-section 1122″ where rigidizable element 1136 is positioned within channel 1150″. The variation of the proximal portion in cross-section 1122 may include a number of access lumens 1152 optionally formed within the body of the device 1120. These lumens 1152 may run through the length of device 1120 and may be used for various applications, e.g., illumination fibers, laparoscopic tools, etc. Although three lumens 1152 are shown in the figure, any number of channels as practically possible may be utilized depending upon the application at hand. FIG. 68C shows another variation in cross-section 1122′″. In this variation, rigidizable element 1136′ may be formed into a semi-circular or elliptical shape to slide within a similarly shaped channel 1150′″. In this example, proximal portion 1122′″ also includes a working channel 1152′ which may be shaped accordingly to fit within the body 1122′″ along with channel 1150′″ to maintain a working channel without having to remove rigidizable element 1136′.

In any of the above examples, the working or rigidizable element channels may be integral structures within the body of working channel 1120. Having an integral structure eliminates the need for a separate lumened structure, e.g., a separate sheath, through which rigidizable element 1136 or any other tools may be inserted. Another variation utilizing multiple channels and multiple rigidizable elements will be described in further detail below. These variations are not intended to be limiting but are merely presented as possible variations. Other structures and variations thereof may be recognized by one of skill in the art and are intended to be within the scope of the claims below.

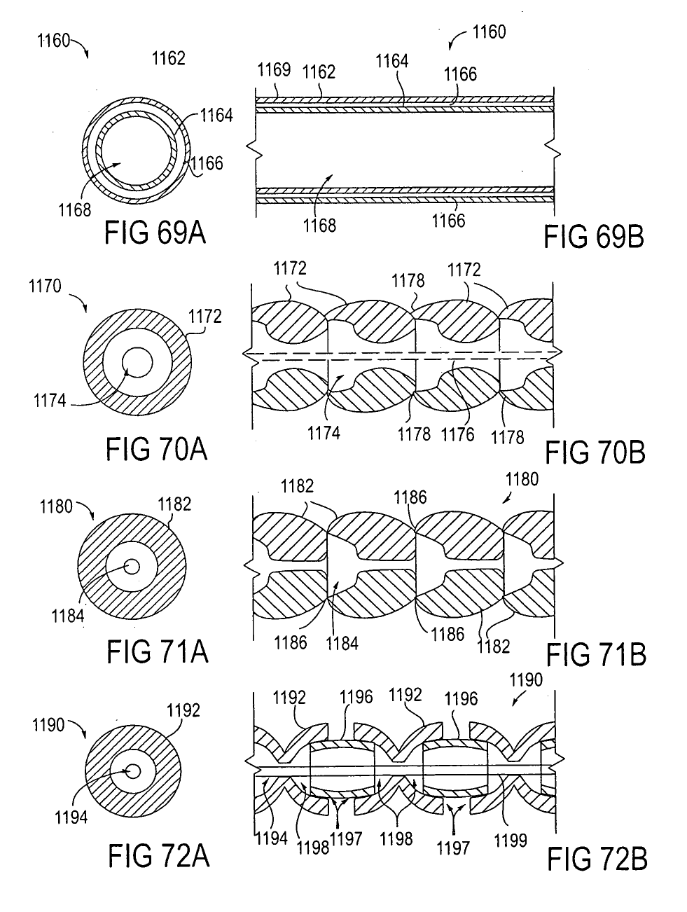

The structure of the rigidizable element may be varied according to the desired application. The following description of the rigidizable element is presented as possible variations and are not intended to be limiting in their structure. FIGS. 69A and 69B show cross-sectioned end and side views, respectively, of a guiding apparatus variation which is rigidizable by a vacuum force applied within the rigidizable element. It is preferable that the rigidizable element is selectively rigidizable, i.e., when the rigidizable element assumes a shape or curve in a flexible state, the rigidizable element may be rigidized to hold that shape or curve for a predetermined period of time. Although the working channel structure of the present invention may utilize a rigidizable element which remains in a relatively flexible shape, it is preferable to have the rigidizable element be selectively rigidizable.

Rigidizable element 1160 may be comprised of two coaxially positioned tubes, outer tube. 1162 and inner tube 1164, which are separated by a gap 1166 between the two tubes. Inner tube 1164 may define an access lumen 1168 throughout the length of the tube to provide a channel for additional tools or other access devices. Both tubes 1162, 1164 are preferably flexible enough to be bent over a wide range of angles and may be made from a variety of materials such as polymers and plastics. They are also preferably flexible enough such that either the outer tube 1162, inner tube 1164, or both tubes are radially deformable. Once rigidizable element 1160 has been placed and has assumed the desirable shape or curve, a vacuum force may be applied to draw out the air within gap 1166. This vacuum force may radially deform inner tube 1164 and bring it into contact with the inner surface of outer tube 1162 if inner tube 1164 is made to be relatively more flexible than outer tube 1162. Alternatively, if outer tube 1162 is made to be relatively more flexible than inner tube 1164, outer tube 1162 may be brought into contact with the outer surface of inner tube 1164.

In another variation, tubes 1162, 1164 may both be made to be flexible such that they are drawn towards one another. In yet another variation, which may be less preferable, a positive force of air pressure or a liquid, e.g., water or saline, may be pumped into access lumen 1168. The positive pressure from the gas or liquid may force the walls of inner tube 1164 radially into contact with the inner surface of outer tube 1162. In any of these variations, contact between the two tubular surfaces will lock the tubes 1162, 1164 together by frictional force and make them less flexible. An elastomeric outer covering 1169, or similar material, may optionally be placed upon the outer surface of outer tube 1162 to provide a lubricious surface to facilitate the movement of rigidizable element 1160 within the endoscopic device. An example of a device similar to rigidizable element 1160 is discussed in further detail in U.S. Pat. No. 5,337,733, which has been incorporated herein by reference in its entirety.

Another variation on the rigidizable element is shown in FIGS. 70A and 70B which show cross-sectioned end and side views, respectively, of a guiding apparatus variation 1170 which is rigidizable by a tensioning member 1176. Tensioned rigidizable element 1170 is shown comprised of a series of individual segments 1172 which are rotatably interlocked with one another in series. Each segment 1172 may contact an adjoining segment 1172 along a contacting lip 1178. Each segment 1172 may further define a channel therethrough which, collectively along with the other segments 1172, form a common channel 1174 throughout a majority of the length of rigidizable element 1170. Segments 1172 may be comprised of a variety of materials suitable for sustaining compression forces, e.g., stainless steel, thermoplastic polymers, plastics, etc.

Proximal and distal segments of rigidizable element 1170 may hold respective ends of tensioning member 1176, which is preferably disposed within common channel 1174 through rigidizable element 1170. Tensioning member 1176 may be connected to a tensioning housing located externally of a patient. During use when the rigidizable element is advanced distally through a working channel of the present invention, tensioning member 1176 is preferably slackened or loosened enough such that rigidizable element 1170 is flexible enough to assume a shape or curve defined by the working channel. When rigidizable element 1170 is desirably situated and has assumed a desired shape, tensioning member 1176 may be tensioned. This tightening or tensioning of member 76 will draw each segment 1172 tightly against one another along each respective contacting lip 78 such that the rigidizable element 1170 becomes rigid in assuming the desired shape. A lubricious covering, e.g., elastomers, etc., may be optionally placed over at least a majority of rigidizable element 1170 to facilitate movement of the rigidizable element 1170 relative to the endoscopic device. A similar concept and design is discussed in further detail in U.S. Pat. No. 5,624,381, which has been incorporated herein by reference in its entirety.

FIGS. 71A and 71B show cross-sectioned end and side views, respectively, of a guiding apparatus variation 1180 which is rigidizable by a vacuum force which interlocks individual segments 1182. Each segment 1182 may be adjoined with adjacent segments by interlocking ball-and-socket type joints which are preferably gasketed at the interfaces 1186 of each connection. Within each segment 1182, with the exception of the distal segment, may be defined a channel which is narrowed at one end and flared at the opposite end. Collectively when the segments 1182 are adjoined into the structure of rigidizable element 1180, each of the individual channels form a common channel 1184 which extends through at least a majority of the segments 1182 along the length of rigidizable element 1180. At the proximal end of rigidizable element 1180 a vacuum pump, which is preferably located externally of the patient, is fluidly connected to common channel 1184. In use, once rigidizable element 1180 is manipulated in its flexible state within the working channel to assume the desired shape or curve, ambient pressure may exist within common channel 1184.

When the rigid shape of rigidizable element 1180 is desired, the pump may then be used to create a negative pressure within common channel 1184 and this negative pressure draws each segment 1182 into tight contact with one another to maintain the desired shape. When the vacuum force is released, each segment 1182 would also be released and would thereby allow the rigidizable element 1180 to be in its flexible state for advancement or withdrawal. Rigidizable element 80 may further be surrounded by an elastomeric or lubricious covering to aid in the advancement or withdrawal of the rigidizable element 80 within the endoscopic device.

FIGS. 72A and 72B show cross-sectioned end and side views, respectively, of yet another guiding apparatus variation 1190 which is optionally rigidizable by either a vacuum force or a tensioning member which interlocks individual segments 1192. Segment 1192 may be in the form of a segmented design with two opposed cups having a common channel 1194 defined therethrough. Between each segment 1192 are ball segments 1196 which interfit along a contact rim or area 1197 within each adjacent segment 1192. Ball segments 1196 preferably contact adjacent cupped segments 96 within receiving channels 1198 defined in each cup. When manipulated in its flexible state, rigidizable element 1190 may be advanced or withdrawn or made to assume a desired shape or curve. When rigidizable element 1190 is to be placed into its rigidized shape, a vacuum force or tensioning member 1199 may be utilized in the rigidizable element 1190 in similar manners as described above. Moreover, rigidizable element 1190 may similarly be surrounded by an elastomeric or lubricious covering to aid in the advancement and withdrawal of the rigidizable element 1190.

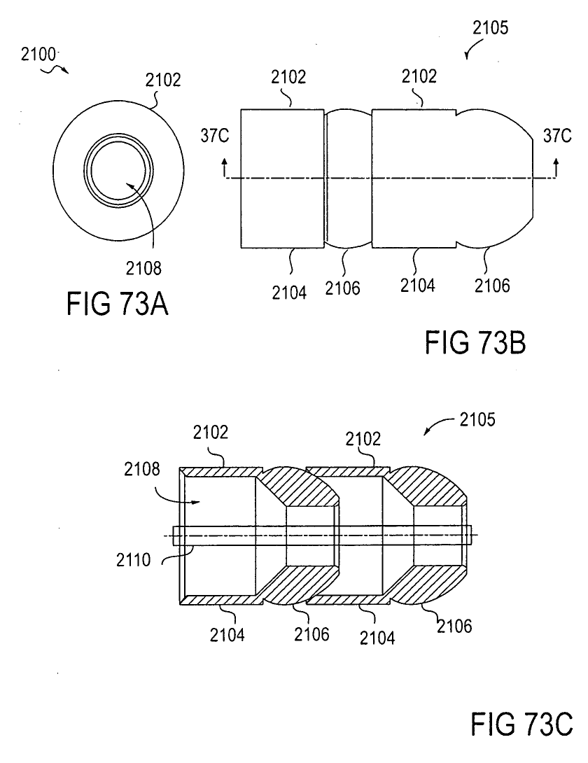

FIGS. 73A and 73B show representative end and side views, respectively, of another guiding apparatus variation 2105. This variation 2105 comprises individual segments 2102 having a uniform sleeve section 2104 in combination with an integrated curved or hemispherical section 2106. Each segment 2102 is collinearly aligned with one another with the sleeve section 2104 receiving the curved section 106 of an adjacent segment 2102; as shown in FIG. 73C, which is the cross-section of rigidizable element 100 from FIG. 73B. The adjacent segments 2102 may rotate relative to one another over the sleeve-hemisphere interface while maintaining a common channel 2108 through the rigidizable element 2105. A tensioning member 2110 may pass through channel 2108 along the length of rigidizable element 2105 for compressing the individual segments 2102 against one another when the entire rigidizable element 2105 is rigidized.

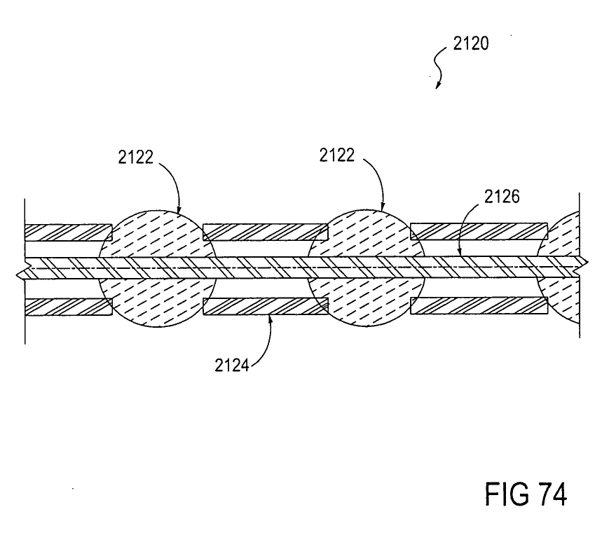

FIG. 74 shows the cross-section of another variation 2120 of the rigidizable rigidizable element apparatus. Representative segments are shown comprising spherical bead segments 2122 alternating with sleeve segments 2124. Each of the bead and sleeve segments 2122, 2124, respectively, may have a channel defined therethrough which allows for a tensioning member 126 to be run through the length of rigidizable element 2120. The alternating segments allow for the rotation of the adjacent segments while the tensioning member 2126 allows for the compression of the segments against one another when the rigidizable element 2120 is to be rigidized in much the same manner as described above.

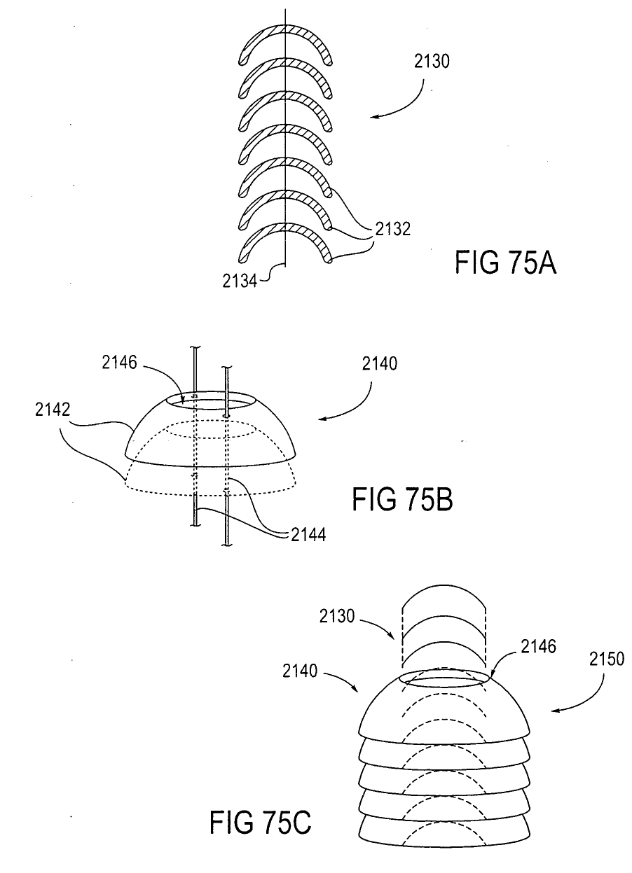

An alternative variation on the rigidizable element is illustrated in FIGS. 75A to 79C, which show a stiffening assembly having separate rigidizable coaxially positioned rigidizable elements. FIG. 75A shows a representative number of nested segments 2132 in nested stiffening assembly 2130. Each nested segment 2132 may be in a number of different configurations, e.g., ball socket joints, stacked ring-like segments, etc., with a tensioning member 2134 passing through each of the segments 2132. For use with nested assembly 2130, an annular stiffening assembly 140 may be seen in FIG. 79B. Annular assembly 2140, of which only a few representative segments are shown, are comprised in this variation of annular segments 2142 which may be stacked or aligned one atop each other. At least one tensioning member 2144, and preferably at least two, may be passed through each of the annular segments 2142. A central area 2146 is defined in each annular segment 2142 such that nested stiffening assembly 2130 may be slidingly placed within the central area 146 defined by the annular stiffening assembly 2140. FIG. 75C shows the stiffening assembly 2130 slidingly positioned within annular stiffening assembly 140 to form the coaxially aligned stiffening assembly 2150.

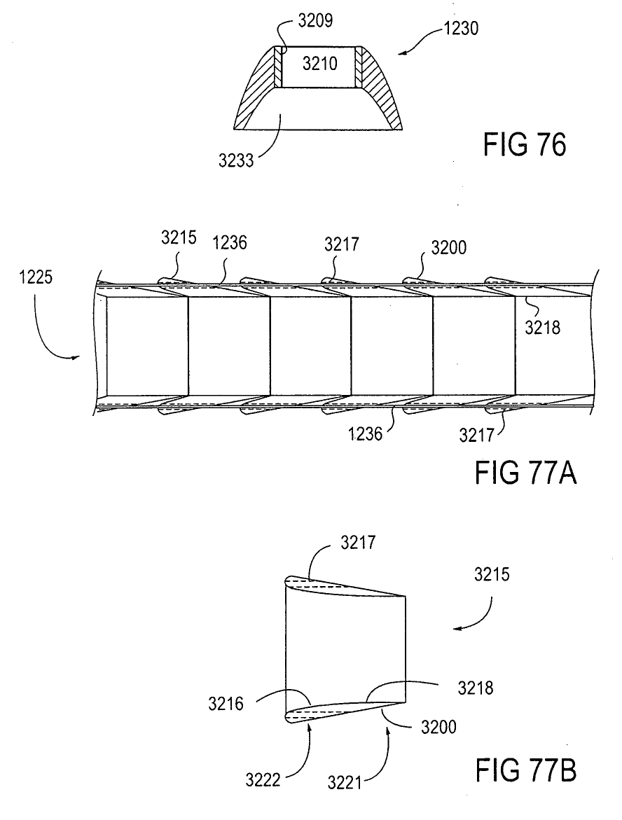

Still further alternative aspects of the rigidizable elements used with embodiments of the working channel of the present invention are described with regard to FIGS. 76 to 85. U.S. Patent Application Publication 2003/0233058 filed Oct. 25, 2003 is incorporated herein by reference.

FIGS. 76, 7A, and 77B illustrate still further alternative structures to facilitate rigidizing an embodiment of a working channel of the present invention. For example, some or all of nestable rigidizable elements 1230 may incorporate hydrophilically-coated polymeric layer 3209, which may be disposed surrounding distal portion 3210 of bore 1233. A plurality of elements 1230 could be arranged along the length of a working channel.

Alternatively, as described in FIGS. 77A and 77B, a working channel embodiment may comprise a multiplicity of frustoconical elements 3215 that, when nested, provide a smooth inner lumen to accommodate an instrument or device therethrough without the need for a separate liner. Each frustoconical element 3215 includes central bore 3216, and at least two or more tension wire bores 3217. Central bore 3216 is defined by cylindrical distal inner surface 3218 that has a substantially constant diameter, and proximal inner surface 3219 that is continuous with distal inner surface 3218.

Proximal inner surface 3219 is slightly curved in a radially outward direction so that, when tension wires 1236 are relaxed, proximal inner surface 3219 can rotate relative to external surface 3220 of an adjacent element. External surface 3220 of each frustoconical element may be straight or contoured to conform to the shape of proximal inner surface 3219, and tapers each element so that distal end 3221 is smaller in outer diameter than proximal end 3222. When frustoconical elements 3215 are nested together, distal inner surface 3218 of each frustoconical element is disposed adjacent to the distal inner surface of an adjoining frustoconical element.

Advantageously, the present configuration provides lumen 1225 with a substantially continuous profile. This permits smooth advancement of an instrument or a device therethrough, and thereby eliminates the need to dispose a separate liner within lumen 1225. To provide a lubricious passageway to further facilitate advancement of the colonoscope, each frustoconical element optionally may incorporate an integral hydrophilic polymeric lining such as polymeric layer 209 described with respect to the preceding embodiment of FIG. 76, or a thin, flexible lining having a hydrophilic coating may be disposed through lumen 1225.

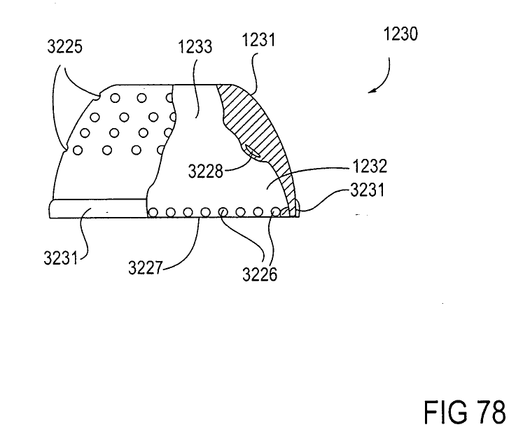

In FIG. 78, yet another alternative structure is described, in which distal surface 1231 of each nestable element is macroscopically textured to increase the friction between adjacent nestable elements 1230 when a compressive clamping load is applied. Illustratively, each element 1230 may incorporate multiplicity of divots 3225 disposed on distal surface 1231, and teeth 3226 that are disposed on proximal surface 1232 adjacent proximal edge 3227. Teeth 3226 are contoured to mate with the multiplicity of divots disposed on an adjacent element. Accordingly, tension applied to a plurality of adjacent rigidizable elements 1230 applies a clamping load to elements 1230 that causes teeth 3226 of each element to forcefully engage divots 3225 of an adjacent element. This reduces the risk of relative angular movement between adjacent nestable elements 1230 when the working channel is shape-locked, which in turn reduces the risk of undesired reconfiguration of the working channel.

Referring now to FIGS. 79 and 80, alternative embodiments of the working channel are described. Unlike previously described embodiments, in which a mechanical mechanism is actuated to impart a clamping load to a multiplicity of nestable elements, the embodiments of FIGS. 79 and 80 use alternative tensioning mechanisms. In particular, the following embodiments comprise a multiplicity of links to which a compressive clamping load may be applied by contraction of shape memory materials.

In FIG. 79, an alternative embodiment of the working channel of the present invention is described. Working channel 3270 includes multiplicity of nestable elements 1230 identical to those described hereinabove. For purposes of illustration, nestable elements 1230 are shown spaced-apart, but it should be understood that elements 1230 are disposed so that distal surface 1231 of each element 1230 coacts with proximal surface 1232 of an adjacent element. Each of nestable elements 1230 has central bore 1233 to accommodate an instrument or a device, and preferably two or more tension wire bores 1235. When assembled as shown in FIG. 79, nestable elements 1230 are fastened with distal and proximal surfaces 1231 and 1232 disposed in a coacting fashion by a plurality of tension wires 3271 that extend through tension wire bores 1235.

In contrast to previous working channel embodiments, tension wires 3271 of the present working channel are made from a shape memory material, e.g., nickel titanium alloy, or an electroactive polymer known in the art. Tension wires 3271 are fixedly connected to the distal end of working channel 3270 at the distal ends and fixedly connected to a handle or conventional tension control system at the proximal ends. When an electric current is passed through tension wires 3271, the wires contract in length, imposing a compressive clamping load that clamp distal and proximal surfaces 1231 and 1232 of nestable elements 1230 together at the current relative orientation, thereby fixing the shape of working channel 3270. When application of electrical energy ceases, tension wires 3271 re-elongate in length to provide for relative angular movement between nestable elements 1230. This in turn renders working channel 3270 sufficiently flexible to negotiate a tortuous path through the colon, other organs or regions of the body.

To provide working channel 3270 with a fail-safe mode that reduces the risk of undesired reconfiguration of the working channel in the event of tensioning mechanism failure, diametrically disposed tension wires 3271 may be coupled in a serial circuit. Accordingly, when one wire fails, the wire disposed diametrically opposite also re-elongates to maintain a symmetrical clamping load within working channel 3270. Alternatively, all tension wires 3271 may be electrically coupled in a serial electrical circuit. Accordingly, when one of the tension wires fails, working channel 3270 returns to the flexible state.

It should be understood that a tension spring (not shown) or damper (not shown) that are familiar to those of ordinary skill may be coupled between the proximal ends of tension wires to maintain the tension wires in constant tension when the working channel is in a shape-locked state. Such constant tension reduces the risk of reconfiguration of the working channel to its flexible state if nestable elements disposed therein slightly shift relative to adjacent nestable elements.

Alternatively, as illustrated in FIG. 80, working channel 3280 may include multiplicity of nestable elements 3281 that are similar to those of the preceding embodiments. For purposes of illustration, nestable elements 3281 are shown spaced-apart, but it should be understood that elements 3281 are disposed so that distal surface 3282 of each element 3280 coacts with proximal surface 3283 of an adjacent element. Each of nestable elements 3280 has central bore 3284 to accommodate an instrument or a device.

When assembled as shown in FIG. 80, nestable elements 3280 are fastened with distal and proximal surfaces 3282 and 3283 disposed in coacting fashion by plurality of thin tension ribbons 3285 that are fixedly connected to nestable bridge elements 3286. Tension ribbons 3285 are made from a shape memory material, e.g., nickel titanium alloy or an electroactive polymer, and may be transitioned from an equilibrium length to a contracted length when electrical current is passed therethrough.

Nestable bridge elements 3286 are disposed within working channel 3280 between a predetermined number of nestable elements 3281. Similar to nestable elements 3281, bridge elements 3286 also comprise central bore 3287 that accommodates an instrument or a device, distal surface 3288 that coacts with proximal surface 3283 of a distally adjacent nestable element, and proximal surface 3289 that coacts with distal surface 3282 of a proximally adjacent nestable element 3281. Each bridge element also incorporates plurality of conductive elements 3290 that are disposed azimuthally around central bore 3287, and that preferably couple tension ribbons 3285 occupying the same angular circumferential position within working channel 3280 in a serial electrical circuit.

When an electrical current is passed through tension ribbons 3285, the ribbons contract in length, imposing a compressive load that clamps distal and proximal surfaces of adjacent nestable elements together at the current relative orientation, thereby fixing the shape of working channel 3280. When the energy source ceases providing electricity, tension ribbons 3285 re-elongate to the equilibrium length to provide for relative angular movement between the nestable elements. This in turn renders working channel 280 sufficiently flexible to negotiate a tortuous path through the colon, another organ or region of the body.

Pursuant to another aspect of the present embodiments, tension ribbons 3285 that are disposed at diametrically opposite circumferential positions may be electrically coupled in a serial circuit. Advantageously, this configuration provides working channel 3280 with a fail-safe mode that reduces the risk of undesired reconfiguration of the working channel in the event that one of the electrical circuits established through the tension ribbons is de-energized.

For example, working channel 3280 of FIG. 80 may be provided with four sets of tension ribbons equidistantly disposed at 90 degree intervals. In the event that tension ribbons Tₐ de-energize, absent electrical communication between tension ribbons Tₐ and tension ribbons T_{c} disposed diametrically opposite thereto, working channel 3280 will spontaneously reconfigure into a new rigidized shape since the tension within the working channel no longer will be symmetrically balanced. The new shape of working channel 3280 may not replicate the selected pathway and thus may cause substantial harm to the patient.

Advantageously, the present invention may reduce the risk of undesired reconfiguration preferably by electrically coupling diametrically disposed tension ribbons in a serial circuit. When tension ribbons Tₐ are de-energized, tension ribbons T_{c} also de-energize to provide working channel 3280 with symmetrical tension, as provided by tension wires Tb and the tension wires disposed diametrically opposite thereto (not shown). In this manner, the working channel retains its desired rigidized shape in the event that the tensioning mechanism malfunctions. To immediately return working channel 3280 to its flexible state in the event that any of the tension ribbons are de-energized, all tension ribbons 3285 may be electrically coupled in a serial circuit.

In an alternative embodiment, tension ribbons 3285 may be electrically coupled to rigidize select regions of the working channel without rigidizing the remainder of the working channel. Illustratively, this may be accomplished by coupling longitudinally adjacent tension ribbons in a parallel circuit, and circumferentially adjacent tension ribbons in a serial circuit.

Of course, it will be evident to one of ordinary skill in the art that, while FIG. 80 depicts tension ribbons 3285 to be disposed within central bores 3284 and 3287, the tension ribbons also may be disposed adjacent external lateral surfaces 3292 of nestable elements 3281 and 3286. Alternatively, the tension ribbons may extend through tension ribbon bores (not shown) that may extend through the distal and proximal surfaces of nestable elements 3281, and be affixed to nestable bridge elements 3286. Still another alternative aspect of the use of shape memory elements in conjunction with working channel embodiments of the present invention is to transition the working channel between stowed and deployed configurations.

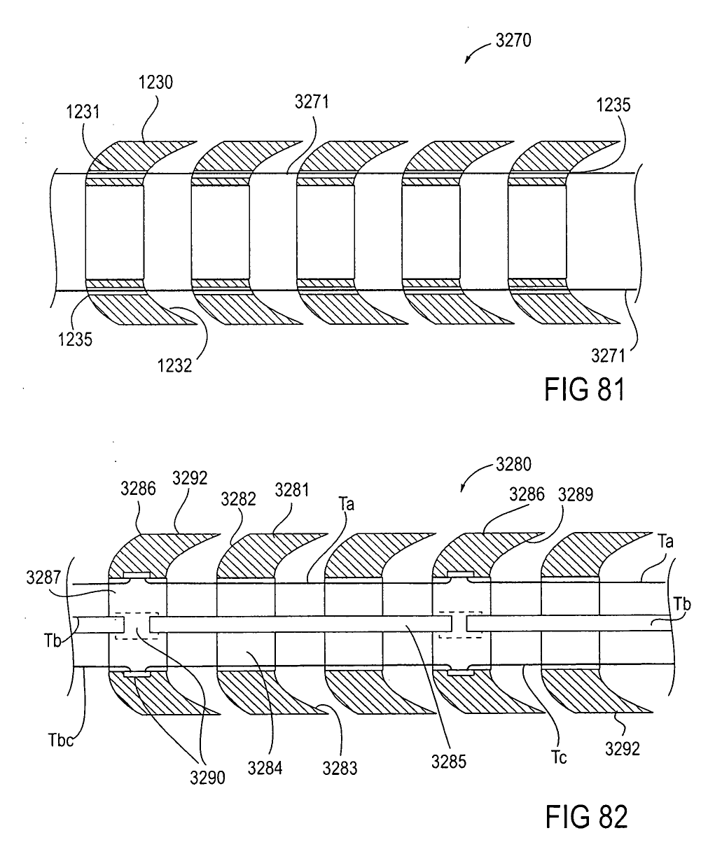

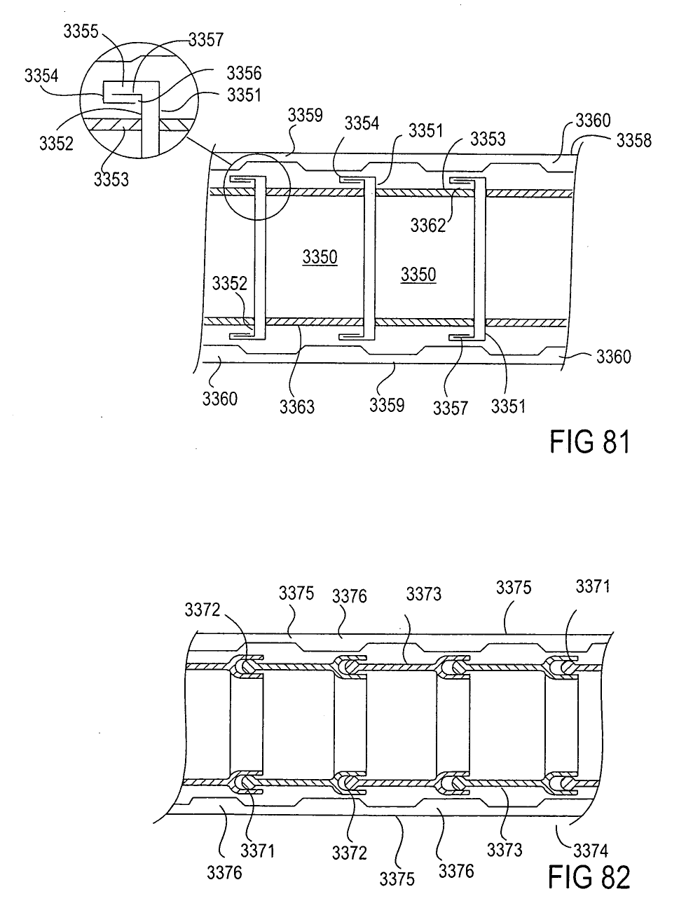

Referring now to FIG. 81, another alternative embodiment of a working channel is described, in which each Grecian link 3350 includes rigid first and second rims 3351 and 3352 disposed at longitudinally opposing ends of flexible body 3353. First rim 3351 comprises U-shaped arm 3354 that defines channel 3355 and opening 3356. Second rim 3352 includes retroflexed arm 3357, which when engaged to first rim 3351 of an adjacent, is disposed within channel 3355 of U-shaped arm 3354 through opening 3356 so that U-shaped arm 3354 and retroflexed arm 3357 are engaged and overlap along the longitudinal axis of the working channel.

Grecian links 3350 are disposed within compressive sleeve 3358, which includes first compressive portions 3359 and second compressive portions 3360. In compressive sleeve 3358, the second compressive portions 3360 are aligned with, and apply a clamping force to, overlapping U-shaped arm 3354 and retroflexed arm 3357 of the first and second rims. It will of course be understood that an working channel in accordance with the principles of the present invention couple alternatively be formed using Grecian links 3350 with other clamping systems known to those of ordinary skill in the art.

Referring now to FIG. 82, yet another alternative embodiment of an working channel suitable for use in the present invention is described. This embodiment comprises joint links 3370 that include ball 3371 and socket 3372 disposed at longitudinally opposing ends of flexible body 3373. When adjacent joint links 3370 are engaged, ball 3371 of one link is disposed within socket 3372 of an adjacent link. When the working channel is flexed, ball 3371 coacts with socket 3372 to provide articulation of the working channel.

Joint links 3370 are disposed within compressive sleeve 3374, which includes first compressive portions 3375 and second compressive portions 3376. Compressive sleeve 3374 is identical in structure and operation to that described above except that second compressive portions 3376 are aligned with, and apply a clamping force to, socket 3372 within which ball 3371 of an adjacent link is disposed. It will of course be understood that a working channel in accordance with the principles of the present invention could alternatively be formed using joint links 3370 and could employ clamping systems known to those of ordinary skill in the art.

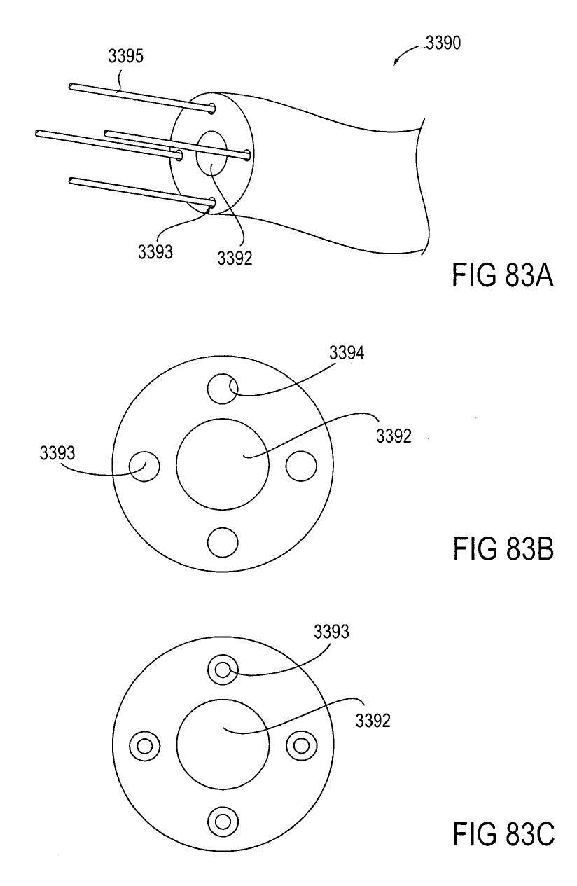

Referring now to FIGS. 83A-83C, an additional alternative embodiment of an working channel suitable for use with the present invention is described. Working channel 3390 comprises elongate body 3391 having central lumen 3392 that accommodates an instrument or a device, and wire lumens 3393 that are defined by cylindrical wire lumen surfaces 3394. Within each wire lumen 3393 is disposed wire 3395 that extends the length of the elongate body. Elongate body 3391 is made from an electroactive polymer known in the art that permits wire lumens 3393 to vary in diameter responsive to electrical energization.

In particular, when an electrical current is passed through elongate body 3391, the diameter of each wire lumen 3393 decreases so that the wire lumen clamps around a respective wire 3395. Preferably, both wires 3395 and wire lumen surfaces 3394 are textured to enhance friction therebetween. This prevents further relative movement between elongate body 3391 and wires 3395, and stiffens working channel 3390. When application of the electrical current ceases, wire lumens 3393 increase in diameter to release wires 3395 so that elongate body 3391 may shift relative to wires 3395. This in turn renders working channel 3390 sufficiently flexible to negotiate a tortuous path through the colon, another organ or a body region.

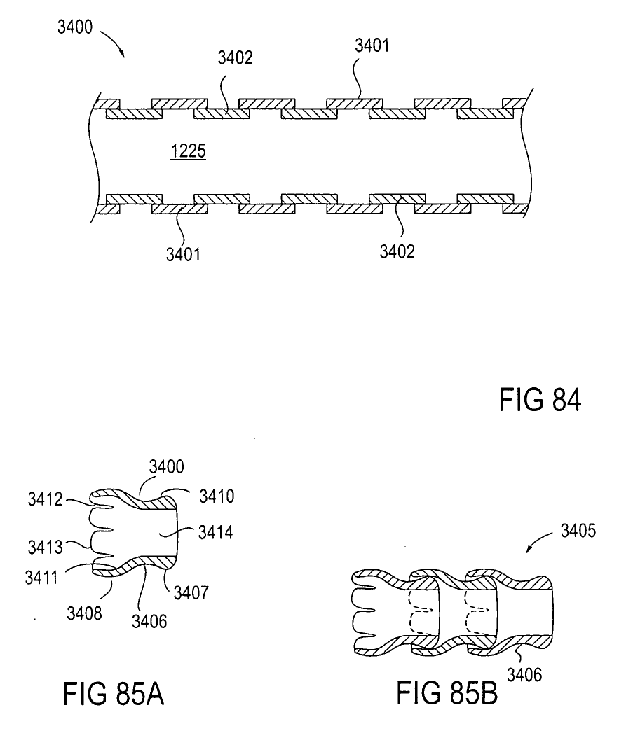

With respect to FIG. 84, yet another alternative embodiment of the working channel is described. Working channel 3400 incorporates a multiplicity of variable diameter links 3401 disposed in overlapping fashion surrounding a multiplicity of rigid links 3402 that provide structural integrity to the working channel. Each link comprises a central bore that defines lumen 1225 of the working channel that is sized, when deployed, to accommodate instruments and devices. Variable diameter links 3401 preferably are manufactured from an electroactive polymer or a shape memory alloy and contract in diameter when energized. When variable diameter links 401 are electrically activated, the variable diameter links tighten about rigid links 3402 to transition working channel 3400 into a shape-locked state. When the variable diameter links are electrically deactivated, the variable diameter links sufficiently soften to return working channel 3400 back to the flexible state.

In a preferred embodiment, variable diameter links 3401 and rigid links 3402 are formed from respective strips of material that are helically wound in an overlapping fashion to form working channel 3400. Alternatively, each link may be individually formed and disposed in an overlapping fashion.

In FIGS. 85A-85B, still another alternative embodiment of an working channel suitable for use with the apparatus of the present invention is illustrated schematically. Working channel 3405 comprises a multiplicity of nestable hourglass elements 3406 that preferably are manufactured from an electroactive polymer or a shape memory alloy, and each have bulbous distal and proximal portions 3407 and 3408 connected by neck 3409. The diameter of neck 3409 is smaller than the maximum diameter of distal portion 3407, which in turn is less than the maximum diameter of proximal portion 3408. The distal portion of external surface 3410 of each hourglass element 3406 is contoured to coact with the proximal portion of internal surface 3411 of a distally adjacent hourglass element. Accordingly, when a multiplicity of hourglass elements are nested together to form working channel 3405, adjacent elements 3406 may move relative to each other when the working channel is in the flexible state.

To reduce friction between adjacent elements during relative movement therebetween, proximal portions 3408 include a plurality of slits 3412 disposed contiguous with proximal edge 3413. Slits 3412 also facilitate contraction of proximal portion 3408 of each element around distal portion 3407 of an adjacent element. Each hourglass element 3406 also has central bore 3414 that accommodates an instrument or a device.

When an electrical current is applied to the multiplicity of nestable hourglass elements 3406, proximal portion 3408 of each element contracts in diameter around distal portion 3407 of an adjacent element. The compressive clamping force there applied prevents relative movement between adjacent elements, thereby shape-locking the working channel. When the nestable elements are deenergized, proximal portions 3408 sufficiently relax to permit relative movement between adjacent nestable elements 3406, and thus permit working channel 3405 to negotiate tortuous curves. For purposes of illustration, it should be understood that the figures of the present application may not depict an electrolytic medium, electrodes, wiring, control systems, power supplies and other conventional components that are typically coupled to and used to controllably actuate electroactive polymers described herein.

While the illustrated embodiments described herein refer to an endoscope, it is to be appreciated that other surgical tools may be adapted to become a rigidized using an embodiment of the present invention. Moreover, while described for use with controllable instruments such as endoscopes, it is to be appreciated that embodiments of the expandable working channels described herein may be used in a variety of medical, industrial and therapeutic applications.

Described here are devices, systems, and methods for navigating, maneuvering, positioning or support for delivering an instrument having an external working channel or the external working channel itself into both open and solid regions of the body. While the illustrated embodiments described to herein refer to delivery of external working channels of the present invention in conjunction with surgical, therapeutic and/or diagnostic procedures related to the colon or the heart, is to be appreciated that these are only illustrative examples.

While some specific examples are provided for a particular organ such as the colon, the invention is not so limited. It is to be appreciated that the term “region” as used herein refers to luminal structures as well as solid organs and solid tissues of the body, whether in their diseased or nondiseased state. Examples of luminal structures or lumens include, but are not limited to, blood vessels, arteriovenous malformations, aneurysms, arteriovenous fistulas, cardiac chambers, ducts such as bile ducts and mammary ducts, fallopian tubes, ureters, large and small airways, and hollow organs, e.g., stomach, small and intestines, colon and bladder. Solid organs or tissues include, but are not limited to, skin, muscle, fat, brain, liver, kidneys, spleen, and benign and malignant tumors. As such, it is to be appreciated that the external working channel embodiments of the present invention have broad applicability to numerous surgical, therapeutic and/or diagnostic procedures.

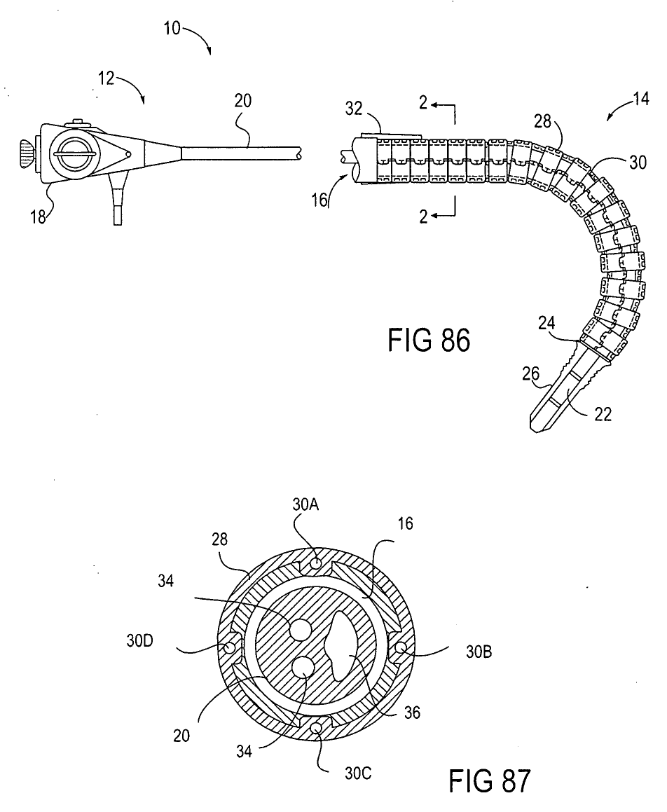

As shown in FIG. 86, a representative illustration of a variation on guide tube assembly 10 is seen partially disassembled for clarity. Assembly 10 generally comprises an endoscope 12 which is insertable within guide tube 14 through guide lumen 16. Endoscope 12 may be any conventional type endoscope having a handle 18 with shaft 20 extending therefrom. The distal end of shaft 20 preferably comprises a controllable distal portion 22 which may be manipulated to facilitate the steering of the device through the body. Endoscope shaft 20 may be slidingly disposed within guide lumen 16 such that controllable distal portion 22 is able to be passed entirely through guide tube 14 and out distal opening 24 defined at the distal end of tube 14.

Alternatively, guide tube 14 may also be used with an endoscope having an automatically controlled proximal portion and a selectively steerable distal portion, as described in further detail below. Such a controllable endoscope may have a distal portion which is manually steerable by the physician or surgeon to assume a shape to traverse an arbitrary curved path and a proximal portion which is automatically controlled by, e.g., a computer, to transmit the assumed shape along the proximal portion as the endoscope is advanced or withdrawn. More detailed examples are described in copending U.S. patent application Ser. No. 09/969,927, which has been incorporated above by reference in its entirety.

Returning to FIG. 86, bellows or covering 26 may cover distal opening 24 of guide tube 14 to prevent the entry of debris and fluids within guide lumen 16. As distal portion 22 of shaft 20 is advanced distally through tube 14 and out of guide lumen 16, covering 26 is preferably configured to expand distally either over or with shaft 20 while maintaining a seal with guide lumen 16. When shaft 20 is retracted within guide lumen 16 or when guide tube 14 is advanced distally relative to shaft 20, covering 26 is preferably configured to retract proximally back over distal opening 24 along with the proximal movement of distal portion 22. The use of covering 26 is optional and may be used to maintain the sterility of guide lumen 16. Covering 26 may also be used to prevent the pinching and tearing of tissue when shaft 20 is withdrawn within guide lumen 16.

Guide tube 14 may be any conventional appropriately flexible conduit which is capable of being rigidized along its entire length. The variation shown in FIG. 86 is comprised of a plurality of individual segments 28 which are linked adjacent to one another via several, i.e., more than one, tensioning wires or elements 30. Segments 28 may be a series of interconnecting ball-and-socket type segments which allow adjacent segments 28 to angularly pivot relative to one another to form an angle for traversing curves. These segments 28 may be rigidized via tensioning elements 30 which may be placed circumferentially about segments 28, as shown in FIG. 87, which is a cross-sectioned view of assembly 10 from FIG. 1. In this variation, there are four tensioning wires 30A, 30B, 30C, 30D which are each placed 90° relative to one another. Although four wires are shown in this example, a fewer number of wires may also be used, e.g., three wires. Each of these wires 30A, 30B, 30C, 30D may be routed through an integral channel or lumen defined in the walls of each segment 28. Moreover, they may be individually manipulated or they may all be manipulated simultaneously to effect a tensioning force for either rigidizing or relaxing guide tube 14 along its length.

FIG. 87 also shows the relative positioning of shaft 20 in relation to segment 28. As seen, shaft 20, which may contain any number of channels 34 for illumination fibers, optical fibers, etc., and working channels 34, is slidingly disposed within guide lumen 16. This variation shows a gap separation between the outer surface of shaft 20 and the inner surface of segment 28. This gap may vary depending upon the diameter of the endoscope being used and the desired cross-sectional area of guide tube 14, but a nominal separation is preferable to allow the uninhibited traversal of shaft 20 within guide lumen 16. An example of a rigidizable conduit structure which may be utilized as part of the present invention is shown and described in further detail in U.S. Pat. No. 5,251,611 to Zehel et al., which is incorporated herein by reference in its entirety.

The outer surface of guide tube 14 preferably has a tubular covering 32 which covers at least a majority of tube 14. Tubular covering 32 may provide a barrier between the debris and fluids of the body environment and the interior guide lumen 16, if also used with covering 26. Moreover, covering 26 may be an integral extension of tubular covering 32 and may accordingly be made from a continuous layer of material. Tubular covering 32 may also provide a lubricous cover to facilitate the insertion and movement of guide tube 14 along the walls of the body lumen as well as to provide a smooth surface inbetween the individual segments 28 to prevent the tissue from being pinched or trapped. Tubular covering 32 may be made from a variety of polymeric materials, e.g., PTFE, FEP, Tecoflex, etc.

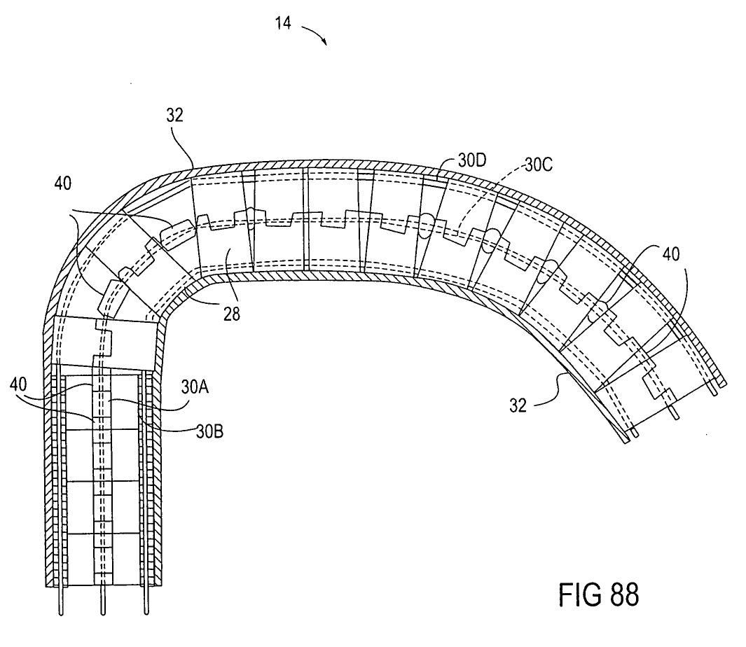

FIG. 88 shows a side view of guide tube variation 14 with a portion of the wall partially removed for clarity. As shown, individual segments 28 are aligned adjacent to one another with interconnecting sleeves 40 placed inbetween. Sleeves 40, in this variation, may be used to provide a pivoting structure to allow guide tube 14 to flex into different positions. Alternatively, segments 28 may be curved ball-and-socket type joints configured to interfit with one another. Tubular covering 32 may also be seen to cover at least the majority of guide tube 14. Optionally, a distal end portion of guide tube 14 may be configured to be controllable such that guide tube 14, like the controllable distal portion 22 of the endoscope 12, may define an optimal path for traversal.

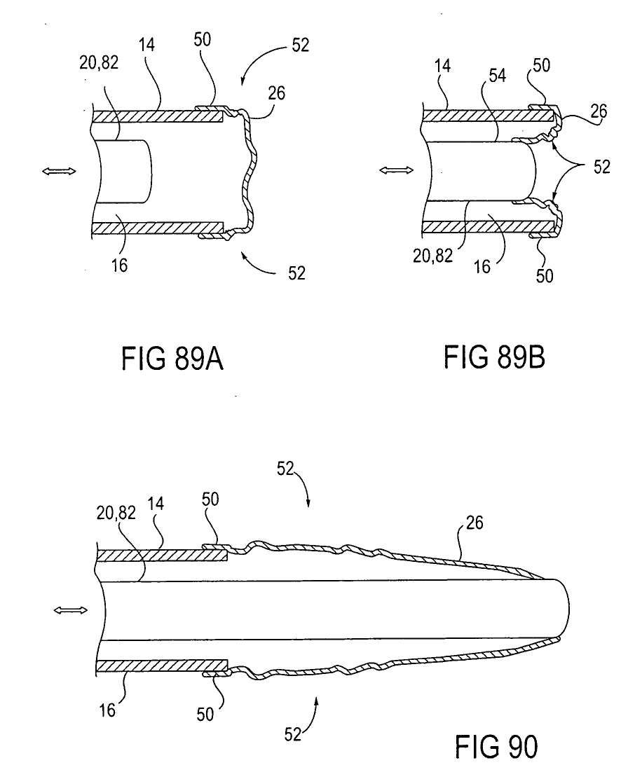

Bellows or covering 26 may optionally be appended to the distal end of conventional endoscope shaft 20 or controllable shaft 82. Throughout the description herein, automatically controllable endoscope 82 may be interchanged with conventional endoscope 12 when used in guide tube 14 as well as with the use of bellows or covering 26. Although descriptions on the method of use may describe use with conventional endoscope 12, this is done for brevity and is not intended to be limiting. The description is intended to apply equally to use with controllable endoscope 80 since the two may be easily interchanged depending upon the desired use and result. FIG. 89A shows one variation in which shaft 20 or 80 is unattached to covering 26 such that endoscope 12 may be freely inserted and withdrawn from guide lumen 16. Covering 26 may be omitted altogether from the assembly but is preferably used not only to help maintain an unobstructed guide lumen 16, but also to prevent the walls of the body lumen from being pinched between the endoscope shaft 20 or 80 and guide tube 14 during advancement of the assembly. As seen in FIG. 89A, covering 26 may be separately attached at attachment region 50 to the outer surface or distal edge of guide tube 14. Covering 26 may also further comprise a gusseted region 52 which allows the covering 26 to be compressed into a small compact profile and expanded much like a bellows during shaft 20 or 80 advancement. When shaft 20 or 80 is withdrawn, gusseted region 52 may allow covering 26 to recompress or reconfigure itself back into its compacted shape. In this variation, covering 26 is unattached to shaft 20 or 80; therefore, once the assembly has reached a predetermined location within the colon, covering 26 may be removed through a working channel within endoscope 12 or the working tools may simply be pierced through covering 26, although this is less preferable, before a procedure may be begin.

FIG. 89B shows another variation where covering 26 may be attached to the endoscope shaft 20 or 80 near or at the distal end of controllable distal portion 22 along attachment region 54. As shaft 20 or 80 is advanced or withdrawn from guide lumen 16, covering 26 remains attached to the endoscope 12. FIG. 90 shows shaft 20 or 80 being advanced to a distal position through guide lumen 16. As shaft 20 or 80 is advanced, gusseted region 52 may be seen expanding to accommodate the distal movement. The gusseted region 52 may be configured to allow shaft 20 or 80 to be advanced to any practical distance beyond guide tube 14, e.g., a few or several inches, depending upon the application. With this variation, shaft 20 or 80 may be extended through guide lumen 16 to this distal position prior to first advancing shaft 20 or 80 within the colon of a patient as well as to allow enough room so that the controllable distal portion 22 may have enough space to be manipulated to assume a desired shape or curve over which guide tube 14 may be advanced over.

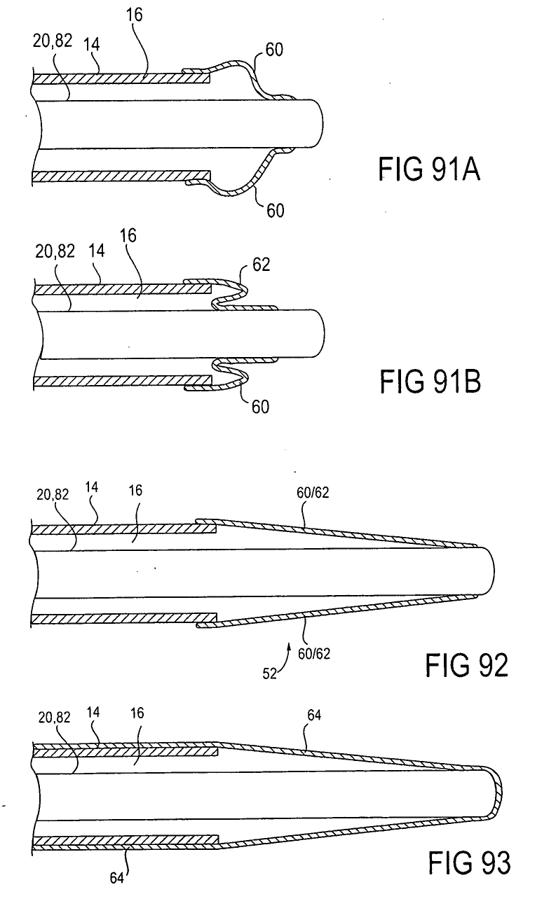

Another variation is shown in FIG. 91A in which covering 60 may be configured as an elastic tubular member. As seen, when endoscope shaft 20 or 80 is in a retracted position, covering 60 may be configured to form a tubular structure when relaxed. As endoscope shaft 20 or 80 is advanced distally, as seen in FIG. 92, covering 60 may stretch along with shaft 20 or 80 to maintain the sterility of guide lumen 16.

Yet another variation is shown in FIG. 91B in which covering 62 may be configured as an elastic rolling diaphragm. When endoscope shaft 20 or 80 is retracted, covering 62 may be configured to revert upon itself such that part of covering 62 may be pulled proximally into guide lumen 16. Such a covering 62 material may comprise any number of elastomers, elastomeric materials, or rubber-type materials, e.g., neoprene or latex. When endoscope shaft 20 or 80 is advanced distally, covering 62 may likewise revert and stretch distally along with shaft 20 or 80, also as shown in FIG. 92.

Alternatively, the covering may simply be a plastic covering or wrapper 64 which is non-elastic, as shown in FIG. 93. Such coverings 64 are conventionally available and may be advanced along with endoscope shaft 20 or 80 and retracted likewise as endoscope shaft 20 or 80 is retracted.

For simplicity of illustration, numerous guide tubes described herein do not illustrate a sheath covering as described above. It is to be appreciated that all guide tubes described herein may be configured to include a sheath or wrapper. The use of a liner or she is particularly important in the protection of the sterile field as discussed below.

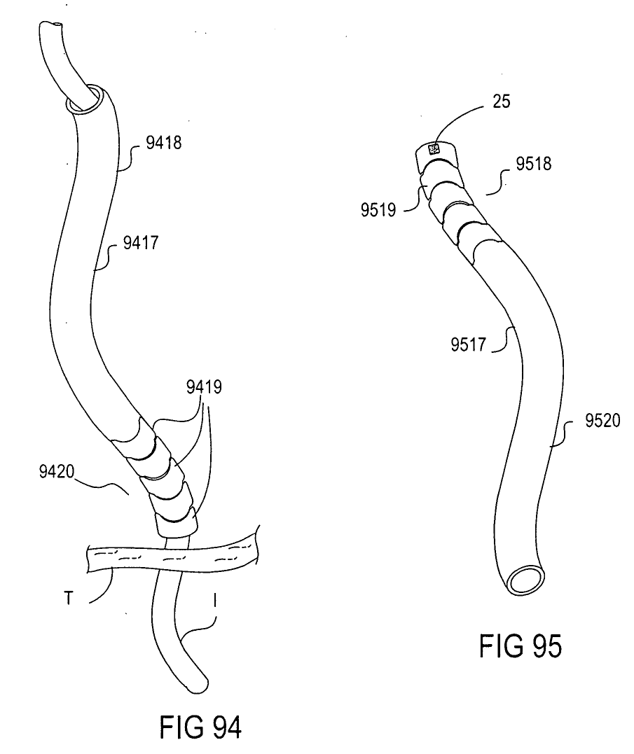

FIGS. 94 and 95 illustrate guide tube embodiments that are partially segmented or only semi-rigidizable. FIG. 94 shows a semi-rigidizable guide tube 9417 having a flexible section 9418 and a selectively rigidizable section 9420 containing segments 9419. The distal end of the guide is placed near the target tissue T. A controllable instrument 1 is present within the lumen of the guide. FIG. 95 illustrates a semi-rigidizable guide 9517 having a segmented, rigidizable distal end 9518. The guide 9517 has a flexible proximal and 9520 and is configured to operate with a datum and position indicator 25.

Semi-rigidizable guides, like partially segmented controllable instruments, have the advantage of simplicity when the surrounding anatomy provides sufficient support for the flexible portion of the instrument or guide. Consider the example where the transluminal procedure involves forming an opening in the wall of the stomach. The flexible proximal portions 9520, 9418 would extend from the mouth through and supported by the esophagus. The rigidizable distal ends would have enough segments 9419, 9519 to provide sufficient curvature, articulation of the stomach walls, and/or access to the desired target location. This example illustrates how the simple design (i.e., fewer segments to control) still retains its functionality.

Multiple Guide Tube Techniques

The rigidizable guide and steerable segmented instrument combination may be advantageously used to perform a wide variety of procedures in the body. One procedure relates to approaching the thoracic cavity by landing the rigidizable overtube onto the stomach, piercing through the stomach wall and advancing the controllable segmented instrument to pierce the diaphragm unaided by an additional rigidizable guide tube. Once through the diaphragm the segmented instrument is navigated, advanced, or otherwise guided into the chest cavity for any procedure that is performed in the thoracic cavity. For example, the segmented instrument working channel or other lumen therein could be used or additional instruments could be provided, for example, for the placement of biventricular leads, or for treatment of atrial fibrillation. Alternatively, a selectively rigidizable guide tube is landed against the stomach wall and after affixing that guide tube, providing an opening in the stomach wall. Thereafter, a second rigidizable guide tube is advanced through the first rigidizable guide tube through the opening in the stomach and to a position on the diaphragm. The second rigidizable guide tube is secured to the diaphragm and an opening in the diaphragm formed. Thereafter a steerable, segmented instrument is advanced navigate through the first and second rigidizable guide tubes to perform any of a variety of trans-diaphragmic procedures within the thoracic cavity. Each of these procedures could be augmented through the use of either or both of the datum and position indicator and the image and mapping system described below.

Another aspect is an overtube inside of an overtube and the external overtube does not leave the stomach. Potentially this overtube anchors to the wall. The internal overtube goes with the scope to the abdominal cavity to support the scope and maintain position. Additional the second scope could be used to anchor onto a second location within the body such as an organ or against the diaphragm wall.

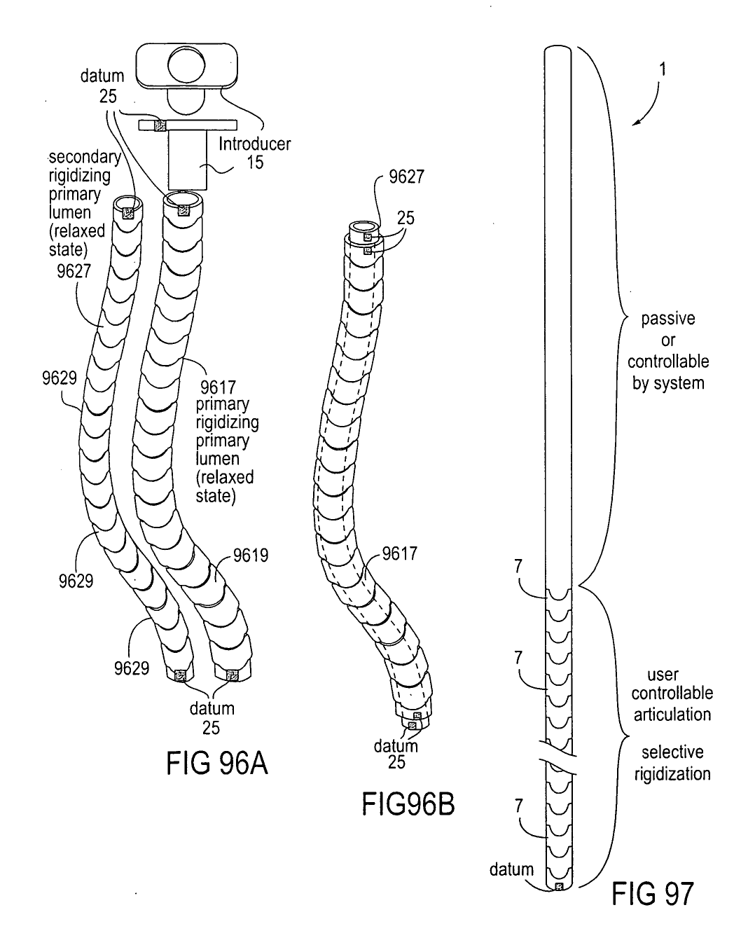

FIGS. 96A, 96B illustrate one possible arrangement for the use of multiple guide tubes. The primary guide tube 9617 is fully segmented and contains a plurality of segments 9619. The primary guide tube 9617 includes a datum and position indicator on the proximal end and the distal end and has a lumen large enough to receive the secondary guide tube 9627. The secondary guide tube 9627 is also fully segmented and has a plurality of segments 9629. The secondary guide tube 9627 is also configured with datum and position indicators 25 on both the proximal and distal ends. FIG. 96B illustrates the secondary guide tube within the lumen of the primary guide tube 9617. The use of multiple datum position indicators provides improved ability to track the shape of each guide tube and its position within the body.

Similar to the semi-rigidizable guide tube described above, FIG. 97 illustrates a partially segmented controllable instrument 1. The partially segmented controllable instrument one contains a plurality of controllable segments 7 on its distal end. The number of controllable segments selected depends upon the particular procedure to be performed by the instrument. The number of controllable segments is selected based on the estimated distance of the desired pathway from the chaff from a transluminal opening to a desired surgical procedure location. Of course additional segments would be added or available should the path be modified or additional surgical location be desired. Like the semi-rigidizable guide, a semi-controllable instrument is simpler to operate because there are fewer controllable segments 7.

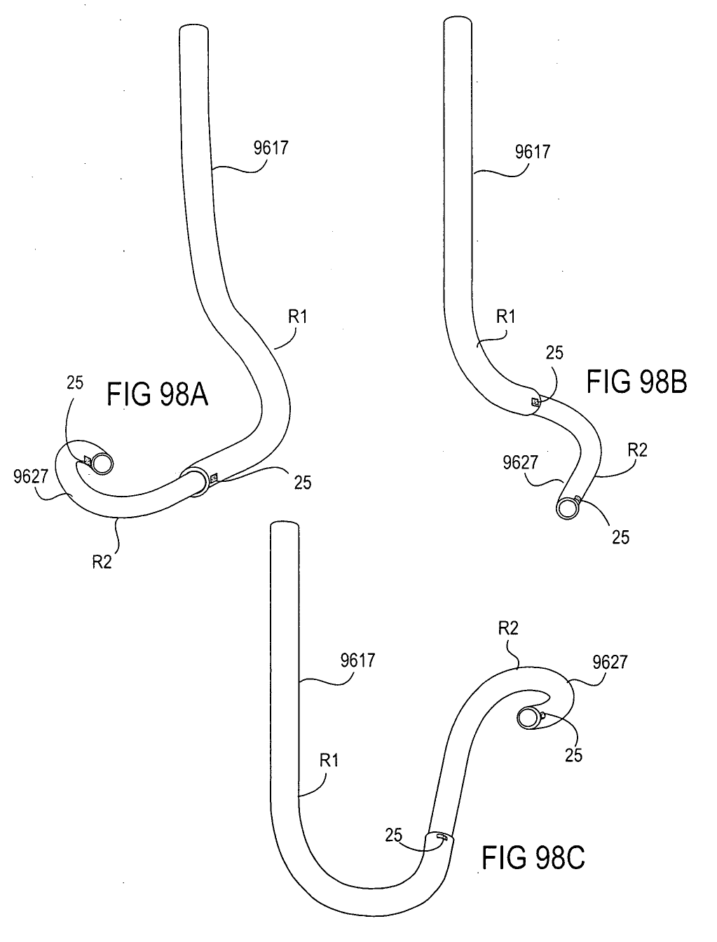

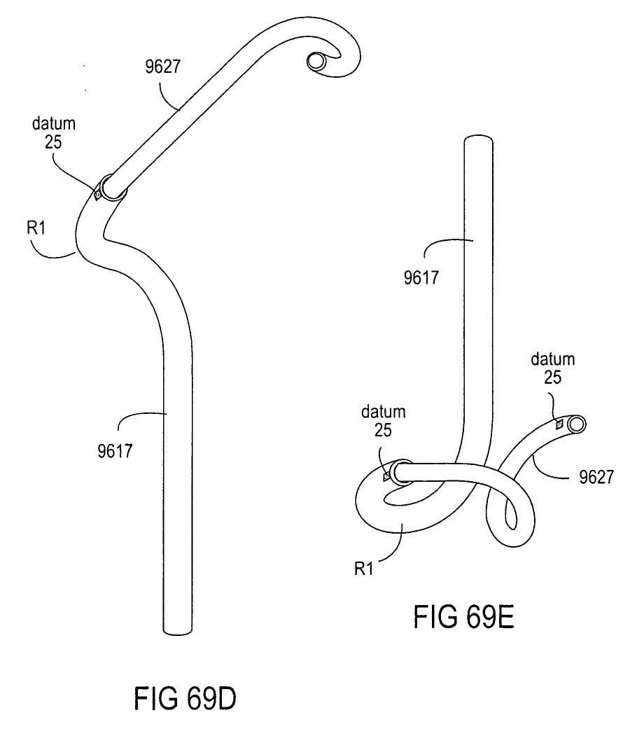

FIGS. 98A through 98E illustrate a wide variety of complex curves that may be obtained using multiple rigidizable guides. In some procedures, the primary rigidizable guide 9617 is maneuvered to anchor against the target tissue for transluminal opening. While not illustrated, the transluminal opening would be at the juncture where the secondary guide tube 9627 exits the lumen of the primary guide 9617. As illustrated by these examples, in a wide variety of anatomically diverse curvatures represented by RI for the primary guide 9617 and R2 for the secondary guide 9627. Numerous surgical pathways may be obtained using guide tubes in this manner.

While the above embodiments are described using a rigidizable guide tube alone or a guide tube having onboard visualization capabilities, it is to be appreciated that other alternatives may be used. For example, the guide tube may be advanced along side a steerable, segmented instrument so that visualization from the instrument is used to position the guide tube against the sidewall. Additionally or alternatively, a steerable segmented instrument could be used alone to grasp the stomach wall and then adjust the segmented sections to provide mechanical advantage that can be applied to reposition the stomach. Thereafter, the working channel of the instrument is used to form the opening in the stomach wall while the stomach is maneuvered away from surrounding tissues or structures.

Sealing may also be accomplished using a seal disposed along a guide tube or other lumen attached to the lumen wall or part of a datum and position indicator, for example. Once the lumen access and/or lumen opening is appropriately sealed, one can inflate the periodontal or other appropriately sealed body cavity. Umbrella sealing design is described below and the use of double balloons has been proposed. The balloons are arranged where one balloon is inside the stomach and another connected balloon is on the outside of the stomach so that when inflated the balloons pressed together against the stomach wall capturing the stomach wall between them.

The guide tube may also be used to provide sealing along the lumen. A sealing ring, such as an inflatable ring on the outer wall of the rigidizable guide tube could be used to seal the esophagus above the opening to the stomach. The inflatable ring could be one of a series of selectable rings based spacing along the guide tube outer wall. One or more rings are inflated depending upon a number of factors such as guide tube position and specific patient anatomy. Additionally or alternatively, an inflatable ring or other sealing means could be advanced along the guide tube outer wall and positioned between the guide tube and a portion of the alimentary canal to seal the stomach. As illustrated below, the use of balloons or other seals to be added to the segmented and of the guide to such that a segmented portion of the guide extends through the transluminal opening to provide guidance.

In alternative embodiment, sealing could be provided in a portion of the lumen of the rigidizable guide tube near the distal end or in a position to provide sealing to gases provided through the opening and into the tissue of interest. In other words, sealing of the guide lumen or steerable instrument may be accomplished using seals on, in or about the distal or sealing end of the instrument or guide or be a separate device provide in the area where sealing is desired.

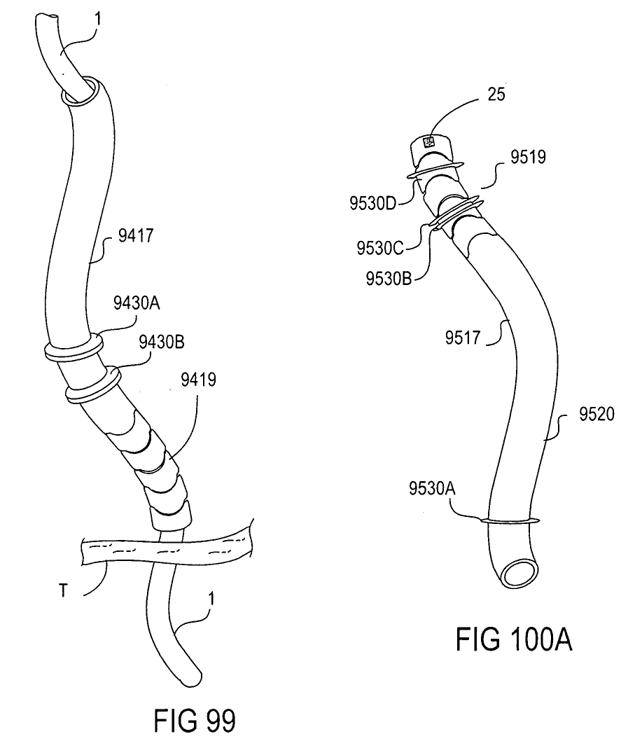

FIG. 99 illustrates a guide tube 9417 with sealing rings 9430A, 9430B on the outer body. Sealing rings may be used to provide a gas tight seal in the lumen through which the guide tube 9417 is positioned. In one illustrative example, where the transluminal procedure occurs in the stomach wall and stomach insufflation is desired, the ratings 9430A and 9430B could be placed a long of the length of the outer wall of the guide tube 9417 such that, when inflated, the rings form a seal with the wall of the esophagus. In an exemplary procedure, a guide tube having sealing rings disposed about or along its outer walls is advanced through a lumen to position for creating a transluminal opening. The rings are inflated to create a seal in support of an insufflation operation or to provide other pressure tight environments as needed.

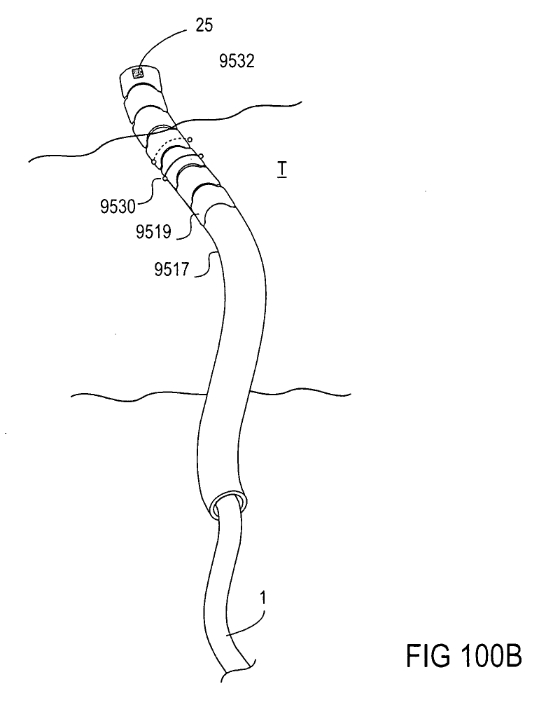

FIG. 100A illustrates a guide tube 9517 having a plurality of seals disposed along its length. In the illustrated example, the guide tube 95 has a seal 9530 a near its proximal end along the flexible portion of this semi-rigidizable guide tube. Additionally, sealing rings 9530B, C and D are placed along the segmented portion of the guide tube.

FIG. 100B illustrates a semi-rigidizable guide tube 9617 having a plurality of segments 9619 and a datum position indicator on its distal end. As illustrated in FIG. 100B, the transluminal opening has been formed in tissue T and the distal end of the guide 9617 extends therethrough. The transluminal opening in tissue T is sealed between ring seal 9430, 9432. In this manner, the segments 9619 distal to ring 9432 may be manipulated to provide further guidance in the body portion accessed by the transluminal opening. An instrument I is disposed within the guide tube lumen and may be used to optimize the desired curvature prior to locking or rigidizing the guide tube 9617 or the distal most segments 9619 (i.e., those the segments beyond the transluminal opening).

The sealing rings in the illustrative example are circular in shape. Other shapes are possible such as, cylindrical shape. The sealing rings may also have a surface texture that allows better sealing based on the surface properties of the lumen to which the seal will engage. The sealing rings are formed from any medical grade polymer capable of expanding under pressure and maintaining the desired sealing pressure.

Datum and Position Indicator and Other Devices to Track Insertion Depth and Location of an Instrument

A datum and position indicator may be used to measure the amount of instrument inserted into the body (a) at the initial opening such as the mouth, the anus or an artificial opening, (b) attached to the wall of the stomach, the gut or other tissue location where a steerable instrument exits the rigidizable guide and is freely moveable or both (a) and (b).

A datum and position indicator is any device used to measure, track or otherwise indicate the length of an instrument or the portion of an instrument passing by, in proximity to or detected by the datum and position indicator and position indicator. A datum and position indicator is a convenient reference point that allows the synchronization of internally generated imaging, externally generated imaging or other forms of data to enable a procedure. One or more datum and position indicators could be used in the procedures described herein. For example, one datum and position indicator could be provided at the mouth on the guide tube to register the steerable instrument entry into the guide tube. The datum and position indicator at the mouth provides a registry for the amount of guide tube that has been dispensed into the body.

Alternatively or additionally, a datum and position indicator could be positioned at the guide tube distal end at or near the landing site. The datum and position indicator could be part of the guide tube or a separate structure. As such, the datum and position indicator could be positioned where the guide tube is landed and/or secured against the stomach wall or other position in the body. In a configuration where there is a datum and position indicator is placed adjacent the distal end of the overtube, then the zero datum and position indicator point or reference point demarks exit from the stomach and entry into the maneuvering space of the body.

The datum and position indicator point is used to determining and controlling the amount of steerable, controllable instrument inserted into the body past the datum and position indicator. In this configuration an overtube enters the mouth and is landed against the stomach wall. The distal end of the overtube is adapted to secure against the stomach wall using the configurations described herein. The distal end of the overtube contains a datum and position indicator sensor to measure, detect, or otherwise indicate the amount, position, or relationship of the segmented instrument that is entering the periodontal cavity. In some embodiments, the segmented instrument could be segmented only on its distal end. In other embodiments, the number of segmented portions of the segmented instrument corresponds to or is more than the length of the segmented instrument that enters the periodontal cavity.

The information on the length of an endoscope or colonoscope inserted into a body organ within a patient may be used to aid in mapping the body organ, anatomical landmarks, anomalies, etc., and/or to maintain real-time knowledge along the entire length of the endoscope position within the body. This is particularly useful when used in conjunction with various endoscopes and/or colonoscopes having a distal steerable portion and an automatically controlled proximal portion which may be automatically controlled by, e.g., a controller. Examples of such devices are described in detail above.

One method for determining endoscopic insertion depth and/or position is to utilize a fully instrumented endoscopic device which incorporates features or elements configured to determine the endoscope's depth of insertion without the need for a separate or external sensing device and to relay this information to the operator, surgeon, nurse, or technician involved in carrying out a procedure. Another method is to utilize a sensing device separate from and external to the endoscope that may or may not be connected to the endoscope and which interacts with the endoscope to determine which portion of the endoscope has passed through or by a reference boundary. The external sensing device may also be referred to herein interchangeably as a datum or datum device as it may function, in part, as a point of reference relative to a position of the endoscope and/or patient. This datum may be located externally of the endoscope and either internally or externally to the body of the patient; thus, the interaction between the endoscope and the datum may be through direct contact or through non-contact interactions.

An instrumented endoscope may accomplish measurement by polling the status of the entire scope (or at least a portion of the scope length), and then determining the endoscope position in relation to an anatomical boundary or landmark such as, e.g., the anus in the case of a colonoscope. The polled information may be obtained by a number of sensors located along the length of the device. Because the sensed information may be obtained from the entire endoscope length (or at least a portion of its length), the direction of endoscope insertion or withdrawal from the body may be omitted because the instantaneous, or near instantaneous, status of the endoscope may be provided by the sensors.

Aside from endoscopes being instrumented to measure insertion depth, other endoscope variations may be used in conjunction with a separate and external device that may or may not be attached to the body and which is configured to measure and/or record endoscope insertion depth. This device may be referred to as an external sensing device or as a datum or datum device. These terms are used interchangeably herein as the external sensing device may function, in part, as a point of reference relative to a position of the endoscope and/or patient. This datum may be located externally of the endoscope and either internally or externally of the body of the patient; thus, the interaction between the endoscope and the datum may be through direct contact or through non-contact interactions. Moreover, the datum may be configured to sense or read positional information by polling the status of sensors, which may be located along the body of the endoscope, as the endoscope passes into the body through, e.g., the anus. The datum may be positioned external to the patient and located, e.g., on the bed or platform that the patient is positioned upon, attached to a separate cart, or removably attached to the patient body, etc.

If the patient is positioned so that they are unable to move with any significant movement during a procedure, the datum may function as a fixed point of reference by securing it to another fixed point in the room. Alternatively, the datum may be attached directly to the patient in a fixed location relative to the point of entry of the endoscope into the patient's body. For instance, for colonoscopic procedures the datum may be positioned on the patient's body near the anus. The location where the datum is positioned is ideally a place that moves minimally relative to the anus because during such a procedure, the patient may shift position, twitch, flex, etc., and disturb the measurement of the endoscope. Therefore, the datum may be positioned in one of several places on the body.

One location may be along the natal cleft, i.e., the crease defined between the gluteal muscles typically extending from the anus towards the lower back. The natal cleft generally has little or no fat layers or musculature and does not move appreciably relative to the anus. Another location may be directly on the gluteal muscle adjacent to the anus.

A determination of the length of an endoscope or colonoscope inserted into a body organ within a patient, or generally into any enclosed space, is useful information which may be used to aid in mapping the body organ, anatomical landmarks, anomalies, etc., and/or to maintain real-time knowledge of the endoscope position within the body. The term endoscope and colonoscope may be used herein interchangeably but shall refer to the same type of device. This is particularly useful when used in conjunction with various endoscopes and/or colonoscopes having a distal steerable portion and an automatically controlled proximal portion which may be automatically controlled by, e.g., a controller. Examples of such devices are described in detail above.

There are at least two different approaches which may be utilized in determining endoscopic insertion depth and/or position when an endoscope has been inserted within the body. One method is to utilize a fully instrumented endoscopic device which incorporates features or elements which are configured to determine the endoscope's depth of insertion and to relay this information to the operator, surgeon, nurse, or technician involved in carrying out a procedure.

Another method is to utilize a sensing device separate from and external to the endoscope and which interacts with the endoscope to determine which portion of the endoscope has passed through or by a reference boundary. The external sensing device may also be referred to herein interchangeably as a datum or datum device as it may function, in part, as a point of reference relative to a position of the endoscope and/or patient. This datum may be located externally of the endoscope and either internally or externally to the body of the patient; thus, the interaction between the endoscope and the datum may be through direct contact or through non-contact interactions.

Instrumented Endoscopes

One method of determination for endoscopic insertion depth and/or position is through an endoscopic device which may be configured to determine its depth of insertion. That is, an endoscopic device may be configured to indicate the portion of the endoscope that has been inserted into a body organ without the need for a separate or external sensing device. This type of determination may reflect an endoscope configured such that its depth measurement is independent of its progress during insertion or withdrawal into the body organ and instead reflects its depth instantaneously without regards to its insertion history.

Such an endoscopic device may accomplish this, in part, by polling the status of the entire scope (or at least a portion of the scope length), and then determining the endoscope position in relation to an anatomical boundary or landmark such as, e.g., the anus in the case of a colonoscope. The polled information may be obtained by a number of sensors located along the length of the device, as described in further detail below. Because the sensed information may be obtained from the entire endoscope length (or at least a portion of its length), the direction of endoscope insertion or withdrawal from the body may be omitted because the instantaneous, or near instantaneous, status of the endoscope may be provided by the sensors. Directional information or history of the endoscope position during an exploratory or diagnostic procedure may optionally be recorded and/or stored by reviewing the endoscope time history of insertion depth.

One variation is seen in FIG. 1OA which shows endoscope assembly 10. Endoscope 12 may be configured to have at least a single circuit 14 wired through the length of the shaft of endoscope 12. Circuit 14 may also be wired through only a portion of the shaft length or through a majority of the shaft length depending upon the desired proportion of the shaft that the operator, surgeon, or technician desires to act as a sensor. The single circuit 14 may thus configure the endoscope 12 to function as a single continuous sensor. Depending upon the type of sensors implemented, as described in further detail below, changes in an output variable received by the sensors may be measured and recorded. The degree of change in the output variable may then be correlated to the length of the endoscope 12 inserted into the body. The change in the output variable may also be based upon varying environmental factors experienced by the endoscope 12. For instance, one example of an environmental factor which may instigate changes in the output variable sensed by the circuit 14 may include pressure sensed from the surrounding tissue, e.g., from the anus, where endoscope 12 is initially inserted into the body. Another factor may include changes in electrical conductivity, e.g., from the tissue, when the endoscope 12 is inserted into the body.

Endoscope 12 may alternatively be configured to detect and correlate the length of the endoscope 12 remaining outside the body rather than inside the body to indirectly calculate the insertion depth. Moreover, the endoscope 12 may additionally detect and correlate both the length of the endoscope 12 remaining outside the body as well as the length of endoscope 12 inserted within the body. Alternatively, endoscope 12 may sense the location of the orifice or anus 20 along the length of the device and then calculate either the length remaining outside the body or the insertion length relative to the position of anus 20.

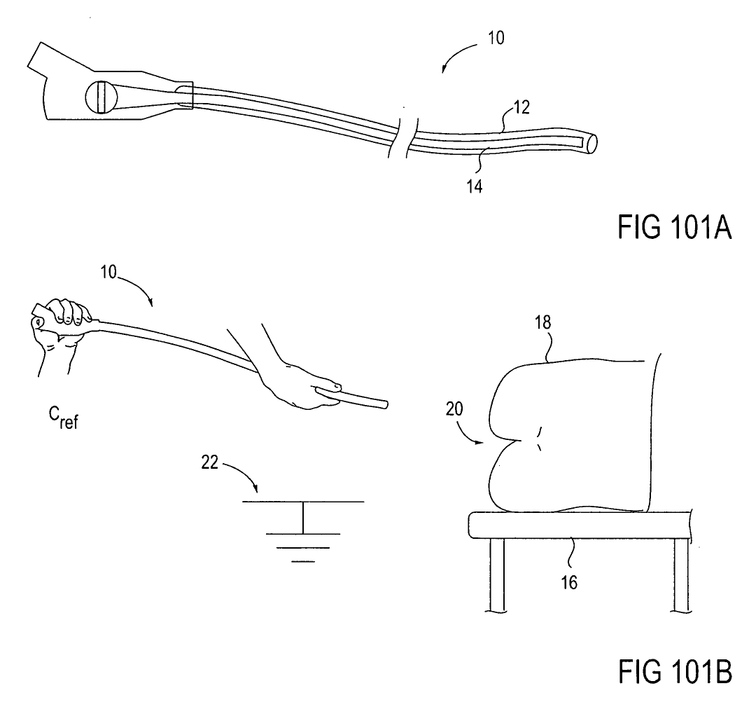

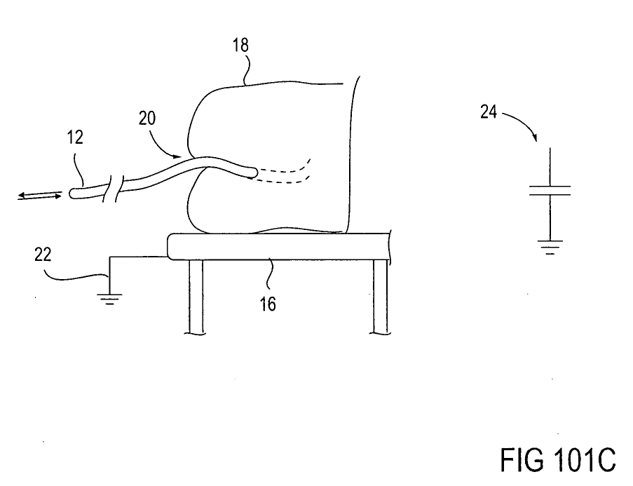

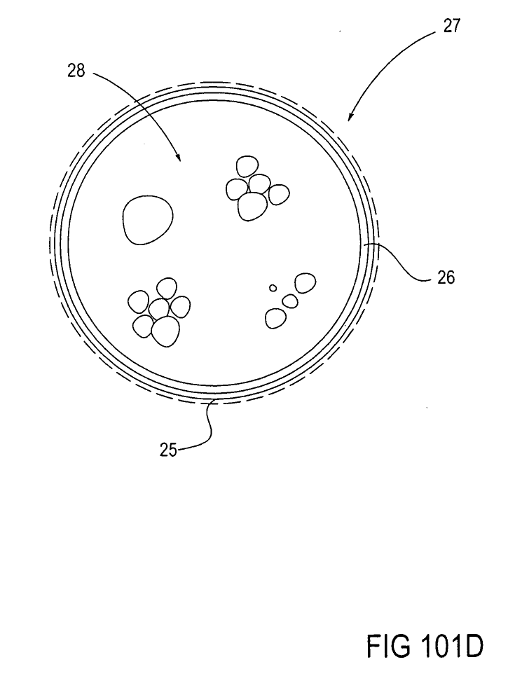

Another example of changing environmental factors leading to a change in an output variable is shown in FIGS. 101B and 101C, which show an example of endoscope assembly 10 configured as a capacitive sensing endoscopic device. As seen in FIG. 101B, patient 18 may be positioned upon table and/or grounding pad 16 which may be connected to electrical ground 22. FIG. 101C shows endoscope 12 inserted within anus 20 of patient 18. Prior to or while endosc6pe 12 is inserted in patient 18, a constant input current may be provided to endoscope 12 and the voltage may be measured in accordance. Endoscope 12 may thus act as a plate within a capacitor while grounding pad 16 placed under patient 18 may function as a second opposing plate to endoscope 12, as represented in the schematic 24. The resulting capacitance between endoscope 12 and grounding pad 16 may be calculated based upon the value of the current, i, over a time period, t, and/or upon the measured difference in phase shift between the input frequency and the resulting frequency. As endoscope 12 is inserted or withdrawn from anus 20, the calculated capacitance will vary according to differences in the dielectric constants between the tissue of patient 18 and that of air. This capacitance change may be constantly monitored and mapped against the length of endoscope 12 to indicate the length of insertion within patient 18.

Another variation on endoscopic sensing may utilize resistivity rather than capacitance. For instance, continuous circuit 14 may be configured into a single printed circuit with an overlay of conductive printed carbon. FIG. 101D shows one variation of a cross-section of endoscope 12 which may be configured as such. As seen, conductive printed carbon layer 25 may be positioned circumferentially within printed flex circuit 26 while surrounding endoscope interior 28. The endoscope 12 may be optionally covered by an outer jacket or sheath 27 to cover the endoscope and its electronics. In use, when the endoscope 12 is inserted into the patient 18 through, e.g., the anus 20, pressure from the surrounding tissue at the point of insertion into the body may force contact between carbon layer 25 and flex circuit 26 within endoscope 12 and thereby close the circuit 14 at the point of insertion. As endoscope 12 is inserted and withdrawn from anus 20, the contact point between carbon layer 25 and flex circuit 26 will vary according to where the pressure is applied at the point of insertion and the resistance of the circuit 14 at any one time may be measured and mapped against the length of endoscope 12 to indicate the length of insertion within anus 20.

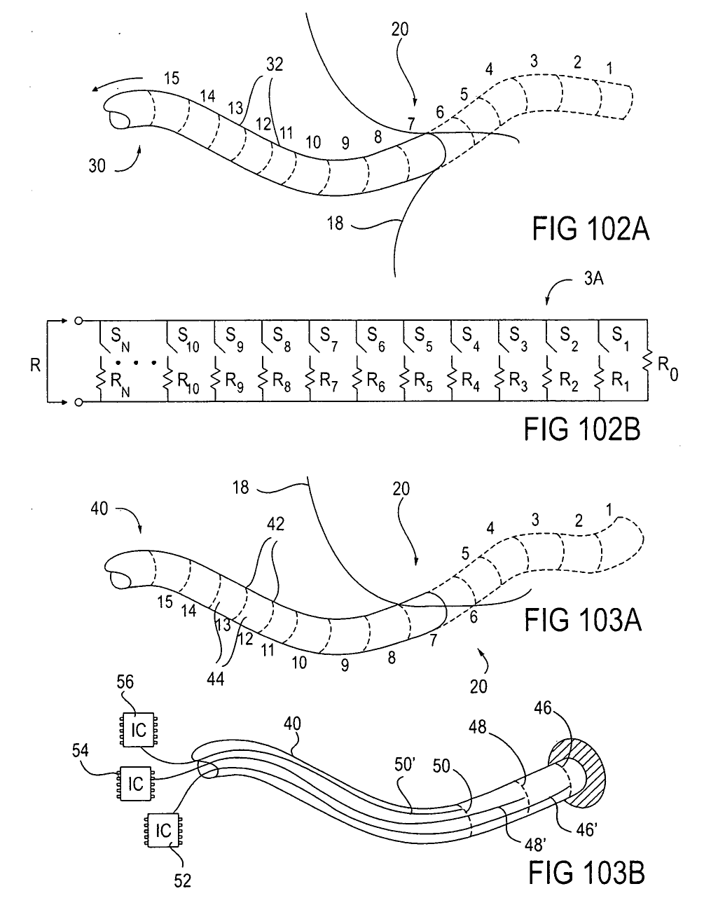

Another variation is shown in FIGS. 102A and 102B, which show an endoscopic device having a series of individual sensors or switches for sensing its insertion depth or position. Endoscope 30 is shown as having a continuous circuit with a plurality of open, individual switches or conductive sections 32 positioned along the length of the device 30. Switches, S.sub.1 to S.sub.N, may be positioned at regular intervals along endoscope 12. The spacing between the switches may vary and may depend upon the desired degree of accuracy in endoscope position determination. Switches may be positioned closely to one another to provide for a more accurate reading, while switches spaced farther apart from one another may provide for a less accurate determination. Moreover, the switches may be positioned at uniform distances from one another, or alternatively they may be spaced apart at irregular intervals, depending upon the desired results. The switches may also take a variety of electrically conductive forms, e.g., membrane switches, force sensitive resistors (FSR), etc.

Another variation on the type of switch which may be used is light-detecting transducers. The switches S.sub.1 to S.sub.N, may be configured as one of a variety of different types of photo-sensitive switches, e.g., photoemissive detectors, photoconductive cells, photovoltaic cells, photodiodes, phototransistors, etc. The switches S.sub.1 to S.sub.N, may be located at predetermined positions along the length of the endoscope 30. As the endoscope 30 is inserted into the patient 18, the change in ambient light from outside the patient 18 to inside the patient 18 may result in a voltage change in the switches inserted within the body 18. This transition may thereby indicate the insertion depth of the endoscope 30 within the body 18 or the length of the endoscope 30 still located outside the body 18. The types of photo-sensitive switches aforementioned may have a current running through them during a procedure, with the exception of photovoltaic switches, which may be powered entirely by the ambient light outside the body 18.

FIG. 102B shows a schematic representation 34 of the device of FIG. 102A. As shown, switches, S.sub.1 to S.sub.N, may be configured such that they are in parallel to one another. Insertion or withdrawal of the endoscope 12 within patient 18 may activate or close a switch through, e.g., interaction with electrically conductive tissue, pressure from the anus closing the switch, changes in moisture or pH, temperature changes, light intensity changes, etc. The closing of a particular switch will vary according to how deep the endoscope 12 is inserted within the anus 20. When a particular switch is electrically activated, a corresponding resistance value, ranging from R.sub.1 to R.sub.N, may be measured and then mapped against the endoscope 12 to indicate the length of insertion.

Another variation is shown in FIGS. 103A and 103B which show an endoscope 40 having a number of sensors positioned along the length of the endoscope 40 at discrete locations. In this variation, a number of sensor wires may be placed along the length of the endoscope 12 such that each wire terminates at subsequent locations along the endoscope 12, as shown in FIG. 103B. Although only three wires are shown, this is merely intended to be illustrative and any number of fewer or additional wires may be utilized depending upon the desired length of the endoscope 12 to be instrumented. The placement of the distal ends of sensor wires 46′, 48′, 50′ may coincide with the number of vertebrae or links of the endoscope 12 structure. The sensor wires 46′, 48′, 50′ may be simply routed through-within the endoscope 12 length or they may be placed along the exterior of the device. The distal ends of the wires may be exposed to allow for communication with the tissue or they may alternatively be each connected to corresponding conductors 42 which divide the endoscope 12 up into a number of segments 44. These optional conductors 42 may be formed in the shape of rings to allow for circumferential contact with the tissue. Each sensor wire 46′, 48′, 50′ may thus be in electrical communication with a corresponding conductor 46, 48, 50, respectively, and so on, depending upon the number of wires and corresponding conductors utilized. The individual sensors may also be networked together on a single bus and more complex networking and placement of sensors may also be implemented to yield additional information, e.g., rotational position of the endoscope 12. The proximal ends of the sensor wires 46′, 48′, 50′ may each be connected to a corresponding processor 52, 54, 56, respectively, such that the length of the endoscope 12 inserted within the anus 20 may be determined by polling the status of each individual sensor wire 46′, 48′, 50′.

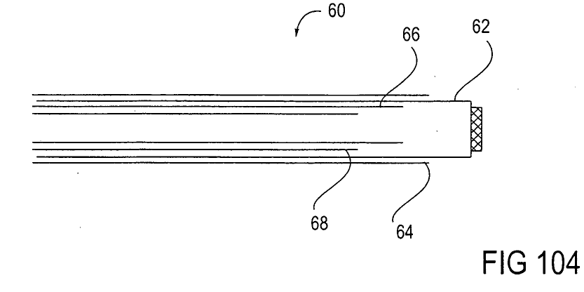

FIG. 104 shows another endoscopic assembly variation 60 in which corresponding pairs of wire sensors may be positioned along an endoscope 62 body. A first pair 64 of wire sensors may extend along the endoscope 62 and terminate at a first distal location; a second pair 66 of wire sensors may also extend along the endoscope 62 and terminate at a second distal location which is proximal of the first distal location; and a third pair 68 of wire sensors may also extend along the endoscope 62 and terminate at a third distal location which is proximal of the second distal location, and so on. Any number of wire pairs may be used and the distances between each of the first, second, third, etc., distal locations may be uniform or irregular, depending upon the desired measurement results. This variation 60 may operate in the same manner as above by measuring which pair of wire sensors is disrupted when inserted or withdrawn from a patient.

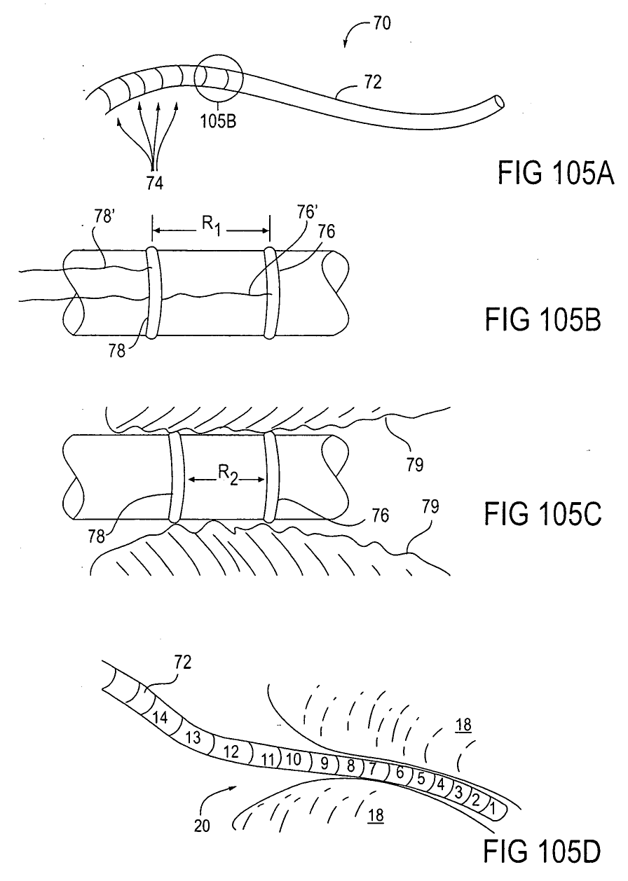

Yet another example is shown in FIGS. 105A to D which shows endoscope assembly 70 which may comprise an endoscope 72 having at least one or more, preferably at least two or more, conductive sensors 74 positioned along the length of endoscope 72. Sensors 74 may be in the shape of rings and may be further configured to measure resistance between each adjacent ring. FIG. 105B is a detailed view of a portion of endoscope 72 which shows first sensor 76 and adjacent second sensor 78. Each sensor 76, 78 may be connected to a separate sensor wire 76′, 78′ such that the electrical resistance, e.g., R.sub.1, between adjacent sensors, e.g., sensors 76, 78, may be measured when contacting a region of tissue. FIG. 105C shows sensors 76, 78 contacting tissue 79. As the endoscope 72 is advanced or withdrawn from the tissue, resistance values between adjacent sensors may be measured to determine the position of the endoscope 72 within the patient 18. As seen in FIG. 105D, resistance values may be subsequently measured between each adjacent sensor, shown as sensors 1, 2, 3, etc., as the device is advanced into patient 18. This may be accomplished, in part, by correlating measured resistance values between sensors where R˜∞ when sensors are measured outside of the body, and R<<∞ when sensors are measured inside the body when surrounded by tissue.

As mentioned above, other output variables aside from pressure or force, capacitance, and resistance measurements may also be employed to determine endoscopic insertion depth. For instance, moisture or pH sensors may be utilized since moisture or pH values change dramatically with insertion into the body. Temperature or heat flux sensing may also be utilized by placing temperature sensors, e.g., thermistors, thermocouples, etc., at varying locations along the endoscope body. Temperature sensing may take advantage of the temperature differences between air and the body. Another alternative may include heating or cooling the interior of the endoscope at ranges above or below body temperature. Thus, the resultant heat flux into or out of the endoscope, depending upon the interior endoscope temperature, may be monitored to determine which portion of the endoscope are in contact with the body tissue. Another alternative may include light sensing by positioning. light sensors at locations along the endoscope body. Thus, light intensity differences may be determined between outside and inside the body to map endoscope insertion depth. Alternatively, sound waves or other pressure waves, ultrasound, inductive proximity sensors, etc., may also be utilized.

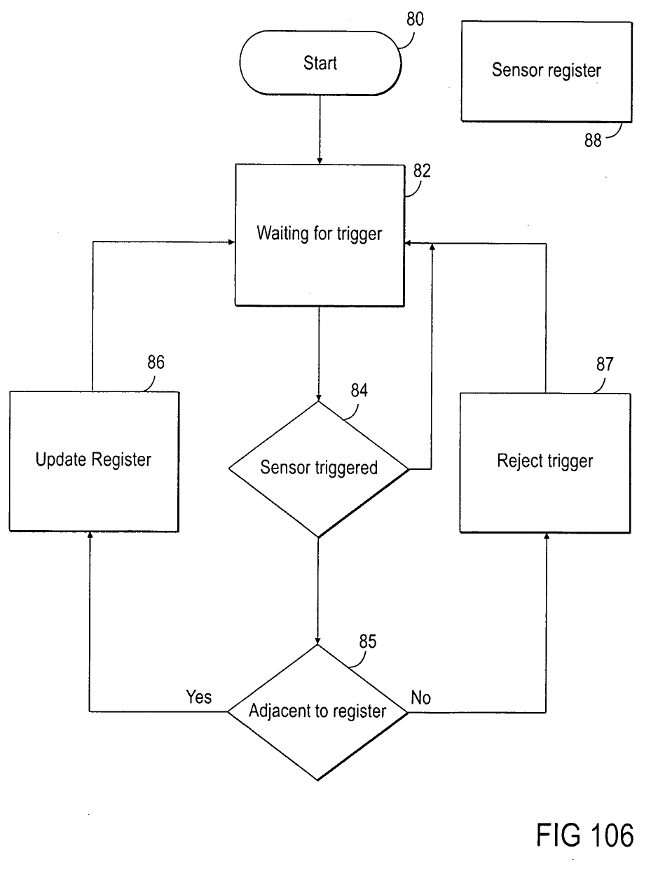

In utilizing sensors positioned upon the endoscope body, an algorithm may be utilized for determining and recording the insertion depth of the endoscope within a patient, as shown in FIG. 106. This variation on an algorithm operates on the general principle that each of the sensors are triggered sequentially as the endoscope is inserted or withdrawn from the patient. A register may be used to record and keep track of the latest insertion depth, i.e., the most recent and valid triggered sensor. The endoscope and algorithm may be configured such that sensor readings that are considered valid are those readings which are triggered by the same sensor or adjacent sensors such that insertion, withdrawal, or no motion may be indicated. Other sensor triggers can be ignored or rejected while valid sensor triggers may cause the register to update.

Such an algorithm may be implemented with any of the devices described above to eliminate false measurements and to maintain accurate insertion depth measurements. Step 80 indicates the start of the algorithm as the endoscope waits for a sensor to be triggered 82. If a sensor has not been triggered 84, the algorithm would indicate a “No” and the device would continue to wait for a trigger signal. Upon an indication that a sensor has been triggered 84, a comparison of the triggered signal takes place to compare whether the sensed signal is from an adjacent sensor 85 by comparing the triggered sensor information to stored register information in sensor register 88. If the triggered signal is not from an adjacent sensor, the signal is rejected as a false signal 87 and the endoscope goes back to waiting for a sensor to be triggered 82. However, if the triggered signal is from an adjacent sensor when compared to the value stored in register 88, register 88 is updated 86 with the new sensor information and the endoscope then continues to wait for another sensor to be triggered 82.

Endoscopes Using External Sensing Devices

Aside from endoscopes being instrumented to measure insertion depth, other endoscopes may be used in conjunction with a separate device configured to measure and/or record endoscope insertion depth. This separate device may be referred to as an external sensing device or as a datum or datum device. These terms are used interchangeably herein as the external sensing device may function, in part, as a point of reference relative to a position of the endoscope and/or patient. This datum may be located externally of the endoscope and either internally or externally to the body of the patient; thus, the interaction between the endoscope and the datum may be through direct contact or through non-contact interactions. Moreover, the datum may be configured to sense or read positional information by polling the status of sensors or transponders, which may be located along the body of the endoscope, as the endoscope passes into the body through, e.g., the anus. Alternatively, the datum may be configured to detect sensors or transponders only within a limited region or area. The datum may be positioned external to the patient and located, e.g., on the bed or platform that the patient is positioned upon, attached to a separate cart, or removably attached either internally or externally to the patient body, etc.

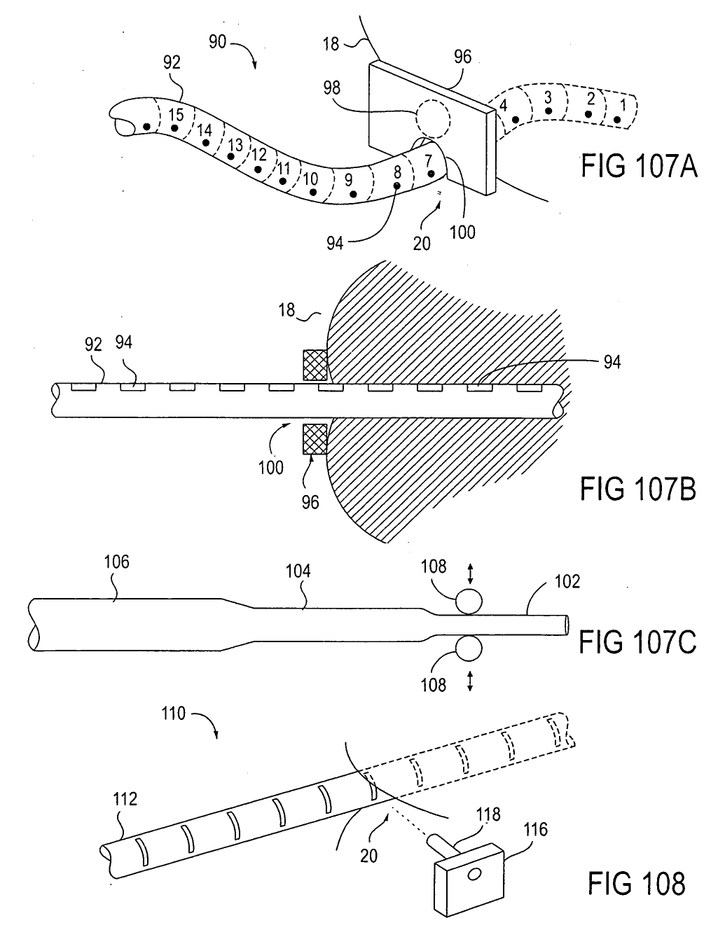

FIGS. 107A and 107B show one variation in using an endoscope assembly 90 in conjunction with external sensing device or datum 96. Datum 96 may be positioned externally of patient 18 adjacent to an opening into a body cavity, e.g., anus 20 for colonoscopic procedures. Datum 96 may accordingly have a sensor or reader 98 located next to opening 100, which may be used as a guide for passage of endoscope 92 therethrough into anus 20. Endoscope 92 may be configured to have a number of tags 94, e.g., sensors, transponders, etc., located along the body of endoscope 92. These tags 94 may be positioned at regular intervals along endoscope 92. The spacing between the tags 94 may vary and may also depend upon the desired degree of accuracy in endoscope position determination. Tags 94 may be positioned closely to one another to provide for a more accurate reading, while tags 94 spaced farther apart from one another may provide for a less accurate determination. Moreover, tags 94 may be positioned at uniform distances from one another, or alternatively they may be spaced apart are irregular intervals, depending upon the desired results. Moreover, tags 94 may be positioned along the entire length of endoscope 92 or only along a portion of it, depending upon the desired results. As shown in FIG. 107B, as endoscope 92 is passed through datum 96 via opening 100 and into anus 20, reader 98 located within datum 96 may sense each of the tags 94 as they pass through opening 100. Accordingly, the direction and insertion depth of endoscope 92 may be recorded and/or maintained for real-time positional information of the endoscope 92.

Any number of technologies may be utilized with tags 94. For instance, one variation may have tags 94 configured as RF identification tags or antennas. Reader 98 may accordingly be configured as a RF receiving device. Each tag 94 may be encoded with, e.g., position information such as the distance of a particular tag 94 from the distal end of endoscope 92. The reader 98 may be configured to thus read in only certain regions or zones, e.g., reader 98 may read only those RF tags passing through opening 100 or only those tags adjacent to anus 20. Alternatively, the RF tags may be configured to transmit the status of, e.g., pressure switches as described above, to datum 96 to determine the length of insertion. Another variation on tags 94 may be to configure the tags for ultrasonic sensing. For example, each tag 94 may be configured as piezoelectric transducers or speakers positioned along the endoscope 92. The reader 98 may thus be configured as an ultrasonic receiver for receiving positional information from tuned transducers or tags 94 each of which relay its positional information. Alternatively, optical sensors may be used as tags 94. In this variation, each tag 94 may be configured as a passive encoded marker located on an outer surface of endoscope 92. These markers may be in the form of a conventional bar code, custom bar code, color patterns, etc., and each may be further configured to indicate directional motion, i.e., insertion or withdrawal. Furthermore, each tag 94 may be configured as active encoded markers, e.g., LEDs which may be blinking in coded patterns. Reader 98 may thus be configured as an optical sensor.

Another alternative may be to configure tags 94 and reader 98 for infrared (IR) sensing in which case IR emitters may be positioned along the length of endoscope 92 such that each IR emitter or tag 94 is configured to emit light at a specific frequency according to its position along the endoscope 92. Reader 98 may thus be configured as an IR receiver for receiving the different frequencies of light and mapping the specific frequency detected against the length of endoscope 92. Yet another alternative may be to have tags 94 configured magnetically such that a magnetic reader in datum 96 can read the position of the device, as described in further detail below.

Yet another alternative may be to configure the datum and endoscope assembly as a linear cable transducer assembly. In this variation, reader 98 may be configured as a transducer having a cable, wire, or some other flexible member extending from reader 98 and attached to the distal end of endoscope 92. While the datum 96 remains external to the patient and further remains in a fixed position relative to the patient, the endoscope 92 may be advanced within the patient while pulling the cable or wire from reader 98. The proximal end of the cable or wire may be attached to a spool of cable or wire in electrical communication with a multi-turn potentiometer. To retract the cable or wire when the endoscope 92 is withdrawn, the spool may be biased to urge the retraction of the cable or wire back onto the spool. Thus, the change of wire length may be correlated to an output of the reader 98 or of the potentiometer to a length of the extended cable and thus the length of the endoscope 92 inserted within the patient.

Yet another alternative may be to mount rollers connected to, e.g., multi-turn potentiometers, encoders, etc., on datum 96. These rollers may be configured to be in direct contact with the endoscope 92 such that the rollers rotate in a first direction when endoscope 92 is advanced and the rollers rotate in the opposite direction when endoscope 92 is withdrawn. The turning and number of revolutions turned by the rollers may be correlated into a length of the insertion depth of endoscope 92.

Yet another alternative may be to use the endoscopes, or any of the endoscopes described herein, in conjunction with conventional imaging technologies which are able to produce images within the body of a patient. For instance, any one of the imaging technologies such as x-ray, fluoroscopy, computed tomography (CT), magnetic resonance imaging (MRI), magnetic field location systems, etc., may be used in conjunction with the endoscopes described herein for determining the insertion depth.

In yet another alternative, the datum may be used to sense the positional information from the endoscope through the use of one or several pressure sensors located on the datum, e.g., datum 96. The pressure sensor may be positioned upon datum 96 such that it may press up against the endoscope 92 as it is advanced or withdrawn. This pressure sensor may be configured, e.g., as a switch, or it alternatively be configured to sense certain features on the endoscope 92, e.g., patterned textures, depressions, detents, etc., which are located at predetermined lengths or length intervals to indicate to the pressure switch the insertion depth of endoscope 92.

Yet another alternative is to sense changes in the diameter of the endoscope body inserted into the patient, as seen in FIG. 107C. The insertion length of the endoscope may have multiple sections each having a unique diameter, e.g., a distal most section 102 may have the smallest diameter and each successive proximal section 104, 106 may have incrementally larger diameters. Alternatively, successive sections may have alternating diameter sizes where a first section may have a first diameter, a second section may have a second larger diameter, and the third section may have a diameter equal to the first diameter or larger than the second diameter, and so on. The differences in endoscopic diameter may be used to detect the endoscopic insertion depth by using a datum 108 which may be configured to maintain contact with the endoscope and move according to the diameter changes of the endoscope, as shown by the arrows. This diameter referencing device and method may be used independently or in conjunction with any of the other methods described herein as a check to ensure that the position of the endoscope concurs with the results using other methods of sensing.

FIG. 108 shows another example in endoscope assembly 110 in which endoscope 112 may have a number of sensors or tags 114 located along the body of the endoscope 112. As endoscope 112 is advanced or withdrawn from anus 20, datum 116, which may be mounted externally of the patient and at a distance from endoscope 112, may have a receiver or reader 118 configured in any of the variations described above. For instance, receiver or reader 118 may be adapted to function as a RF receiver, ultrasonic receiver, optical sensor, or as any of the other variations described above, to read only those tags 114 adjacent to anus 20 and to map their position on the endoscope 112 and thus, the length of insertion.

If reader 118 were configured as an optical sensor, it may further utilize a light source, e.g., LED, laser, carbon, etc., within datum 116. This light source may be utilized along with a CCD or CMOS imaging system connected to a digital signal processor (DSP) within reader 118. The light may be used to illuminate markings located at predetermined intervals along endoscope 112. Alternatively, the markings may be omitted entirely and the CCD or CMOS imaging system may be used to simply detect irregularities normally present along the surface of an endoscope. While the endoscope is moved past the light source and reader 118, the movement of the endoscope may be detected and correlated accordingly to indicate insertion depth.

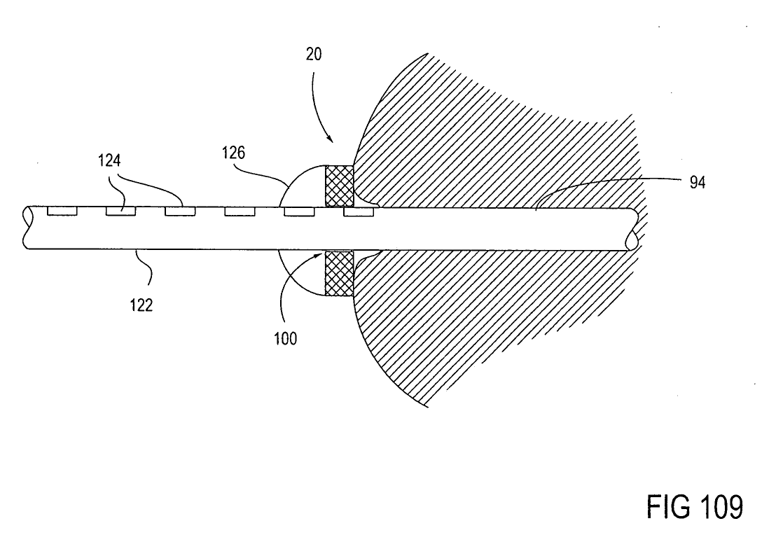

FIG. 109 shows another variation with endoscope assembly 120 in which endoscope 122 may have a number of sensors 124 located along the length of endoscope 122. These sensors 124 may be configured as Hall-effect type sensors, as will be described in greater detail below. The datum 126 may be configured as a ring magnet defining an endoscope guide 128 therethrough such that the magnetic field is perpendicularly defined relative to the sensors 124. Thus, sensors 124 may interact with magnet 126 as they each pass through guide 128. As a Hall sensor 124 passes through datum 126, the sensor 124 may experience a voltage difference indicating the passage of a certain sensor through datum 126. These types of sensors will be described in greater detail below.

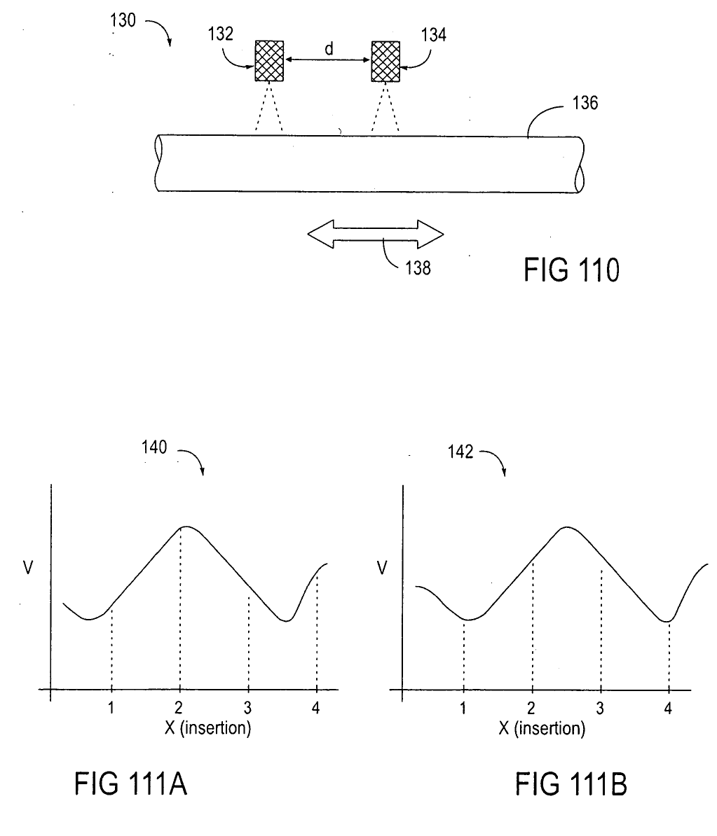

In order to determine the direction of the endoscope when it is either advanced or withdrawn from the patient, directional information may be obtained using any of the examples described above. Another example is to utilize at least two or more sensors positioned at a predetermined distance from one another. FIG. 110 shows one variation illustrating sensor detection assembly 130 with first sensor 132 and second sensor 134. First and second sensors 132, 134 may be positioned at a predetermined distance, d, from one another. As endoscope 136 is advanced or withdrawn past sensor assembly 130, the direction of travel 138 of endoscope 136 may be determined by examining and comparing the signals received from each sensor 132, 134. By determining which sensor has a rising edge or input signal first received relative to the other sensor, the direction of travel 138 may be determined. As shown in FIG. 111A, plot 140 generally illustrates signals received from first sensor 132. From position x=1 to position x=2, a rise in the signal is measured thus sensing a peak in advance of the signal measured from position x=l to position x=2 in plot 142, which is the signal received from second sensor 134, as seen in FIG. 111B. Thus, a first direction of travel, e.g., insertion, may be indicated by the relative comparisons between signals in plots 140 and 142. If endoscope 136 were traveling in the opposite direction, e.g., withdrawal, second sensor 134 would sense a peak in advance of first sensor 132.

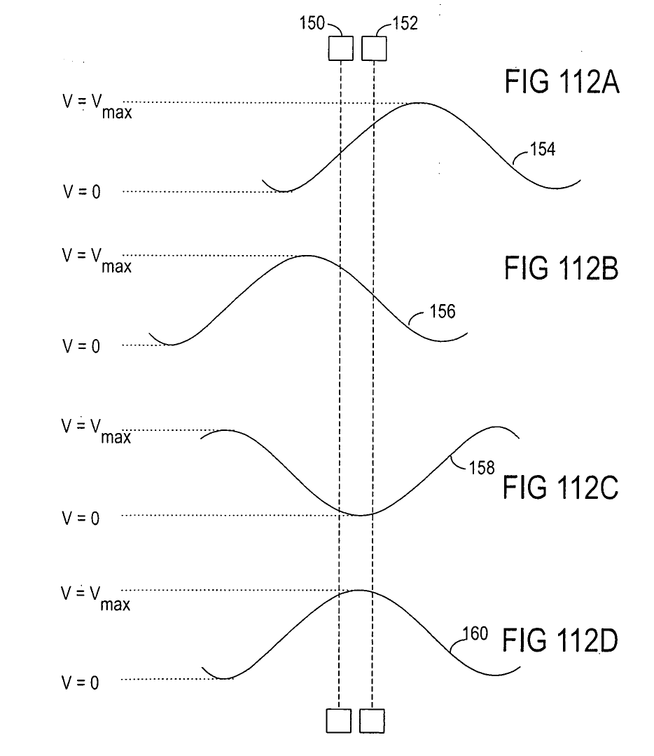

A more detailed description for determining the endoscope's direction of travel follows below. FIGS. 112A to 112D illustrate various cases for determining endoscopic direction of travel using first sensor 150 and second sensor 152. First and second sensors 150, 152 are preferably at a predetermined distance from one another while an endoscope is passed adjacent to the sensors. For the purposes of this illustration, a direction to the right shall indicate a first direction of travel for an endoscope device, e.g., insertion into a body, while a direction to the left shall indicate a second direction of travel opposite to the first direction, e.g., withdrawal from the body.

FIG. 112A shows a situation in which first sensor 150 measures a voltage less than the voltage measured by second sensor 152, as indicated by plot 154. If first and second sensors 150, 152 both measure a decrease in voltage, this may indicate a motion of the endoscope to the right while an increase voltage in both first and second sensors 150, 152 may indicate a motion of the endoscope to the left. FIG. 112B shows another situation in which first sensor 150 measures a voltage greater than the voltage measured by second sensor 152, as indicated by plot 156. If first and second sensors 150, 152. both measure an increase in voltage, this may indicate a motion of the endoscope to the right. However, if both first and second sensors 150, 152 measure a decrease in voltage, this may indicate a motion of the endoscope to the left.

FIG. 112C shows another situation where first sensor 150 measures a voltage equal to a voltage measured by second sensor 152, as shown by plot 158. In this case, if first sensor 150 measures an increase in voltage prior to second sensor 152 also measuring an increase in voltage, this may be an indication of the endoscope moving to the right. On the other hand, if second sensor 152 measures an increase prior to first sensor 150 measuring an increase in voltage, this may indicate movement of the endoscope to the left. FIG. 112D shows a final situation in plot 160 where first sensor 150 again measures a voltage equal to a voltage measured by second sensor 152. In this case, the opposite to that shown in FIG. 12C occurs. For instance, if the voltage measured by first sensor 150 decreases prior to the voltage measured by second sensor 152, this indicates a movement of the endoscope to the right. However, if second sensor 152 measures a voltage which decreases prior to a decrease in voltage measured by first sensor 150, this may indicate a movement of the endoscope to the left.

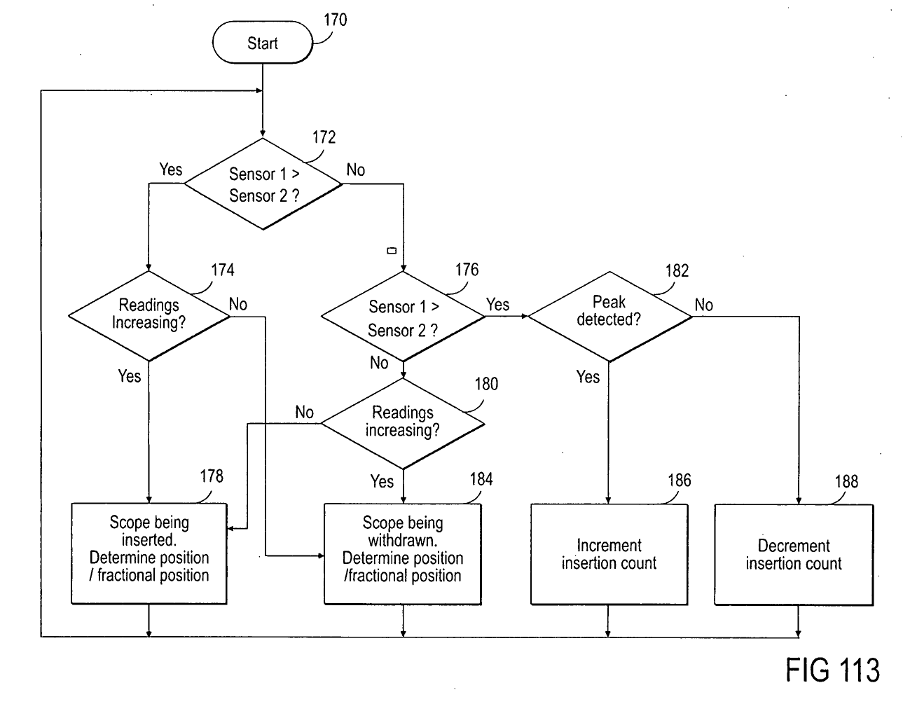

FIG. 113 shows one variation of an algorithm which may be implemented as one method for determining whether an endoscope is being advanced or withdrawn from the body. FIG. 113 illustrates how the various determinations described above may be combined into one variation for an algorithm. As seen, the algorithm begins with step 170. In step 172 an initial step of determining whether first sensor 150 measures a voltage greater than second sensor 152 is performed. If first sensor 150 does measure a voltage greater than second sensor 152, then a second determination may be performed in step 174 where a determination may be made as to whether the voltages measured by both sensors 150, 152 are increasing or not. If both voltages are increasing, step 178 may indicate that the endoscope is being inserted. At this point, the position of the endoscope and its fractional position, i.e., the distance traveled by the endoscope since its last measurement, may be determined and the algorithm may then return to step 172 to await the next measurement.

If, however, first sensor 150 does not measure a voltage greater than second sensor 152 in step 172, another determination may be performed in step 176 to determine whether the voltages measured by sensors 150, 152 are equal. If the voltages are not equivalent, the algorithm proceeds to step 180 where yet another determination may be performed in step 180 to determine if both voltages are increasing. If they are not, then step 178 is performed, as described above. If both voltages are increasing, then step 184 may indicate that the endoscope is being withdrawn. At this point, the position of the endoscope and its fractional position, i.e., the distance traveled by the endoscope since its last measurement, may again be determined and the algorithm may then return to step 172 to await the next measurement.

In step 176, if the voltages measured by first sensor 150 and second sensor 152 are equivalent, then the algorithm may wait to determine whether a peak voltage is detected in step 182. If a peak voltage is detected, step 186 increments the insertion count. However, if a peak is not detected, then step 188 decrements the insertion count. Regardless of whether the insertion count is incremented or decremented, the algorithm may return to step 172 to await the next measurement.

Endoscopes Using Magnetic Sensing Devices

One particular variation on measuring endoscopic insertion depth may utilize magnetic sensing, in particular, taking advantage of the Hall effect. Generally, the Hall effect is the appearance of a transverse voltage difference in a sensor, e.g., a conductor, carrying a current perpendicular to a magnetic field. This voltage difference is directly proportional to the flux density through the sensing element. A permanent magnet, electromagnet, or other magnetic field source may be incorporated into a Hall effect sensor to provide the magnetic field. If a passing object, such as another permanent magnet, ferrous material, or other magnetic field-altering material, alters the magnetic field, the change in the Hall-effect voltage may be measured by the transducer.

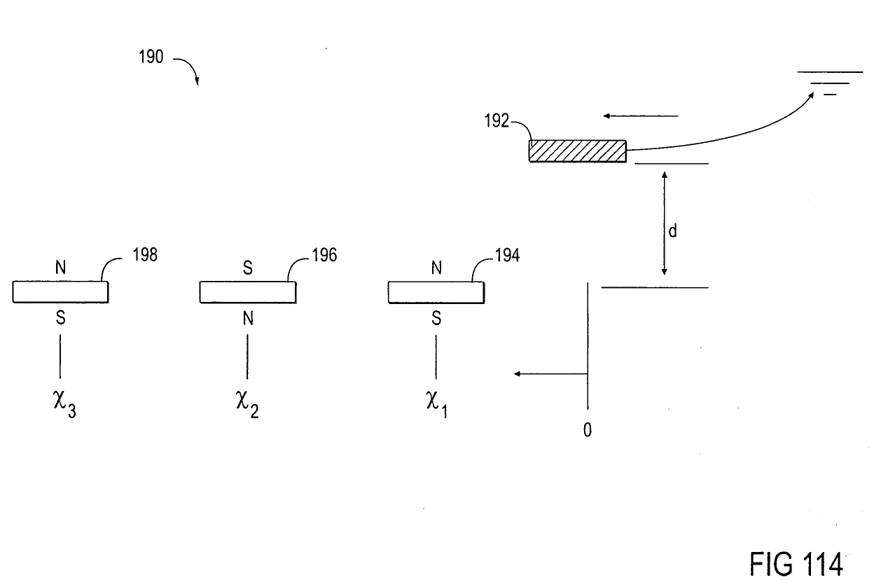

FIG. 114 illustrates generally Hall effect sensor assembly 190 which shows conductor or sensor 192 maintained at a distance, d, as it is passed over magnets 194, 196, 198 at distances x.sub.1, x.sub.2, x.sub.3, respectively. Each magnet may be positioned such that the polarity of adjacent magnets is opposite to one another or such that the polarity of adjacent magnets is the same. As sensor 192 is passed, voltage differences may be measured to indicate which magnet sensor 192 is adjacent to.

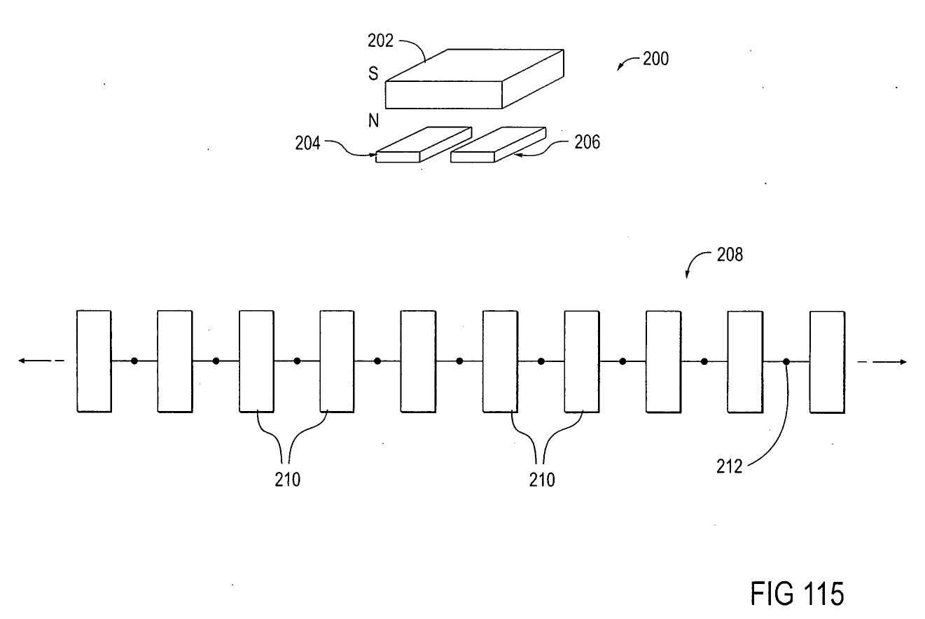

FIG. 115 shows one variation illustrating the general application for implementing Hall effect sensors for endoscopic position measurement. As shown, sensor assembly 200 illustrates one variation having magnet 202 with first sensor 204 and second sensor 206 adjacent to magnet 202. Magnet 202 may be a permanent magnet or it may also be an electromagnet. First and second sensors 204, 206 are connected to a power supply (not shown) and are positioned from one another at a predetermined distance. Both sensors 204, 206 may also be located at a predetermined distance from magnet 202. A general representation of endoscope 208 is shown to reveal the individual links or vertebrae 210 that may comprise part of the structure of the endoscope, as described in further detail in any of the references incorporated above. Each vertebrae 210 is shown as being schematically connected to adjacent vertebrae via joints 212 which may allow for endoscope articulation through tortuous paths. Endoscope 208 may be passed by sensor assembly 200 at a predetermined distance as it is inserted or withdrawn from an opening in a patient. Each or a selected number of vertebrae 210 may be made of a ferrous material or other material that may alter or affect a magnetic field or have ferrous materials incorporated in the vertebrae 210. Thus, as endoscope 208 passes first and second sensors 204, 206, the ferrous vertebrae 210 may pass through and disrupt a magnetic field generated by magnet 202 and cause a corresponding voltage measurement to be sensed by sensors 204, 206. Direction of travel for endoscope 208, i.e., insertion or withdrawal, as well as depth of endoscope insertion may be determined by applying any of the methods described above.

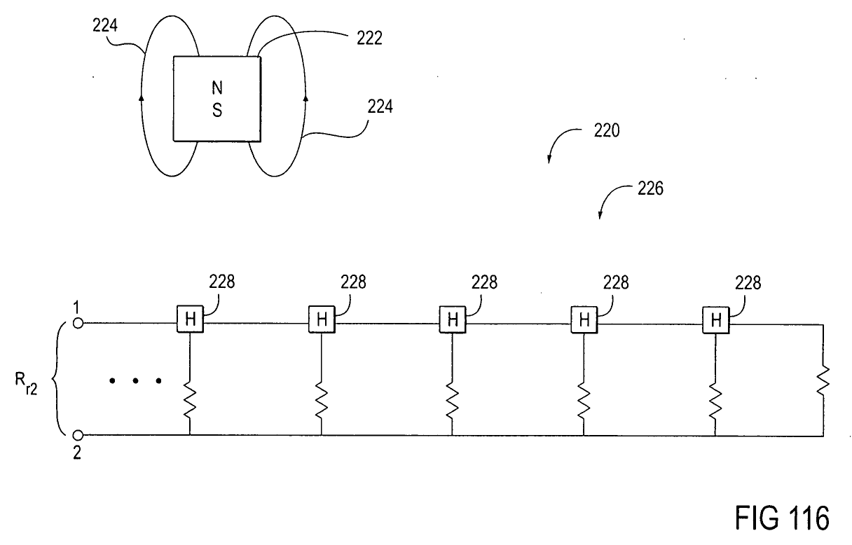

Another variation is shown in FIG. 116 which illustrates a schematic representation 220 of Hall effect sensing in which the sensors may be located on the endoscope 226 itself. Magnet 222 may be positioned adjacent to, e.g., the anus of a patient, such that endoscope 226 passes adjacent to magnet 222 when inserted or withdrawn from the patient. Endoscope 226 may have a number of discrete Hall switches 228 positioned along the body of endoscope 226. As endoscope 226 passes magnet 222, the magnetic field lines 224 may disrupt a switch 228 passing adjacently. Hall switches 228 may be bipolar, unipolar, latched, analog, etc. and may be used to determine the total resistance RI 2 in order to determine insertion length of the endoscope 226.

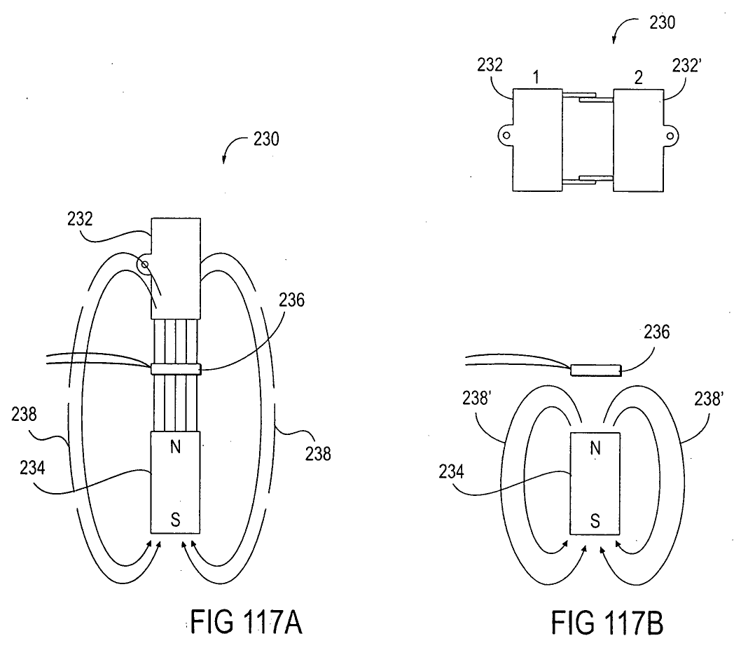

FIGS. 117A and 117B show another variation for Hall sensor positioning. FIG. 117A shows a sensor assembly 230 adjacent to an individual vertebrae 232 of an endoscope. A single vertebrae 232 is shown only for the sake of clarity. As seen, when vertebrae 232 is directly adjacent to magnet 234, magnetic flux lines 238 are disrupted and are forced to pass through sensor 236. Flux lines 238 passing through sensor 236 may cause a disruption in the current flowing therethrough and may thus indicate the passage of the endoscope. FIG. 117B shows the assembly of FIG. 117A when endoscope 230 has been advanced or withdrawn fractionally such that magnet 234 is positioned between adjacent vertebrae 232 and 232′. When a vertebra is not immediately adjacent to magnet 234, flux lines 238′ may return to their normal undisturbed state such that sensor 236 is also undisturbed by magnetic flux. The resumption of current within sensor 236 may indicate that endoscope 230 has been moved relative to sensor assembly 230.

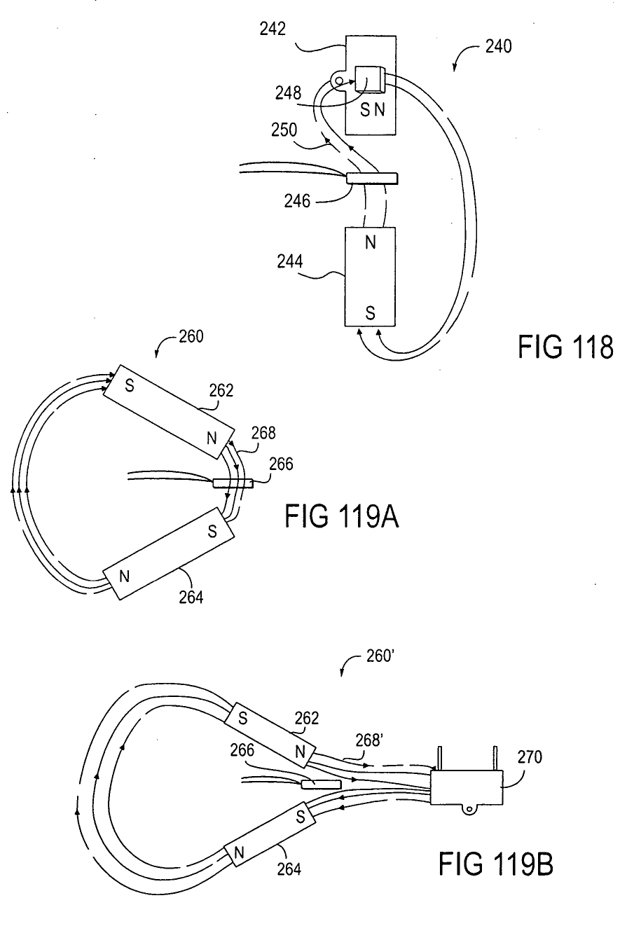

FIG. 118 shows another variation in assembly 240 where a discrete magnet 248 may be positioned on individual vertebrae 242 to produce a more pronounced effect in sensor measurement. Magnets 248 may be positioned along the longitudinal axis of the endoscope for creating a uniform magnetic field radially about the endoscope. Discrete magnets 248 may be permanent magnets or they may alternatively be electromagnets. In either case, they may be placed on as many or as few vertebrae or at various selected positions along the endoscope body depending upon the desired measurement results. As shown, when vertebrae 242 having discrete magnet 248 mounted thereon is brought into the vicinity of magnet 244, the interaction between the magnets produces an enhanced flux interaction 250 such that Hall sensor 246 is able to sense a more pronounced measurement. The polarity of each individual magnet 248 located along the endoscope body may be varied from location to location but the polarity of adjacent magnets on the endoscope body are preferably opposite to one another.

Alternatively, a number of magnets each having a unique magnetic signature may be placed at predetermined positions along the length of the endoscope. Each magnet 248 may be mapped to its location along the endoscope so when a magnet having a specific magnetic signature is detected, the insertion depth of the endoscope may be correlated. The magnets 248 may have unique magnetic signatures, e.g., measurable variations in magnetic field strength, alternating magnetic fields (if electromagnets are utilized), reversed polarity, etc.

FIGS. 119A and 119B show yet another variation in assembly 260 in which more than one magnet may be used in alternative configurations. A first magnet 262 may be positioned at an angle relative to a second magnet 264 such that the combined flux lines 268 interact in accordance with each magnet. Thus, the polarity of each magnet 262, 264 may be opposite to one another as shown in the figures. Sensor 266 may be positioned such that the undisturbed field lines 268 pass through sensor 266. As vertebrae 270 is passed adjacent to sensor 266, the disturbed flux lines 268′, as shown in assembly 260′ in FIG. 119B, may be altered such that they no longer pass through sensor 266 due to the interaction with vertebrae 270. Alternatively, the field lines 268 passing through sensor 266 may be altered in strength as vertebrae 270 passes.

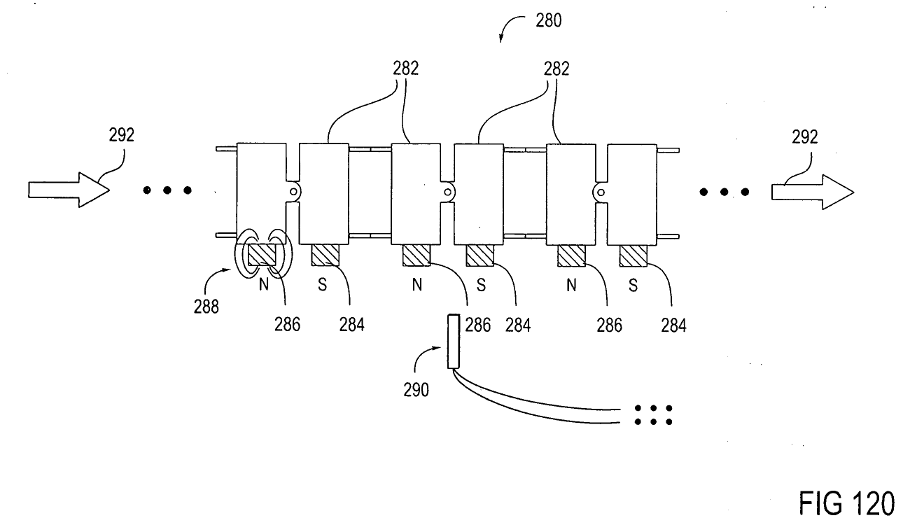

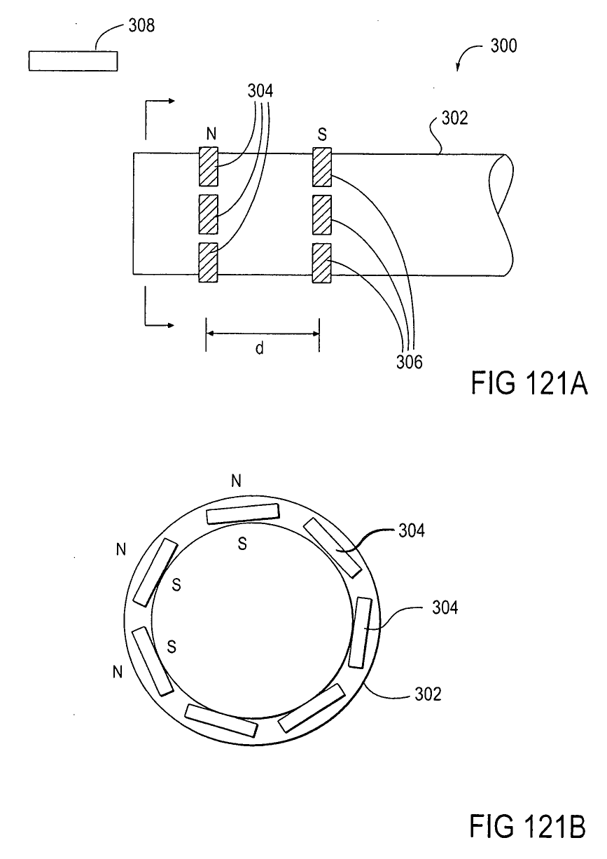

FIG. 120 shows yet another variation in which discrete magnets may be placed on each individual vertebrae of an endoscope assembly. As shown, sensor assembly 280 shows only the vertebrae 282 of an endoscope for clarity. Discrete magnets 284 having a first orientation may be placed on alternating vertebrae 282 while magnets 286 having a second orientation may be placed on alternating vertebrae 282 between magnets 284. Thus, when the endoscope is moved, e.g., along the direction of travel 292, flux lines 288 having alternating directions on each vertebrae 282 can be sensed by sensor 290. The measured alternating flux lines may be used as an indication of endoscope movement in a first or second direction. Each of the magnets may be positioned along the periphery of the vertebrae on a single side; however, they may also be positioned circumferentially, as described below in further detail. FIGS. 121A and 121B show side and cross-sectional views, respectively, of another alternative in magnet positioning. FIG. 121A shows a side view of endoscope assembly 300 in which a number of magnets 304 having a first orientation may be positioned circumferentially about endoscope 302. A number of magnets 306 having a second orientation opposite to the first orientation may also be positioned circumferentially about endoscope 302 separated a distance, d, longitudinally away from magnets 304. With discrete magnets positioned circumferentially about endoscope 302, the rotational orientation of endoscope 302 becomes less important as it passes sensor 308 in determining the insertion depth of the device. FIG. 121B shows a cross-sectional view of the device of FIG. 121A and shows one example of how magnets 304 may be positioned about the circumference. Although this variation illustrates magnets 304 having a “N” orientation radially outward and a “S” orientation radially inward of endoscope 302, this orientation may be reversed so long as the adjacent set of circumferential magnets is preferably likewise reversed. Moreover, although seven magnets are shown in each circumferential set in the figure, any number of fewer or more magnets may be used as practicable.

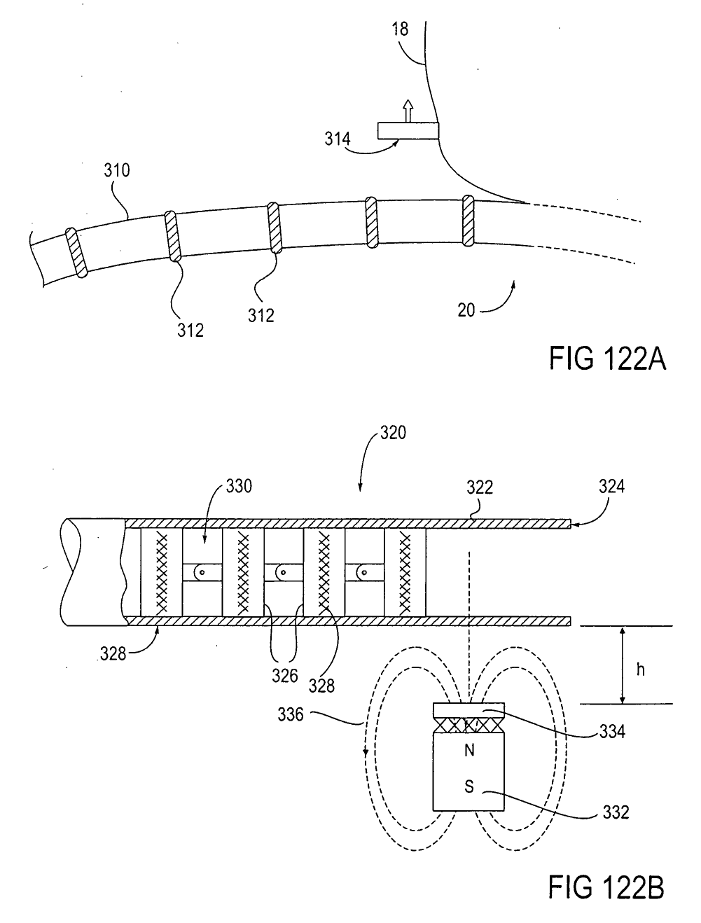

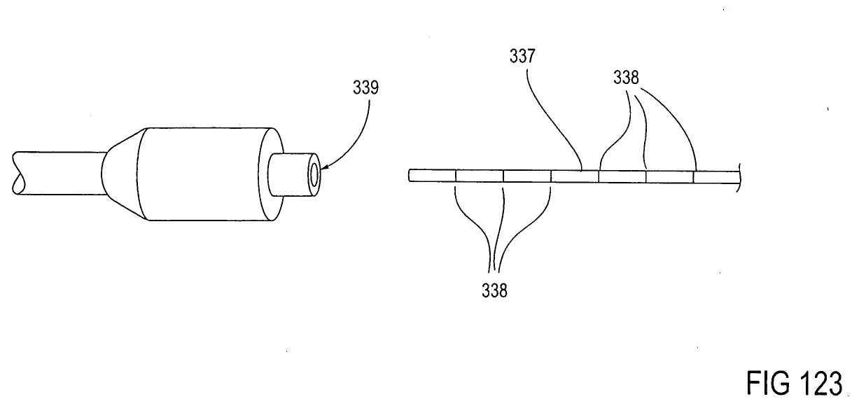

FIG. 122A shows yet another variation in which endoscope 310 may have discrete circumferentially positioned magnets 312 placed at each vertebrae 312 on an outer surface of the endoscope 310. As endoscope 310 is passed into anus 20, Hall sensor 314 may be positioned adjacent to anus 20 such that sensor 314 is able to read or measure the discrete magnets 312 as they pass into anus 20. FIG. 122B shows yet another variation in which endoscope assembly 320 may have endoscope 322 in which individual vertebrae 326 may have some ferromagnetic material 328 integrated or mounted onto or within the vertebrae 326. The ferromagnetic material 328 may be in the form of a band, coating, or other non-obstructive shape for integration onto vertebrae 326 or for coating over portions of vertebrae 326. A sheath or skin 324 may be placed over the vertebrae 326 to provide for a lubricious surface. Between vertebrae 326, non-magnetic regions 330 may be maintained to provide for the separation between vertebrae 326 and between ferromagnetic material 328. Moreover, ferromagnetic material 328 may be applied retroactively not only to endoscopes having vertebrae, but also other conventional endoscopes for which a determination of insertion depth is desired. As endoscope 322 passes magnet 332, sensor 334 may detect disturbances in flux lines 336 as the regions having the ferromagnetic material 328 passes. Additionally, endoscope 322 may be passed at a distance, h, from sensor 334 which is sufficiently close to enable an accurate measurement but far enough away so as not to interfere with endoscope 322 movement.

FIG. 123 shows yet another variation in which conventional endoscopes may be used with any of the Hall sensor datum devices described herein. As shown, elongate support or tool 337 may have a number of magnets 338, or ferrous material or other materials that may alter or affect a magnetic field, positioned along the tool at predetermined intervals. Magnets 338 may be positioned along the length of tool 337 such that the adjacent magnets are either alternating in polarity or uniform in polarity. Furthermore, magnets 338 may be made integrally within the tool 337 or they may be made as wireforms or members which may be crimped about tool 337. Tool 337 may be positioned within the working lumen 339 of any conventional endoscope for use with a datum device as described herein. The inclusion of the tool 337 may then enable the determination of insertion depth of a conventional or instrumented endoscope. If a conventional endoscope is used, tool 337 may be securely held within the working lumen 339 during an exploratory procedure. Tool 337 may optionally be removed during a procedure to allow for the insertion of another tool and then reinserted within lumen 339 at a later time to proceed with the insertion and/or withdrawal of the endoscope.

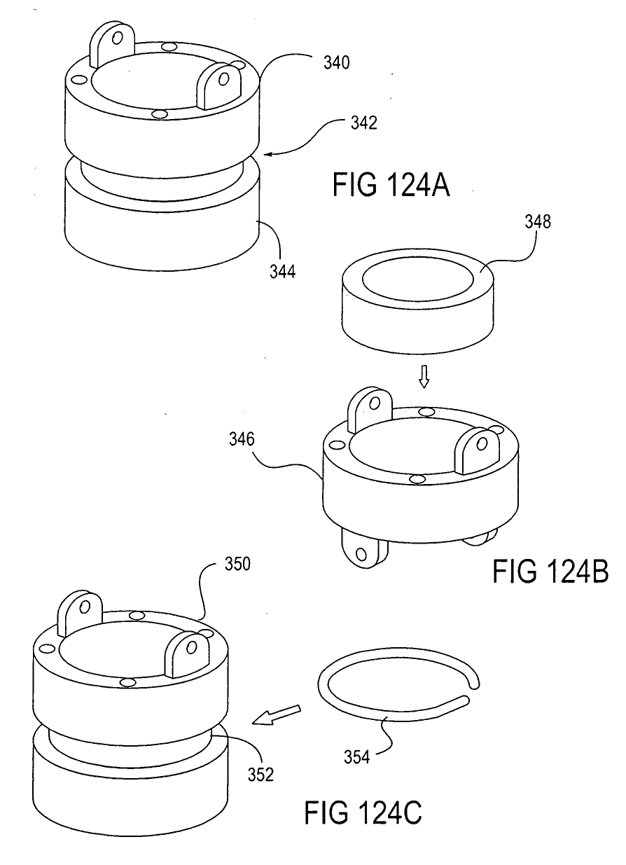

FIGS. 124A to 124C show perspective views of alternative variations for attaching permanent magnets, ferrous materials, or other materials that may alter or affect a magnetic field, onto individual vertebrae. FIG. 124A shows one variation in which vertebrae 340 may be manufactured with a notch or channel 342 circumferentially defined along its outer surface 344. A ring made of a ferrous material or other material that may alter or affect a magnetic field, such as permanent magnets, may be placed within notch 342. FIG. 124B shows another variation in which a formed ring 348 made of a permanent magnet or other such materials may be separately formed and attached onto vertebrae 346. FIG. 124C shows yet another variation in which a wire form 354 made from a ferrous material or other material that may alter or affect a magnetic field, such as a permanent magnet, may be placed within notch 352 of vertebrae 350. Alternatively, ferrous powder may be molded into a circumferential shape and placed within notch 352. Another alternative may be to simply manufacture the entire vertebrae from a ferrous metal or simply cover a vertebrae or a portion of the vertebrae with a ferrous coating.

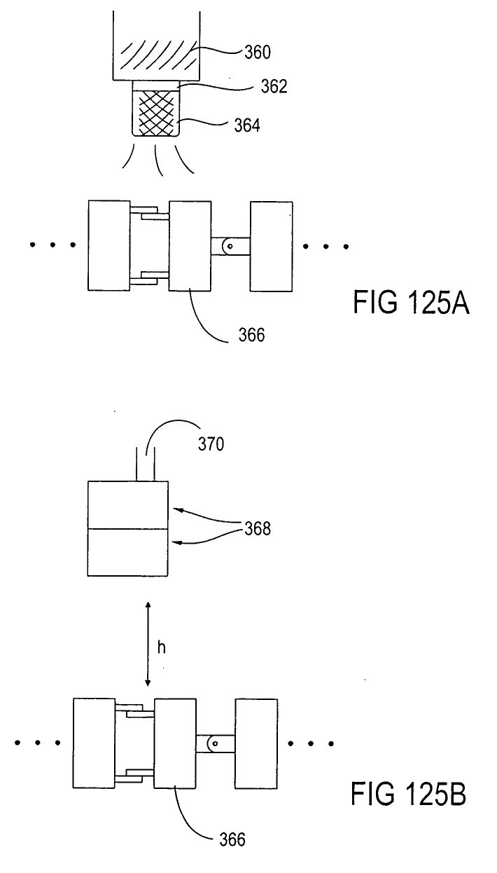

Another alternative for utilizing Hall sensors is seen in FIGS. 125A and 125B. The variation in FIG. 125A may have a fixed platform 360 upon which a magnet 364 may be mounted. A pressure sensor or microforce sensor 362 may be placed between magnet 364 and platform 360. As an endoscope is passed adjacent to magnet 364, the magnet 364 may be attracted to vertebrae 366 as it passes adjacently. Vertebrae 366 may optionally include ferrous materials or other materials that may alter or affect a magnetic field as described above to enhance the attraction and/or repulsion. As magnet 364 is pulled or repulsed by the magnetic force, pressure sensor 362 may record the corresponding positive or negative force values for correlating to endoscope insertion depth. FIG. 125B shows another example in which magnets 368 may be attached to a pressure gauge 370, e.g., a Chatillon® gauge made by Ametek, Inc. As the endoscope passes magnets 368 at some distance, h, the attraction and/or repulsion between magnets 368 and vertebrae 366 may be accordingly measured by gauge 370 and similarly correlated to endoscope insertion depth.

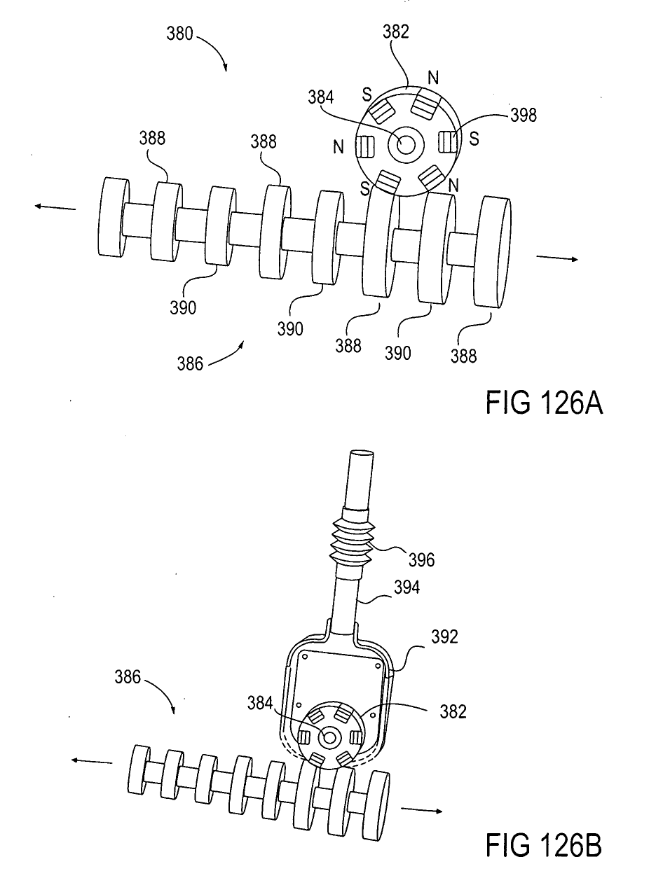

Yet another variation is shown in FIGS. 126A and 126B in assembly 380. Rather than utilizing the linear motion of an endoscope past a static datum, a rotatable datum 382 may be used to record insertion length. Datum wheel 382 may be configured to rotate about pivot 384 while sensing the movement of endoscope 386, which shows only schematic representations of the vertebrae for clarity. The datum wheel 382 may have a number of magnets 398 incorporated around the circumference of wheel 382. Each magnet may be arranged in alternating pole configurations or alternatively in the same pole arrangement. Each of the magnets 398 are also preferably spaced apart from one another at intervals equal to the linear distances between the magnets 388, 390 or permanent magnet located along the body of endoscope 386. Ferrous materials, or materials that may otherwise alter a magnetic field, may be used in place of the permanent magnets. As endoscope 386 is moved past datum wheel 382, wheel 382 rotates in corresponding fashion with the linear movement of endoscope 386 past the datum 382.

The rotation of datum wheel 382 that results when endoscope 386 is moved past can be sensed by a variety of methods. One example includes rotary optical encoders, another example includes sensing the movement of magnets 398 on datum wheel 382 as they rotate relative to a fixed point as measured by, e.g., Hall effect sensors or magnetoresistive sensors. As datum wheel 382 rotates with the linear movement of endoscope 386, datum wheel 382 may directly touch endoscope 386 or a thin material may separate the wheel 382 from the body of endoscope 386. FIG. 26B shows one variation of an assembly view of datum wheel 382 which may be rotatably attached to housing 392. Housing 392 may be connected to stem or support 394, which may extend from housing 392 and provide a support member for affixing datum wheel 382 to the patient, an examination table, a stand, or any other platform. Support 394 may also be used to route any cables, wires, connectors, etc., to housing 392 and/or datum wheel 382. The associated sensors and various support electronics, e.g., rotary encoders, magnetic field sensors, etc., may also be located within housing 392. Support 394 may further include an optional flexible joint 396 to allow datum wheel 382 to track the movement of endoscope 386 as it passes into or out of a patient.

Examples of External Sensing Devices

The external sensing devices, or datum, may function in part as a point of reference relative to a position of the endoscope and/or patient, as described above. The datum may accordingly be located externally of the endoscope and either internally or externally to the body of the patient. If the patient is positioned so that they are unable to move with any significant movement during a procedure, the datum may function as a fixed point of reference by securing it to another fixed point in the room, e.g., examination table, procedure cart, etc. Alternatively, the datum may be attached directly to the patient in a fixed location relative to the point of entry of the endoscope into the patient's body. The datum variations described herein may utilize any of the sensing and measurement methods described above.

For instance, for colonoscopic procedures the datum may be positioned on the patient's body near the anus. The location where the datum is positioned is ideally a place that moves minimally relative to the anus because during such a procedure, the patient may shift position, twitch, flex, etc., and disturb the measurement of the endoscope. Therefore, the datum may be positioned in one of several places on the body.

While the embodiments and specific examples described above relate to endoscopic procedures, it is to be appreciated that the techniques, devices, and systems used may be adapted for use within the body as part of a datum and position indicator as well as adapted for use in transluminal procedures.

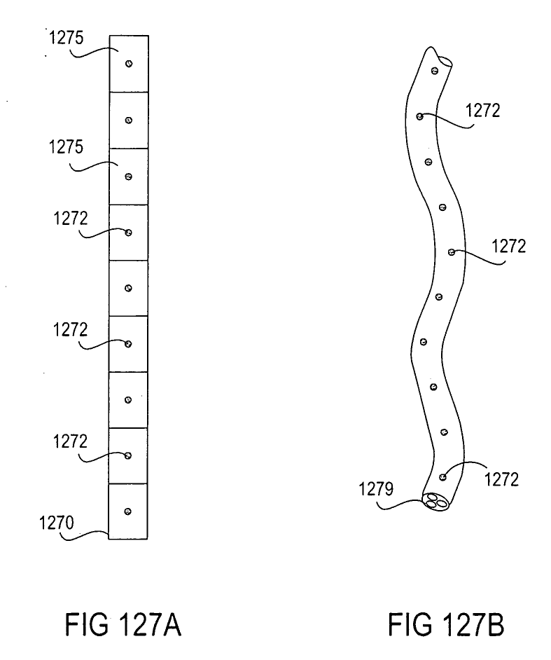

In the most general way, data and position indicator is a reader of a position or proximity sensor placed on an instrument. So long as the reader can detect the position and/or passage of the instrument, the datum aspect is provided in the position of the instrument is known relative to the reader, here he datum and position indicator. As such, even a conventional instrument can be equipped to operate in a system utilizing datum and position indicators and mapping control systems described herein. FIG. 127A illustrates a flexible strip 1270 divided into sectors 1275. Within each sector 1275 is a position or detection indicator 1272. A position or detection indicator 1272 is any device keyed to register with the datum and position indicator as described in greater detail below. FIG. 127B illustrates the flexible strip 1270 in place on a conventional endoscope 1279. Note that after application of the flexible strip 1270, position indicators 1272 are distributed at regular intervals along the length of the endoscope. In this way, it is a datum and position indicator would be able to determine the length of into scope 1279 that has passed the datum point. The amount of scope that has passed the datum point would then be available for mapping and control aspects described herein.

The coded strip 1270 could be applied to any instrument. Once a coded strip is added to conventional instrument, then that instrument may be detected by the datum and position indicator. Application of the coded strip 1270 would then make a conventional scope “DPI reader ready”.

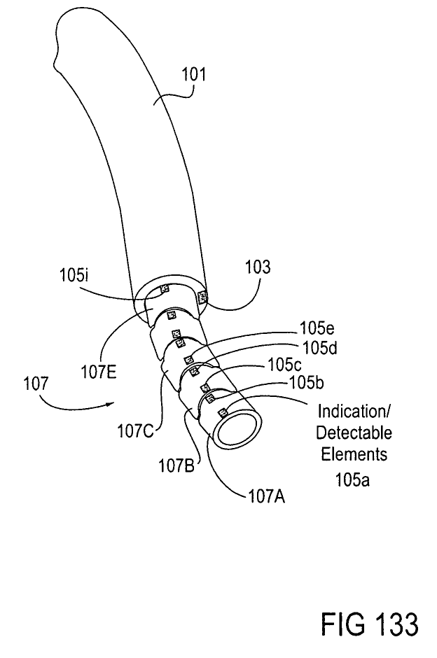

As illustrated in FIG. 133 below, the coded position and/or detection elements could also be designed and built into the instrument itself. It is also envisioned that position and/or detection elements may be added to existing scope structure at specific points (i.e., hinges, joints or other structural elements) or incorporated into scope elements such as into the skin, vertebra or joints

Datum and Position Indicator and Imaging System

The position and datum indicator may be used as part of position detection and control system to provide information for position registration and detection of a controllable instrument moving within the body and/or relative to the datum position indicator.

FIG. 128 is a diagram of an exemplary surgical instrument navigation system 10. In accordance with one aspect of the present invention, the surgical instrument navigation system 10 is operable to visually simulate a virtual volumetric scene within the body of a patient, such as an internal body cavity, from a point of view of a surgical instrument 12 residing in the cavity of a patient 13. To do so, the surgical instrument navigation system 10 includes a surgical instrument 12, a data processor 16 having a display 18, and a tracking subsystem 20 that may also house articulation elements or additional controls to assist in the manipulation and articulation of the surgical instrument 12. The surgical instrument navigation system 10 may further include (or accompanied by) an imaging device 14 that is operable to provide image data to the system. The imaging device 14 may be any suitable medical imaging modality as described herein.

The surgical instrument 12 may be an instrument or instruments that are flexible, steerable, controllable, rigidizable and combinations thereof or other instruments as described herein. The surgical instrument 12 is modified to include one or more tracking sensors that are detectable by the tracking subsystem 20. It is readily understood that other types of surgical instruments (e.g., a guide wire, a pointer probe, a stent, a seed, an implant, an endoscope, etc.) are also within the scope of the present invention. It is also envisioned that at least some of these surgical instruments may be wireless or have wireless communications links. It is also envisioned that the surgical instruments may encompass medical devices which are used for exploratory purposes, testing purposes or other types of medical procedures including transluminal procedures and other procedures described herein.

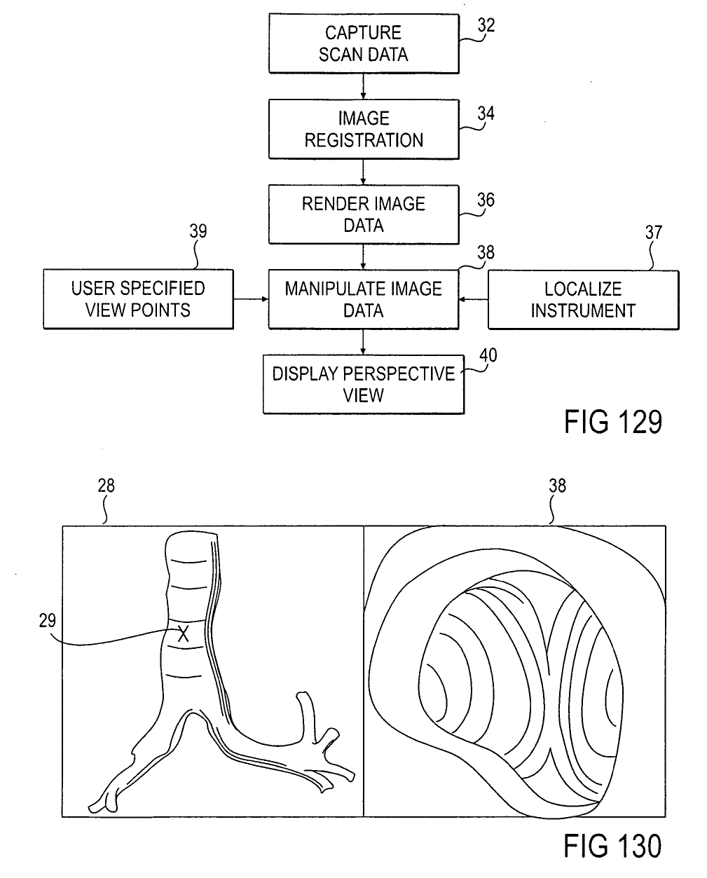

Referring to FIG. 129, the imaging device 14 is used to capture scan data 32 representative of an internal region of interest within the patient 13. The scan data may be obtained prior to surgery on the patient 13. In this case, the captured scan data may be stored in a data store associated with the data processor 16 for subsequent processing. However, one skilled in the art will readily recognize that the principles of the present invention may also extend to scan data acquired during surgery. It is readily understood that scan data may be acquired using various known medical imaging devices 14, including but not limited to a magnetic resonance imaging (MRI) device, a computed tomography (CT) imaging device, a positron emission tomography (PET) imaging device, a 2D or 3D fluoroscopic imaging device, and 2D, 3D or 4D ultrasound imaging devices. The scan data may be multidimensional including two-dimensional and three-dimensional scan data. In the case of a two-dimensional dimensional ultrasound imaging device or other two-dimensional image acquisition device, a series of two-dimensional data sets may be acquired and then assembled into volumetric data as is well known in the art using a two-dimensional to three-dimensional conversion.

A dynamic reference frame 19 is attached to the patient proximate to the region of interest within the patient 13. The functionality of the dynamic reference frame 19 may be provided in a stand-alone component or part of another component as described herein, such as a datum position indicator, a guide tube, or other component or instrument. To the extent that the region of interest is a vessel or a cavity within the patient, it is readily understood that the dynamic reference frame 19 may be placed within the patient 13. To determine its location, the dynamic reference frame 19 is also modified to include tracking sensors detectable by the tracking subsystem 20. The tracking subsystem 20 is operable to determine position data for the dynamic reference frame 19 as further described below.

The scan data is then registered as shown at 34. Registration of the dynamic reference frame 19 generally relates information in the scan data to the region of interest associated with the patient. This process is referred to as registering image space to patient space. Often, the image space must also be registered to another image space. Registration is accomplished through knowledge of the coordinate vectors of at least three non-collinear points in the image space and the patient space. Registration may be accomplished using any conventional image registration technique.

Registration for image guided surgery can be completed by different known techniques. First, point-to-point registration is accomplished by identifying points in an image space and then touching the same points in patient space. These points are generally anatomical landmarks that are easily identifiable on the patient. Second, surface registration involves the user's generation of a surface in patient space by either selecting multiple points or scanning, and then accepting the best fit to that surface in image space by iteratively calculating with the data processor until a surface match is identified. Third, repeat fixation devices entail the user repeatedly removing and replacing a device (i.e., dynamic reference frame, etc.) in known relation to the patient or image fiducials of the patient. Fourth, automatic registration by first attaching the dynamic reference frame to the patient prior to acquiring image data. It is envisioned that other known registration procedures are also within the scope of the present invention, such as that disclosed in U.S. Ser. No. 09/274,972, filed on Mar. 23, 1999, entitled “NAVIGATIONAL GUIDANCE VIA COMPUTER-ASSISTED FLUOROSCOPIC IMAGING”, which is hereby incorporated by reference.

During surgery, the surgical instrument 12 is directed by the surgeon to the region of interest within the patient 13. The tracking subsystem 20 employs electromagnetic sensing to capture position data 37 indicative of the location and/or orientation of the surgical instrument 12 within the patient. The instrument, via its control system, may also provide position, shape and other information including position information derived from the articulation system of the instrument (i.e., such as detailed above with regard to the connector system for a controllable articulating instrument). The instrument information may also be provided relative to the dynamic reference frame 19. The tracking subsystem 20 may be defined as a localizing device 22 and one or more electro-magnetic sensors 24 may be integrated into the items of interest, such as the surgical instrument 12. In one embodiment, the localizing device 22 is comprised of three or more field generators (transmitters) mounted at known locations on a plane surface and the electromagnetic sensor (receivers) 24 is further defined as a single coil of wire. The positioning of the field generators (transmitter), and the sensors (receivers) may also be reversed, such that the generators are associated with the surgical instrument 12 and the receivers are positioned elsewhere. Although not limited thereto, the localizing device 22 may be affixed to an underneath side of the operating table that supports the patient. Alternatively, the localizing device may be provided on the dynamic reference frame 19. In one embodiment, the dynamic reference device 19 is a datum position indicator described herein adapted to include field generators or other positioning systems described herein.

In operation, the field generators generate magnetic fields which are detected by the sensor. By measuring the magnetic fields generated by each field generator at the sensor, the location and orientation of the sensor may be computed, thereby determining position data for the surgical instrument 12. Although not limited thereto, exemplary electromagnetic tracking subsystems are further described in U.S. Pat. Nos. 5,913,820; 5,592,939; and 6,374,134 which are incorporated herein by reference. In addition, it is envisioned that other types of position tracking devices are also within the scope of the present invention. For instance, non line-of-sight tracking subsystem 20 may be based on sonic emissions or radio frequency emissions. In another instance, a rigid or semi-rigid surgical instrument, such as a rigid endoscope, rigidizable or semi-rigidizable guide to may be tracked using a line-of-sight optical-based tracking subsystem (i.e., LED's, passive markers, reflective markers, etc).

Position data such as location and/or orientation data from the tracking subsystem 20 is in turn relayed to the data processor 16. The data processor 16 is adapted to receive position/orientation data from the tracking subsystem 20 and operable to render a volumetric perspective image and/or a surface rendered image of the region of interest. The volumetric perspective and/or surface image is rendered 36 from the scan data 32 using rendering techniques well known in the art. The image data may be further manipulated 38 based on the position/orientation data for the surgical instrument 12 received from tracking subsystem 20. Specifically, the volumetric perspective or surface rendered image is rendered from a point of view which relates to position of the surgical instrument 12. For instance, at least one electromagnetic sensor 24 may be positioned at the tip of the surgical instrument 12, such that the image is, rendered from a leading point on the surgical instrument. In this way, the surgical instrument navigation system 10 of the present invention is able, for example, to visually simulate a virtual volumetric scene of an internal cavity from the point of view of the surgical instrument 12 residing in the cavity or from the point of view of the dynamic reference frame 19. It is readily understood that tracking two or more electro-magnetic sensors 24 which are embedded in the surgical instrument 12 enables orientation of the surgical instrument 12 to be determined by the system 10.

As the surgical instrument 12 is moved by the surgeon within the region of interest, its position and orientation are tracked and reported on a real-time basis by the tracking subsystem 20. The volumetric perspective image may then be updated by manipulating 38 the rendered image data 36 based on the position of the surgical instrument 12 or the position of the surgical instrument relative to the dynamic reference frame or datum position indicator. The manipulated volumetric perspective image is displayed 40 on a display device 18 associated with the data processor 16. The display 18 is preferably located such that it can be easily viewed by the surgeon during the medical procedure. In one embodiment, the display 18 may be further defined as a heads-up display or any other appropriate display. The image may also be stored by data processor 16 for later playback, should this be desired.

It is envisioned that the primary perspective image 38 of the region of interest may be supplemented by other secondary images. For instance, known image processing techniques may be employed to generate various multi-planar images of the region of interest. Alternatively, images may be generated from different view points as specified by a user 39, including views from outside of the vessel or cavity or views that enable the user to see through the walls of the vessel using different shading or opacity. In another instance, the location data of the surgical instrument may be saved and played back in a movie format. It is envisioned that these various secondary images may be displayed simultaneously with or in place of the primary perspective image.

In addition, the surgical instrument 12 may be used to generate real-time maps corresponding to an internal path traveled by the surgical instrument or an external boundary of an internal cavity. Map showing the advancement of the instrument along any desired, pre-selected path may also be displayed. The desire to pre-selected path may be generated during pre-surgical planning for a patient specific transluminal procedure as described herein. Real-time maps may be generated by continuously recording the position of the instrument's localized tip, its full extent, its position, shape or state of articulation. A real-time map is generated by the outermost extent of the instrument's position and minimum extrapolated curvature as is known in the art. The map may be continuously updated as the instrument is moved within the patient, thereby creating a path or a volume representing the internal boundary of the cavity. It is envisioned that the map may be displayed in a wire frame form, as a shaded surface or other three-dimensional computer display modality independent from or superimposed on the volumetric perspective image 38 of the region of interest. It is further envisioned that the map may include data collected from a sensor embedded into the surgical instrument, such as pressure data, temperature data or electro-physiological data. In this case, the map may be color coded to represent the collected data. It is also envisioned that the map may be generated to show instrument movement within a cavity having an access through or in proximity to a datum position indicator.

FIG. 130 illustrates another type of secondary image 28 which may be displayed in conjunction with the primary perspective image 38. In this instance, the primary perspective image is an interior view of an air passage within the patient 13. The secondary image 28 is an exterior view of the air passage which includes an indicia or graphical representation 29 that corresponds to the location of the surgical instrument within the air passage. In FIG. 130, the indicia 29 is shown as crosshairs. It is envisioned that other indicia may be used to signify the location of the surgical instrument in the secondary image. As further described below, the secondary image 28 is constructed by superimposing the indicia 29 of the surgical instrument onto the manipulated image data 38.

Referring to FIG. 131, the display of an indicia of the surgical instrument on the secondary image may be synchronized with an anatomical function, such as the cardiac or respiratory cycle, of the patient. In certain instances, the cardiac or respiratory cycle of the patient may cause the surgical instrument to flutter or jitter within the patient. For instance, a surgical instrument positioned in or near a chamber of the heart will move in relation to the patient's heart beat. In this instance, the indicia of the surgical instrument will likewise flutter or jitter on the displayed image. It is envisioned that other anatomical functions which may effect the position of the surgical instrument within the patient are also within the scope of the present invention.

To eliminate the flutter of the indicia on the displayed image, position data for the surgical instrument is acquired at a repetitive point within each cycle of either the cardiac cycle or the respiratory cycle of the patient. As described above, the imaging device is used to capture volumetric scan data 42 representative of an internal region of interest within a given patient. A secondary image may then be rendered 44 from the volumetric scan data by the data processor.

In order to synchronize the acquisition of position data for the surgical instrument, the surgical instrument navigation system 10 may further include a timing signal generator 26. The timing signal generator 26 is operable to generate and transmit a timing signal 46 that correlates to at least one of (or both) the cardiac cycle or the respiratory cycle of the patient 13. For a patient having a consistent rhythmic cycle, the timing signal might be in the form of a periodic clock signal. Alternatively, the timing signal may be derived from an electrocardiogram signal from the patient 13. One skilled in the art will readily recognize other techniques for deriving a timing signal that correlate to at least one of the cardiac or respiratory cycle or other anatomical cycle of the patient.

As described above, the indicia of the surgical instrument 12 tracks the movement of the surgical instrument 12 as it is moved by the surgeon within the patient 13. Rather than display the indicia of the surgical instrument 12 on a real-time basis, the display of the indicia of the surgical instrument 12 is periodically updated 48 based on the timing signal from the timing signal generator 26. In one exemplary embodiment, the timing generator 26 is electrically connected to the tracking subsystem 20. The tracking subsystem 20 is in turn operable to report position data for the surgical instrument 12 in response to a timing signal received from the timing signal generator 26. The position of the indicia of the surgical instrument 12 is then updated 50 on the display of the image data. It is readily understood that other techniques for synchronizing the display of an indicia of the surgical instrument 12 based on the timing signal are within the scope of the present invention, thereby eliminating any flutter or jitter which may appear on the displayed image 52. It is also envisioned that a path (or projected path) of the surgical instrument 12 may also be illustrated on the displayed image data 52.

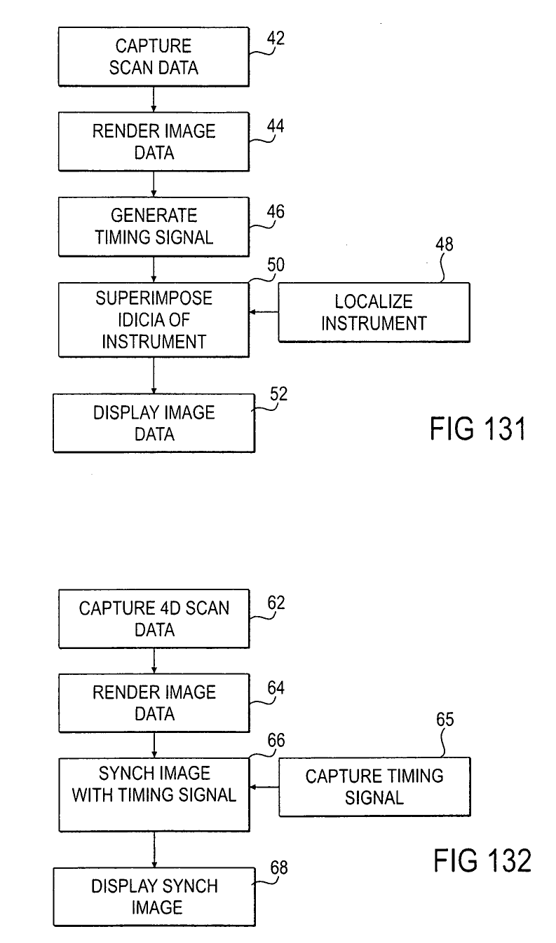

In another aspect of the present invention, the surgical instrument navigation system 10 may be further adapted to display four-dimensional image data for a region of interest as shown in FIG. 132. In this case, the imaging device 14 is operable to capture volumetric scan data 62 for an internal region of interest over a period of time, such that the region of interest includes motion that is caused by either the cardiac cycle or the respiratory cycle of the patient 13. A volumetric perspective view of the region may be rendered 64 from the volumetric scan data 62 by the data processor 16 as described above. The four-dimensional image data may be further supplemented with other patient data, such as temperature or blood pressure, using coloring coding techniques.

The surgical instrument navigation system of the present invention may also incorporate atlas maps. It is envisioned that three-dimensional or four-dimensional atlas maps may be registered with patient specific scan data or generic anatomical models. Atlas maps may contain kinematic information (e.g., heart models) that can be synchronized with four-dimensional image data, thereby supplementing the real-time information. In addition, the kinematic information may be combined with localization information from several instruments to provide a complete four-dimensional model of organ motion. The atlas maps may also be used to localize bones or soft tissue which can assist in determining placement and location of implants, or to further coordinate transluminal procedures described herein. U.S. Pat. No. 6,892,090 titled “Method and Apparatus for Virtual Endoscopy” to Verard et al., is incorporated herein by reference in its entirety.

Additional aspects of image guided surgery may also be used to provide imaging and instrument control and guidance for transluminal procedures. As such, embodiments of the present invention also relate to methods and devices for registering an anatomical region with images of the anatomical region, verifying registration of an anatomical region, and dynamically referencing the anatomical region, including the use of datum position indicators to facilitate registration, and one particular example, the registration of a segmented, controllable instrument or guide tubes described above.

Image Guided Surgery (IGS), also known as “frameless stereotaxy” has been used for many years to precisely locate and position therapeutic or medical measurement devices in the human body. Proper localization including position and orientation of these devices is critical to obtain the best result and patient outcome.

Some image guided surgery techniques use an externally placed locating device, such as a camera system or magnetic field generator together with an instrument containing a trackable component or “position indicating element” that can be localized by a locating device or tracking system (collectively referred to hereinafter a “tracking device”). These position indicating elements are associated with a coordinate system and are typically attached to instruments such as surgical probes, drills, microscopes, needles, X-ray machines, etc. and to the patient. The spatial coordinates and often the orientation (depending on the technology used) of the coordinate system associated with the position indicating elements can be determined by the tracking device in the fixed coordinate system (or fixed “frame of reference”) of the tracking device. Many tracking devices may be able to track multiple position indicating elements simultaneously in their fixed frame of reference. Through geometrical transformations, it is possible to determine the position and orientation of any position indicating element relative to a frame of reference of any other position indicating element.

A variety of different tracking devices exist, having different advantages and disadvantages over each other. For example, optical tracking devices may be constructed to enable the highly accurate position and orientation of a tool equipped with position indicating elements to be calculated. However, these optical tracking devices suffer from line-of-site constraints, among other things. Electromagnetic (EM) tracking devices do not require a line-of-sight between the tracking device and the position indicating elements. Electromagnetic tracking devices may therefore be used with flexible instruments where the position indicating elements are placed at the tip of the instruments. One disadvantage, however, is that electromagnetic tracking devices are subject to interference from ferromagnetic materials and conductors. This interference may degrade accuracy when such ferromagnetic materials or conductors are placed in the proximity of position indicating elements or EM tracking devices. Other known tracking devices include, but are not limited to, fiberoptic devices, ultrasonic devices and global positioning (“time of flight”) devices.

By combining data obtained from a tracking device and a position indicating element with preoperative or intraoperative scans (such as for example, x-rays, ultrasounds, fluoroscopy, computerized tomographic (CT) scans, multislice CT scans, magnetic resonance imaging (MRI) scanning, positron emission tomographic (PET) scans, isocentric fluoroscope images, rotational fluoroscopic reconstructions, intravascular ultrasound (IVUS) images, single photon emission computer tomographer (SPECT) systems, or other images), it is possible to graphically superimpose the location of the position indicating element (and thus any surgical instrument having a position indicating element) over the images. This enables the surgeon to perform an intervention/procedure more accurately since the surgeon is better able to locate or orient the instrument during the procedure. It also enables the surgeon to perform all or part of the procedure without the need for additional x-rays or other images, but instead to rely on previously acquired data. This not only reduces the amount of ionizing radiation the surgeon and patient are exposed to, but can speed the procedure and enable the use of higher fidelity images than can not normally be acquired intra-operatively. Surgical plans may also be annotated onto these images (or indeed used without the images) to be used as templates to guide medical procedures.

Image Guided Surgery can be most effectively performed only if an accurate “registration” is available to mathematically map the position data of position indicating elements expressed in terms of the coordinate system of the tracking device, i.e., “patient space,” to the coordinate system of the externally imaged data, i.e., “image space” determined at the time the images were taken. In rigid objects such as the skull or bones, one method of registration is performed by using a probe equipped with position indicating elements (therefore, the probe itself is tracked by a tracking device) to touch fiducial markers (such as, for example, small steel balls (x-spots) made by the Beekley Corporation, Bristol, Conn.) placed on the patient to obtain the patient space coordinates of the fiducials. These same fiducials are visible on an image such as, for example, a CT scan and are identified in the image space by indicating them, for example, on a computer display. Once these same markers are identified in both spaces, a registration transformation or equivalent mathematical construction can be calculated. In one commonly used form, a registration transformation may be a 4.times.4 matrix that embodies the translations, magnification factors and rotations required to bring the markers (and thus the coordinate systems) in one space in to coincidence with the same markers in the another space.

Fiducial markers used for registration can be applied to objects such as bone screws or stick-on markers that are visible to the selected imaging device, or can be implicit, such as unambiguous parts of the patient anatomy. These anatomical fiducials might include unusually shaped bones, osteophytes or other bony prominence, features on vessels or other natural lumens (such as bifurcations), individual sulci of the brain, or other markers that can be unambiguously identified in the image and patient. A rigid affine transformation such as the 4×4 matrix described above may require the identification of at least three non-collinear points in the image space and the patient space. Often, many more points are used and a best-fit may be used to optimize the registration. It is normally desirable that fiducials remain fixed relative to the anatomy from the time of imaging until the time that registration is complete. In one example, the anatomical fiducial is provided by a datum and position indicator. Because the datum and position indicator may be placed in any position along the lumen or within a portion of the body, it is useful in the precise placement of instruments and in planning access pathways for medical procedures.

Registration for image-guided surgery may be done by different methods. Paired-point registration is described above and is accomplished by a user identifying points in image space and then obtaining the coordinates of the corresponding points in patient space. Another type of registration, surface registration, can be done in combination with, or independent of, paired point registration. In surface registration, a cloud of points is digitized in the patient space and matched with a surface model of the same region in image space. A best-fit transformation relating one surface to the other may then be calculated. In another type of registration, repeat-fixation devices may be used that involve a user repeatedly removing and replacing a device in known relation to the patient or image fiducials of the patient.

Automatic registration may also be done. Automatic registration may, for example, make use of predefined fiducial arrays or “fiducial shapes” that are readily identifiable in image space by a computer. The patient space position and orientation of these arrays may be inferred through the use of a position indicating element fixed to the fiducial array. Other registration methods also exist, including methods that attempt to register non-rigid objects generally through image processing means.

Registrations may also be performed to calculate transformations between separately acquired images. This may be done by identifying “mutual information” (e.g., the same fiducial markers existing in each space). In this way, information visible in one image, but not the other, may be coalesced into a combined image containing information from both. In the same manner, two different tracking devices may be registered together to extend the range of a tracking device or to increase its accuracy.

Following registration, the two spaces (patient and image) are linked through the transformation calculations. Once registered, the position and orientation of a tracked probe placed anywhere in the registered region can be related to, for example, a scan of the region. Typically the tracking device may be connected to a computer system. Scans may also be loaded onto the computer system. The computer system display may take the form of a graphical representation of a probe or instrument's position superimposed onto preoperative image data. Accordingly, it is possible to obtain information about the object being probed as well as the instrument's position and orientation relative to the object that is not immediately visible to the surgeon. The information displayed can also be accurately and quantitatively measured enabling the surgeon to carry out a preoperative plan more accurately.

An additional concept in image guided surgery is that of “dynamic referencing.” Dynamic referencing can account for any bulk motion of the anatomy relative to the tracking device. This may entail additional, position indicating elements, or other techniques. For example, in cranial surgery, position indicating elements that form the dynamic reference are often attached directly to the head or more typically to a clamp meant to immobilize the head. In spine surgery, for example, a dynamic reference attached (via a temporary clamp or screw) to the vertebral body undergoing therapy is used to account for respiratory motion, iatrogentic (e.g., doctor-induced) motion caused by the procedure itself, as well as motion of the tracking device. In an analogous manner, the tracking device itself may be attached directly to the anatomy, moving with the anatomy when it moves. For example, a small camera may be attached to a head-clamp so that movement of the head would produce movement of the camera, thus preserving registration.

“Gating” may also be used to account for motion of the anatomy. Instead of continually compensating for motion through dynamic referencing, “gated measurements” are measurements that are only accepted at particular instants in time. Gating has been used in, for example, cardiac motion studies. Gating synchronizes a measured movement (e.g., heartbeat, respiration, or other motion) to the start of the measurement in order to eliminate the motion. Measurements are only accepted at specific instants. For example, gating during image guided surgery of the spine may mean that the position of a tracked instrument may be sampled briefly only during peak inspiration times of a respiratory cycle.

Both registration and use of an image guided surgery system in the presence of anatomical motion (such as that which occurs during normal respiration) is generally regarded as safer and more accurate if a dynamic reference device is attached prior to registration (and/or if gating is used). Instead of reporting the position and orientation of a position indicating element of a tracked instrument in the fixed coordinate system of the tracking device, the position and orientation of the position indicating element of the tracked instrument is reported relative to the dynamic reference's internal coordinate system. Any motion experienced mutually by both the dynamic reference and the tracked instrument is “cancelled out.”

In some embodiments, the integrated system may include a referencing device. In some embodiments data may be sent and received between the referencing device and computer element. The referencing device may, inter alia, aid in providing image data, location data, position data, coordinate data, and/or motion data regarding an anatomical region of the patient. The referencing device may otherwise enable dynamic referencing of an anatomical region of a patient, (including soft tissues and/or deformable bodies). In one embodiment, the functionality of the referencing device is provided by a datum and position indicator.

In one embodiment, the integrated system may include a tracking device. The tracking device may include an electromagnetic tracking device, global positioning system (GPS) enabled tracking device, an ultrasonic tracking device, a fiber-optic tracking device, an optical tracking device, a radar tracking device, or other type of tracking device. The tracking device may be used to obtain data regarding the three-dimensional location, position, coordinates, and/or other information regarding one or more position indicating elements within an anatomical region of the patient. The tracking device may provide this data/information to the computer element.

In one embodiment, the integrated system may include an imaging device. The imaging device may send and receive data from the integrated system. In one embodiment, the imaging device may be used to obtain image data, position data, or other data necessary for enabling the apparatus and processes described herein. The imaging device may provide this data to the computer element. The imaging device may include x-ray equipment, computerized tomography (CT) equipment, positron emission tomography (PET) equipment, magnetic resonance imaging (MRI) equipment, fluoroscopy equipment, ultrasound equipment, an isocentric fluoroscopic device, a rotational fluoroscopic reconstruction system, a multislice computerized tomography device, an intravascular ultrasound imager, a single photon emission computer tomographer, a magnetic resonance imaging device, or other imaging/scanning equipment

Other devices and or elements such as, for example, temperature sensors, pressure sensors, motion sensors, electrical sensors, EMG equipment, ECG equipment, or other equipment or sensors may be part of or send and receive data from the integrated system.

Those having skill in the art will appreciate that the invention described herein may work with various system configurations. Accordingly, more or less of the aforementioned system components may be used and/or combined in various embodiments. It should also be understood that various software modules and control application that are used to accomplish the functionalities described herein may be maintained on one or more of the components of system recited herein, as necessary, including those within individual tools or devices. In other embodiments, as would be appreciated, the functionalities described herein may be implemented in various combinations of hardware and/or firmware, in addition to, or instead of, software.

The imaging and navigation system provides systems and methods for registration of an anatomical region of a patient, verification of the registration of the anatomical region, and dynamic referencing of the anatomical region, wherein the anatomical region may include soft tissue and/or deformable bodies.

In one embodiment, the imaging and navigation system may use a conduit within an anatomical region of a patient to, inter alia, aid in providing image information and position information from within the anatomical region. This conduit may supply sufficient coordinate information regarding the anatomical region to be used for registration of the anatomical region. For example, a coronary artery surrounding the heart may provide sufficient topographical coordinate information regarding the heart to be used as a conduit for registration by a method of the invention.

In one embodiment, a conduit as used herein may include a naturally existing conduit within the anatomical region such as, for example, an artery, vein, or other vessel of the circulatory system; a bronchial tube or other vessel of the respiratory system; a vessel of the lymphatic system; an intestine or other vessel of the digestive system; a urinary tract vessel; a cerebrospinal fluid vessel; a reproductive vessel; an auditory vessel; a cranial ventricle; an otolaryngological vessel; or other naturally occurring conduit existing within the anatomical region of interest.

In some embodiments, an “artificial conduit” may be created within the anatomical region such as, for example, a percutaneous puncture of tissue within the anatomical region by a cannula such as might be caused by a hypodermic needle. The process of insertion of this cannula may, in turn, form an artificial conduit within the anatomical region.

In other embodiments, a conduit may include a manufactured conduit that may be placed within the anatomical region such as, for example, a guide tube, a catheter, hollow endoscope, a tubular vascular guidewire, or other manufactured conduit that may be inserted into the anatomical region of interest. In some embodiments, a manufactured conduit and a naturally existing or artificial conduit may be used together. For example, a catheter, cannula, or tube may be navigated inside a naturally existing vessel of the anatomical region. In some embodiments, a first manufactured conduit may be inserted within a second manufactured conduit, which may in turn be inserted into the anatomical region, an artificial conduit within the anatomical region, or within a naturally existing conduit within the anatomical region. One or more connections may be made between the conduits and the lumen of the body or portion of the body. Those connection points (selected by the user) may then in turn be used to provide additional imaging, control and navigation information into the system for display or use by the user to control instruments in the body.

In some embodiments, a manufactured conduit may be inserted within an anatomical region to at least partially fill and/or conform to the dimensions of a space within that anatomical region. For example, a catheter or other conduit may be fed into a cavity within an anatomical region, such that the catheter coils, bends, folds, or otherwise “balls up” (without obstructing any lumens therein) inside the cavity, thus at least partially filling the volume of, or conforming to the dimensions of, the cavity. The methods described herein may then be performed using the catheter as it exists within the cavity.

In some embodiments, artificial conduits may used in conjunction with natural conduits and/or manufactured conduits (described below). For example, an artificial conduit may be created (e.g., with a needle) in certain tissue (e.g., skin, connective tissue, or other tissue) to reach a natural conduit within the anatomical region (e.g., vein) or to insert a manufactured conduit (e.g., catheter).

In one embodiment, the invention provides a registration device for registration of an anatomical region of a patient. As described below, the registration device may be part of, or be operatively connected to, an integrated system for registration, verification of registration, dynamic referencing, navigation, and/or other functions (hereinafter “integrated system”), which is described in detail below.

FIG. 133 illustrates a registration device 101 according to an embodiment of the invention. Registration device 101 may include a tube, catheter, vascular guidewire, or other device that may be inserted into a conduit within the anatomical region to be registered. In the illustrated embodiment, the registration device 101 is a guide tube.

In some embodiments, registration device 101 may be freely slidable in within a conduit or portion of a body cavity (i.e., in a cavity accessed using a transluminal procedure described herein). In some embodiments, registration device 101 may be temporarily fixed within a conduit using one or more fixating elements such as, for example, balloons, deployable hooks, cages, stiffening wires, or other fixation elements or techniques described herein.

In one embodiment, registration device 101 may include at least one position indicating element 103. Position indicating element 103 may include an element whose location, position, orientation, and/or coordinates relative to a tracking device may be determined and recorded. As such, the position of position indicating element 103 within the conduit, and thus the position of at least one point of the conduit within the anatomical region of the patient, may be determined. Position indicating element 103 may include a device whose position may be detectable by a tracking device in the frame of reference of the tracking device. For example, position indicating element 103 may include a coil that may produce a magnetic field that is detectable by an electromagnetic tracking device. In one embodiment, position indicating element 103 may include a coil that detects a magnetic field emitted by the electromagnetic tracking device. In some embodiments position indicating elements and their position in the frame of reference of a tracking device may be enabled by “Hall Effect” transducers or superconducting quantum interference devices (SQUID). In other embodiments, position indicating element 103 may include an element whose position is detectable by a global positioning system (GPS) enabled tracking device, an ultrasonic tracking device, a fiber-optic tracking device (e.g., Shape-Tape, MEasurand, Inc., Fredricton, New Bruswick), an optical tracking device, or a radar tracking device. Other types of position indicating elements and/or tracking devices may be used. In one embodiment, the tracking device used to detect the position of position indicating element 103 may be part of, or operatively connected to, an integrated system.

Registration device 101 may contain one or more detectable elements (not shown). In one embodiment, detectable elements may be placed on or adjacent to position indicating element 103, such that the location of detectable elements may be correlated to the location and/or orientation of position indicating element 103 as disclosed in U.S. Pat. No. 6,785,571, which is incorporated herein by reference in its entirety. Detectable elements may include radio-opaque elements or elements that are otherwise detectable to certain imaging modalities such as, for example, x-ray, ultrasound, fluoroscopy, computerized tomography (CT) scans, positron emission tomography (PET) scans, magnetic resonance imaging (MRI), or other imaging devices. Detectable elements may enable the detection and/or visualization of certain points of reference of registration device 101 within a conduit residing in an anatomical region of a patient, which may aid in registration, verification of registration, dynamic referencing, navigation, and/or other uses.

In the illustrated embodiment, a controllable instrument 107 is extended through a lumen provided in the registration device 101. The controllable instrument 107 contains one or more detectable elements 105a-i placed along segments 107A-107E. In one embodiment, detectable elements may be placed along the controllable instrument 107 such that the location of detectable elements 105a-g may be correlated to the location and/or orientation of position indicating element 103 as disclosed in U.S. Pat. No. 6,785,571, which is incorporated herein by reference in its entirety. Detectable elements 105 may include radio-opaque elements or elements that are otherwise detectable to certain imaging modalities such as, for example, x-ray, ultrasound, fluoroscopy, computerized tomography (CT) scans, positron emission tomography (PET) scans, magnetic resonance imaging (MRI), or other imaging devices. Detectable elements may enable the detection and/or visualization of certain points of reference of registration device 101 within a conduit residing in an anatomical region of a patient, which may aid in registration, verification of registration, dynamic referencing, navigation, and/or other uses. In this way, the shape and position of the controllable instrument may be obtained relative to the registration device 101.

In one embodiment, position indicating element 103 may be located at or near the tip of registration device 101. In other embodiments, multiple position indicating elements may be located at various points along the length of registration device 101. In another alternative embodiment, the position indicating element is located adjacent an opening formed in a lumen as part of a transluminal procedure or other procedure.

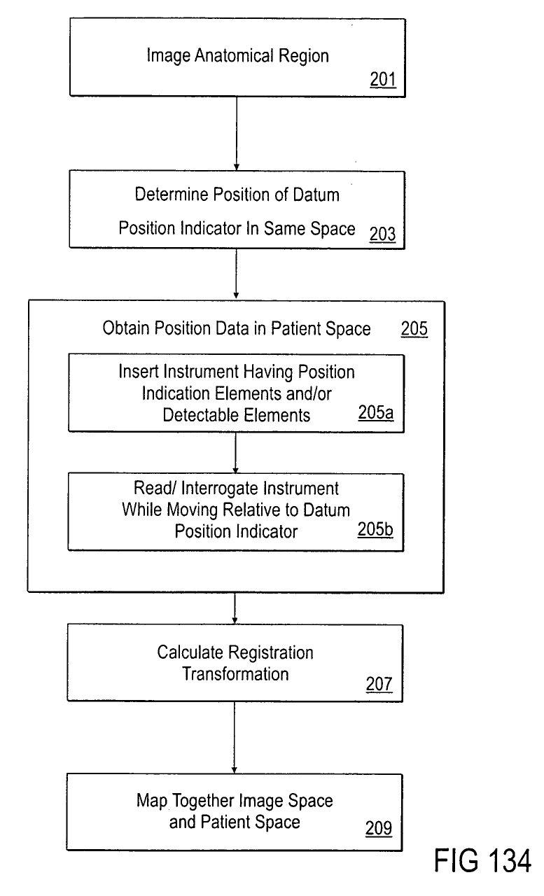

FIG. 134 illustrates an exemplary process 200 according to an embodiment of the invention, wherein registration of an anatomical region of a patient may be performed. In an operation 201, one or more images of the anatomical region of the patient and/or the conduit within the anatomical region may be obtained by an imaging device. An imaging device may include, for example, an x-ray device, an ultrasound device, a fluoroscopic device, a computerized tomography (CT) device, a positron emission tomography (PET) device, a magnetic resonance imaging (MRI) device, an isocentric fluoroscope, a rotational fluoroscopic reconstruction system, a multislice computerized tomography device, an intravascular ultrasound imager, a single photon emission computer tomographer, or other imaging device. In some embodiments, the imaging device may be part of, connected to, and/or exchange data with an integrated system.

In an operation 203, position information regarding the path of the conduit within the anatomical region may be obtained in the frame of reference of the image(s) taken in operation 201 (i.e., the path of the conduit in “image space”). In one embodiment, the path of the conduit may be obtained through a segmentation process in which the images are examined for the conduit and connected regions within the images (that are identified as the conduit) may be coalesced to determine the spatial pathway of the conduit in the coordinate system of the images. Several such methods are known in the art such as, for example, those outlined by L. M. Lorigo in Lorigo et al., CURVES: Curve Evolution for Vessel Segmentation, 5 Medical Image Analysis 195-206 (2001). This step also includes determining the position of a datum position indicator in the image space.

In an operation 205, the spatial pathway of the conduit in the frame of reference of the patient (i.e., in the “patient space”) may be obtained. In one embodiment, this spatial pathway (or “position data”) may be obtained via a registration device (similar to, or the same as, registration device 101 of FIG. 1) that is inserted into the conduit, wherein the registration device includes at least one position indicating element.

In one embodiment, the registration device may contain a position indicating element at its tip. In an operation 205a, and instrument having position indication elements and/or detectable elements may be inserted into the conduit within in the anatomical region of the patient. In an operation 205b, the registration device may then sample the coordinates of the position indicating elements included within the controllable instrument as the controllable instrument is moved within the conduit, resulting in position information regarding the path and shape of the controllable instrument within the anatomical region in the frame of reference of the tracking device (this may also be referred to as the frame of reference of the patient, i.e., the “patient space”). The operation 205 be also may include the use of the registration device (i.e., datum position indicator) to read/interrogate the position or detectable elements on the controllable instrument while the controllable instrument is moving relative to the datum position indicator.

In other embodiments, the registration device may contain multiple position indicating elements along its length. In these embodiments, the registration device may be inserted into the conduit within the anatomical region of the patient. The coordinates of the multiple position indicating elements may then be detected by a tracking device while the position indicating elements are either moved or kept stationary within the conduit, resulting in position information regarding the path of the conduit within the anatomical region in the frame of reference of the tracking device (i.e., the patient space). In one embodiment, if the registration device contains multiple position indicating elements and their coordinates are sampled within the conduit as the conduit is moving (e.g., movement affecting the anatomical region that in turn affects the conduit), enhanced tempero-spatial information regarding the movement of the patient space may be obtained.

In an operation 207, a registration transformation may be calculated. In some embodiments a registration transformation may include a registration transformation matrix or other suitable representation of the registration transformation.

A transformation is a mathematical tool that relates coordinates from one coordinate system to coordinates from another coordinate system. There may be multiple methods to calculate the registration transformation. One exemplary registration transformation calculation method may include “brute force” approach. A brute force approach may involve treating the pre-registration image data and the registration position data as completely independent datasets and manually attempting to match the two datasets by altering each translation, rotation, and scaling parameter in turn to create the best match. This however, may be inefficient.

Another exemplary method may include an Iterative Closest Point (ICP) algorithm, one version of which is described in U.S. Pat. No. 5,715,166, incorporated herein by reference in its entirety.

Another exemplary registration transformation calculation method is known as singular valued decomposition (SVD) in which the same point locations are identified in each coordinate system (e.g., the image space and the patient space). Other imaging systems, techniques and procedures may also be employed such as those described in US Patent Application Publication US 2004/0024309 filed Apr. 30, 2003 titled “System For Monitoring The Positions of A Medical Instrument With Respect to A Patient's Body” by Maurice R. Ferre, et al.; US Patent Application Publication US 2002/0077544 filed Sep. 20, 2001 titled “Endoscopic Targeting Method and System” by Ramin Shahidi.; U.S. Patent Application Publication US 2006/0036162 filed Jan. 27, 2005 titled “Method and Apparatus For Guiding A Medical Instrument to A Subsurface Target Site in a Patient” by Ramin Shahidi et al., and U.S. Patent Application Publication US 2006/0173287 filed Dec. 19, 2003 titled “Method and Arrangement for Tracking A Medical Instrument” by Joerg Sabczynski, et al., each of which is incorporated herein by reference in its entirety.

In an operation 209, the image information of the anatomical region (image space) and the position information of the path of the conduit within the anatomical region (patient space) may be registered or mapped together using the registration transformation. The registration or mapping may be performed by bringing the coordinates of the anatomical region derived from the image data (the image space) into coincidence with the coordinates of the conduit within the anatomical region derived from the tracking device/position indicating element (the patient space). In some embodiments, additional coordinate sets may also be “co-registered” with the image and tracking device data. For example, a magnetic resonance image dataset may be first co-registered with a computerized tomography dataset (both image space), which may in turn be registered to the path of the conduit in the frame of reference of the patient (patient space).

The result of mapping the image space data and the patient space data together may include or enable accurate graphical representations (e.g., on the original image data, surgical plan or other representation) of an instrument or other tool equipped with a position indicating element through the anatomical region. In some embodiments, this navigation may enable image guided surgery or other medical procedures to be performed in/on the anatomical region. Additionally, graphical representations may also be prepared for the position of the datum position indicator, the proposed surgical path, and the actual path taken, for example. User interface and controls will allow the user to indicate a new desired path and the new desired path may be provided as an input to a control system used to articulate or manipulate the steerable, controllable instrument.

In an operation 311, the one or more position indicating elements may be moved within the anatomical region as their positions are sampled by the tracking device. The transformed location (as calculated using the registration transformation of operation 307) of the one or more position indicating elements as they are moved may be displayed on the image. Errors in the registration may be indicated by movement of the one or more position indicating elements outside of the registered path within the anatomical region (e.g., such as outside a conduit registered within the anatomical region). The absence of errors may be used to verify that desired track is being followed.

The location of the one or more position indicating elements within the anatomical region may then be imaged using an imaging device such as, for example an x-ray device, ultrasound device, fluoroscopy device, computerized tomography (CT) device, positron emission tomography (PET) device, magnetic resonance imaging (MRI), or other imaging device. The visualized location of the position indicating elements within the anatomical region may then be compared to points within the anatomical region as obtained by a registration. Discrepancies between the images of the position indicating elements and the points obtained by the registration may be indicative of errors in the registration. In one embodiment, this operation may be performed entirely numerically and automatically, e.g., through the use of a computer to compare the two paths.

In one embodiment, a controllable instrument or other component may include a position indicating element or device whose position may be detectable in a frame of reference of a datum and position indicator. For example, a position indicating element may include a coil that may produce a magnetic field that is detectable by an electromagnetic tracking device location on or incorporated into a datum and position indicator. In one embodiment, a datum and position indicator includes an electromagnetic tracking device that detects a magnetic field emitted by position indicating element placed on an instrument (see FIG. 1). In some embodiments position indicating elements and their position in the frame of reference of a tracking device may be enabled by “Hall Effect” transducers or superconducting quantum interference devices (SQUID). In other embodiments, position indicating elements may include elements whose position is detectable by a global positioning system (GPS) enabled tracking device, an ultrasonic tracking device, a fiber-optic tracking device, an optical tracking device, a radar tracking device or RFID. Other types of position indicating elements and/or tracking devices may be used. In one embodiment, the tracking system used to detect the position of position indicating elements may be part of, or operatively connected to, an integrated system.

The datum and position indicator may contain a pressure sensor, an electromyograph (EMG) sensor, an electrocardiograph (ECG) sensor or other devices or sensors, which may be used to gate the sampling of the reference sensors, to measure blood pressure, air pressure, or other quality or characteristic of the body.

The datum and position indicator may also be used to dynamically reference an anatomical region of a patient. In some embodiments, one or more of the devices and/or processes described herein may be used with each other in various combinations. For example, a datum position indicator alone or optionally attached to a guide tube and a controllable instrument may be used to perform registration and referencing of an anatomical region. Those having ordinary skill in the art will realize that similar devices and techniques according to the invention may be used in the lung to map out pulmonary pathways, in the colon to map out parts of the digestive system, the urethra to map out the urinary system, or in other areas of the human or mammalian anatomy to map or image other areas.

A guide tube may be introduced into a patient through an orifice (natural or artificial) of the patient such as, for example, the mouth, the nose, the urethra, the anus, the vagina, an incision into the circulatory system, the diaphragm or the esophagus, a manufactured channel created during a surgical procedure, or other orifice (whether naturally existing or created) in the patient. The datum position indicator may be introduced through a portal, over a guidewire or any other method as known in the art. Datum and position indicator may be introduced through an orifice into a lumen or region of the patient such as, for example the bronchial tree, the digestive tract, the esophagus, or other regions of the anatomy of the patient.

Datum and position indicator may be placed using conventional techniques (e.g., fluoroscopy) to a position in the body. A placement target may include something of interest such as, for example, a stenosis, an aneurysm, a tumor, a polyp, a calcification, or other element or condition of interest. Is should be noted that the placement target may exist in another anatomical region of the body but the target is selected in a lumen that will provide access to the target area of interest. Additionally, the target need not exist in the precise anatomical system in which datum and position indicator and other elements of the invention are placed, but may be nearby, such as a tumor present in the same or adjacent tissue to that being monitored by datum and position indicator. By selecting specific locations for the placement of the datum and position indicator and hence the location of the opening creating for a transluminal procedure, more precise access to target regions may be obtained.

Datum and position indicator may be fixed in place to prevent motion within the lumen using the techniques described herein or other fixation techniques. The datum and position indicator may be fixed in place in through the use of an inflatable member such as, for example, a balloon. In some embodiments, datum and position indicator may be fixed using deployable cages, hooks, insertable stiffening wires, vacuum devices, helical catheter arrangement designed to maintain datum and position indicator location within an anatomical region or conduit therein, or other methods known in the art or described elsewhere in this application. In some embodiments, datum and position indicator may be fixed as described herein or using known fixation techniques at several locations or continuously along its length, not just the tip, so that the datum position indicator and the instrument (guide tube, flexible tube or other lumen attached to it) datum does not move independently of the anatomy or change its shape once placed.

As discussed above, a controllable instrument may be inserted into a lumen of datum or in proximity to a datum and position indicator. The controllable instrument may contain multiple position indicating elements enabling position information of position indicating elements and ultimately, the lumen to be determined.

In one embodiment, the 2D anatomical region may be optionally co-registered with a preoperative image, where applicable. For example, if a pre-operative scan (e.g., MRI, abdominal or thoracic cavity image, or other scan) were conducted and revealed a tumor or other lesion, the preoperative scan may be co-registered with an image taken for registration purposes prior to registration with the position of the datum position indicator.

In some embodiments, during registration, a three dimensional path of the center of the registration device (the “centerline”) may be calculated in the coordinate system of the previously obtained images of the anatomical region. In some embodiments, a three-dimensional (3D) map of the anatomical area of interest and/or the location of at least part of datum and position indicator may be constructed during this calculation. Simultaneous biplane fluoroscopy, multi-slice CT, or other fast 3D image acquisition of the anatomical region may be used, in conjunction with images (those mentioned above or other images) of the anatomical region to construct the 3D map and/or the location of at least part of the datum and positioning device and surrounding anatomical features. A 3D mathematical map of the structure of the anatomical region to be navigated beyond the tip of datum and position indicator 701 may also be constructed using the images (those mentioned above or other images) or scan information of the anatomical region (particularly if a contrast agent or other imaging assistance agents has been introduced therein). In some embodiments, the image data (e.g., the 3d model/map) regarding the anatomical region and the lumen, opening and cavity beyond may be expressed as a 3D spline, parametric equations, voxels, polygons, coordinate lists, or other indicators of the walls or path of the component structures, or simply a “skeleton” of the central axis (centerline) of the component tubes and structures, existing in the coordinate system of the image devices.

Other surrounding areas of interest may also be incorporated into the map. In vascular surgery, for example, the 3D map may include the vessels enhanced at the end of the datum and position indicator showing the path to a tumor, lesion, or area of investigation or interest such as, for example, the location of a prior biopsy sample or other testing.

The 3D map of an instrument relative to or within a datum and position indicator may be reconstructed from images constrained within datum and position indicator, from the image of datum and position indicator itself, from images taken from the instrument, and/or from other images or source of information regarding the 3D path. This 3D path may form the coordinates of the path of the instrument in the “image space” or coordinate system of the imaging device.

During registration of an anatomical region, a tracking device on the instrument may be activated and the coordinates of the instrument's path in a coordinate system of the datum and position indicator or the coordinates in the frame of reference of a coordinate system created by controllable instrument (if used) may be determined. In some embodiments, this may be accomplished by sliding the instrument through datum and position indicator while a tracking device (on the datum position indicator, a device in the body or external to the body) simultaneously samples the coordinates of the instrument, the shape of the instrument or other position/orientation information from the instrument. This essentially retraces the image space path that had been traversed and provides a corresponding set of position data in the “patient space” or coordinate system of the tracking system. The position data obtained may also be expressed as a 3D spline, parametric equations along the registration tube, coordinate lists, or other suitable formats.

A registration transformation may then be calculated. As noted herein, there may be multiple methods to calculate the registration transformation. In an exemplary registration transformation calculation method, the (x, y, z) positions may be parameterized as a function of distance along the path traveled by the instrument. In one embodiment, at the start of position data collection, instrument is located within datum and position indicator at the time of imaging (e.g., x-ray and/or other imaging data) to determine its path (i.e., S(0)). As the instrument is moved within and beyond the datum and position indicator, position indicating element moves a distance, S(t), which can be estimated from the (x, y, z) position of position indicating element using the incremental Euclidian distance, i.e. sqrt((x.sub.k-x.sub.i).sup.2+(y.sub.k-y.sub.i).s−up.2+(z.sub.k-z.sub.i).sup.2), where Pk=(x.sub.k, y.sub.k, z.sub.k) are the position indicating element coordinates at the k.sup.th sample and Pi=(x.sub.i, y.sub.i, z.sub.i) are the sensor coordinates of the i.sup.th sample. In general, the criteria for selecting i and k may be as follows:

1 set S=0 set sample=0 NEXT_i: set i=P (sample) NEXT_k: set sample=sample+1 set k=P (sample) set time=sample+1 if sqrt((x.sub.k−x.sub.i).sup.2+(y.sub.k−y.sub.i).sup.2+(z.sub.k−z.sub.i).sup.2)>threshold distance {S=S+sqrt((x.sub.k−x.sub.i).sup.2+(y.sub.k−y.sub.i).sup.2+z.sub.k−z.sub.i).sup.2)//distance from sample 0 to sample k.//calculate corresponding point in image space, of distance S from the start set sample=sample+1 if more samples: go to NEXT_i }else {if more samples: go to NEXT_k}

Once position indicating element moves more than a predefined amount (the threshold distance), S can be calculated from the image data showing the path of the instrument. Unless this is done, noise will be continually added to the estimate of S, and the estimates of S will always be higher than the correct measurements. At corresponding values of S, the data from the image data (image space) is matched to the position indicating element space data (patient space), producing a high quality paired point matching at locations all along the instrument path beyond the datum and position indicator.

Having determined an image space set of coordinates of the path of datum and position indicator and a patient space set of coordinates of the same path, registration may be performed between the position data of the patient space and the imaging data of the patient space. As discussed, this registration may involve calculation of a transformation matrix to bring the two sets of data from different coordinate systems into coincidence with one another. In one embodiment, additional coordinate sets may also be “co-registered” with the image and tracking coordinates. In a non-rigid registration, the registration matrix may be allowed to vary over time and location in the registered region.

Once an anatomical region has been registered, a tube, a navigation device, therapeutic tools, needles, probes, flexible endoscopes, stents, coils, drills, ultrasound transducers, pressure sensors, or indeed any flexible or rigid device that is equipped with a position indicating element may be inserted into the respective conduit and used for navigation purposes, for a therapeutic or other medical procedure, or for other purposes. In one embodiment, the registration may be used to generate or highlight an image wherein the navigable conduit is visible. The position indicating element of the device or tool may be tracked by a tracking device, and the position of the device or tool may be displayed in the generated or highlighted image, enabling navigation. Additionally, verification of the registered area according to the methods described herein (or other methods) may also be performed.

In some embodiments (e.g., where contrast agent was injected into regions distal to the tip of a datum and position indicator or tube used for registration), regions distal to the tip of the datum and position indicator may be displayed in an image and navigated as well.

In some embodiments, the invention may include a computer-implemented integrated system (“integrated system”) for performing one or more of the methods described herein, including any of the features, function, or operations described herein (as well as other methods such as, for example, therapeutic, diagnostic, or other methods). The integrated system may also enable any of the devices, elements, or apparatus described herein (as well as other apparatus).

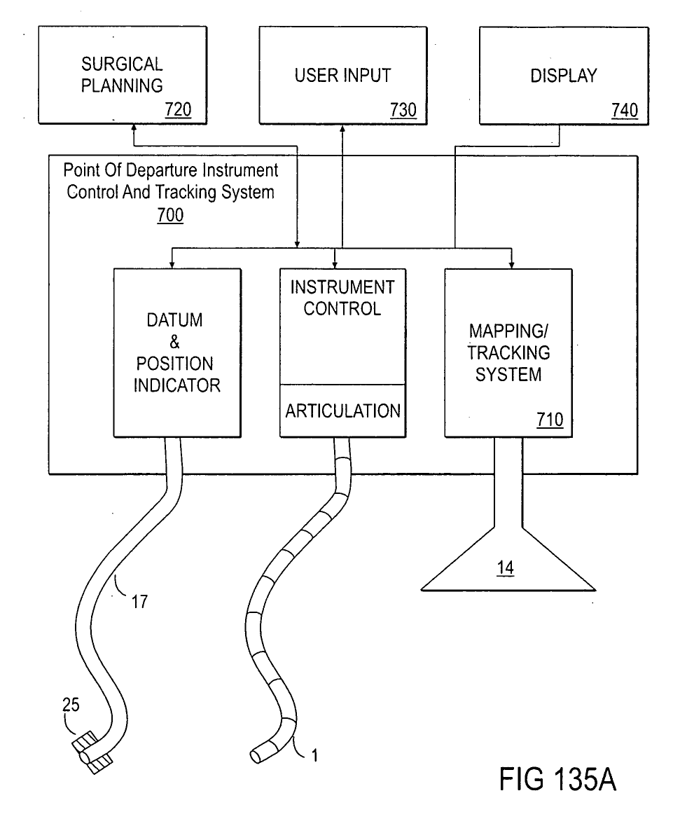

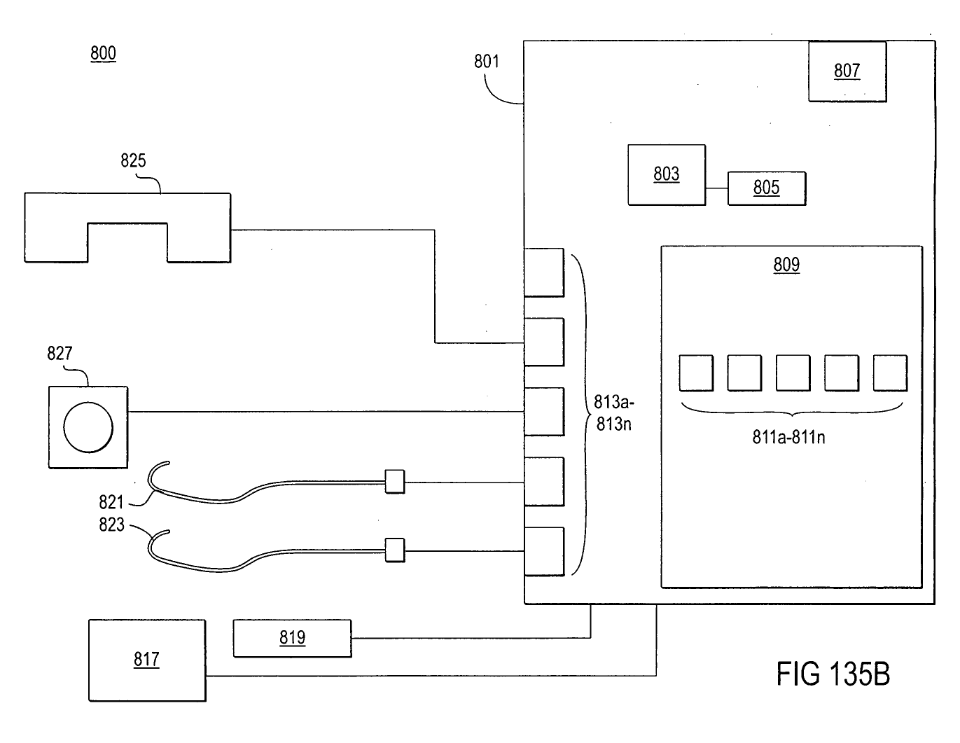

FIG. 135A illustrates a block diagram of a point of departure instrument control and tracking system 700 according to one embodiment of the present invention. The point of departure instrument control and tracking system 700 brings together all the various components and subsystems described herein to provide improved methods of control and instrument placement during transluminal procedures. Because the optimal transluminal opening for a given procedure will vary from patient to patient and may or may not have a natural anatomical landmark, the point of departure instrument control and tracking system provides a landmark (i.e., a datum position indicator) that may be used for other systems to provide additional information to the user to improve the transluminal procedure. The point of departure instrument control and tracking system integrates the operation of the datum and position indicator, instrument control including articulation and control of the instrument, and the mapping and tracking systems described herein. The point of departure instrument control and tracking system 700 also provides a display 740 and receives user input 730 and surgical planning 720 input. As indicated by the arrows, the system 700 also provides feedback to the user as well as to a surgical planning system or program.

FIG. 135B is another exemplary illustration of an integrated system according to an embodiment of the invention. In one embodiment, an integrated system 800 includes a computer element 801. Computer element 801 may include a processor 803, a memory device 805, a power source 807, a control application 809, one or more software modules 811a-811n, one or more inputs/outputs 813a-813n, a display device 817, a user input device 819, and/or other elements.

Computer element 801 may include one or more servers, personal computers, laptop computers, or other computer devices. Computer element 801 may receive, send, store, and/or manipulate any data necessary to perform any of the processes, calculations, or operations described herein (including any of the features, functions, or operations described in FIGS. 2, 3, or 5. Computer element 801 may also perform any processes, calculations, or operations necessary for the function of the devices, elements, or apparatus described herein.

According one embodiment, computer element 801 may host a control application 809. Control application 809 may comprise a computer application which may enable one or more software modules 811a-811n

In some embodiments, computer element 801 may contain one or more software modules 811a-811n enabling processor 803 to receive, send, and/or manipulate imaging data regarding the location, position, and/or coordinates of one or more instruments, devices, detectable elements, position indicating elements, or other elements of the invention inside an anatomical region of a patient. This imaging data may be stored in memory device 805 or other data storage location.

In some embodiments, one or more software modules 811a-811n may also enable processor 803 to receive, send and/or manipulate data regarding the location, position, orientation, and/or coordinates of one or more position indicating elements or other elements of the invention inside the anatomical region of the patient. This data may be stored in memory device 805 or other data storage location.

In some embodiments, one or more software modules 811a-811n may also enable processor 803 to calculate one or more registration transformations, perform registration (or mapping) of coordinates from two or more coordinate systems according to the one or more transformation calculations, and produce one or more images from registered data. In some embodiments, images produced from image data, position data, registration data, other data, or any combination thereof may be displayed on display device 817.

In some embodiments, one or more software modules 811a-811n may also enable processor 803 to receive, send, and/or manipulate data regarding the location, orientation, position, and/or coordinates of one or more position indicating elements for use in constructing a rigid-body description of an anatomical region of a patient. In some embodiments, one or more software modules 811a-811n may enable processor 803 to create of dynamic, deformable, and/or other models of an anatomical region of the patient, and may enable the display of real time images regarding the anatomical region. In some embodiments, these images may be displayed on display device 817.

In one embodiment, integrated system 800 may include a registration device 821 (the same as or similar to registration device 101 of FIG. 133). In some embodiments, registration device 821 may be operatively connected to computer element 801 via an input/output 813. In other embodiments, registration device 821 need not be operatively connected to computer element 801, but data may be sent and received between registration device 821 and computer element 813. Registration device 821 may, inter alia, aid in providing image data, location data, position data, and/or coordinate data regarding an anatomical region of the patient or one or more elements of the invention within the anatomical region of the patient. The registration device may otherwise enable registration of the anatomical region the patient, (including soft tissues and/or deformable bodies).

In one embodiment, integrated system 800 may include a referencing device 823 (the same as or similar to referencing device 101 of FIG. 133). In some embodiments, referencing device 823 may be operatively connected to computer element 801 via an input/output 813. In other embodiments, referencing device 823 need not be connected to computer element 801, but data may be sent and received between referencing device 823 and computer element 813. Referencing device 823 may, inter alia, aid in providing image data, location data, position data, coordinate data, and/or motion data regarding an anatomical region of the patient or one or more elements of the invention within the anatomical region of the patient. Referencing device 823 otherwise enables dynamic referencing of an anatomical region of a patient, (including soft tissues and/or deformable bodies).

In one embodiment, integrated system 800 may include a tracking device 825. In one embodiment, tracking device 825 may be operatively connected to computer element 825 via an input/output 813. In other embodiments, tracking device 825 need not be operatively connected to computer element 825, but data may be sent and received between tracking device 825 and computer element 813. Tracking device 825 may include an electromagnetic tracking device, global positioning system (GPS) enabled tracking device, an ultrasonic tracking device, a fiber-optic tracking device, an optical tracking device, a radar tracking device, or other type of tracking device. Tracking device 825 may be used to obtain data regarding the three-dimensional location, position, coordinates, and/or other information regarding one or more position indicating elements within an anatomical region of the patient. Tracking device 825 may provide this data/information to computer element 801.

In one embodiment, integrated system 800 may include an imaging device 827. In one embodiment, data may be sent and received between imaging device 827 and computer element 813. This data may be sent and received via an operative connection, a network connection, a wireless connection, through one or more floppy discs, or through other data transfer methods. Imaging device 827 may be used to obtain image data, position data, or other data necessary for enabling the apparatus and processes described herein. Imaging device 827 may provide this data to computer element 813. Imaging device 827 may include x-ray equipment, computerized tomography (CT) equipment, positron emission tomography (PET) equipment, magnetic resonance imaging (MRI) equipment, fluoroscopy equipment, ultrasound equipment, an isocentric fluoroscopic device, a rotational fluoroscopic reconstruction system, a multislice computerized tomography device, an intravascular ultrasound imager, a single photon emission computer tomographer, a magnetic resonance imaging device, or other imaging/scanning equipment

Other devices and or elements such as, for example, temperature sensors, pressure sensors, motion sensors, electrical sensors, EMG equipment, ECG equipment, or other equipment or sensors may be included in and/or may send and receive data from integrated system 800. Additionally, any therapeutic diagnostic, or other medical tools or devices may also be included in and/or may send and receive data from integrated system 800.

In one embodiment, the various instruments and/or devices described herein may be interchangeably “plugged into” one or more inputs/outputs 813a-813n. In some embodiments, the software, hardware, and/or firmware included integrated system 800 may enable various imaging, referencing, registration, navigation, diagnostic, therapeutic, or other instruments to be used interchangeably with integrated system 800.

Those having skill in the art will appreciate that the invention described herein may work with various system configurations. Accordingly, more or less of the aforementioned system components may be used and/or combined in various embodiments. It should also be understood that various software modules 811a-811n and control application 809 that are used to accomplish the functionalities described herein may be maintained on one or more of the components of system recited herein, as necessary, including those within individual medical tools or devices. In other embodiments, as would be appreciated, the functionalities described herein may be implemented in various combinations of hardware and/or firmware, in addition to, or instead of, software. U.S. Patent Application Publication US 2005/0182319 titled “Method and Apparatus for Registration, Verification and Referencing of Internal Organs” to Neil David Glossop is incorporated herein by reference.

Other details of imaging, registration and other details are provided in the following patent and publication, each of which is incorporated herein by reference in its entirety: U.S. Patent application publication 2002/0077544 to Shahidl; U.S. Patent application publication 2006/0036162 to Shahidl; U.S. Ser. No. 2006/0173287 to Sabcyznski et al.; U.S. Patent application publication 2005/0182319 and U.S. Pat. No. 6,892,090 to Verard et al.

In addition to described embodiments, a datum and position indicator may also include the use of any proximity sensors and position indicators, such as, for example, capacitance proximity sensors and probes, photoelectric sensors, inductive sensors, magnetic sensors, ultrasound sensors and RFID-based tracking systems. Data and position and indicator embodiments encompass both contact and non-contact position detectors between datum and instrument passing adjacent to the datum. Alternative configurations include orientations where an instrument: (a) passes through a continuous ring datum; (b) passes through a partial ring datum; (c) passes along side a datum or (d) moves within a detectable zone of a proximity sensor or other detector on the datum position indicator. The datum and position indicator could be placed inside or outside of the body. Surfaces on a datum position indicator may be adapted to the point of transluminal entry, the surrounding tissue or structures where the datum position indicator will be used.

As has been previously illustrated, a datum and position indicator may also be incorporated into a rigidizable controllable overtube that is affixed to the wall of a lumen, such as the stomach wall. The distal end of a rigidizable overtube containing a datum and position indicator at a fixation point indicates the amount of, or length of, an instrument that has passed through the transluminal opening. Additional datum and position indicator configurations include a first datum and position indicator point located on the first rigidizable tube. This first datum and position indicator could be located at the entry point in the body, such as the mouth or at the exit point of the first rigidizable tube, i.e., the distal end of the first or primary rigidizable tube. A second rigidizable guide tube (i.e., the secondary guide tube) may be used having a second datum and position indicator to indicate the entry into or exiting from the second rigidizable guide tube.

In another alternative embodiment, a datum and position indicator could be provided independently of the rigidizable guide tube or it could be integrated into the guide tube. The datum and position indicator landing pad could contain a base adapted for securing to a portion of the body and a datum and position indicator to provide access through the pad and adapted to receive a segmented controllable instrument.

Alternatively, the datum and position indicator landing pad could include a suturing mechanism or a filling mechanism to form and or close an opening made to allow a segmented instrument to advance through the datum and position indicator into a portion of the body.

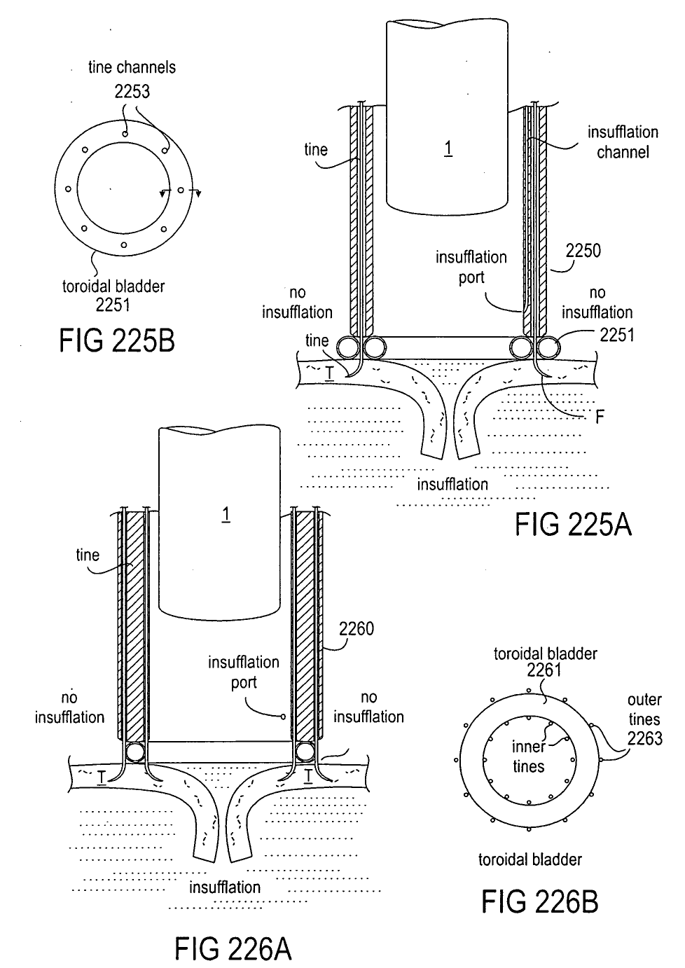

Numerous alternative configurations are available for the datum position indicator. As described above the datum position indicator may be configured for placement adjacent the anus as would be suited for trans-colonic procedures. The datum position indicator I also have adaptable surfaces as provided by the inflatable bladder in FIGS. 225 and 226 described below. The use of the bladder would help seal the datum position indicator to the tissue and be used to make up for differences in shape of lumen wall—rounded, various curvature and non-planar shapes.

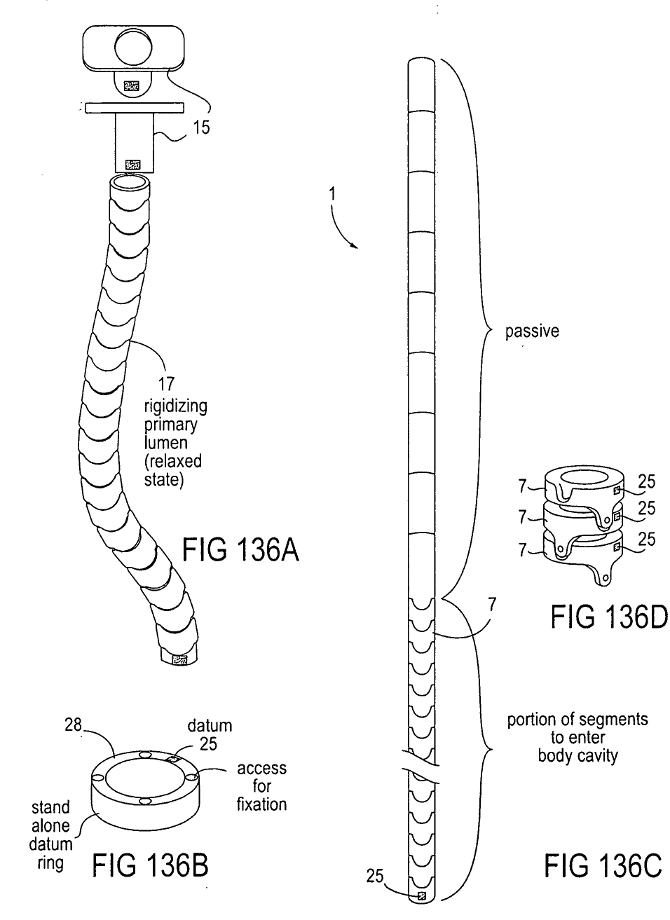

FIG. 136A illustrates the datum on mouthpiece introducer as well as the Rigidizable guide. FIG. 136B illustrates a stand-alone fixation and datum ring that may be provided during the transluminal procedure independent of other instruments. FIG. 136C illustrates datum 25 attached to segments 7 on a controllable instrument 1. The datum and position indicators or position indicators he located on the distal end or on each individual segment 7 as indicated.

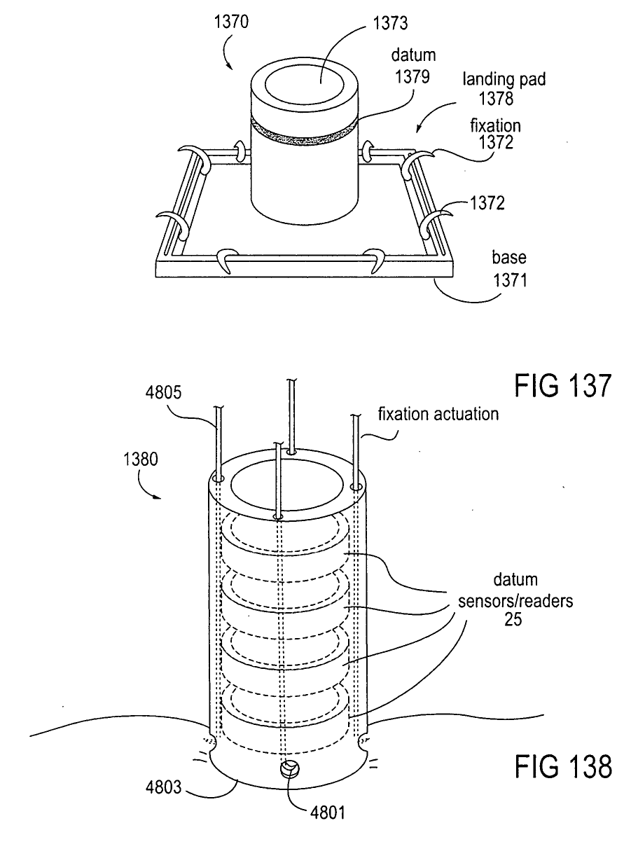

FIG. 137 illustrates an integrated datum and position indicator 1370 having its own fixation system. The landing pad 1378 includes a base 1371 having a flexible framework to contour to the target tissue for the transluminal procedure. The base 1371 supports the datum 1379 as well as the fixation elements 1372 and their actuation mechanism (not shown). In use, the integrated datum and position indicator 1370 is provided to the target area and held in place using fixation elements 1372. Thereafter instruments to create the transluminal opening and/or issuance to perform a transluminal procedure are provided through the lumen 1373. Instruments passing through lumen 1373 are registered, tracked, or recognized by datum 1379.

FIG. 138 illustrates an alternative stand-alone datum position indicator 1380. Datum and position indicator 1380 includes a fixation element 4801 on the distal end 4803. Position and tracking sensors are placed within a plurality of readers 25 located along the length of the indicator 1380. The distal end 4803 is affixed to the target tissue using fixation elements 4001 activated by fixation actuation 4805.

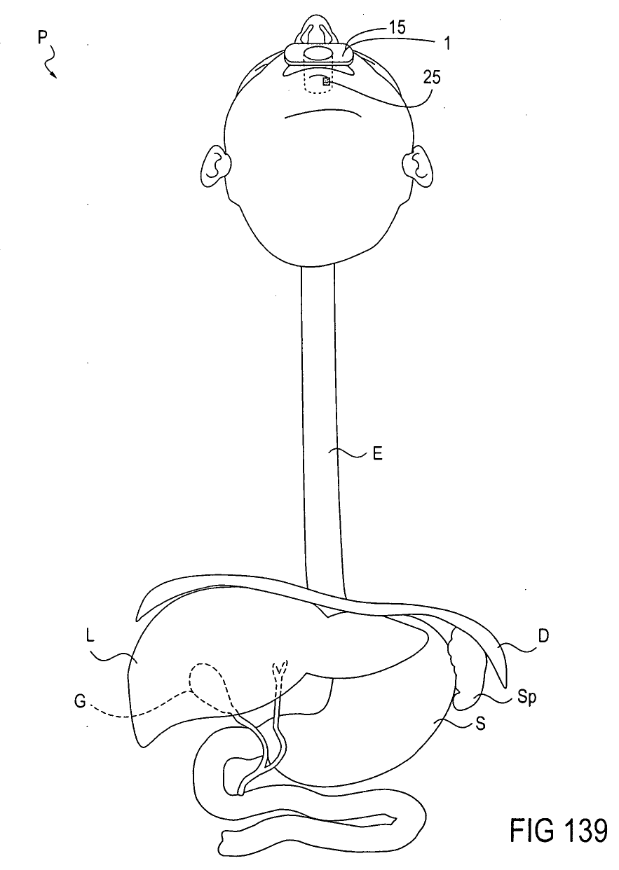

FIG. 139 illustrates the datum 25 on the introducer mouthpiece 15. Positioning a datum and position indicator at the entrance of the mouth provides a stable platform for instruments entering the esophagus and transiting to the stomach. Because a datum position indicator attached to the stomach wall may move during a transluminal procedure, measurements and readings provided by a datum and position indicator at a stable location, like the mouth, can provide useful information to ensuring accurate mapping, tracking and control functionality of the system.

Datum and position indicators according to the present invention may also be utilized with multiple guide tips. FIGS. 96 and 97 above are illustrated examples of multiple guide tubes having s datum and position indicators.

Additionally, because of the individualized nature of each procedure depending upon the patient's physiology and the specific procedure being performed and that the desired path may be unique for each patient, the DPI may be provided in a wide array of alternative embodiments and with a variety of different functional capabilities. There are embodiments having one or more additionally functionality—may be a part of a flexible tube, a rigidizable or semi-rigidizable guide tube, or as a standalone component transported by another instrument

The distal end of the rigidizable guide may be provided with an integrated tissue cutter and datum and position indicator reader. For example, in a viewing the distal end of a rigidizable guide tube and on there could be a number of concentric rings of various instrumentation. For example, the outermost ring could include a vacuum port to apply vacuum to the distal end to allow it to seat against and secure to the tissue wall. The next innermost ring could be a cutter or some device used to form the opening in the stomach wall and interior of the cutting ring could be a datum and position indicator which could be fully enclosed or simply edge mounted on the end to re-register the passage of the amount of the controllable instrument that moves past the datum and position indicator. Alternatively the datum and position indicator could also be a ring, adapted to size an adapted to receive the steerable controllable instrument.

As illustrated in examples that follow, a datum and position indicator may be combined with one or more functions in support of a transluminal procedure. Examples of features that may be incorporated into a datum and position indicator are, by way of example and not limitation:
(a) features to create and maintain a sterile field such as liner storage and deployment;
(b) sterile field preparation such as rinsing and flushing the target tissue after it has been isolated by the datum and position indicator structure;
(c) tissue grasping, and fixation and anchoring to secure the datum position indicator lumen to the target tissue prior to or after creating a transluminal opening;
(d) datum housing to provide point of departure instrument information;
(e) tools to create transluminal openings;
(f) tools to close transluminal openings;
(g) utilizing the lumen of the datum and position indicator as an access pathway for other tools and instruments (i.e., use of side rinse channel as a tool access port)

As an additional feature to ensure safe and proper target tissue opening, embodiments of the datum and position indicator may also include instruments or components to measure or detect surrounding tissue (i.e., 1D, 2D or 3D imaging capabilities).

For example, a datum and position indicator may optionally include an ultrasound transducer or other detection or imaging system to provide additional information about attachment site. These components may have the volumetric imaging capabilities described above to create a volume about the datum and position indicator to aid in positioning instruments within volume and performing procedures. An ultrasound probe is provided to measure lumen wall thickness at selected opening site to ensure that fixation elements are driven into the tissue to the desired depth. As such the ultrasound probe may be used as input to system to select proper depth for fastener penetration. The ultrasound probe may be used to identify structure, tissue, organs on the opposite side of the wall to confirm location of target tissue opening site—i.e., compare to expected opening site from previous surgical planning. The ultrasound probe may also act as a safety device to ensure proximity, spacing, distance or other position information for organs, tissue, structure and other anatomy that is behind the target opening location.

Datum and Position Indicator Configurations and Examples

As illustrated above, the datum and position indicator may be a stand alone component as illustrated in FIGS. 137, 138. In this configuration, the datum and position indicator is attached to the target tissue by itself. Thereafter, instruments, devices and/or tools are guided to its lumen and passed through it.

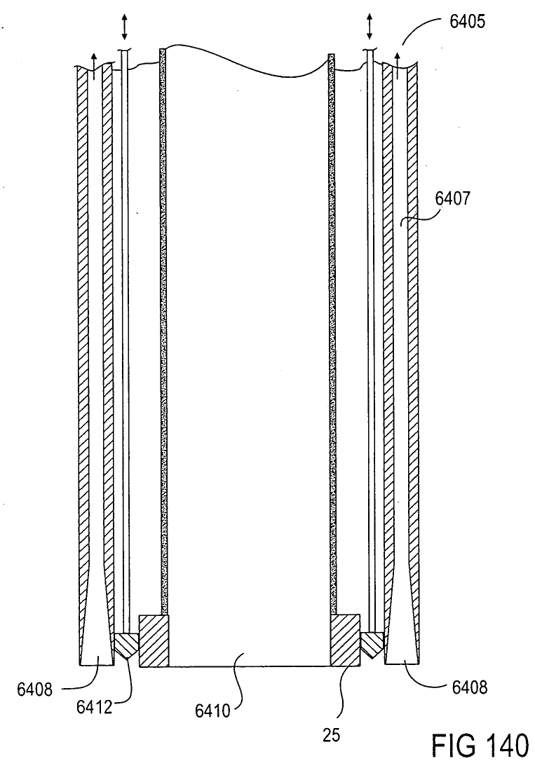

FIG. 140 illustrates a datum and position indicator 25 formed in the distal end of the guide tube 6405. The distal end of guide tube 6405 includes a vacuum port 6408 and a fixation element 6412. The datum and position indicator 25 is adjacent the guide to bloom and 6410 at the distal end. A vacuum channel 6407 can ask the vacuum ports 6408 to a vacuum source (not shown). In operation, the guide tube 6405 would be maneuvered within the body to a position in adjacent the target tissue. Next, vacuum would be applied through the vacuum port 6408 to engage in secure the distal end of guide to 6405 to the target tissue wall. Once secured to the tissue wall, fixation element 6402 can be activated to engage with and anchor the distal end of the guide to to the target tissue wall. Once the distal end is secured to the tissue wall, the vacuum source can be turned off and the fixation elements 6412 allowed to hold the guide tube in place against the target tissue. Alternatively, the guide tube could be held in place using a vacuum port 6408 and then fixation elements 6412 could be configured to provide a controlled transluminal opening in the target tissue. It is to be appreciated that fixation elements 6412 and other fixation or tissue anchoring embodiments disclosed herein may be coated with sterioids or other pharmacological agents to speed healing and reduce scarring of the transluminal opening formed in the target lumen wall opening.

Attachment at Landing Site

One advantage of using a rigidizable overtube adapted on the distal end to grip the tissue is that the guide tube may be manipulated to provide mechanical advantage to the grip tissue. For example, it is possible to maneuver the rigidizable guide tube through the mouth via esophagus and into the stomach to a position opposite the stomach wall from the liver or other organ. The fixed guide tube could then be used to apply manipulating force to the stomach to alter the placement or the stomach relative to adjacent structures. Using the tissue gripping means disposed on the distal end of the rigidizable guide tube the stomach lining may be gripped and maneuvered away from the liver or other organ. Thereafter, cutting means may be advanced through the guide tube and use to cut or provide an opening in the stomach lining. In contrast to conventional cutting systems the inventive device and methods allow the stomach lining to be pulled away from surrounding tissue and organs so that when the stomach lining is cut the adjacent tissue or organ is in a safe position. The rigidizable guide may be placed against the lesser curvature of the stomach, affixed to the stomach lining using non-penetrating fixation implements as described herein. Once secured to the stomach lining, the stomach may be maneuvered away from surrounding tissues prior to forming an opening in the stomach wall. Once the hole is formed in the side wall the tissue can be released back or held in that position while the instruments are advanced through the rigidizable scope now in a locked position to provide a stable guide for precise placement of the instruments to access the organs as desired. In one embodiment, such configuration is used to access the liver or gall bladder.

Additional techniques to hold the guide tube to the stomach wall include the use of vacuum ports, staples, hooks, barbs or other mechanical gripping devices or fasteners.

In addition, there is a combination at the tip of the overtube of an anchoring mechanism to secure the wall of the organ such as the stomach. For example, the tip of the overtube could be provided with suction and staples to secure the tissue.

Another option is to have several hooks, for example, NiTi hooks, that would be pushed out from the edge of the tip as the means to anchor to the wall. These hooks would be curved so that they may for example, curve within and not curved within the stomach wall tissues so that they pierced to enter and pierced to exit the stomach wall on the same side or can pierce to enter at one side pierced to exit on the second side and then reenter to engage on the second side without harm to adjacent tissue or structures.

A variety of gripping or securing means may be provided on the distal end of the rigidizable scope. The rigidizable guide tube distal end therefore includes embodiments having attachment or securing implements to fix the position of the guide tube to the bodily tissue or structure. A variety of suitable gripping or securing means are described in the following patents or patent publications that are hereby incorporated by reference in their entirety: U.S. Pat. Nos. 5,443,484; 5,613,937; 5,865,791; 5,964,782; 6,183,486; 6,206,696; 6,228,023; 6,506,190; 6,663,640; 2001/0001825; 2002/0107531; 2002/0120254; 2003/0078604; 2004/0087831; 2004/0054335; 2003/0144694; 6,716,196; 6,663,639; 6,139,522; 6,068,637; 5,702,412; 5,577,993; 5,573,496; 5,488,958; 5,407,427; 5,582,577; 5,681,341; 5,873,876; 5,925,064; 5,928,264; 5,984,896; 6,110,187; 6,123,667; 6,620,098; 6,626,930; 6,698,433; 6,743,220; 2002/0032415; 2002/0099410; 2003/0176883; 2004/0087967; 2004/0093023; 2004/0116949; 2004/0133220; and 2004/0133229.

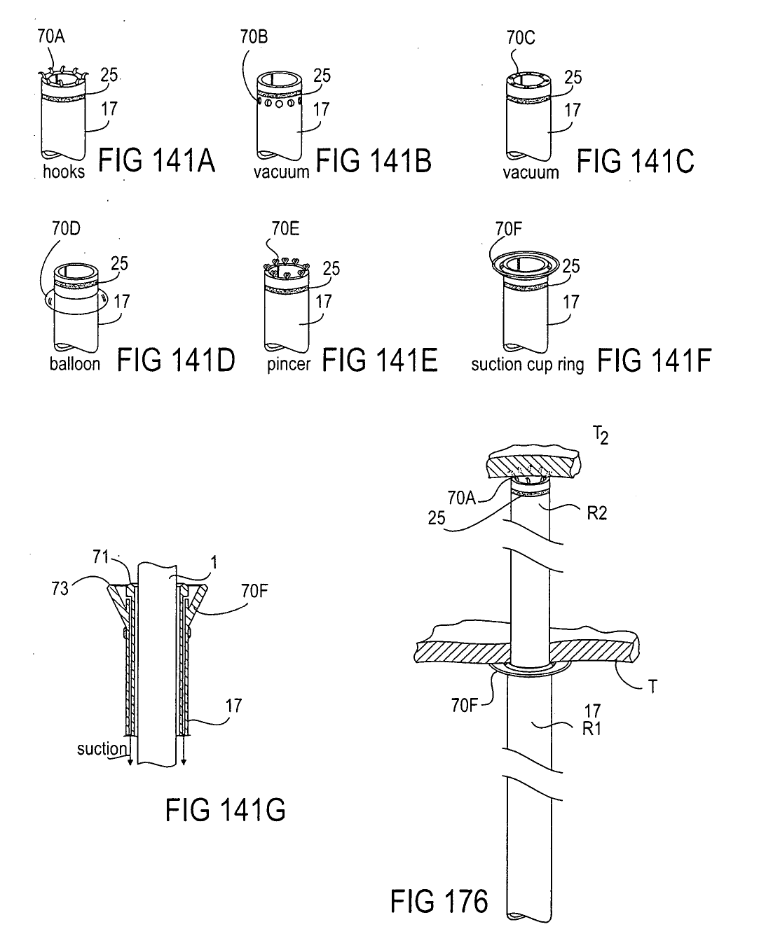

A wide variety of distal fixation means are possible as illustrated in FIGS. 141A-141H. All illustrate an exemplary guide tube 17 and datum 25 for reference. FIG. 140A illustrates the use of distal hooks 70A that extend out from the distal end of the guide tube 17. FIG. 141B illustrates sidewall vacuum ports on the distal end of the guide to 17. The use of sidewall vacuum ports 70B enables the guide to sidewall to engage with the lumen while leaving the entirety of the distal end available for other purposes. As described below, sidewall vacuum ports 70B and sidewall fixation may also be used during transluminal procedures involving an empty stomach (i.e., without the installation). FIG. 141C. illustrates distal end of vacuum ports 70C. FIG. 141D illustrates a balloon 70D located on the distal end of the guide tube. FIG. 141E illustrates a plurality of pinchers or tissue grippers 70E located on the distal end of the guide tube 17. FIG. 141F illustrates a suction cup ring on the distal end of the guide tube 17. FIG. 141G is a section view of FIG. 141F. As best seen in FIG. 141G, the suction cup ring 70F creates a circular vacuum port 73 when seated against the target tissue. Also when seated against a target tissue, ring blade 71 may be actuated to create a uniform transluminal opening in the target tissue. A controllable intimate one is shown passing through the transluminal opening created by blade 71.

The Use of Tines and Rotational Anchoring Mechanisms

Another attachment mechanism for securing the rigidizable tube against the tissue wall is a pincher-type device with moveable tines. When the pincher is positioned against the tissue and then advanced into the tissue, usually by rotation the tines relative to the tissue, the tines are driven into the tissue. As a result, the tissue is gripped in two or more separate positions in a controllable manner. The rotation of the tines determines the depth into which the tines penetrate. It is to be appreciated that some tine embodiments described herein fully engage into tissue by rotating less than one revolution and do so without penetrating completely through the target tissue. The tines may also be adapted to move toward each other when a mechanical or other motive mechanism is activated. As such when activated the at least one tine and the tissue it grips is advanced towards the other tine and the tissue attached to it. When positioned at the distal or landing end of a rigidizable guide tube, the tissue is attached to the rigidizable guide tube by piercing, gripping and joining the tissue together. The tines and other fixation elements described herein may be designed to penetrate only the surface of the tissue, deep into the tissue without piercing the tissue and to pierce the tissue. It is to be appreciated that a single design adequate to pierce tissue could be used but with controlled activation so that the tissue fixation could be at the surface, deep into but not piercing through the tissue or piercing through the tissue. Control of the depth of penetration could be controlled by limiting or increasing the amount of tine rotation.

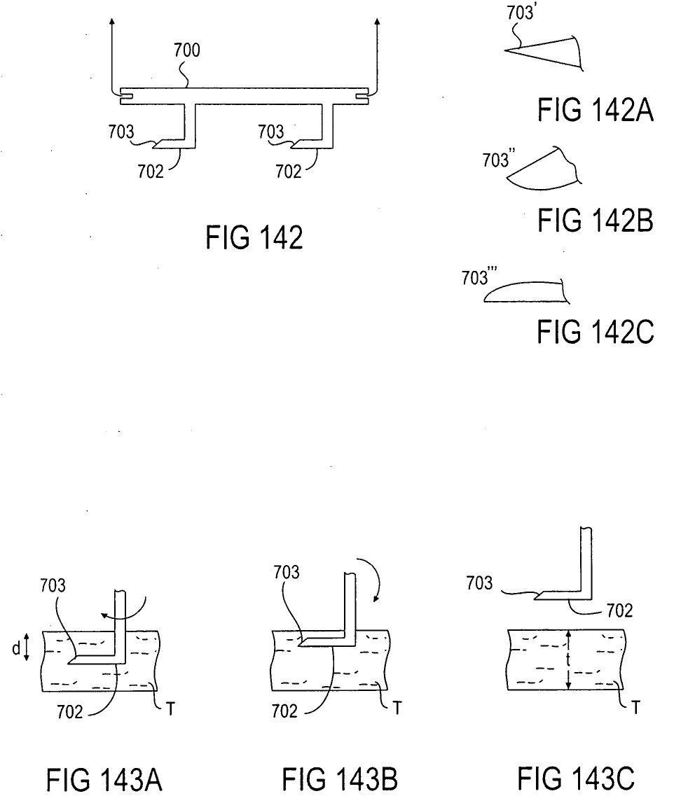

FIG. 142 illustrates an embodiment of an engagement ring 700 with two tines 702 extending therefrom. When placed on an instrument, the engagement ring 700 could be situated in a cylindrical channel within the distal end of the instrument such that the engagement tip 703 at each tine 702 is withdrawn from the distal end of the instrument. In this way the tines are prevented from inadvertent engagement during manipulation of the instrument. The engagement tip 703 may have a wide variety of designs depending upon the depth of penetration, the amount of rotation, and the thickness of the tissue to be engaged. FIGS. 142A, B and C illustrate three different tine penetration element configurations, 703′, 703″ and 703′″, respectively. FIGS. 143A-C illustrate the engagement of the tine 702 and penetration to a depth d within a tissue T having a depth t. FIG. 143A illustrates a tine 702 is advanced towards the surface of tissue T by advancing an instrument housing the tines (not shown) towards the tissue surface. FIG. 143B illustrates the engagement of tine tip 703 with the tissue as the tine ring is rotated. FIG. 143C illustrates the completion of the rotation of the tine and that the tine engagement tip 703 has penetrated to a depth d within the tissue. It is to be appreciated that the tines 702 as well as other anchoring and fixation features described in this application may be adapted to engage with the lumen wall thickness of the transluminal procedure in which they are used. In many cases, the target wall thickness is between 3 mm and 6 mm depending on the specifics of the target wall. For example, the thickness of the stomach wall ranges between about 4 mm and about 6 mm. As such, fixation and anchoring devices intended for use in the stomach without piercing the stomach wall would be adapted to penetrate and engage within the range of 4 to 6 mm. It is to be appreciated that the use of sensors on the date them and position indicator, such as for example, an ultrasound transducer, may assist the user in determining the actual tissue thickness at the target wall location. Knowledge of the actual or measured tissue thickness may then be used by a physician or operator to adjust the amount of tissue penetration provided by the fixation element.

In one representative embodiment, a guide tube has on its distal end a circular ring or a semi-circular ring having a plurality of tines that when twisted in one direction are adapted to advance into and engage adjacent tissue. When advanced in the opposite direction or when turned in the opposite direction, the tines withdraw from and pull out of the tissue. If stowed in the sidewall of the guide tube, then the tine ring or the tines are actuated to rotate out from a recess in the guide tube sidewall. As they rotate out, the tines engage into the tissue, thereby securing the distal end of the rigidizable tube against the tissue. The tine shape, size, depth of engagement, dimensions and other factors are specially adapted to pierce completely through the tissue that the rigidizable tube is to engage against. In alternative embodiments, the tine shape, size, depth of engagement, dimensions and other factors are specially adapted to engage only superficially in the tissue near the distal end of the rigidizable tube. In yet another alternative, the tine shape, size, depth of engagement, dimensions and other factors are specially adapted to engage fully within but not penetrate through the tissue against which the rigidizable guide tube will be landed.

A tissue anchor described below is adapted for passage through a lumen formed in, for example, a fixation element lumen in a guide tube, datum position indicator or other component adapted to be fixed to the wall of a lumen in support of transluminal procedures. One or more tissue anchors may be arranged around a portion of an instrument to engage that instrument to the lumen wall. One or more tissue anchors may be arranged around the distal end of a guide tube, or a datum position indicator as illustrated herein. This enables the tissue anchor to be advantageously used to anchor the guide tube, datum position indicator or other component to a lumen at the desired location in support of a transluminal procedure. The target tissue may be, for example, peritoneum, pleura, endocardium, organ capsules, skin or any other tissue depending upon the selected target for creating the opening in support of a transluminal procedure. The size of the engagement elements in a particular tissue anchor may be selected to remain within and affixed to a lumen wall or to penetrate through a lumen wall. Scaling a tissue anchor to the appropriate size is done using conventional techniques.

In the description that follows, tissue anchors will be described in use with a guide tube. It is to be appreciated that the tissue anchors and the other fixation elements described herein may be used to secure other components to a lumen wall or other portion of the anatomy. The guide tube, or other device to be anchored is inserted into a lumen of a patient undergoing a surgical procedure. Tissue grasping features at a distal end of the tissue anchor are manipulated to be positioned adjacent and move into the target tissue to anchor the guide tube, datum and position indicator or other component into a desired position so that a surgeon may more readily perform the intended surgical procedure. The tissue grasping end of the tissue anchor may be adapted to articulate at an angle by controlling the opposite end such that the tissue grasping end or surface becomes substantially orthogonal to the surface of the target tissue to facilitate grasping the target tissue.

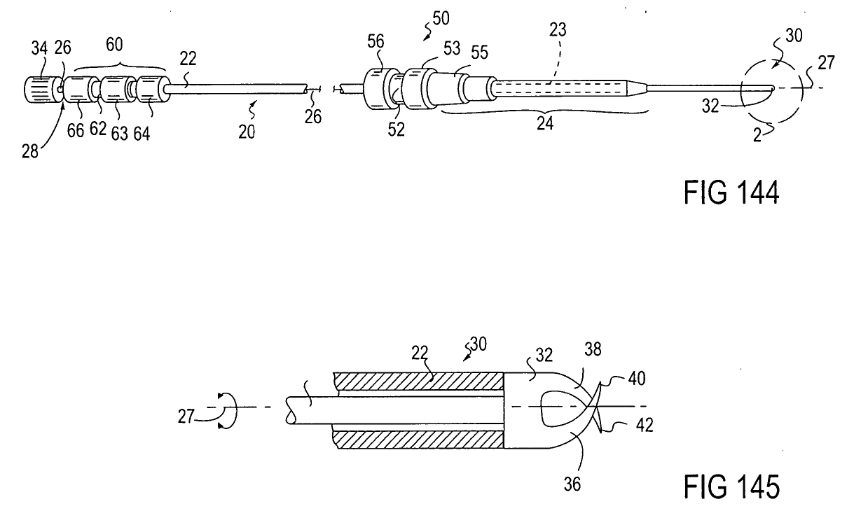

FIG. 144 is a perspective view of one embodiment of a tissue anchor and includes a manipulation and locking device for use with a guide tube or datum position indicator. FIG. 145 is an enlarged view of the region encircled by line 2 of FIG. 144. Tissue anchor 20 includes an elongated outer tube 22 adapted to be insertable into a channel of the guide tube. An elongated, semi-rigid shaft 26, defining a longitudinal axis 27, is received within tube 22. The shaft 26 includes a proximal end or remote end 28 and a distal end or working end 30. The distal end 30 includes a tissue grasping member 32 for grasping and anchoring a target tissue (not shown). In the illustrative example shown in FIG. 145, the tissue grasping member 32 includes two nearly horizontally opposing prongs 36, 38, which are each helically formed, having tips 40 and 42, respectively. The proximal end 28 of shaft 26 may be connected to a knob 34 or other device for manipulating the shaft 26 and the tissue grasping member 32.

Tissue anchor 20 may include an axial lock 50 that locks the outer tube 22 relative to the guide tube to prevent relative axial movement therebetween. In one embodiment, axial lock 50 includes a housing 52 having a standard Luer connector 53 for securing the axial lock 50 to the proximal end 55 of catheter 24. The axial lock 50 further includes a glandular member (not shown) disposed between the housing 52 and the outer tube 22. A cap 56, when screwed onto the housing 52, compresses the glandular member in a tight sealing engagement relative to the outer tube 22 to reduce any axial movement therebetween. This tight sealing engagement is also effective in reducing the possibility of leakage of fluids or the injected gas from the body cavity. Such a locking device is typically termed a Touhy-Borst, which may be purchased from the Becton-Dickinson Corporation, Franklin Lakes, N.J., U.S.A. It is to be appreciated that other suitable locks which axially lock the outer tube 22 relative to the guide tube may be used.

The tissue anchor 20 may also include a rotational lock 60 constructed and arranged to rotationally lock outer tube 22 and the shaft 26. In this illustrative example, rotational lock 60 is of a similar construction to axial lock 50. It should be understood, however, that rotational lock 60 may be any suitable locking device or arrangement adapted for rotationally locking two concentric members. Accordingly, the rotational lock 60 includes a housing 62 having a standard Luer connector 63 formed within housing 62. A plug 64 is secured to outer tube 22 and is adapted for insertion into the Luer connector 63 for securing the outer tube 22 to the housing 62. The rotational lock 60 further includes a glandular member (not shown) formed within the housing 62. The shaft 26 passes through the glandular member and is attached to the knob 24. A cap 66, having an opening to allow the shaft 26 to pass therethrough, is also provided. When cap 66 is screwed onto the housing 62, the glandular member is compressed in a tight sealing engagement relative to the shaft 26 to reduce the rotational movement thereof, as well as to reduce the possibility of leakage of fluids or gas.

During a surgical procedure, guide tube is inserted into a body cavity of a patient. The tissue anchor 20 is then inserted into the guide tube (if not already present in a sidewall fixation channel) and positioned such that the distal end 30 having the tissue grasping member 32 touches the target tissue. One or more tissue anchors may be disposed on the distal end or tissue contacting portion of the guide tube or datum position indicator. Next, the knob 34, together with the shaft 26, is rotated, for example, in a counter-clockwise manner, such that the nearly horizontally opposing tips 40, embed into and secure the guide tube against the target tissue. The rotational lock 60 is then locked such that any additional rotation of the shaft 26 relative to the outer tube 22 is reduced. This, in return, reduces any inadvertent releasing of the target tissue. Once the desired guide tube axial displacement is achieved, the axial lock 50 is locked to reduce any further axial movement of the tissue anchor 20 relative to the guide tube and to reduce any leakage therebetween. To release the target tissue, the shaft 26 is rotated in an opposite direction, for example, clockwise, whereby the prongs 36, 38 of the tissue grasping member 32 release from the target tissue allowing the guide tube or datum position indicator to be withdrawn. In one embodiment, the movement of all tissue anchors 20 may be synchronized to engage and disengage the target tissue simultaneously. Alternatively, each individual tissue anchor may be controlled independently.

The outer tube 22 has an outer diameter sized to accommodate the guide tube tool channel and an inner diameter sized to accommodate the shaft 26. In one embodiment, the outer tube 22 has an outside diameter approximately equal to that of a typical catheter needle (for example, 17 gage or 0.058 in.) and has a wall thickness of about 5-10 mills, although a thicker or thinner wall may be suitable. Also, although the shaft 26 is shown and described as a semi-rigid cylindrically-shaped shaft, the shaft may be formed of a cable or a tube of any cross-sectional shape, and may be stiff or flexible and may be made of any suitable material, including, for example, stainless steel or plastic. The tissue grasping member 32 may be connected to the shaft by any suitable means such as crimping or brazing.

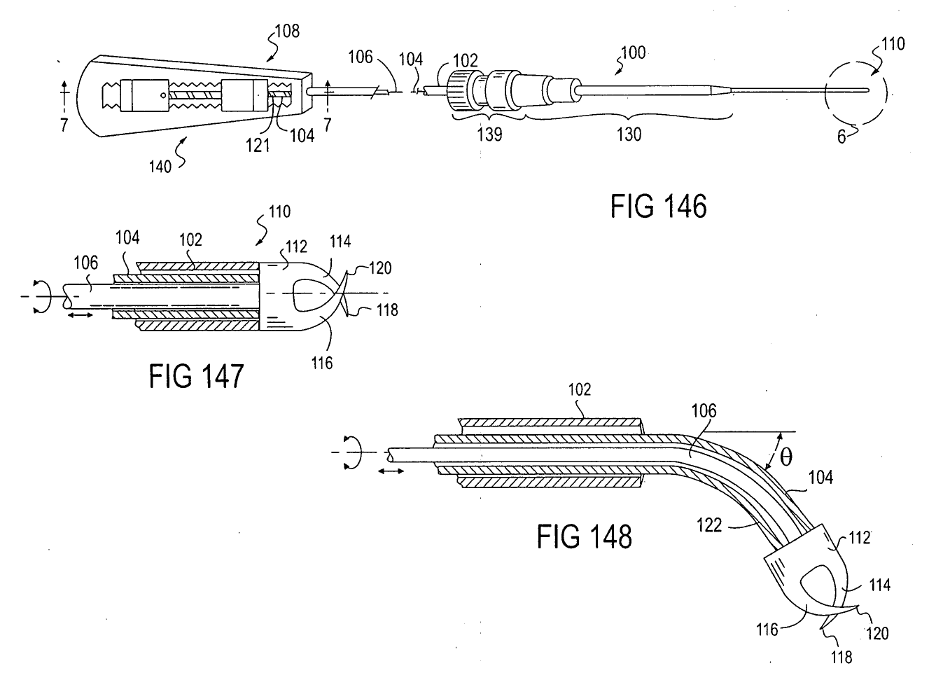

FIG. 146 is a perspective view of this embodiment of a tissue anchor configured to articulate. In this embodiment, tissue anchor 100 includes an outer tube 102, an inner tube 104 disposed within the outer tube 102 adapted for axial movement therein, and a flexible shaft 106 disposed within the inner tube 104 for rotational movement therein. The shaft 106 includes a proximal end 108 and a distal end 110. A tissue grasping member 112 is attached to the shaft 106 at the distal end 110. In this illustrative example of tissue anchor 100, tissue grasping member 112 is similar to that described above with respect to tissue anchor 20. Tissue grasping member 112 includes two nearly horizontally opposing prongs 114, 166, which are helically formed, such that when the tissue grasping member 112 is rotated, the tips 118, 120 of the prongs 116, 118, respectively, embed into the target tissue for anchoring. The inner tube 104 has a proximal end 121 and a distal end 122.

In the embodiment described with reference to FIGS. 146-150, the inner tube 104 is formed of a shape-memory material. Such a material may be spring steel, Nickel-Titanium, plastic or any other material now or later developed that has the characteristic of significantly deflecting and returning to a desired rest position.

The shaft 106 is formed of a flexible material such as stainless steel, plastic or any other suitable material. The material chosen is sufficiently flexible to allow rotation of the shaft 106 about is axis when the shaft is in a bent configuration, as will be appreciated hereinafter.

When the inner tube 104 is retracted within the outer tube 102, the two remain substantially coaxial with each other (as shown in FIG. 147, which represents an enlarged view of distal end 110 in a retracted position encircled by line 6 of FIG. 146). However, when the inner tube 104 is moved such that its distal end 122 emerges from the outer tube 102, the distal end 122 bends at an angle .theta. relative to the outer tube 102 (as shown in FIG. 148, which represents an enlarged view of distal end 110 in an extended position encircled by line 6 of FIG. 146). The inner tube 104 bends because it is formed with a shape memory material with its rest position having a bend with a maximum angle theta. Once retracted into the outer tube 102, the inner tube 104 is in a biased position wherein the inner tube 104 is in the substantially coaxial alignment relative to the outer tube 102. Thus, the amount of angular deflection of the inner tube 104 relative to the outer tube 102 is determined by the amount of extension of the inner tube 104 relative to the outer tube 102. To change the angle .theta., the inner tube 102 is positioned to a desired axial displacement relative to the outer tube 102. According to the present invention, the angle theta. may range from about 0.degree. to 180.degree., although a range from about 0.degree. to 90.degree. is preferable.

In use, the tissue anchor 100, if not already present in the tool channel of a guide tool sidewall, is inserted into a guide tube tool channel and is positioned such that the tissue grasping member 112 is in proximity to the target tissue. However, in contrast to the example of FIGS. 144 and 145, if the tissue grasping member 112 is not initially substantially orthogonal to the surface of the target tissue, the tissue anchor is retracted slightly relative to the guide tube and the inner tube 104 is axially displaced relative to the outer tube 102, such that the inner tube 104 articulates relative to the outer tube 102, until the tissue grasping member 112 becomes substantially orthogonal to and in contact with the surface of the target tissue. Once in this position, the shaft 106 is rotated such that the prongs 114, 116 of the tissue grasping member 112 embed into the target tissue to anchor the guide tube. Once embedded, the tissue anchor 100 is retracted relative to the guide tube, to lift the target tissue to a desired position and anchor the guide tube thereto. One or more tissue anchors 100 may be used to anchor the distal tip of the guide tube. The tissue anchors 100 may be activated simultaneously or independently.

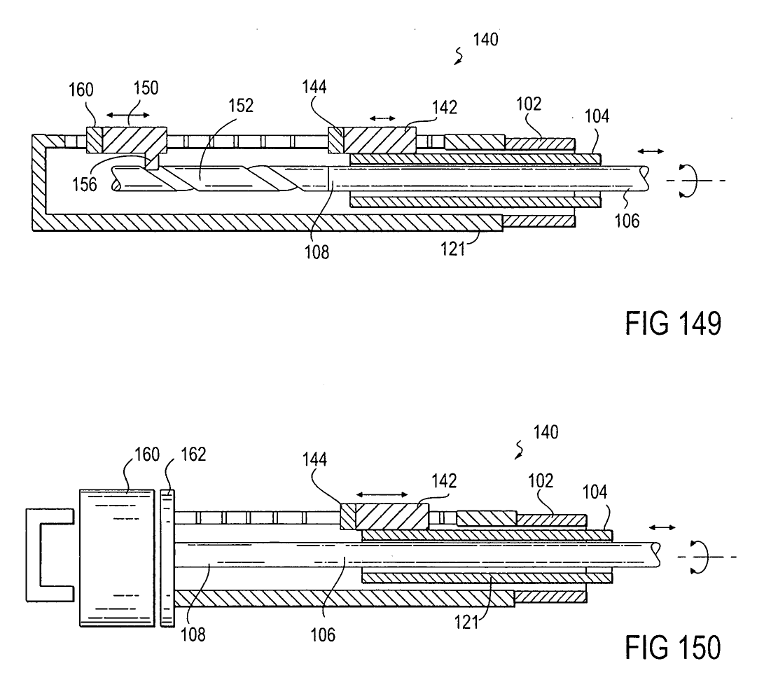

Continuing with reference to FIG. 146, an axial lock 139 locks the outer tube 102 to the catheter 130 to prevent relative axial movement. Such an axial lock 139 is similar to axial lock 50 described with reference to FIG. 144. The tissue anchor 100 may also include additional locks to lock the inner tube 104 relative to the outer tube 102 and to lock the shaft 106 relative to the outer tube 102, as will be fully described with reference to FIGS. 149 and 150.

In the embodiment described with reference to FIGS. 146-150, the outer tube 102 has an outer diameter sized to fit within the guide tube fixation channel and inner diameter sized to accommodate both the inner tube 104 and the shaft 106. In a preferred embodiment, the outer tube has an outer diameter of about 17 gage (0.058 in.) and a wall thickness of about 5-10 mills, although a larger or smaller diameter or a thicker or thinner wall may be suitable. Also, although the shaft 106 is shown and described as a flexible, cylindrically-shaped shaft, the shaft may be formed of a cable or a tube or any cross-sectional shape and may be made of stainless steel or plastic. The tissue grasping member 32 may be connected to the shaft by any suitable means such as crimping or brazing.

Although the embodiment described with reference to FIGS. 146-150 includes the helically formed prongs 114, 116, it is to be appreciated that the tissue grasping member 112 may be formed with the opposing spring-like prongs, or take the form of other fixation elements or structures described herein. In such an embodiment, although not shown, the shaft may be axially moveable relative to the inner tube to cause the tissue grasping member to open and receive the target tissue as well as to grasp and hold the target tissue into a desired position.

Referring now in particular to FIG. 149, one embodiment of a handle 140 of the tissue anchor 100 of FIG. 146 is illustrated. Handle 140 is positioned along with or is included within the guide tube controller or other system controller to actuate the locking mechanisms as needed. The actuator 142 is housed within the handle 140 and is adapted to move axially relative thereto, thereby causing the inner tube 104 to move axially relative to the outer tube 102.

A second sliding actuator 150 may be provided in the handle 140 which translates linear motion of the actuator 150 to rotational motion of the shaft 106. This may be accomplished with a helix 152 formed on the proximal end 108 of the shaft 106, and a cam follower 156 formed on the actuator 150. Thus, as the actuator 150 slides relative to the handle 140, the cam follower 156 forces the shaft 106 to rotate. It is to be appreciated that the helix 152 may be integrally formed with the proximal end 108 or may be a separate member attached to the proximal end 108, as desired.

Again, an axial lock 160 may be formed on the actuator 150 to lock the actuator 150 relative to the handle 140 to reduce any translation relative therebetween, which would ultimately result in a rotation of the shaft 106.

Although the actuators 142, 150 are housed within a handle 140, as shown in the embodiments with respect to FIGS. 146-149, those skilled in the art will recognize in view of this disclosure that other actuating mechanisms may be used, including a multiple plunger arrangement as previously described or a nested actuator arrangement whereby the second sliding actuator 150 is housed within the first sliding actuator 142. This nesting arrangement may be desirable because, when attempting to move the inner tube without causing the shaft to rotate, both actuators must move in unison. Only when rotation of the shaft is desired is the second actuator moved independent of the first.

FIG. 150 illustrates another embodiment of the handle 140, wherein a knob 160 may be attached to the shaft 106 and housed within the handle 140 rather than provide the second sliding actuator 150. The shaft 106 may be rotated by rotating the knob 160 relative to the handle 140. A rotational lock 162 may be provided to lock the rotation of the shaft 106.

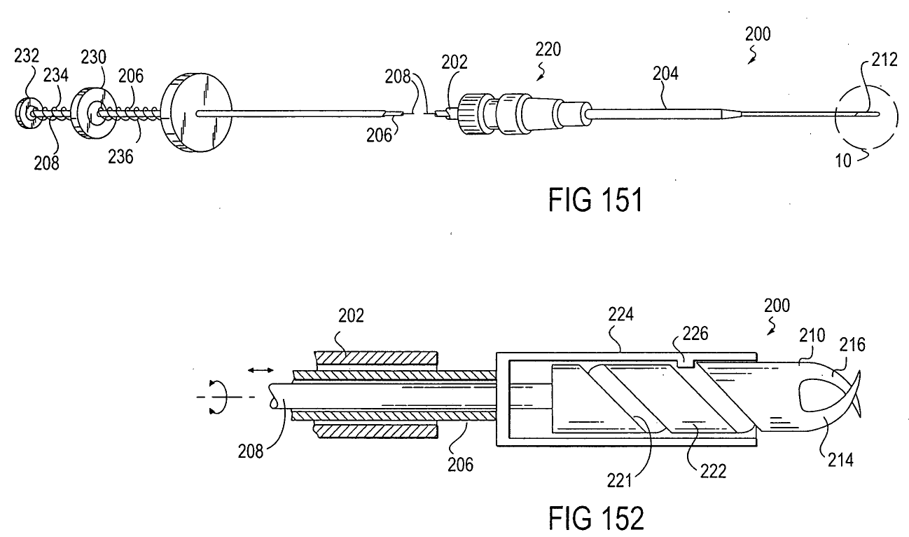

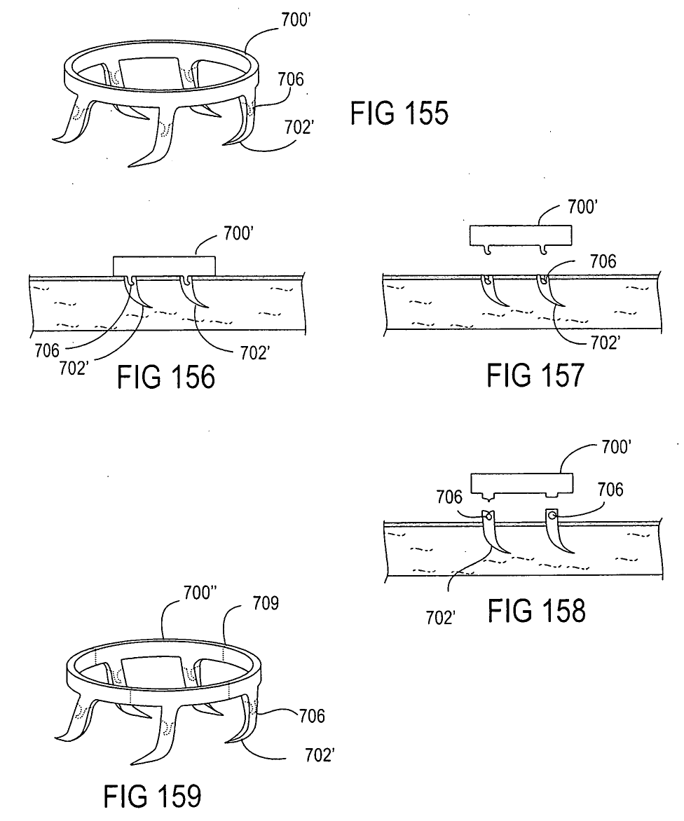

The embodiment described with reference to FIG. 149 shows the helix 152 which translates linear motion to rotational motion formed at the proximal end 108 of the shaft. However, as shown in the embodiment of FIGS. 151 and 152, the helix may be formed at the distal end of the tissue anchor. In this illustrative example of FIG. 151, a tissue anchor 200 is shown in perspective. Tissue anchor 200 includes an outer tube 202 adapted to be inserted into a guide tube fixation channel, an inner tube 206 disposed within the outer tube 202 and a flexible shaft 208 disposed within the inner tube 202. An axial lock 220 may be used to axially lock the outer tube 202 relative to the catheter 204, as described with reference to FIGS. 144-150. As best shown in FIG. 152 which is an enlarged view of the area encircled by line 10 of FIG. 151, a tissue grasping member 210 is formed at the distal end 212 of the shaft 208. The tissue grasping member 210 includes helically formed prongs 214, 216, as described with reference to FIGS. 144-150.

The tissue grasping member 210 is formed with a helix, such as a helical groove 221 in a body 222, which is received within a housing 224. The housing 224 is formed with a cam follower 226, which is adapted to engage the groove 221. The housing 224 is attached to the inner tube 206 if an articulating tissue anchor is employed, as in this example. Thus, when the shaft 208 moves axially relative to the inner tube 206, the tissue grasping member 210 rotates relative to the housing 224, thereby causing the prongs 214, 216 of the tissue grasping member 210 to grasp the target tissue. Those skilled in the art will recognize in view of this disclosure that although the body of the tissue grasping member is formed with a helix and the cam follower is formed on the housing, the opposite may be true, wherein the housing may include a helix, which may be a groove or a raised portion, and the cam follower may be formed on the tissue grasping member.

As described above with reference to the embodiment of FIGS. 146-150, the inner tube 206 of the embodiment of FIGS. 151 and 152 is formed of a shape-memory material such as spring-steel, Nickel-Titanium, plastic or any other material now or later developed that has the characteristic of significantly deflecting and returning to a desired rest position. Also, the shaft 208 is formed of a flexible material such as stainless steel, plastic or any other suitable material. The material chosen is sufficiently flexible to allow rotation of the shaft 208 about its axis when the shaft is in a bent configuration.

Movement of the inner tube 206 and the shaft 208, in the example of FIGS. 151 and 152, is accomplished through a nesting plunger-type arrangement, wherein a first plunger 230 is attached to the inner tube 206 and a second plunger 232 is attached to the shaft 208. A first and second spring 234, 236 may be used to bias the axial position of the inner tube 206 and the rotational position of the shaft 208 to desired rest positions, respectively. In a preferred embodiment, the rotational rest position may be such that the tissue grasping member 210 is in a tissue grasping rotational orientation. Thus, when the plunger 232 is depressed, the tissue grasping member 210, may rotate in a direction opposite the direction of the prongs 214, 216 such that the target tissue cannot be grasped. Upon release of the plunger 232, the spring 234 will push the plunger 232 axially, thereby causing the tissue grasping member 210 to rotate into a position such that the prongs 214, 216 may grasp the target tissue. It is to be appreciated that the mechanisms described here and for the rotation, locking and manipulation of the tissue anchors may be applied and/or adapted for use with other fixation elements described herein.

As discussed with reference to the embodiment of FIGS. 146-150, the outer tube 202 of the embodiment of FIGS. 151 and 152 has an outer diameter sized to fit within the guide tube fixation channel and an inner diameter sized to accommodate both the inner tube 206 and the shaft 208. In a preferred embodiment, the outer tube has an outer diameter of about 17 gage (0.058 in.) and has a wall thickness of about 5-10 mills, although a larger or smaller diameter or a thick or thinner wall may be suitable. Also, although the shaft 208 is shown and described as a flexible cylindrically-shaped shaft, the shaft may be formed of a cable or tube of any cross-sectional shape and may be made of stainless steel or plastic. The tissue grasping member 32 may be connected to the shaft by any suitable means such as crimping or brazing.

According to another aspect of the invention, it may be advantageous to use a plurality of tissue anchors of the present invention during a surgical procedure so as to stabilize a tissue structure for reconstruction, for example. In addition, a plurality (four, for example) may be used to stabilize the heart during a “beating heart” procedure. Other applications of a single or multiple tissue anchors according to the present invention will be readily apparent to those skilled in the art.

In addition, as should be apparent to those skilled in the art in view of this disclosure, any of the disclosed devices, as well as any other suitable device, used to actuate the inner tube or the shaft, may be used in any of the embodiments. Also, although the helix shown in the examples described herein used to translate linear motion to rotational motion is in the form of a groove or a raised portion, a spring may be used as the helix. Thus, as used herein, the term helix means any helically shaped form or helical member used to transform linear motion to rotational motion. U.S. Pat. No. 6,228,023 titled “Tissue Pick and Method for Use in Minimally Invasive Surgical Procedures” to Zaslavsky et al., filed Feb. 17, 1999 is incorporated herein by reference in its entirety.

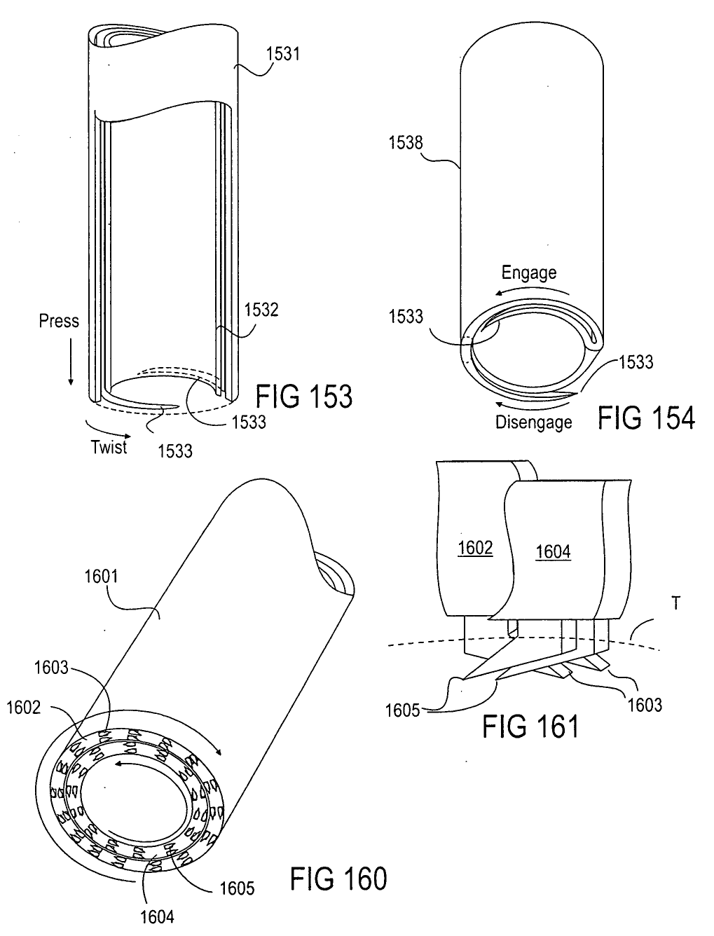

FIGS. 153 and 154 illustrate additional tine engagement member embodiments. FIG. 153 illustrates a lumen 1531 having a recess 1532 formed in its distal end. The recess 1532 exists within the side wall of the lumen 1531 and houses tines 1533 while the lumen 1531 is transiting to the site for creating a transluminal opening, the tines 1533 remain within recess in 1532 so that the tines do not inadvertently engage and/or harm tissue other than the targeted tissue. Once the distal end of the limit of 1531 is placed at the target tissue side, and actuation mechanism (not shown) advances the tines out of the recess in 1532 and, with a twisting motion indicated by the arrow into engagement with the target tissue. Lumen 1538 does not have a recess in 1532 to house times 1533. Tines 1533 remain on the distal end of the lumen 1538. In one specific embodiment, the lumens 1538, 1531 are guide tubes adapted as illustrated to utilize times 1533 to engage with and secure to the target tissue area.

FIGS. 155-159 illustrate alternative rotational engagement rings 700′ and 700″ having pre-designed fracture points. As best seen in FIG. 155 engagement ring 700′ has fracture points 706 along the shaft of each of the tines 702′. The fracture point 706 is intended to separate the tine from the engagement ring in such a way as to leave a feature with which to engage a suture or other closure device to facilitate closing the transluminal opening. FIG. 156 illustrates how tines 702′ engage with tissue as described above. However, as best seen in FIG. 157 and 158, the engagement ring 700′ breaks away from the tines 702′ leaving the suture or closure engagement features 706. FIG. 159 illustrates an engagement ring 700″ having a fracture point 709 in addition to fracture line 706. Fracture line 709 allows the engagement ring to be expanded and break as a result of dilation procedures applied to the transluminal opening, such as the dilation procedures described below. In one embodiment, the tines 702′, 702″ are formed from a biocompatible, biodegradable material that will dissolve over time. Alternatively or additionally, the tines 702′ may be coated with a pharmacological agent that promotes healing of the transluminal opening.

In contrast to lumen 1531, 1538, the lumen 1601 of the embodiment of FIG. 160 has multiple small tines instead of a pair of large tines. Elongate body 1601 defines a lumen 1608. An outer tine ring 1602 and an inner tine ring 1604 are positioned on the distal end 1607 of the elongate body 1601. The outer tine ring 1602 has a plurality of tines 1603 arranged in pairs around the perimeter of the ring 1602. Similarly, the inner tine ring 1604 has a plurality of tines 1605 arranged in pairs around the perimeter of the ring 1602. In the illustrated configuration the outer tine ring 1602 rotates in a clockwise direction (in the direction of the arrow) to engage with tissue. The inner tine during 1604 rotates in a counterclockwise direction (in the direction of the arrow) in order to engage with tissue. FIG. 161 illustrates the engagement of tines 1603, 1605 into tissue T.

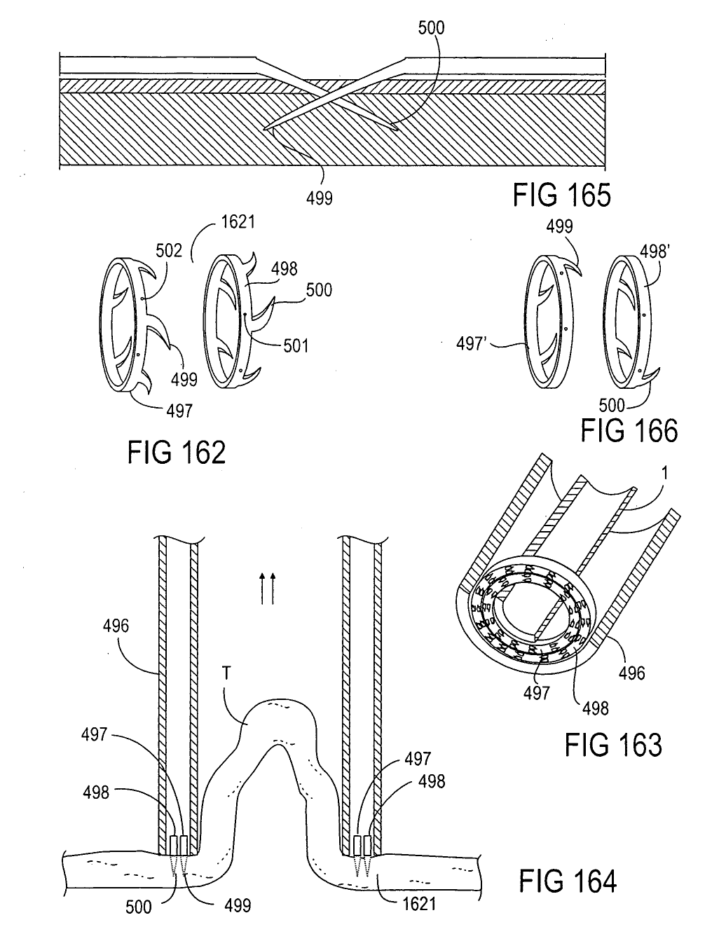

In contrast to earlier tine embodiments where the tines run in a somewhat parallel but intersecting pathway, the tines illustrated in FIGS. 162-166 rotate in a nearly orthogonal engagement pathway to the target tissue surface. FIGS. 162-166 show a two-piece tine ring tissue anchor 1621 having a fastening flange and integrally formed staple members. In this case, the fastening flange of the device is formed of two concentric cylindrical flange rings 497, 498. The tines may completely encircle the rings as shown in FIG. 162 or only partially encircle the rings as shown in FIG. 166. A plurality of interlocking staple members 499, 500 extend from the distal edges of both cylindrical flange rings 497, 498 as best seen in FIGS. 163 and 164. Preferably, the staple members 499, 500 are integrally formed with the cylindrical flange rings 497, 498 but may be separate components. As best seen in FIG. 162, the staple members 499 of the inner flange ring 497 are angled so that they spiral downward from the ring 497 in a clockwise direction. The staple members 500 of the outer flange ring 498 are oppositely angled so that they spiral downward from the ring 497 in a counterclockwise direction. Corresponding locking features 501, 502 on the inner surface of the outer flange ring 498 and on the outer surface of the inner flange ring 497 are capable of locking the two flange rings 498, 497 together in a fixed position. Indentations on one flange ring, with corresponding detents on the other flange ring are one of the many possibilities for the locking features 501, 502. When engaged in the tissue, the tines 499, 500 penetrate as best shown in FIG. 165.

In use, the tissue anchor 1621 is applied to the target tissue by, in one example, separately placing first the outer flange ring 498, then the inner flange ring 497 to secure the end of the guide tube 496. The target wall tissue T is drawn into the lumen of guide tube 496 using vacuum as indicated by the arrow. When the locking features 501 of the outer ring 498 coincide with the locking features 502 of the inner ring 497, the outer 498 and inner 497 rings become locked together. As the flange rings 497, 498 are rotated in opposite directions, the staple members 499, 500 of the inner 497 and outer rings 498 penetrate the vessel walls in opposite directions as shown in FIG. 165, effectively locking the guide tube anchoring device to the target vessel wall.

Alternatively, the inner 497 and outer rings 498 of the tissue anchor can be applied simultaneously to grasp the tissue wall at the target wall site T by then pressing the staple members 499, 500 into the tissue wall T while counter-rotating the inner 497 and outer 498 rings. This could best be done with an instrument that holds and rotates the inner 497 and outer 498 rings mechanically. Once held by vacuum, a transluminal opening may be formed in the tissue T using the techniques described herein. Then, as illustrated in FIG. 163, a steerable instrument 1 may be advanced through the lumen in the guide tube 496 and through the transluminal opening.

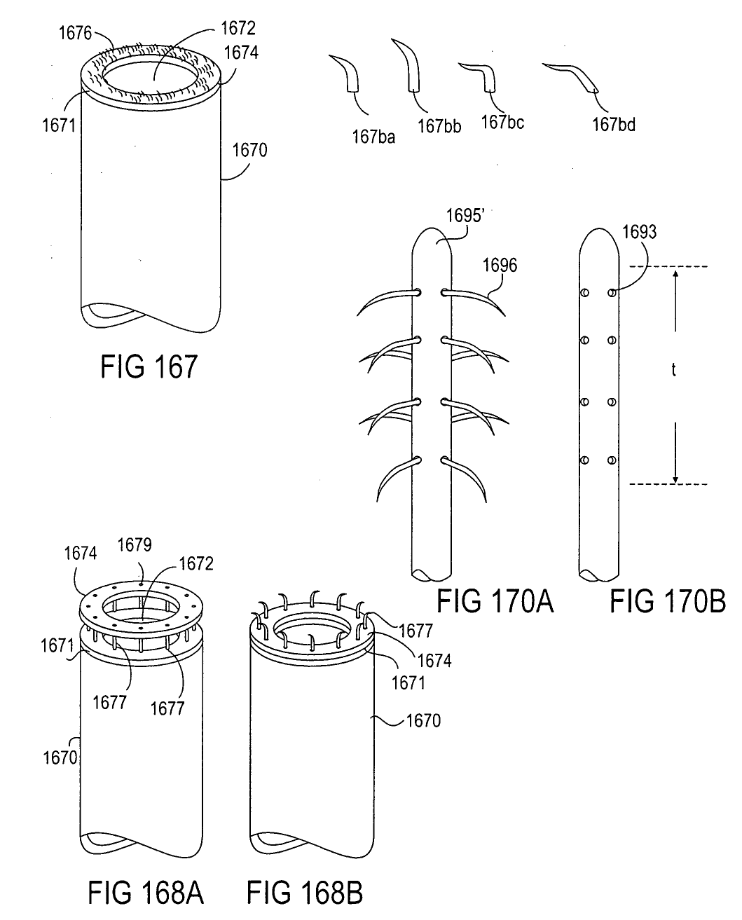

FIGS. 167 through 168B illustrate another distal end attachment embodiment. As best seen in FIG. 167, the guide tube 1670 has a distal end 1671 and a lumen 1672 extending there through. An annular disk 1674 around the circumference of the distal end 1671 contains a plurality of micro-barbs, micro-wires or other small features 1676 shaped to engage with target tissue when the disc 1674 is applied to the target tissue. FIGS. 167a-167d illustrate a wide variety of micro-barb, micro hook and micro-wire configurations that may be placed on the disc 1674. It is to be appreciated that the various alternative features 1676 penetrate into tissue less than 6 mm.

In contrast to the fixed features contained on disk 1674 in FIG. 167, the embodiment illustrated in FIG. 168 a has a retractable disk 1674 with a plurality of apertures 1679 arrayed around its circumference. When disk 174 is in the extended position illustrated in FIG. 168A, the engaging portions of the features 1677 are within the apertures 1679. As such, the configuration illustrated in FIG. 168A is a convenient way to manipulate the guide tube 1670 while preventing inadvertent tissue engagement with the features 1677. When the guide tube 1670 is positioned in a desired location, the engagement features 1677 are engaged by simply withdrawing the disc 1674 back towards the distal end 1671 as best seen in FIG. 168B. This action advances the features 1677 out through the aperture 1679 and into engagement with the targeted tissue. This action secures the guide tube 1670 into position with the target tissue. Apertures 1679 are arranged in a simple circular pattern in the embodiment illustrated in FIGS. 168A and B. Other aperture/feature patterns are possible such as multiple circular arrangements or clusters of features at particular points or other patterns that may be suited to a particular transluminal target site.

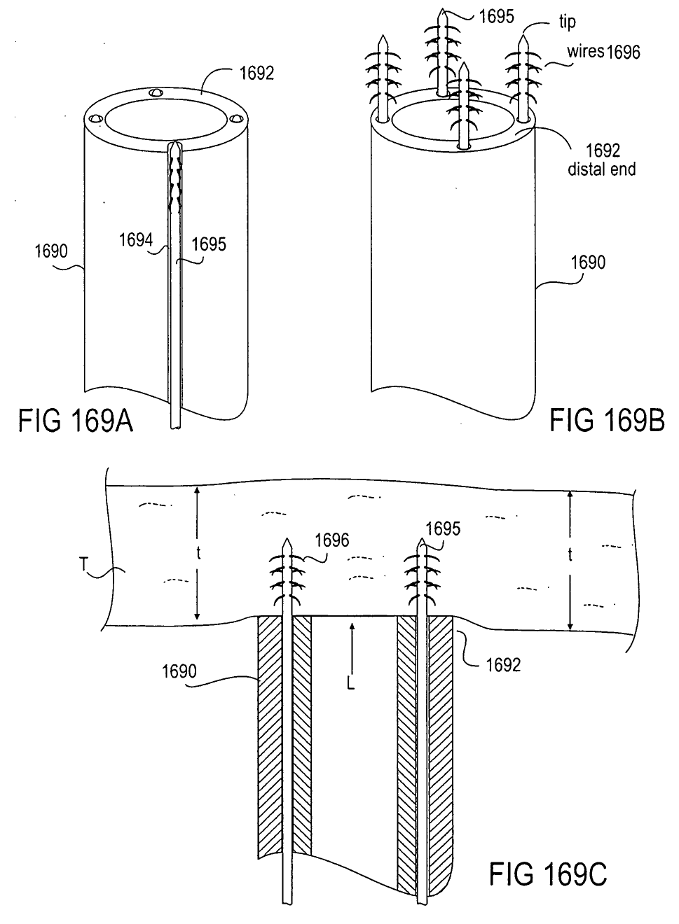

The ability to withdraw a fixation element into the side wall of the guide tube is also illustrated in FIGS. 169A through 169C. As is best seen in FIG. 169A, guide tube 1690 has a distal end 1692 and a fixation element channel 1694 within the side wall. FIG. 169A illustrates the fixation elements 1695 in a stowed condition within the fixation element channel 1694. In the stowed condition, the distal end 1695 of the fixation element is below the distal end 1692. As described in previous embodiments, the absence of fixation elements on the distal end of the guide tube or other instrument helps reduce the likelihood of an inadvertent puncher or engagement of tissue surrounding the target tissue site. The fixation element 1695 is shown in a stowed condition inside of the fixation element channel 1694 (FIG. 169A). As is best seen in FIG. 169B and 169C a plurality of small wires 1696 are fixed to the distal end of the fixation element 1695. When the fixation element 1695 is advanced out of the fixation element channel 1694 both the fixation element tip 1695 and the wires 1696 engage with the surrounding target tissue as best seen in FIG. 169C.

An alternative fixation element 1695′ having a plurality of retractable wires 1696 is illustrated in FIGS. 170A and 170B. In this embodiment, the wires 1696 are withdrawn into the interior of the fixation element 1695′. When deployed, the wires 1696 are advanced out of apertures 1693 in the body of the fixation element 1695′.

In the illustrated embodiment of FIG. 169C, the depth of penetration of the distal tip 1695 and the engagement reach of the wires 1696 is selected so that the tip1695 does not penetrate through the tissue thickness t nor do the wires 1696 engage so far into the tissue to actually block of the lumen pathway L that will be created through the guide tube using a transluminal opening procedure described herein.

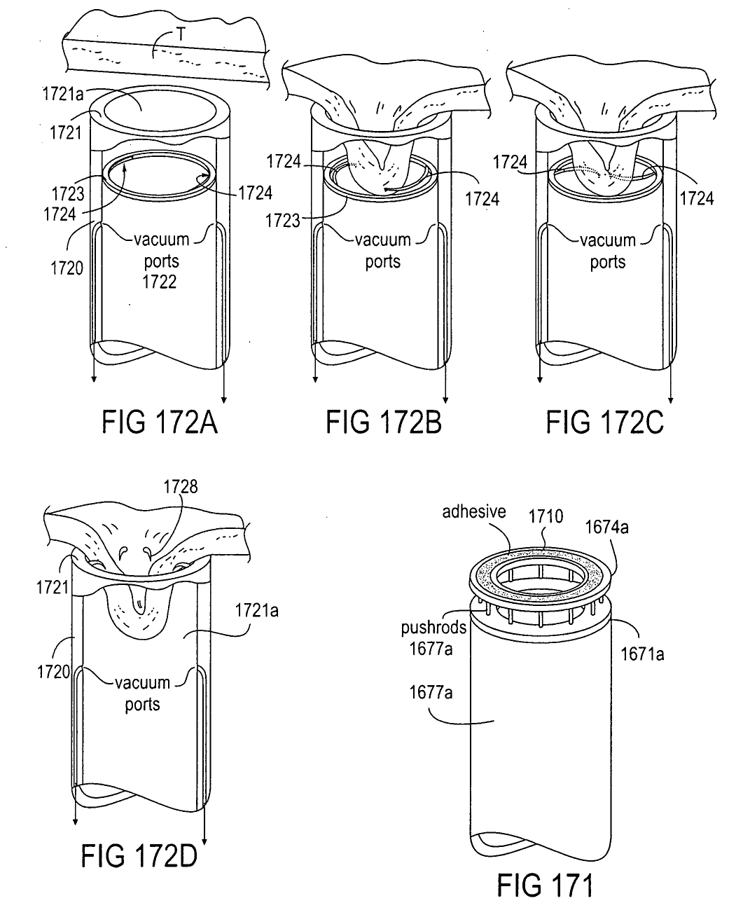

FIG. 171 illustrates a slightly modified guide tube 1670 from the embodiment illustrated above in FIG. 168. In the embodiment illustrated in FIG. 171 push rods 1677a extend through the distal end 1671a and engage with an annular plate 1674a. The push rods 1677a move the annular plate 1674a from a stowed position against the distal end 1671a and the extended position as illustrated. In the illustrated embodiment, the annular plate 1674a is coated with an adhesive 1710 that is used to seal the distal end of the guide to the 1672 the target tissue wall. The adhesive 1710 maybe any adhesive used in the medical arts for joining tissue. A solvent may later be applied to dissolve the adhesive and free the guide tube 1670 from the target tissue.

FIGS. 172A through 172D illustrate alternative tissue fixation devices. The guide tube 1720 has a distal end 1721 and the lumen 1721a. Vacuum ports 1722 are disposed within the side wall of the guide tube and are used to apply vacuum to the guide tube lumen 1721a. A sidewall gripper set 1723 is positioned within the sidewall of the guide tube lumen proximal to the distal end 1721. The sidewall gripper set 1723 includes a pair of engagement elements 1724 that are best seen in FIG. 172C. Once vacuum is applied to the lumen 1721a and the distal end 1721 is close enough, the target tissue T will be drawn into the guide tube lumen 1721a as best seen in FIG. 172B. The engagement elements 1724 move in an arc pattern across the guide tube lumen axis to secure the target tissue T. In contrast, the guide tube 1720 embodiment illustrated in FIG. 172D includes side wall-mounted fixation elements 1728. The side wall-mounted fixation elements 1728 are stowed within the side wall of the guide to bloom in and operate to engage the target tissue by moving in a direction that follows that generally follow the guide tube lumen access. Both the engagement elements 1724 and 1728 utilize the lumen side wall for engaging the target tissue thereby leaving the distal end 1721 free for other tasks in support of a transluminal or other procedure.

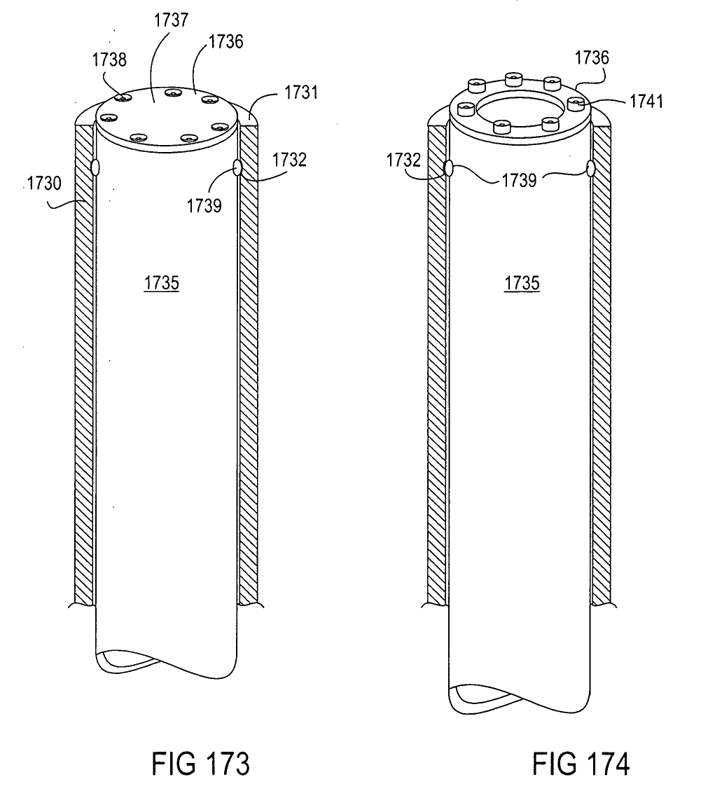

FIGS. 173 and 174 illustrate alternative embodiments of the guide tube applicators 1735. The guide tube 1730 has a distal end 1731 and an engagement element 1732 in the sidewall. The engagement element 1732 is configured to engage with a complementary engagement element 1739 in the outer wall of the applicator 1735. The applicator 1735 has a distal end 1732 and a distal surface 1737. In the embodiment of FIG. 173, the applicator 1735 has push rods 1738 beneath the surface and configured to extend up through the surface 1737 through the opens shown. In one embodiment, a sterile adhesive patch is attached to the surface 1737 and then the applicator is advanced distally, until the engagement features 1739/1732 prevent further passage along the guide tube lumen. Note that the guide tube feature 1732 may be placed at any position along the lumen to vary the exact location of the applicator 1735 within the guide tube. In an instance where the surface 1737 contains a patch to be applied to the target tissue to aid in maintaining and creating a sterile field, then the feature 1732 would be positioned as shown so that a sterile adhesive patch placed in the surface 1737 would be placed in contact with tissue by advancing the guide tube to wards and into contact with the tissue. Once in position, the push rods 1738 are advanced to press the patch against and secure it to the target tissue. It is to be appreciated that the adhesive patch will be the same size as or slightly larger than the guide tube lumen so that the entire target tissue within the guide tube lumen is covered. Applying a sterile adhesive patch to the target wall may provide a simple and efficient way to provide a sterile environment. The transluminal opening is then created by cutting through both the tissue and the underlying, un-sterilized tissue.

The applicator illustrated in FIG. 174 has a plurality of nozzles 1741 directed towards the target tissue. The nozzles 1741 could be used to spray a sterilizing chemical or to spray on a sterilized coating or sealant that would provide a sterile barrier. A spray on form of the liquid skin products sold under the trade names NuSkin, liquid Band-Aid and Liquid Gloves and the like may be suitable for this purpose. The guide tube engagement feature may also be positioned more proximal than illustrated. In this manner, a small chamber is created in the distal end of the guide tube (i.e., this example envisions that the guide tube is secured to the target tissue but that the transluminal opening has not been created). The chamber is bounded by the guide tube lumen, the target tissue wall and the applicator distal end 1736. The applicator could also be used in the illustrated configuration and used generally to spray sterilizing fluids, sealants or other compounds as needed.

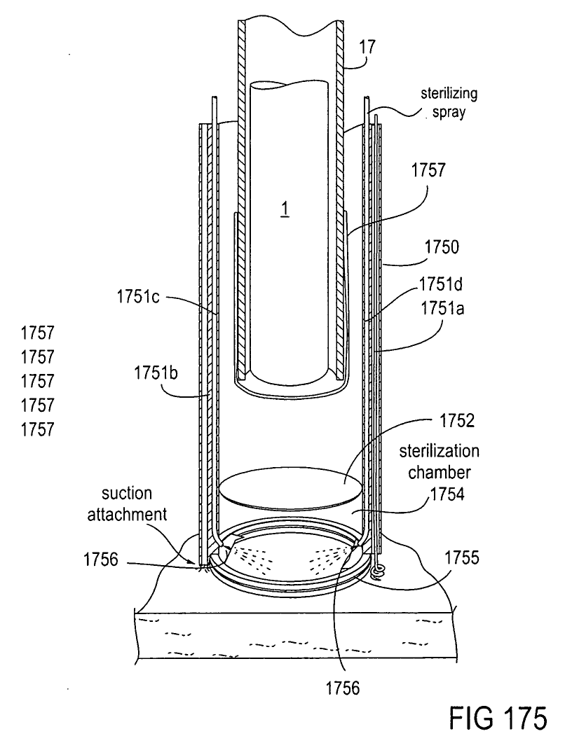

FIG. 175 illustrates a guide tube 1750 engaging a lumen wall. A seal or diaphragm 1753 is disposed in distal end of the guide tube and completely seals the guide tube lumen. When the distal end of the guide tube is secured to the target tissue a sterilization chamber 1754 is formed in the distal end of the guide to between the tissue, the diaphragm 1753 and the walls of the guide to lumen. The guide tube 1750 includes sidewall channels for securing the distal end of the guide to lumen wall. Sidewall channel 175 la includes a helix shaped gripper engaged with the lumen wall. Sidewall channel 1751b is configured to secure against the tissue wall using vacuum. Fixation elements used within the fixation channels may be the same type or of different types. Sidewall channels 1751C and D are in communication with the sterilization chamber 1754. Sterilization fluid, chemicals, sealants, or other materials may be provided using the sidewall channels 1751C, D to sterilize or otherwise prepare the target tissue to be opened in preparation for a transluminal procedure. Also illustrated in FIG. 175 is a cutter attachment 1755. The cutter attachment 1755 is disposed on the distal end of the guide tube 1750 and has a pair of blades 1756 that reside within the side wall. The cutter attachment 1755 is used to perform the transluminal opening by releasing or activating the blades 1756 to form an opening in the target tissue. In a preferred embodiment, the blades 1756 are not activated until the target tissue has been sterilized, sealed, or otherwise treated in preparation for a transluminal procedure. Also present within the guide tube lumen is a second guide tube 17 containing a steerable instrument 1. A sheath 1757 extends around the distal end of the guide tube 17 maintaining the sterility of the guide tube lumen and the sterility of the instrument 1. Once sterilization is complete and the transluminal opening has been created, the guide tube and instrument are advanced against and rapture the diaphragm 1753 and enter into the body cavity now accessible via the trans luminal opening.

It is to be appreciated that when multiple guide tubes are used, the guide tube may be secure to tissue using any number of different fixation methods and mechanisms. FIG. 176 illustrates a primary guide tube 17 secured to tissue T using the suction ring 70F. The secondary guide tube 19 is secured to secondary tissue T2 using hooks 70A.

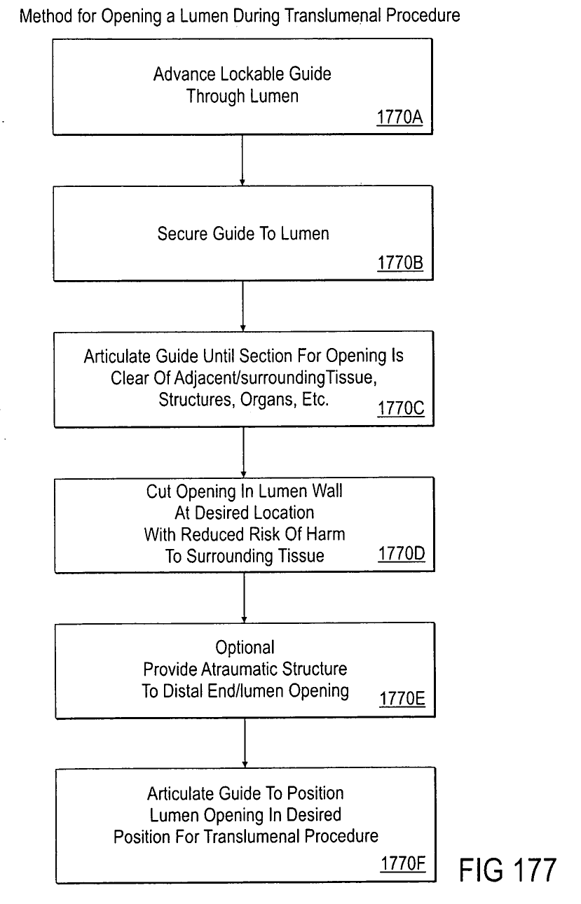

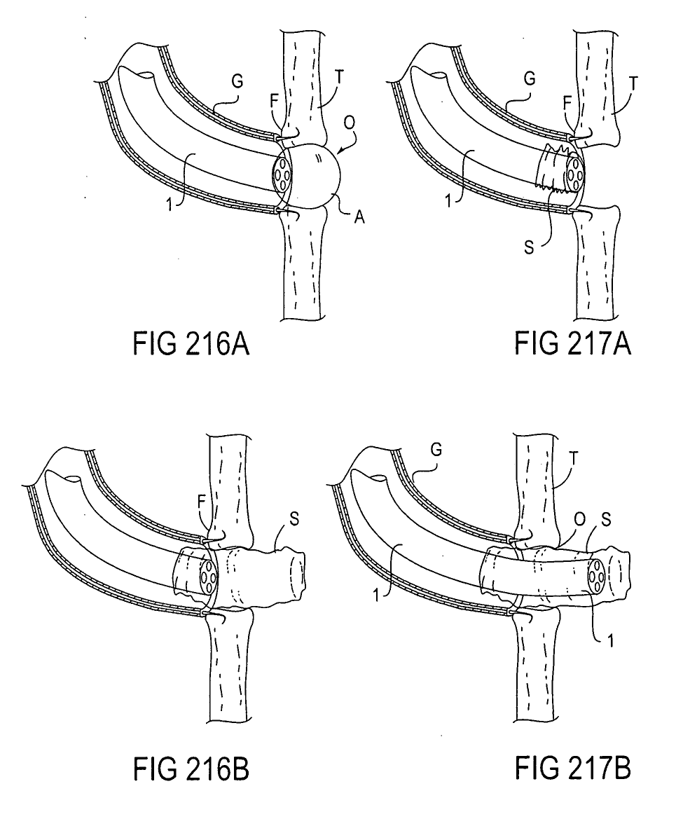

A method for reducing the likelihood of inadvertent organ or tissue damage while piercing a wall in the body is illustrated in the flow chart 1770 in FIG. 177. At step 1770A, advance a lockable guide through lumen. Next, at step 1770B, secure the guide to the lumen. At step 1770C one would articulate the guide until the desired section for opening is clear of adjacent/surrounding tissue, structure, etc. Thereafter, at step 1770D, cut opening in lumen wall as desired location with reduced risk of harm to surrounding tissue. The option step 1770 provides an atraumatic structure to the distal end of the opening. Exemplary atraumatic structures are illustrated in FIGS. 216 and 217 below. Thereafter, at step 1770F, articulate the guide to position lumen opening in desired position for transluminal procedure. After manipulating the wall into position, to now allow access of a steerable instrument through the opening. Advance the instrument through the guide and through the opening in the wall and then advance the instrument through the rigidizable guide in the opening.

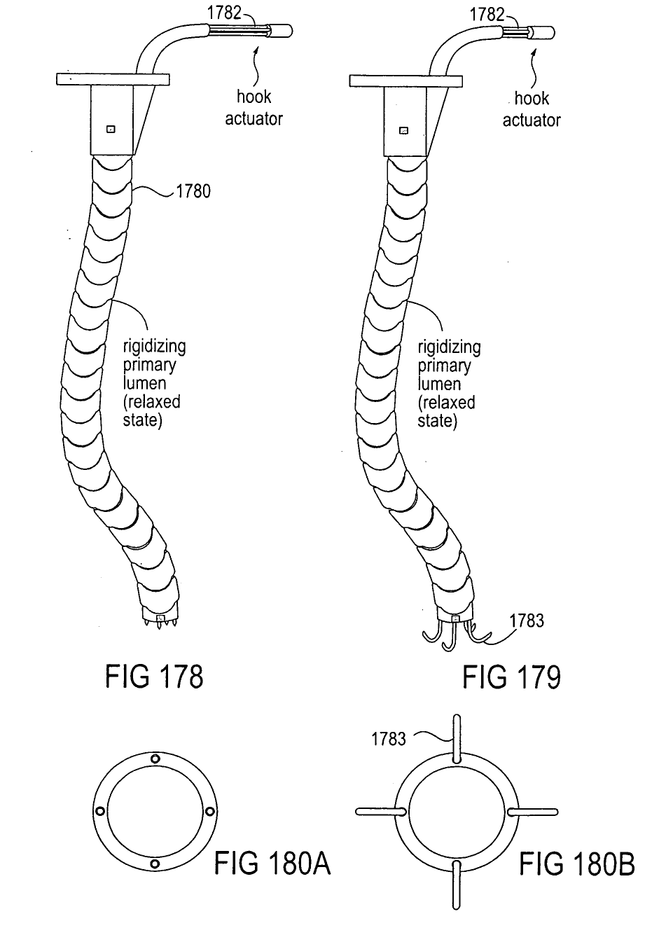

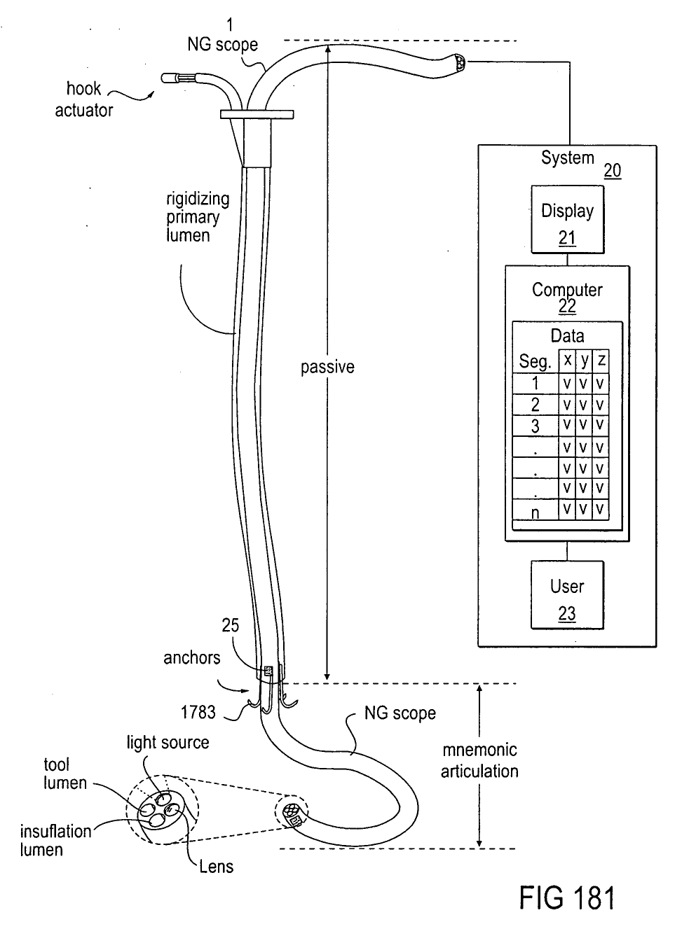

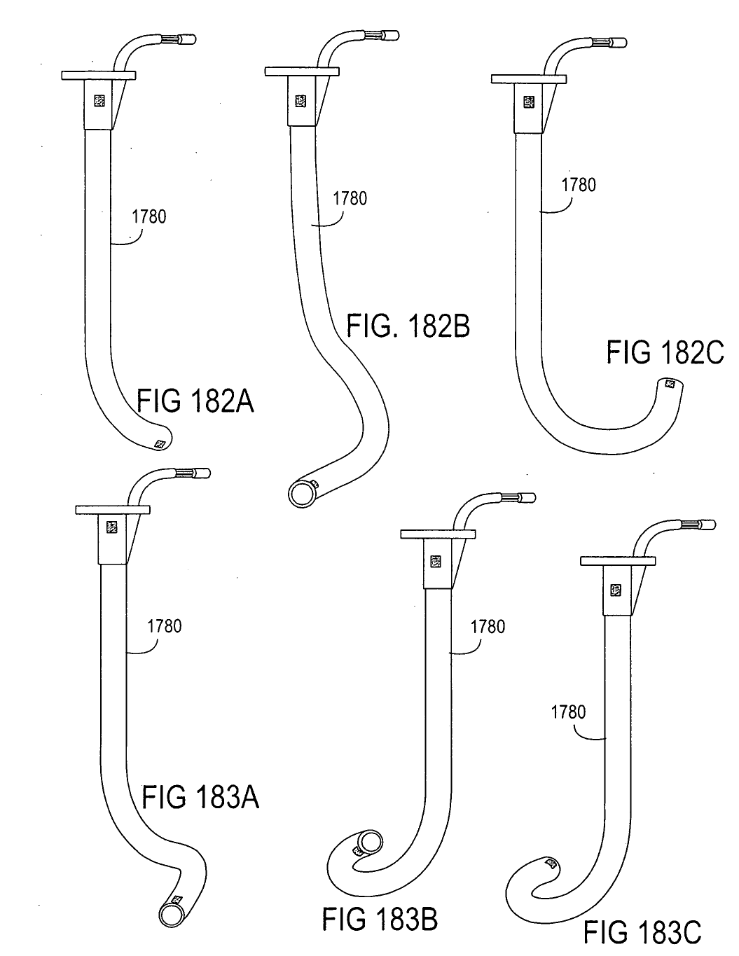

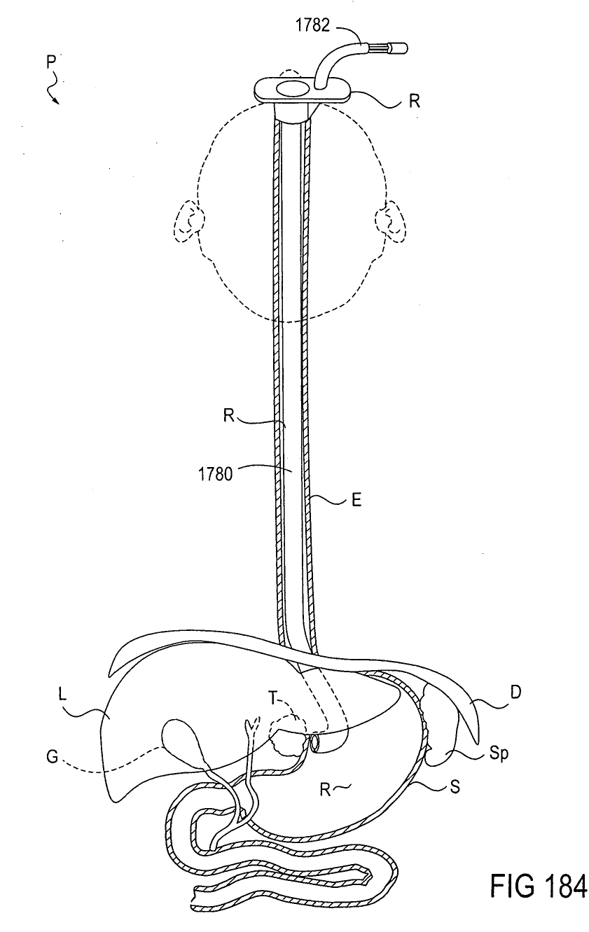

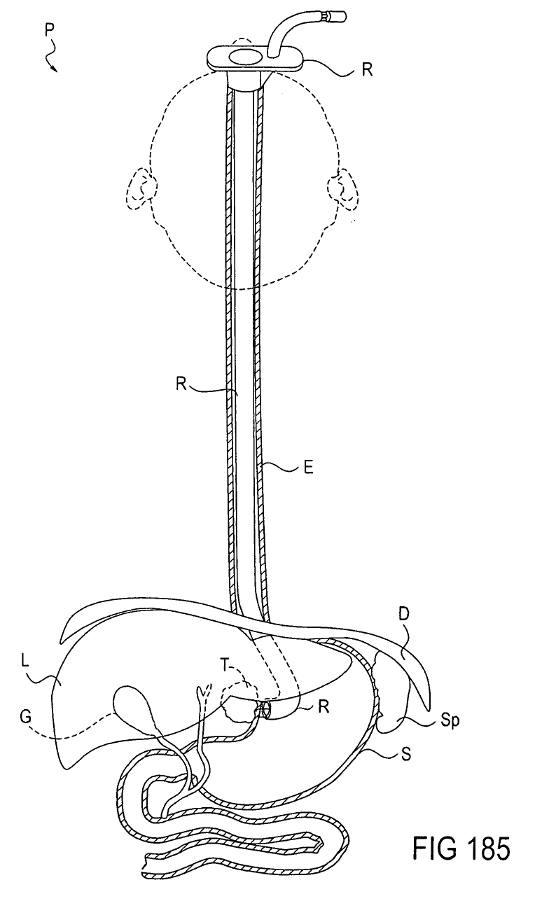

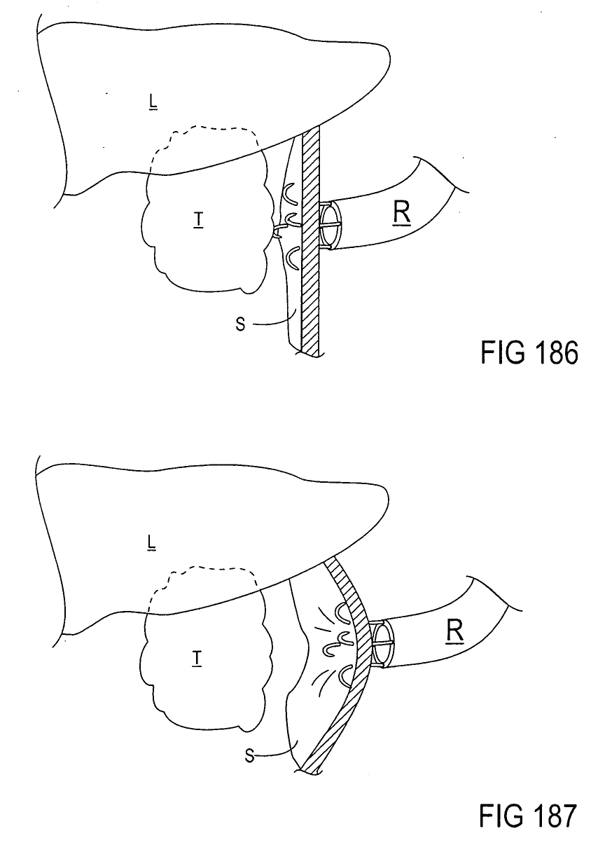

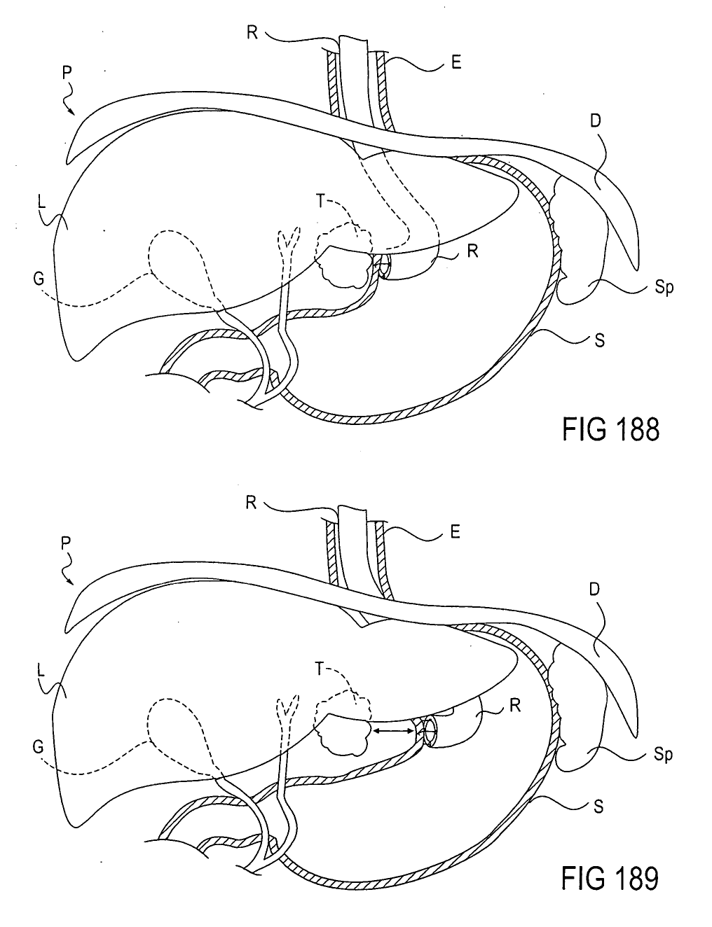

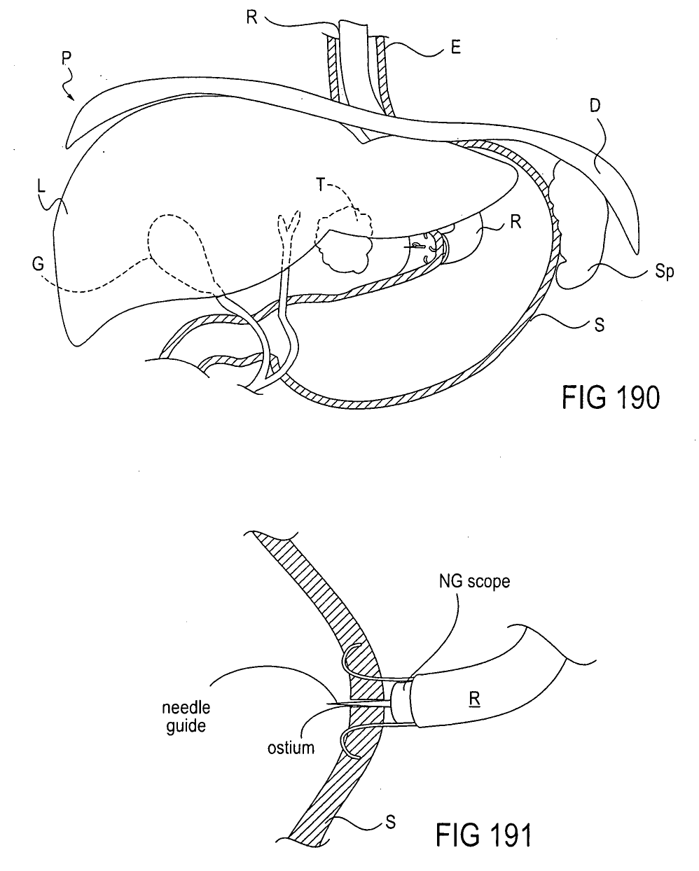

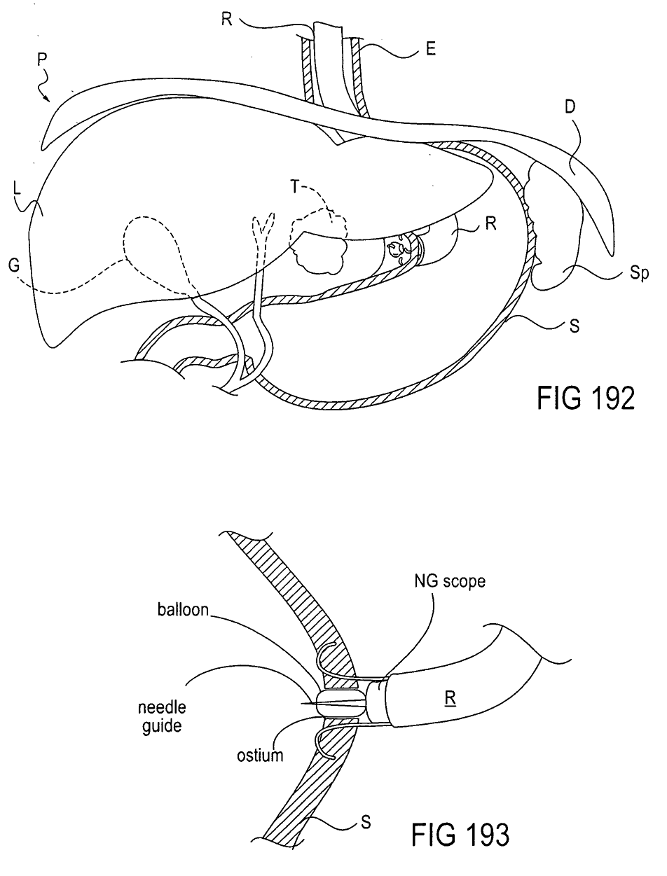

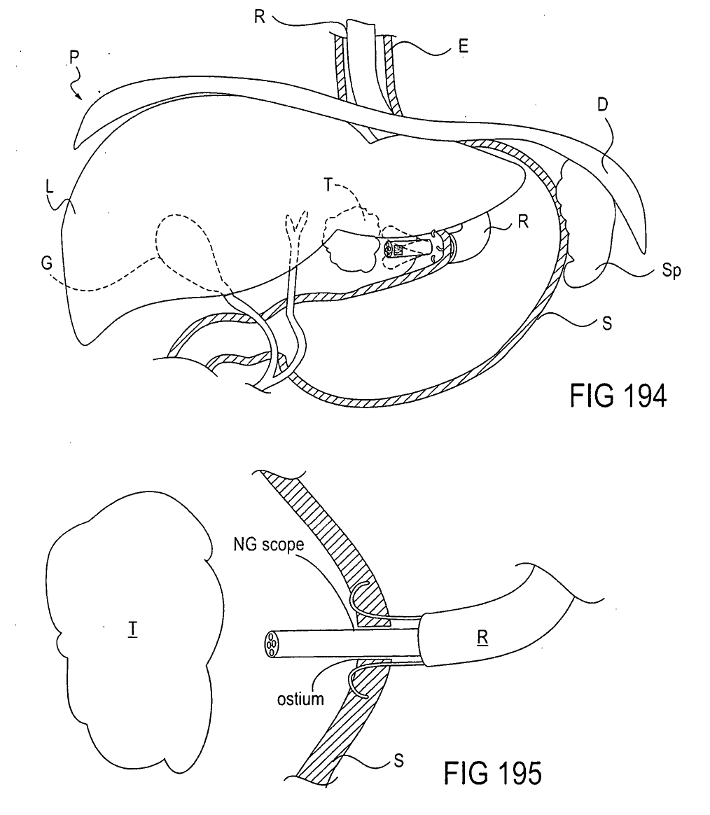

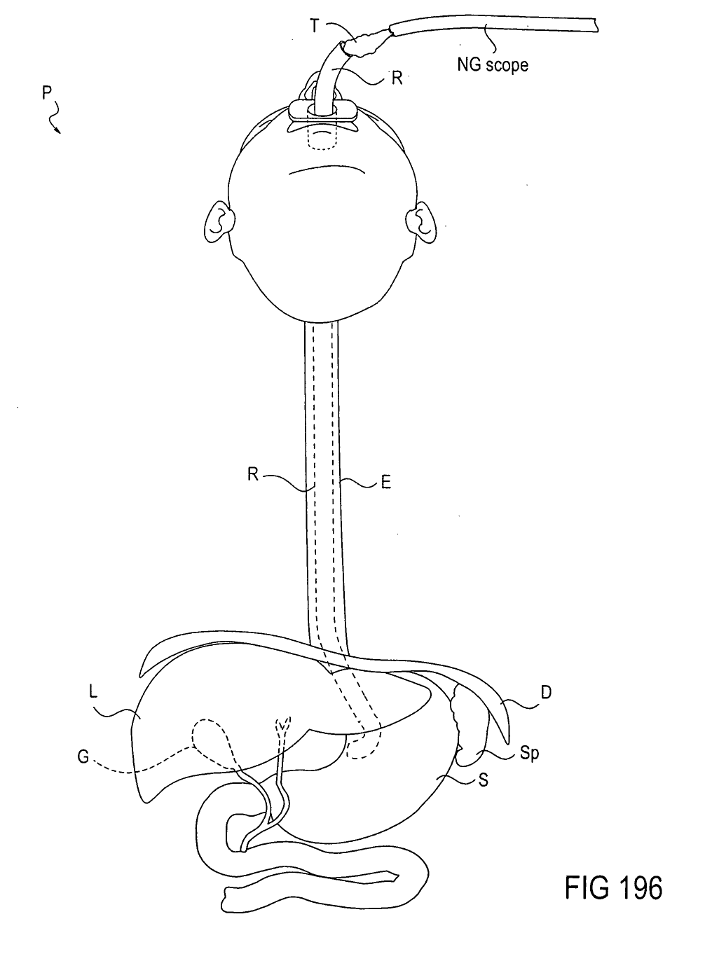

FIGS. 178 through 196 illustrate the use of a guide lumen with distal fixation similar to other fixation techniques described herein. In addition, the steps illustrated are similar to the gall bladder removal detailed above with regard to FIGS. 1-15B. The differences as related to the flow chart 1770 will be described. In FIG. 186, the guide R is attached to the tissue to with draw the tissue back from the tumor T as shown in FIG. 187 and 189. Note that the procedure to open using a needle (described above) is not attempted until after the target open tissue is withdrawn as best seen in FIG. 190 and remained during the open step 1770D as shown in FIG. 192. Next, the tumor is removed in FIGS. 194, 195 and 196 as described above with regard to the exemplary guide tube procedure.

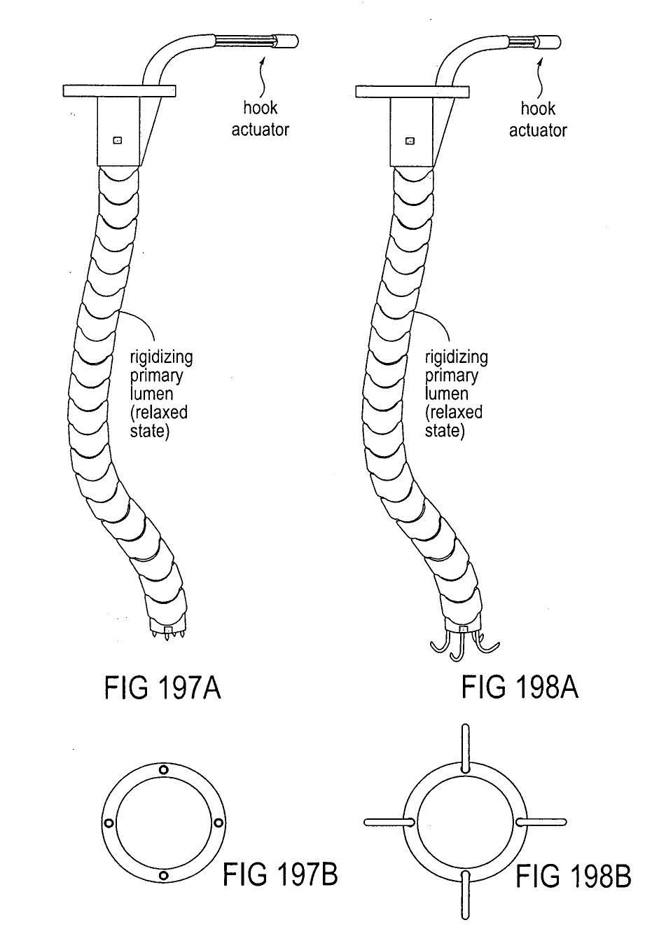

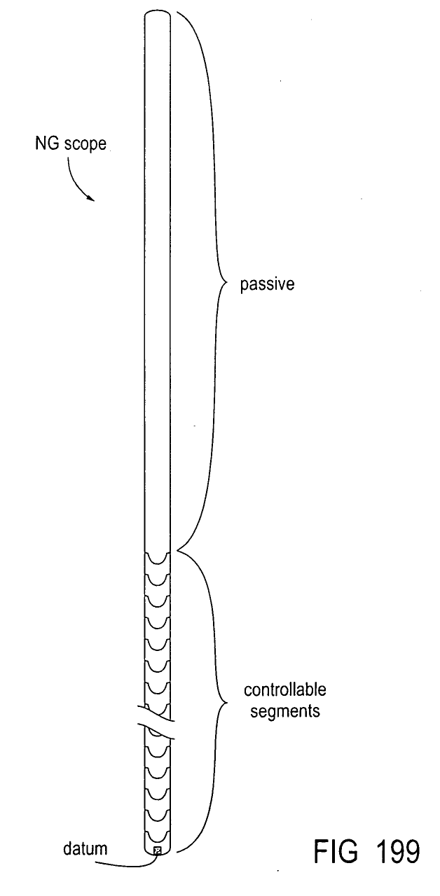

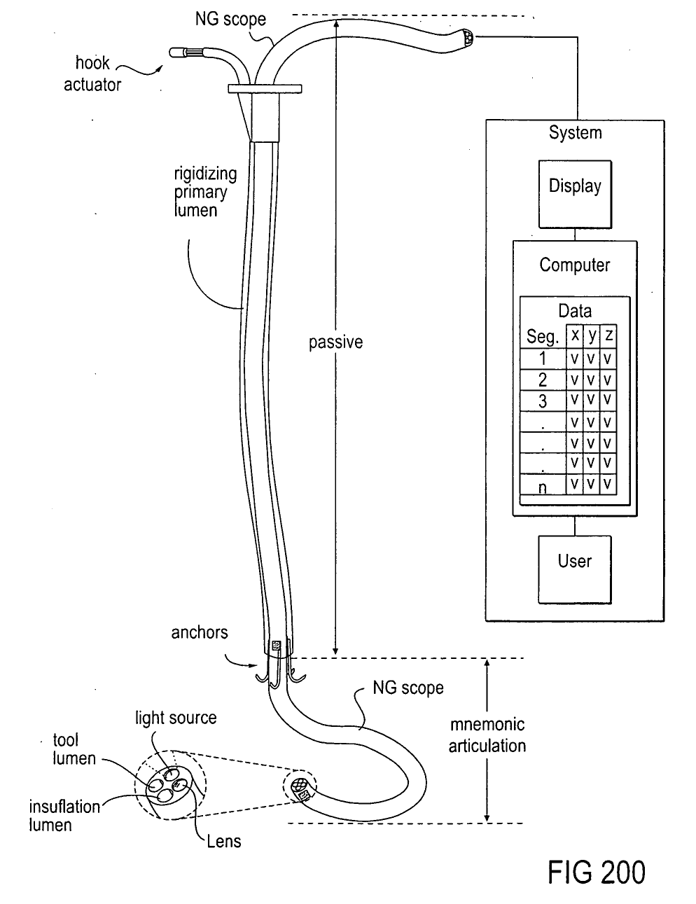

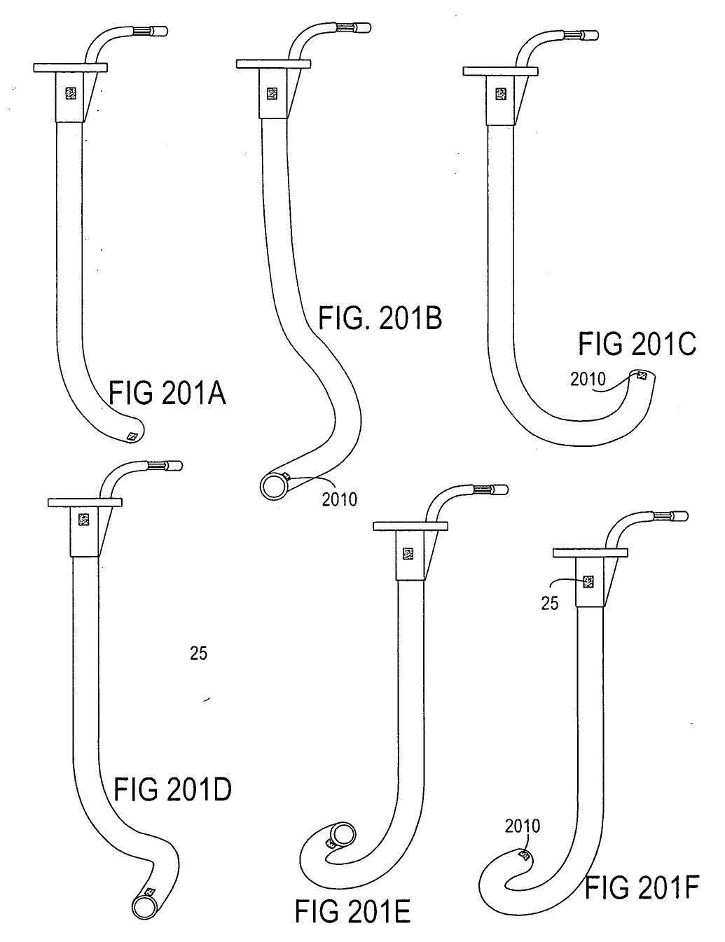

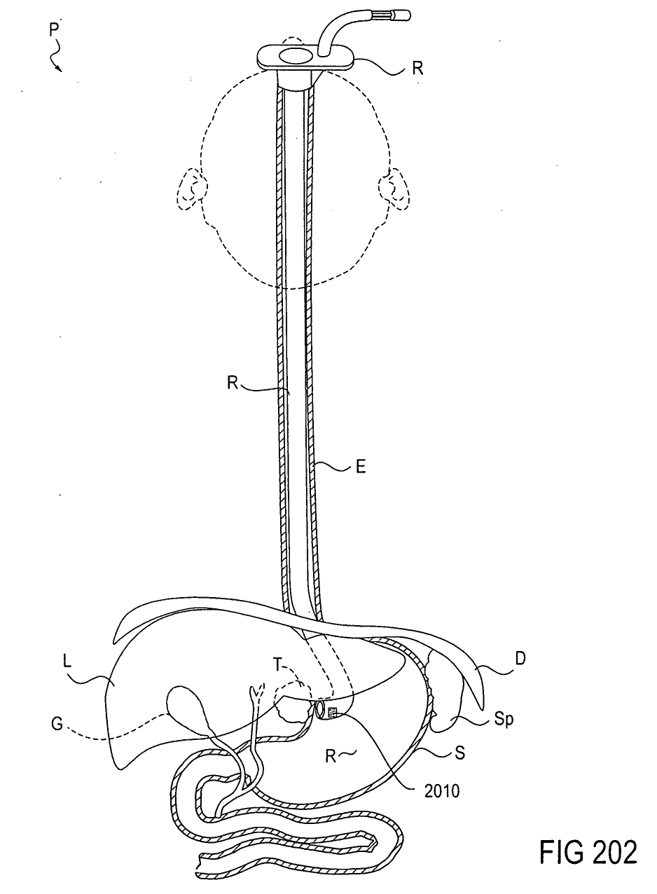

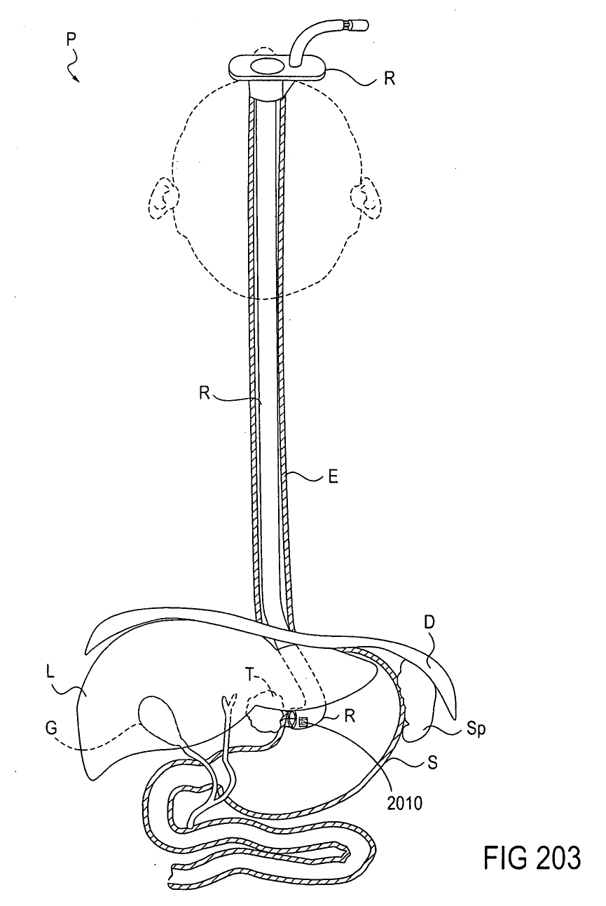

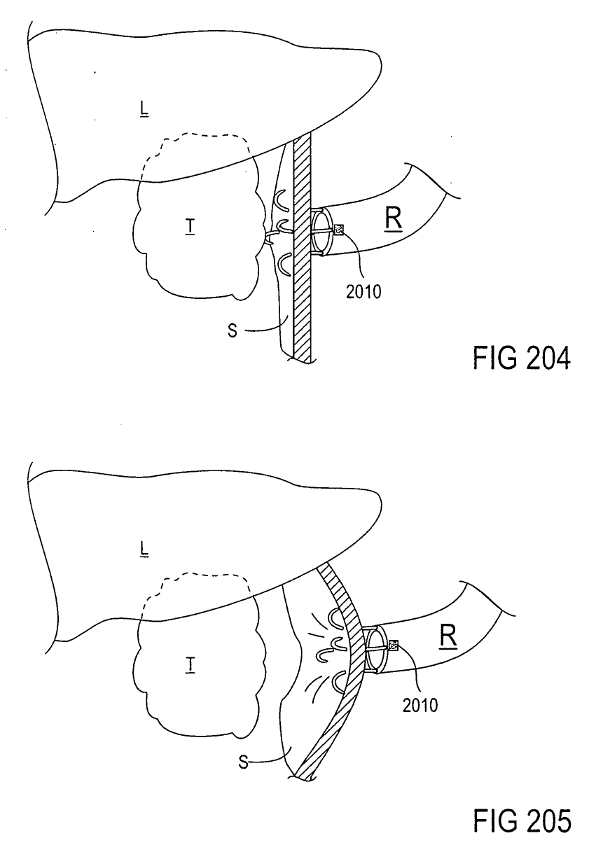

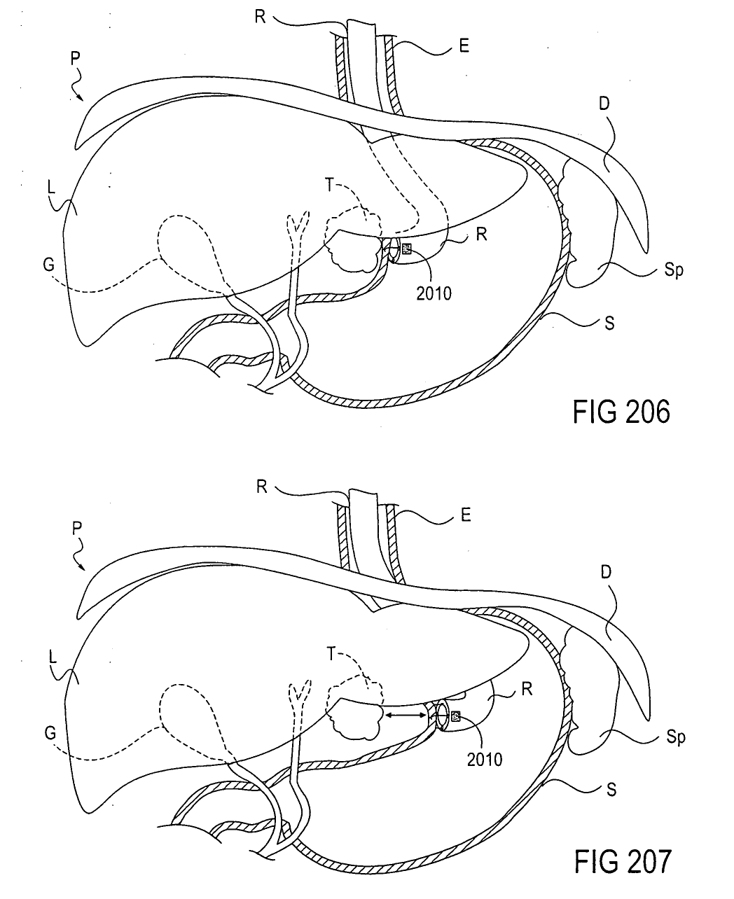

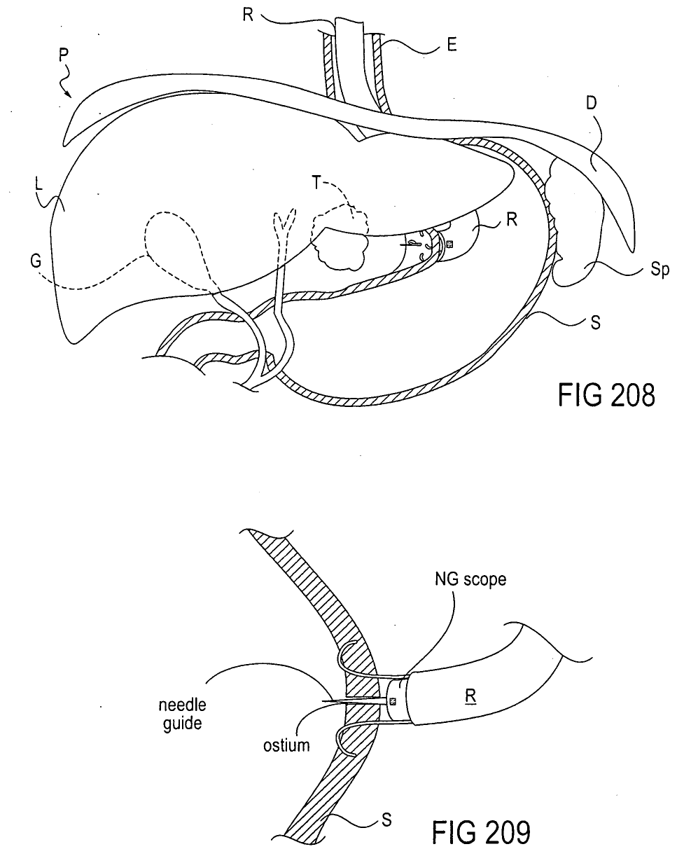

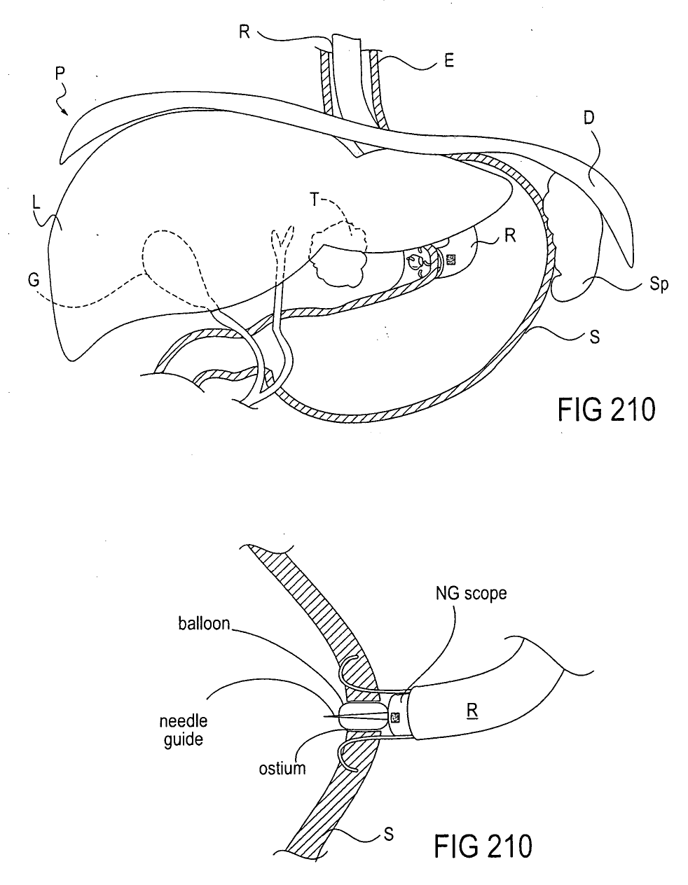

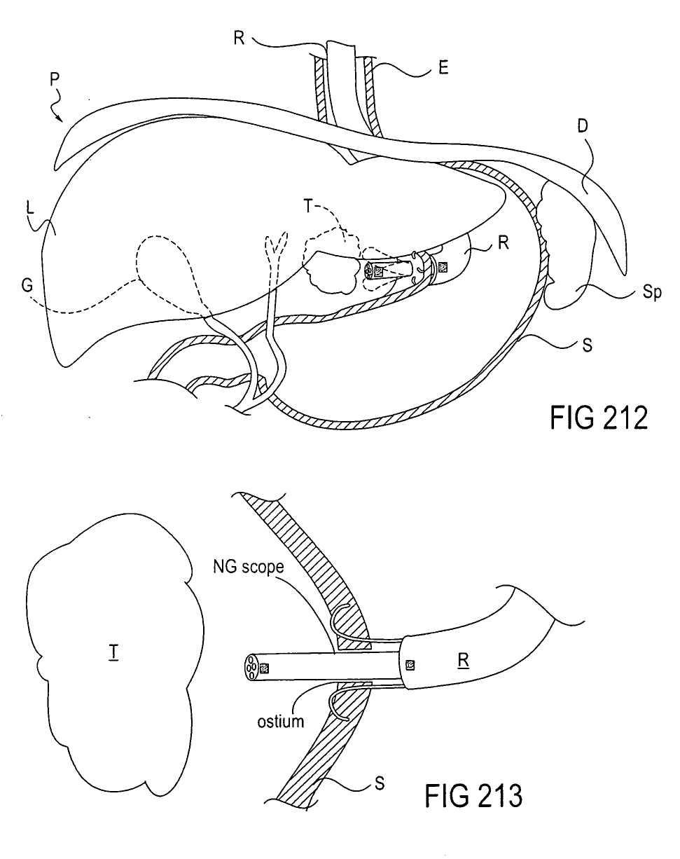

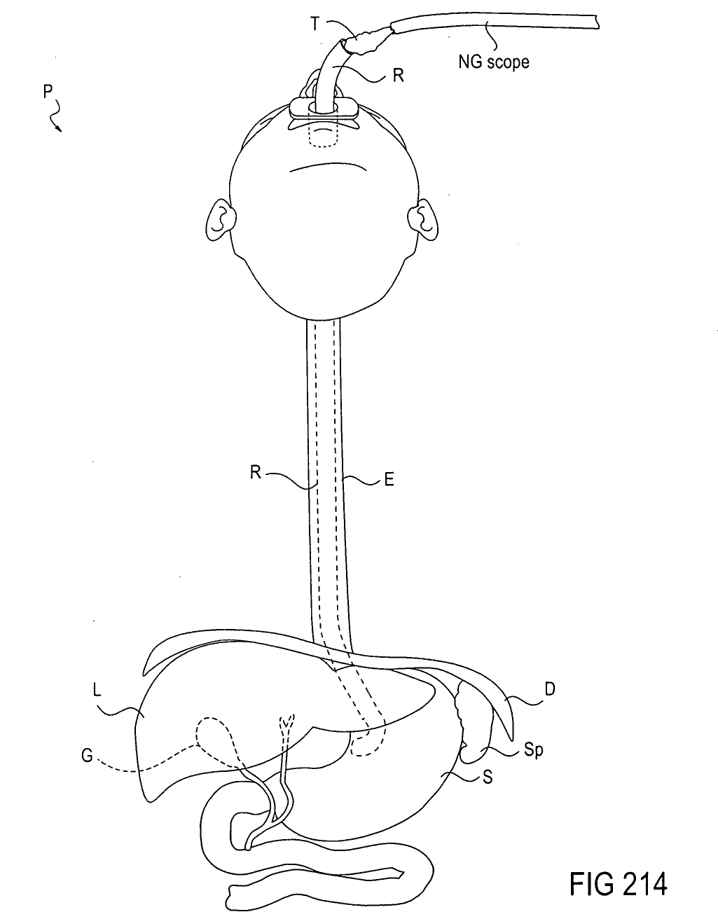

FIGS. 197A through 214 illustrate the use of a guide lumen with distal fixation similar to other fixation techniques described herein as well as the removal of a tumor T as described above with FIGS. 178-196. In addition, the steps illustrated are similar to the gall bladder removal detailed above with regard to FIGS. 1-15B. The key difference is the inclusion of an ultrasound sensor 2010 on the distal end of the guide tube and/or instrument. In operation, the guide R is placed near the target tissue as illustrated in FIG. 202 and used to determine the approximate thickness of the lumen wall, the approximate distance to the tumor T, and/or other characteristics of interest. After confirming that there was sufficient clearance from the tumor T to engage the fixation elements as shown in FIG. 204, the guide is articulated to pull the target open tissue from the tumor T. As best seen in FIG. 207 the sensor 2010 is again used to confirm tumor placement prior to the open procedure. Thereafter the open procedure and tumor removal proceeds as illustrated in FIGS. 208-214 and described herein.

FIGS. 215A-D illustrate an procedure intended to manipulate an empty stomach as an alternative to sealing and insufflating the stomach. FIG. 215A illustrates an guide tube G advancing into the empty stomach S. The guide tube is configured with vacuum ports P on the sidewall as described above in FIG. 141B. The necked down condition of the empty stomach is idea for being sucked up against ports P and the sidewall of the guide G. Advance the guide G into empty stomach and use side wall fixation as shown in FIGS. 215A and 215B. Once engaged, rotate the target lumen to position so that opening may be performed on distal end as shown in FIGS. 215C and 215D. The sidewall fixation could be used to rotate, articulate or other wise manipulate the empty stomach clear of other structures. While described with specific interest in the empty stomach, the guide tube articulation (see FIGS. 2A-2F, for example) may be used to move both the empty and insufflated stomach clear of other structures. Additionally or alternatively, the sidewall of the guide may employ other forms of suction, hooks, coloctomy fasteners and other engagement devices described herein to use the outer guide tube wall to engage and manipulate a lumen.

As described above in FIG. 177, an atraumatic element may optionally be advanced through the transluminal opening to help push tissue, structure and clear a path for the instrument attached to the atraumatic element. FIG. 216 a guide tube G attached to tissue T so that its lumen is aligned with th transluminal opening O. FIG. 216 also illustrates an atraumatic element embodiment in the form of a transparent ball A. Because the ball A is transparent, the imaging and visualization capabilities of the instrument 1 (attached to the ball A) may be used to guide or advance the ball B to make a path for the instrument 1.

FIGS. 217A-217C illustrate an atraumatic element that is an expandable and/or inflatable sleeve S. The sleeve S is made from materials described above for sheaths, transparent, medical grade plastic or stretchable polymer. After the open procedure is completed (FIG. 217A), the sleeve S is expanded or inflated through the opening O to being to a traumatically displace the tissue, structures and/or organs closest to the transluminal opening O. Once the path is cleared by the sleeve (and visable to the optics in instrument 1 since the sleeve is transparent) the instrument 1 is advanced through the sleeve S along the created path as shown in FIG. 217C.

Another solution for performing the transluminal opening is to form one or more intersecting cut lines so that the opening is made but the tissue remains available for a later closing procedures following the completion of the procedures using the controllable segmented instrument and the guide tube. For example, a cross-cut could be used whereby forming in a circular opening for flaps for contiguous flaps that will open out to allow an instrument to pass through and yet when the procedure is completed the four flaps may be brought together and then secured in a cross fashion to close them up. Could be a cross (X) cut or intersecting arcs as described below and illustrated in cutting devices elsewhere in this application. Such cutting features may also be part of integrated instrument or stand alone instrument.

Perforation of tissues using screws, RF knife, needle, cross-lay to cut tissue into 4 or more flaps. Microwave cutting techniques, laser cutting techniques, local applications of chemicals to lacerate burn or otherwise form opening within the tissue

Alternatively, anchors could be provided against the tissue at on or more predetermined locations about a hole so that when the hole is formed the anchor or staple points are then used to manipulate the tissue to form the opening. In one embodiment, the anchor staples are the 12 o'clock, 3 o'clock, 6 o'clock and 9 o'clock positions. In another alternative, the staples are positioned at a 45° angle within a cross-cut piece of tissue. In each of these embodiments as the screw is advanced through the tissue the stapled tissue is moved apart.

A cross-cut could be used to form an opening in the tissue and may provide advantages to later closure and healing as the flaps formed from the cross-cut are simply brought together. The width of the cross cuts being to adjust the size of the resulting opening. Cross-cut is merely an example for a plurality of radially expending cuts that together provide the desired access. Non-circular partial cuts may also be used depending upon the desired opening shape to be formed.

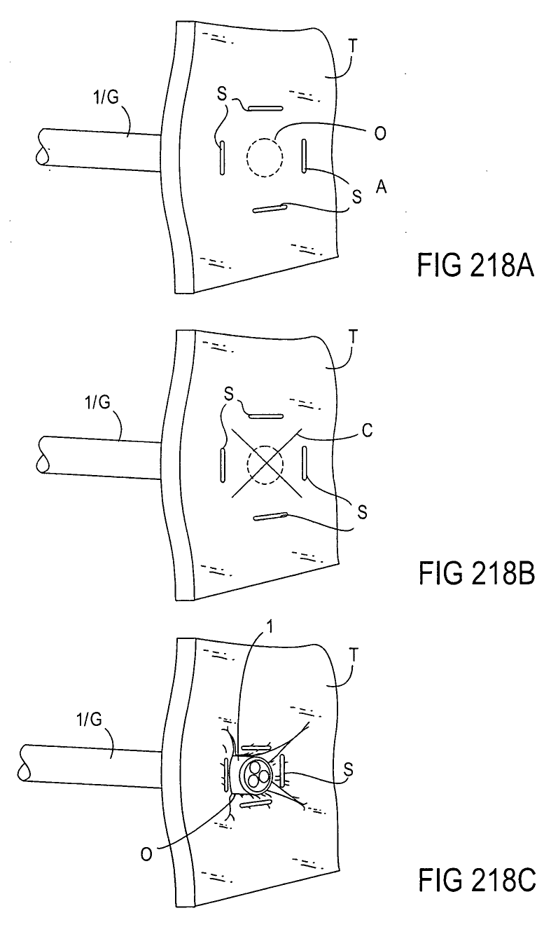

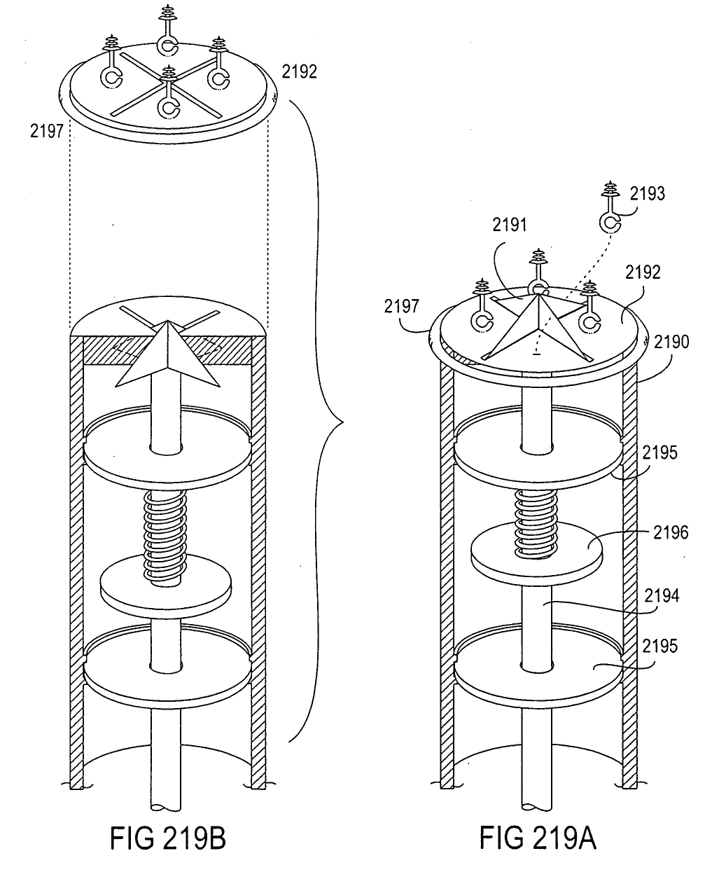

One area of interest in all surgery and of growing interest in transluminal procedures is the formation of and the healing of the transluminal opening. The various illustrations that follow address some of the needs for precise, repeatable and simple open procedures. An particularly for open procedures that will heal quickly and without post operative complication. FIG. 218-218C illustrate an open procedure that employs suture attachments S that are positioned about the projected open target (dashed area in FIG. 218A). Thereafter, the open incision C is made in a cross shaped cut to produce four or nearly four even flaps that may be retracted (as shown in FIG. 218C) or otherwise positioned to make the opening O available for the procedure.

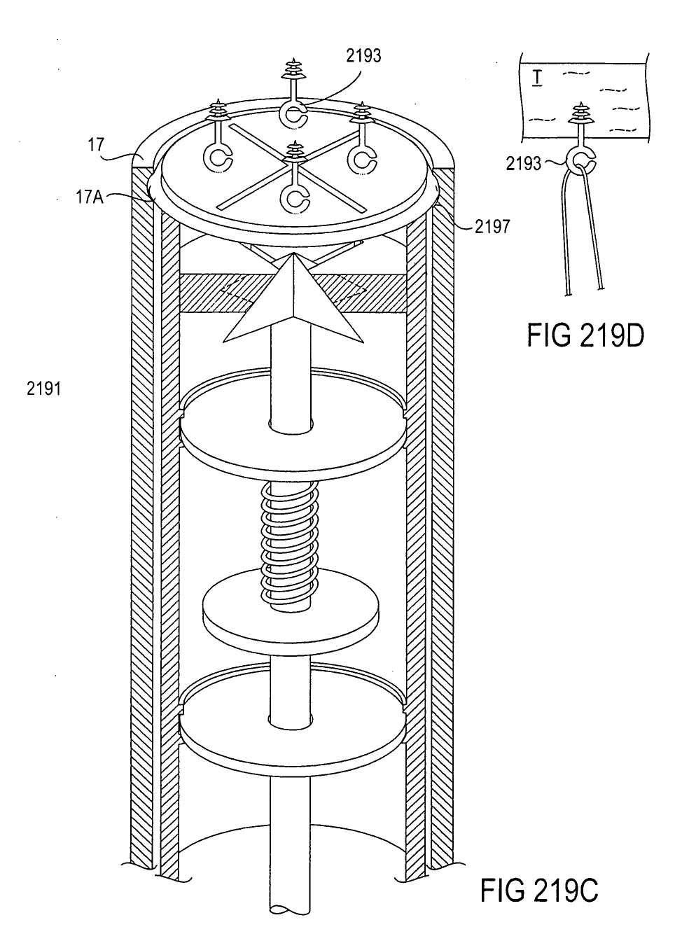

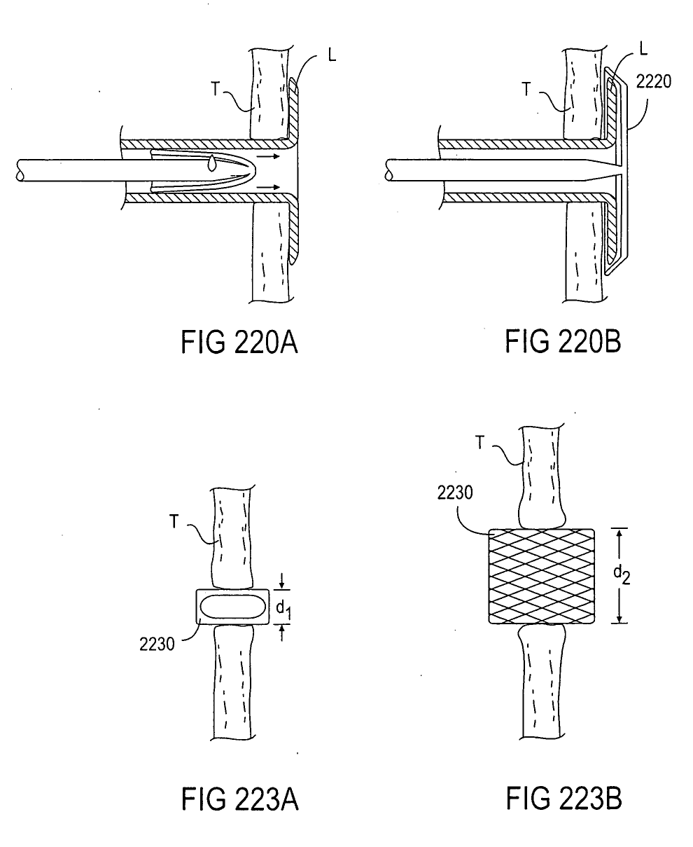

FIGS. 219A-C illustrate different views of a cutter assembly 2190. As illustrated in FIG. 219A, the cutter assembly includes a housing and a distal end 2192 with an opening for cutting blade 2191. Pushing the shaft 2194 advances the blade 2191 past the surface 2192 to form transluminal openings with the precise cross shape cut. The cross shape cut is for illustration since blades of various different shapes may be used depending upon the particular type of opening desired. A guide tube engagement feature 2197 is provided in the distal end to mate with a complementary feature in the guide tube (see feature 17A in FIG. 219C). Stops 2195 limit the travel of the shaft 2194 because the block 2196 will contact the more distal block. The spacing between the blocks 2195 and the position of the engagement feature 2197 may be used to provide very precise depth of cut for creating transluminal openings. Also shown on surface 2192 are closure features 2193. The feature 2193 is attached to the surface 2193 where indicated by the dashed lines. The features 2193 are intended to attached to the lumen tissue using the shaped tips. When the blade assembly 2190 is withdrawn the features shear off the surface and remain in the tissue as seen in FIG. 219D. After the procedure, the C shaped openings in the features 2192 may be used for closure procedures as illustrated by passing suture S through the feature 2193 in FIG. 219D.

An umbrella configuration could be used wherein the umbrella is advanced through the opening made in the lumen into an open position thereby forming a fill able lumen thereafter the screw would be reversed back out of the opening then that action would bring the umbrella into intimate contact or into sealing relation with the wall thus providing a lumen through the wall that is now sealed to support the application of pressure. An umbrella configuration would keep the anchors and tissue folded back so that lumen opening remains clear. As such, the flaps created after performing the transluminal opening may be held back using an umbrella like seal 2220 as best seen in FIG. 220B. The umbrella seal 2220 may be advanced though the guide lumen (FIG. 220A) and then opened to seal the transluminal opening as best seen in FIG. 220B.

Several alternative dilation techniques may also be used to assist in forming transluminal openings and alternatives to create opening in tissue at landing site of the guide tube.

There could also be a cutting implement positioned on the distal end of the steerable, segmented instrument advanced through the selectively rigidizable guide tube. Upon reaching the location for creating the transluminal opening, there are numerous hybrid implements that form and then dilate an opening in tissue. Dilation cutter embodiment could be the cutter as part of an expanding helical design so that the further in the helical member is advanced the larger the diameter the opening is so thereby allowing one to create a hole and then open the created hole all in a single step.

Dilation of the transluminal opening once formed seeks to resolve the question of how to open the transluminal hole. Balloon dilation to open the hole in the side of the stomach as well as balloon dilation may be available using some of the techniques described by Kalloo, et al. incorporated by reference above.

In some cases, the formed opening is large enough to provide access to other instruments needed to conduct a procedure. In some alternative tissue opening techniques, the tissue may be opened and subsequently dilated or by using an inventive opening device form and dilate an open in an integrated procedure. After forming the opening, dilate the opening to allow additional tools to pass.

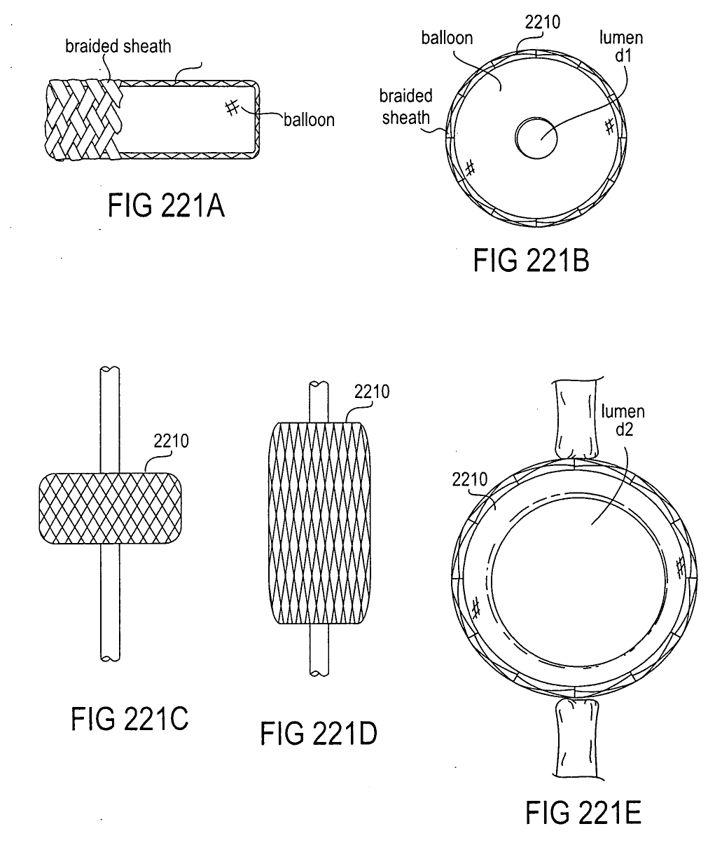

Pneumatic muscle 2210 may be used for dilation. FIGS. 221A-221E illustrate various views of a stowed and deployed pneumatic muscle 2210 used to create an opening in a lumen wall. Pneumo-muscle is a material that flattens and expands when exposed to the appropriate activation energy. For example, an elongated, cylindrical pneumo-muscle may be advanced into a small diameter hole as seen in FIG. 221C. Once in the desired position, the elongated, cylindrical pneumo-muscle is activated causing it to (a) decrease in height/length and (b) increase in diameter as seen in FIGS. 221D and 221E. The resulting increase in diameter is used to increase the size of the opening and allow access of additional tools or instrument or other implements. The difference in lumen diameter is illustrated in FIG. 221B as d1 before activation and d2 in FIG. 221E after activation. Lumen diameter d2 is greater than d1.

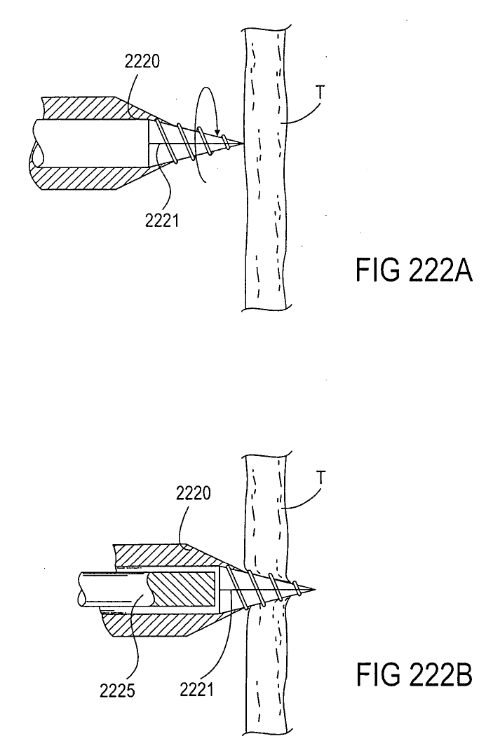

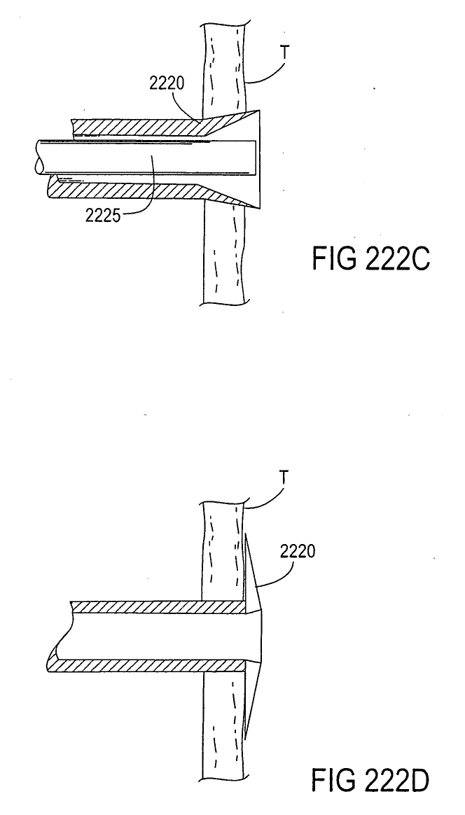

FIGS. 222A-222D illustrate the use of a split screw 2220 to create an opening in a lumen wall. The lumen wall may also be opened using a screw to form the opening in the wall and then one may open the screw to create the opening. As is make clear in the FIGS. 222B, it is a screw 2220 having splits 2221. The screw 2220 rotated as indicated by the arrows in FIG. 222A. The rotation advances the screw through the wall as shown in FIG. 222B. Once through the wall, a pin 2225 or other devices inserted into the middle of or a lumen running through the screw to cause the split portion 2221 to open out as shown in FIG. 222C and provide larger access into the adjacent space.

Additionally, as best seen in FIGS. 222C and 222D when the pin 2225 is advanced through the screw, the segments 221 open up and flatten against the lumen wall as best seen in FIG. 222D. Additionally, once the screw 2220 is in the open position an umbrella 2220 or other flap-based sealing device is advanced through the open portion of the screw. Once the umbrella passes the screw portion it opens and then seal back against the screw thereby filling back against the wall as best seen in FIG. 220B. Alternatively, the screw and umbrella configuration could be used wherein the umbrella is advanced through the screw into an open position thereby forming a fill able lumen thereafter the screw would be reversed back out of the opening then that action would bring the umbrella into intimate contact or into sealing relation with the wall thus providing a lumen through the wall that is now sealed to support the application of pressure.

Other techniques to open the lumen wall. Place the screw against the wall of the tissue and then us the screw end to form the hole. Next, advance catheter through the rigidizable tube having on its distal end a cross-cut instrument or other cutting instrument. Alternatively, the screw may be equipped with blades within the lead screw. For example, the screw may have a cross-shaped knife on the top. In use, one would land with the rigidizable guide tube, apply suction to hold the tissue there and then cut. In another alternative. a coil of wire which could be rigid wire or SMA wire as it is advanced through the tissue it cuts the tissue. In a shaped memory alloy version of this technique the coil of wire is formed from a coil that when activated the coil expands. The coil also has an expanding diameter or an expanding helix or other shape. As a result, as it is advanced into the tissue the tissue is pulled apart. In the embodiment of the rigid coil environment, the rigid coil has an expanding diameter or alternatively a expanding helix. As the rigid coil is advanced through and the tissue advances up to helix the tissue is pulled apart into ever expanding opening based on the size and dimension of helix. Any of these coil embodiments could have on their tip a short needle tip that is used to cut through the tissue.

The concept of using a screw is analogous to a dry wall screw. Drywall screws are originally slotted screws that are advanced and thereafter a pin or other spreading device is advanced through the middle of the split screw to cause it to flare out and form a larger opening. The same principal is applied to the examples here.

In yet another alternative, the helix or rigid coil or other coil embodiments could be a hollow needle and control the shape of the opening and remove tissue as it enters the hollow tip. In this way the hollow needle is used to remove a tiny core of material that once completely removed forms an opening in the tissue.

In yet another alternative opening procedure, a stent may be used to create an opening as illustrated in FIGS. 223A and 223B. As illustrated in the figures, the use of an expanding stent or other expanding structure or expanding scaffold may is used to create an opening in a lumen wall. Insert a closed down or stowed stent through an opening formed in a wall or using a stent to form an opening in the wall. FIG. 223A illustrates a stent 2230 in a stowed configuration within an opening in tissue T. In this configuration the diameter of the opening and the stent is dl. Thereafter, as best seen in FIG. 223B, deploy the stent 2230 to open the wall and also provide structural support for instrument passing through the lumen. Once deployed, the stent and the lumen opening expand to diameter d2 that is greater than diameter d1. Such structural support could be provided in combination with other devices described herein. For example, the stent could be used in combination with the datum and position indicator landing pad, the rigidizable overtube or steerable segmented instrument.

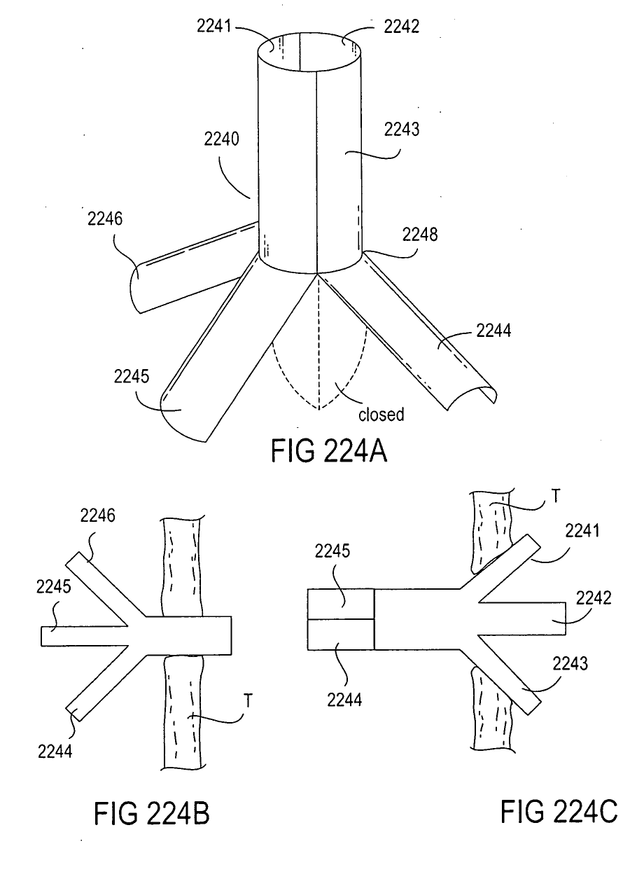

Another useful in creating a lumen opening is the split tube or the three corner opening . FIGS. 224A-224C illustrate a flex point opener in operation. The solid tube 2240 with hinges, between flaps 2243 and 2244, 2246 and 2241 and so forth. From the closed position (phantom in FIG. 224A) lift up one side to engage a hinge 2248 between opposing flaps. When that side is lowered the opposite side will open. In operation, the opening is cut, the lifter 2240 slides in, lift to lock (as in FIG. 224A and B), raise side to create opening and also push back tissue against wall and keep lumen opening clear (FIG. 224C). The lifter 2240 could be used with a stand alone datum in an insulated stomach so that you have enough room, or, alternatively, the lifter 2240 could be resized to fit other portions of the anatomy. As such, in operation, a flared insert 2240 having a cylindrical end hinged 2248 to a flared end where the cylindrical end is inserted into a small hole. After insertion as shown in FIG. 224B, the proximal flared end is advanced to bring the flared portions together which causes the cylindrical end to then open out. This is best seen in FIG. 224C where there the lifter 2240 is shown forming a wider opening on the distal end and forming a portion of the lumen on the proximal end that was previously flared.

Sealing Methods and Devices

Once the transluminal hole is appropriately sealed, one can inflate the periodontal cavity. An umbrella sealing design could be used. Alternatively, a double balloon where one balloon is inside the stomach and another connected balloon is on the outside of the stomach so that when inflated the balloons pressed together against the stomach wall capturing the stomach wall between them. Additionally, a sealing ring, such as an inflatable ring on the outer wall of the rigidizable guide tube could be used to seal the esophagus above the opening to the stomach. The inflatable ring could be one of a series of selectable rings based spacing along the guide tube outer wall. One or more rings are inflated depending upon a number of factors such as guide tube position and specific patient anatomy. Additionally or alternatively, an inflatable ring or other sealing means could be advanced along the guide tube outer wall and positioned between the guide tube and a portion of the alimentary canal to seal the stomach.

In alternative embodiment, sealing could be provided in a portion of the lumen of the rigidizable guide tube near the distal end or in a position to provide sealing to gases provided through the opening and into the tissue of interest. In other words, sealing of the guide lumen or steerable instrument may be accomplished using seals on, in or about the distal or sealing end of the instrument or guide or be a separate device provide in the area where sealing is desired.

In another alternative to seal the guide tube to the lumen wall, a deflated bladder may be provided that is inflated after the guide tube is secured to the lumen wall. FIGS. 225A-226B illustrate two alternative bladder configurations. In guide tube 2250, fixation tines T pass through the sealing bladder 2251 as best seen in FIG. 225A. The tine channels 2253 are visible in the bottom view in FIG. 225B. In contrast, guide tube 2260 engages the lumen wall with tines that pass on the outside of the inflatable bladder 2261. The tines pass around the outer perimeter of the bladder 2261 as seen in FIG. 226B.

While the bladder/tine configuration is different, the bladder operates to seal the guide tube to the lumen wall in a similar fashion. First, an un-inflated bladder on distal end of the guide tube lands on the lumen wall. Next, press against bladder (i.e., deform it) so as to engage the tines into and secured to the lumen wall. If needed by the tine design, twist to engage tines fully. Once the tines are fully engaged, inflate bladder to seal the distal end to the lumen wall. As described above in FIGS. 220A-B, an umbrella seal 2220 may be spread around opening or use an umbrella type seal described above to line the opening once created.

Sealing techniques also include a closed umbrella that is advanced through the small opening in the wall and once the umbrella passes the wall, it opens out to where by pulling in the proximal direction the umbrella sit against the wall to provide a seal. Additionally, a second seal may be provided on the inside of the wall that is pushed down against the opening and is also used to fill against the umbrella.

We may use full circle or partial circle umbrella style seals. Partial circle umbrellas include those with less than a full circular coverage or multiple non-overlapping sectors or flaps. In an embodiment where a screw or helix is used to make or dilate an opening, an umbrella with fill or partial flaps could be advanced through the opening and deployed to form a seal. Similarly, one could use a screw to open and then anchor against the tissue wall. After anchoring, provide an umbrella or other sealing device to open against the anchor and seal or configure a seal, restriction or other closure device within the screw to act as a seal.

Creating and Maintaining a Sterile Field

A multi-function applicator may be used to create and/or maintain the sterile field. This may be done as an alternative to sterilization. Instead of sterilizing the target lumen, just seal the tissue area by spraying on sealant or applying a bandage over the area. As described above with FIG. 173 there may be a technique to apply a bandage over target site to provide sterility. After placing an adhesive bandage or patch and applying it to the tissue to seal, the transluminal opening is cut. In addition, the back (portion facing the guide tube) of the patch could have features to ease closure. Those features may include suture rings, hooks, barbs and other elements to aid in closing the opening at the completion of the transluminal procedure.

The spray nozzles described above with regard to FIG. 174 may also be used to provide a sterile rinse solution or other chemical treatment, apply liquid band aid or apply some other sealant to provide a sterile field in the region around the transluminal opening.

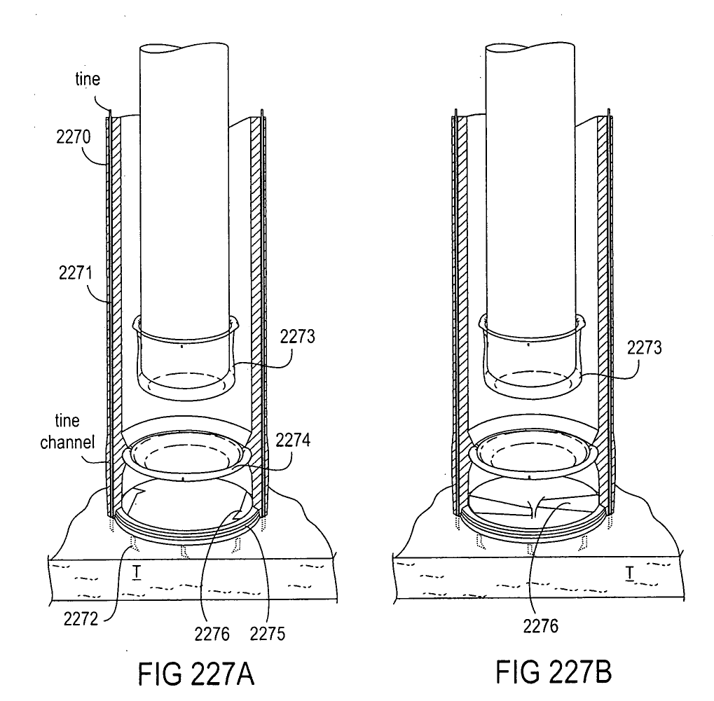

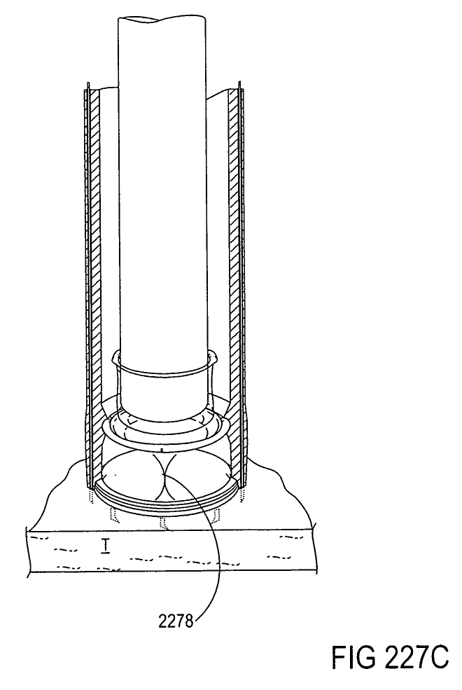

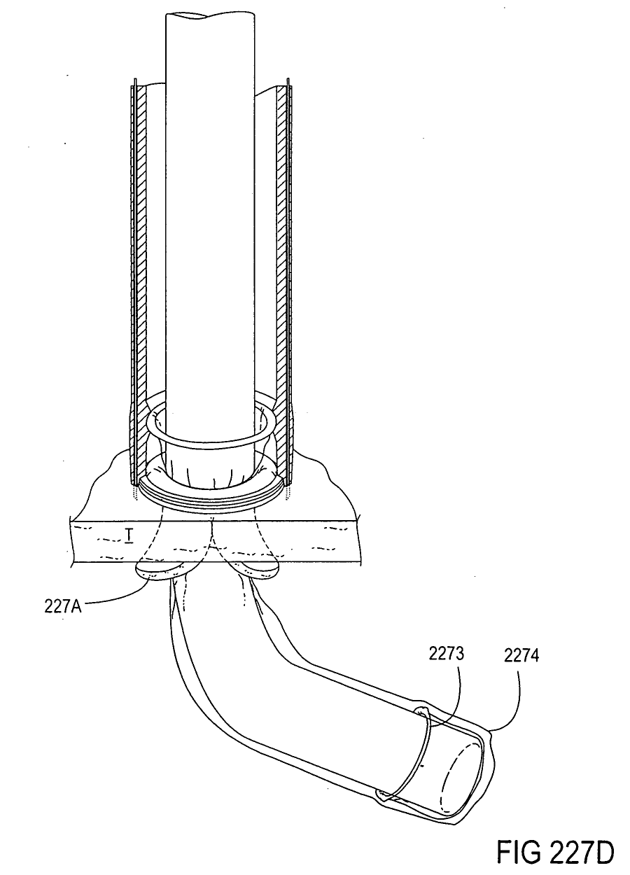

FIGS. 227A-D illustrate the operation of an integrated fixation and opening guide tube 2270. The guide tube 2270 includes a tine activation channel 2271 connected to the tines 2272 that operate through the distal end of the guide tube. A cutter system 2275 is within the side wall of the guide tube and has a pair of arc shaped blades 2276. A sheath 2274 is also provided at the distal end of the guide tube. The sheath 2274 unrolls as an instrument advances through the guide tube lumen towards and through the opening. The sheath 2274 may also be used to provide a sterile barrier for sterilization procedures as described above. As shown in FIG. 227B, the blades 2276 are used to form the transluminal opening 2278 in the tissue T. The intersecting arc or rounded cross shape open pattern is best seen in FIG. 227C. Thereafter, the instrument I is advanced through the sheath 2274. The sheath unrolls and remains covering the distal end of the instrument 1. FIG. 227D shows unrolling the sheath as the instrument 1 advances through the opening.

In addition, there are provided inventive applications for trans-luminal procedures including the use of an overtube that also maintains a sterile field of operation.

Use of Liners and Sheaths to Maintain a Sterile Field

The internal part of the overtube can be kept sterile such that a sterile scope will maintain its sterility as it goes through into the peritoneal cavity. Once the procedure is over we can then leave the overtube attached to the wall and withdraw the scope and then insert a separate device to the overtube. For example, a stapler with a small CCD or optic wire provisionalization as an option to seal the port of entry. Additionally, we then incorporate the rim or the edge of the overtube pressure sensors to ensure sure the seal maintains the contact with the suction and/or staples so that it maintains sterility of the attachment.

In another alternative, the rigidizable overtube used in trans-gastric applications is used to provide a sterile field for access into the body. A cover may be provided on the the scope with an overtube that is sterile on the inside but is not sterile on the outside so we would fill the tip and have a sterile closed tube. Then as the the tube is inserted through the wall, the inside of the tube maintains the sterility. As the controllable instrument advances through the tube within the sterile liner, and it thereafter maintains a sterile environment up to the point that the tissue is pierced.

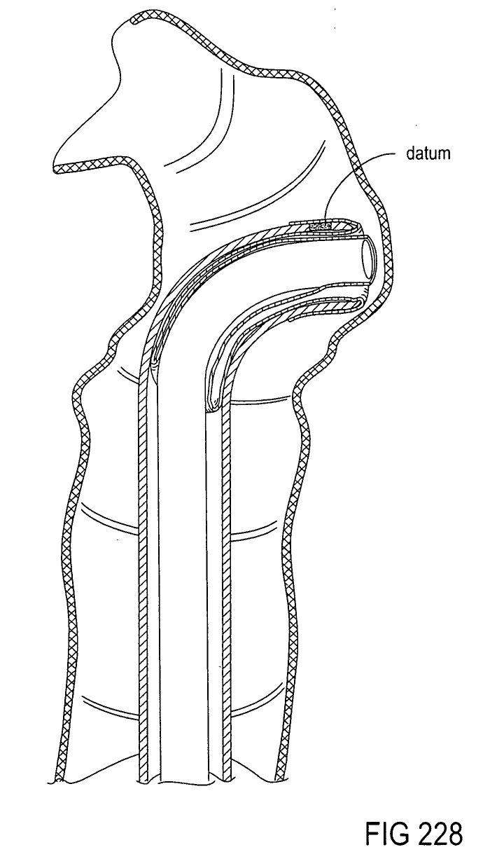

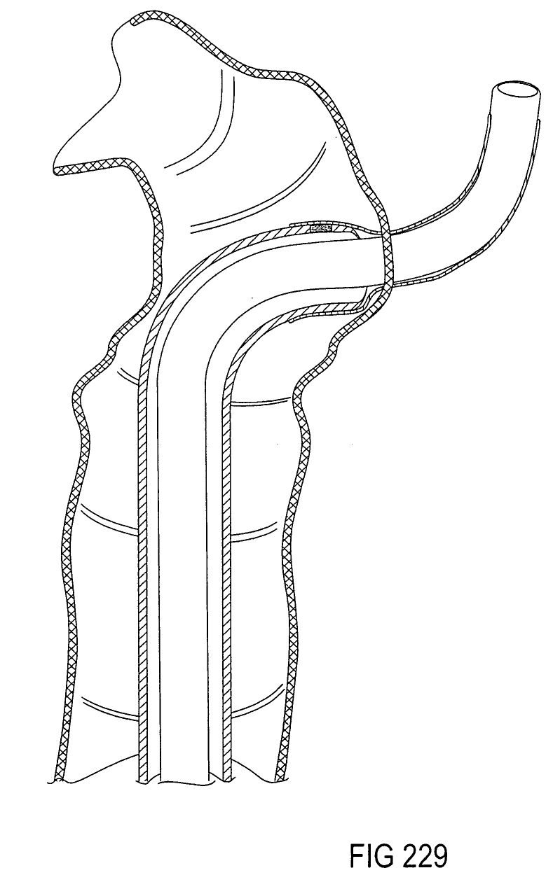

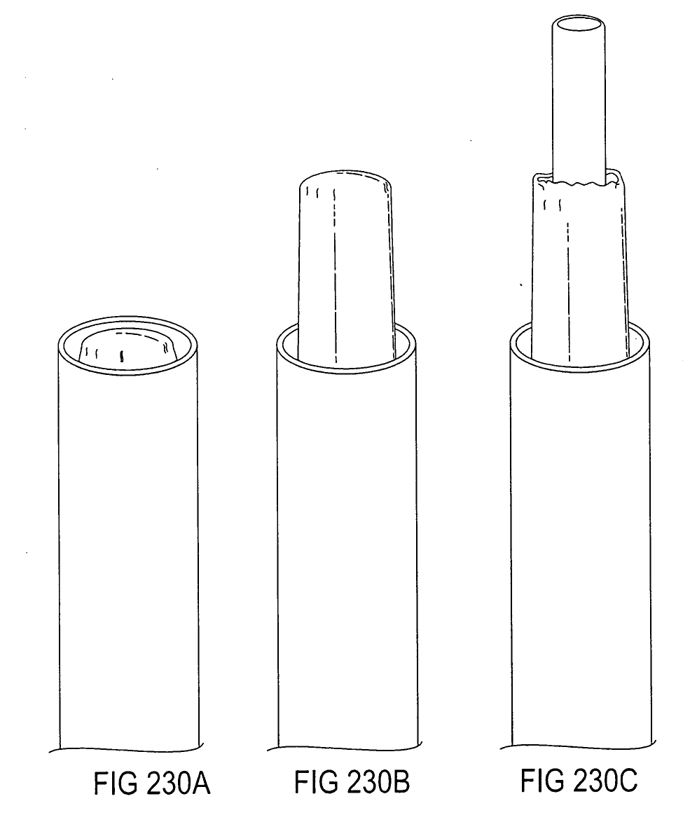

A sheath may be applied to instrument prior to introduction into guide or through lumen. FIG. 228 illustrates a guide tube having a sheath stowed in the distal end that is deployed as an instrument is advanced through the guide tube lumen and then may be later opening within an accessed body cavity or location as shown in FIG. 229. FIGS. 230A-C also illustrate the use of sheaths that are used initially within the guide tube as illustrated in FIG. 230A or advanced from the guide as shown in FIG. 230B. Once in the desired position, the sheath may be pierced as shown in FIG. 230C.

The techniques and instruments described herein may also be used in procedures having a combination of internal and externally provided devices. One example would be a transluminal procedure used to guide an instrument to access or manipulate externally provided devices or implements.

Another advantageous combination of the rigidizable endoscope is used within the body to provide a navigation pathway or a selectively steerable segmented instrument. A device to be used within the body is passed through the skin with a scope adjacent the position of the scope now inside the body. For example, one could introduce the device through the skin using a small needle or trocar or introducer. The device introduced to the skin is then manipulated or secured from inside the body using the steerable segmented instrument. Thereafter the steerable instrument may be used to manipulate the delivery, use or employment of the device within the body. The examples of devices that may be used in this technique include for example, a stent, an implantable device, a pacing lead, or other pharmacological materials or agents, staples, barbs, or other implantable devices.

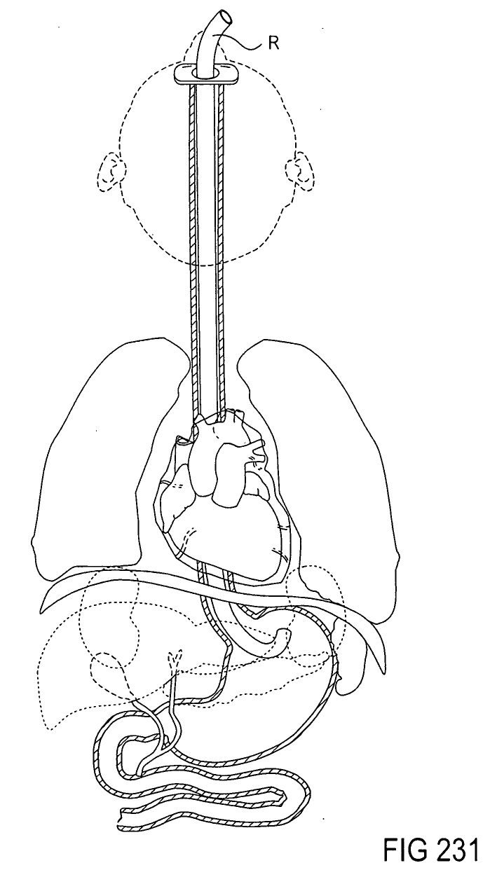

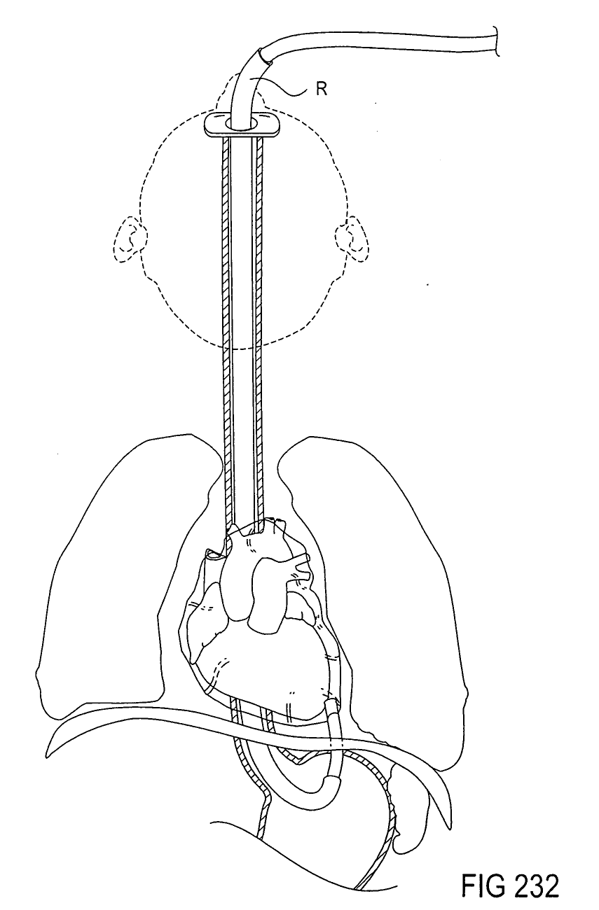

The instruments, systems and methods described herein provide for new procedures enabled by the inventive devices and methods. The rigidizable guide and steerable segmented instrument combination may be advantageously used to perform a wide variety of procedures in the body. One procedure relates to approaching the thoracic cavity by landing the rigidizable overtube onto the stomach, piercing through the stomach wall and advancing the controllable segmented instrument to pierce the diaphragm unaided by an additional rigidizable guide tube as best seen in FIGS. 231 and 232. Once through the diaphragm the segmented instrument is navigated, advanced, or otherwise guided into the chest cavity for any procedure that is performed in the thoracic cavity. For example, the segmented instrument working channel or other lumen therein could be used or additional instruments could be provided, for example, for the placement of biventricular leads, or for treatment of atrial fibrillation.

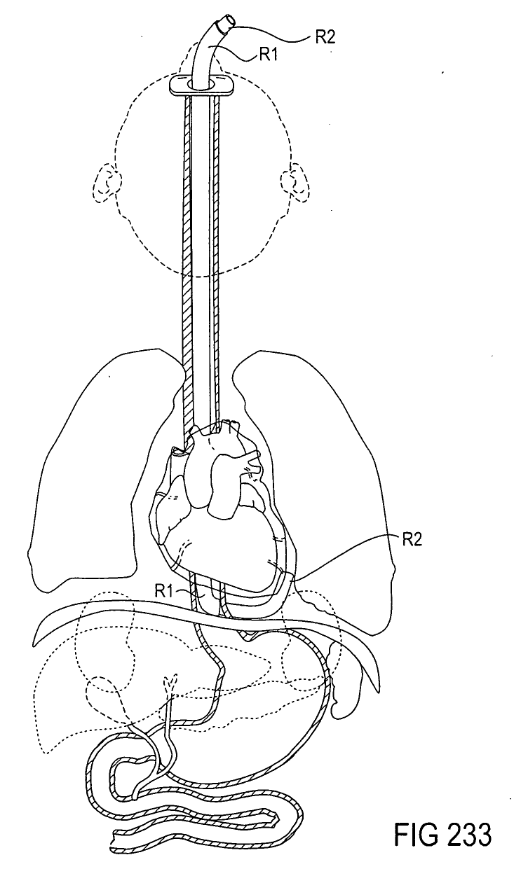

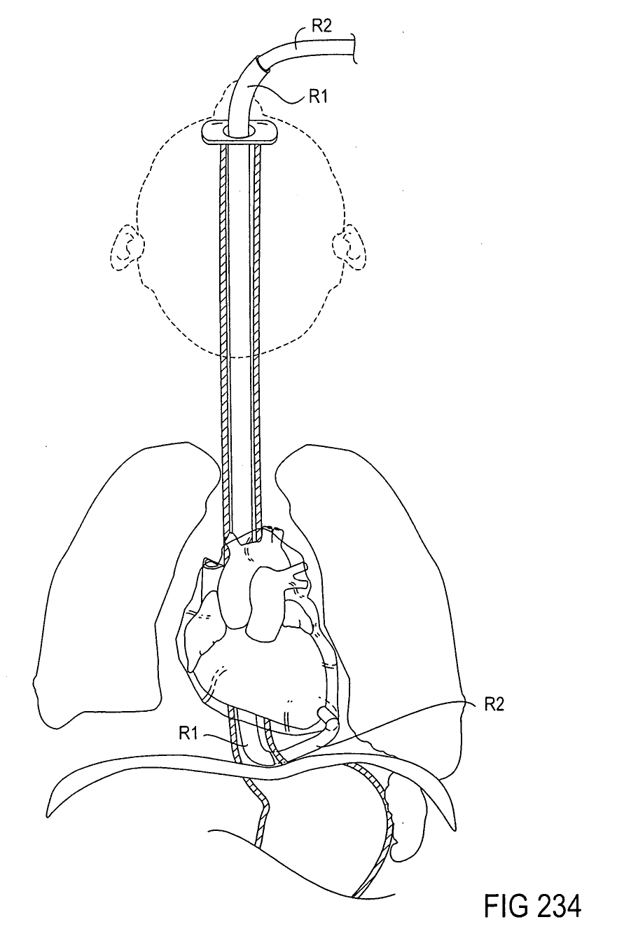

FIGS. 233 and 234 illustrate how multiple rigidizable guides may be used for trans-esophageal and trans-diaphragm access to the heart and/or other organs of the thoracic cavity. Alternatively, a selectively rigidizable guide tube is landed against the stomach wall and after affixing that guide tube, providing an opening in the stomach wall. Thereafter, a second rigidizable guide tube is advanced through the first rigidizable guide tube through the opening in the stomach and to a position on the diaphragm. The second rigidizable guide tube is secured to the diaphragm and an opening in the diaphragm formed. Thereafter a steerable, segmented instrument is advanced navigate through the first and second rigidizable guide tubes to perform any of a variety of trans-diaphragm procedures within the thoracic cavity.

Another concept is the use of one or more rigidizable guide tubes to provide a trans-gastric-diaphragmic access to the thoracic cavity. First rigidizable overtube could be landed against the stomach wall thereafter a second rigidizable guide tube advanced through the first passes through the stomach wall and is advanced into an engaging position with the diaphragm. The second rigidizable tube distal end could be sealed against the diaphragm tissue in a number of ways. For example, 2 magnets placed on opposite sides could be used for sealing. Alternatively balloons could be used to seal the rigidizable scope against the diaphragm. These concepts alternatively the sealing techniques described herein could also be used to seal either or both of the rigidizable guide tubes 1 and 2 described for the transgastric trans-diaphragm seal. As such this provides a transgastric trans-diaphragm thoracic surgical technique and access. Using these and other technique described herein enables a new access port of access methods through the thoracic cavity.

Another access point provided by embodiments of present inventions include a trans-esophageal-trans-diaphragm access method. This method provides trans-diaphragm access through the esophagus rather than the stomach. In this method a rigidizable overtube is advanced through the esophagus until it is inferior to the diaphragm. Thereafter one or more rigidizable tubes could be used to sit the, to provide access through and secure against the inner wall of the esophagus thereafter advanced through the first rigidizable scope a second rigidizable scope that is anchored to the diaphragm and then access form through the diaphragm using the second stage described herein and using these first and second rigidizable scopes an access pathways provided into the thoracic cavity that is transesophageal and transdiaphragmic. Alternatively a single rigidizable endoscope could be used for this and other techniques. The single transesophageal transdiaphragmic rigidizable tube could be dimensioned in size with various sections and locking mechanisms to be adapted for this particular physiology. It is to be appreciated that longer and less articulable sections may be used in the esophageal portion while a smaller and more articulating or more bendable sections may be used in the portion of the rigidizable tube that exits the esophagus and is attached to the diaphragm. As such, as with the steerable segmented instruments, the selectively rigidizable guides may also contain segments of various sizes depending upon the specific application, physiology and anatomy.

Uses in Natural and Artificial Openings

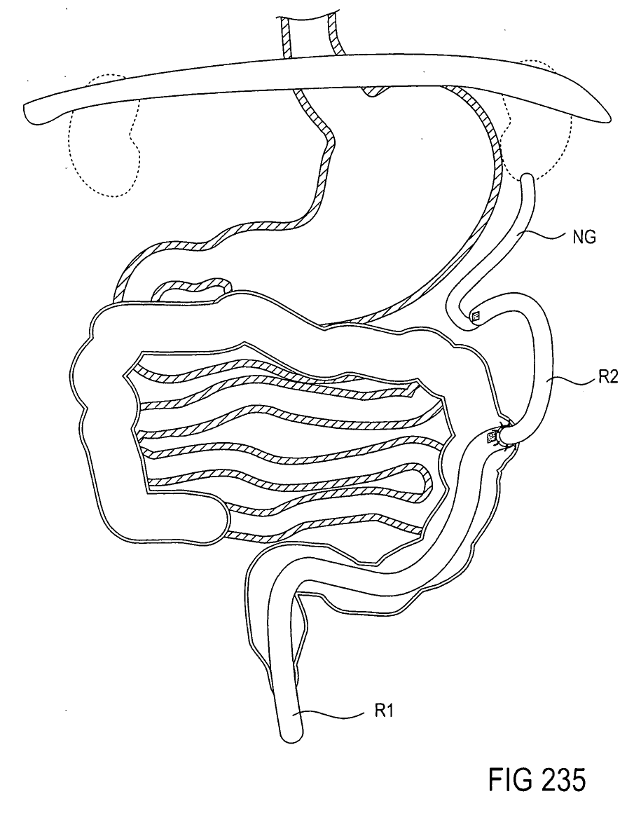

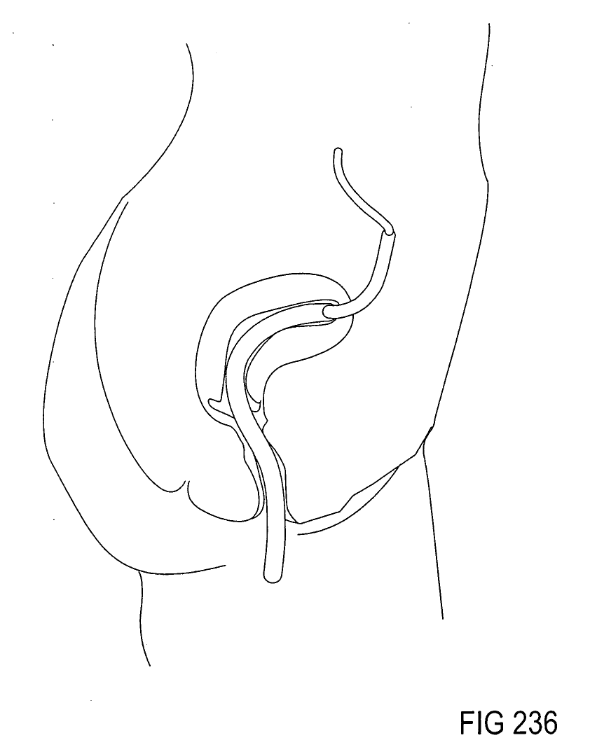

The embodiments described herein have primarily used applications for the use of controllable rigidizable guide tubes and a steerable segmented instruments. It is to be appreciated that the transgastric applications and uses within the gastrointestinal tract or the gut are nearly exemplary of some of the uses for the combination techniques described herein. It is to be appreciated that any opening or orifice either natural or artificial formed in the body may be used to provide the access points or the anchoring positions for the rigidizable guide tubes described herein. For example, as as illustrated in FIG. 236, the embodiments of the present invention may be used transvaginally, transuterully, transcervixally. For example, an embodiment of the rigidizable tube may be advanced up through the vagina and anchored against the uterine wall (FIG. 236). Thereafter a controllable steerable instrument may be advanced through the guide tube and an opening formed in the uterus to provide additional techniques within the uterie or cervix cavity. It is to be appreciated that any opening in the body whether natural or artificially created may be used as an access port or embodiments of the present invention. For example and as illustrated in FIG. 235, instruments described herein may be advanced through the colon, attached to the colon wall and thence into the body cavity in a trans-colonic access way.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for performing a transluminal procedure, comprising:
securing a datum and position indicator to a wall of a target lumen;
forming an opening in the wall;
advancing an instrument through the opening; and
tracking the advancement of the instrument using the datum and position indicator.

2. The method of claim 1 wherein the forming step comprises forming the opening with an instrument coupled to the datum and position indicator.

3. The method of claim 1 wherein the advancing step comprises advancing the instrument through a guide lumen in the datum and position indicator.

4. The method of claim 3 further comprising piercing a sheath extending across the guide lumen while advancing the instrument through the lumen in the datum and position indicator.

5. The method of claim 3 further comprising unrolling a sheath contained in the datum and position indicator while advancing the instrument through the guide lumen.

6. The method of claim 1 further comprising rigidizing a guide tube coupled to the datum and position indicator before tracking the advancement of the instrument.

7. The method of claim 1 further comprising sterilizing the wall of the target lumen after securing the datum and position indicator for the wall of the target lumen.

8. The method of claim 1 wherein the tracking step comprises providing instrument tracking information to a system used to monitor the progress of the instrument.

9. The method of claim 1 further comprising controlling articulation of the instrument using information from the tracking step.

10. Apparatus for performing a transluminal procedure comprising:
a cutting tool; and
a datum and position indicator comprising a luminal wall attachment mechanism and an instrument tracking mechanism adapted to monitor passage of an instrument through a luminal wall opening formed by the cutting tool.

11. The apparatus of claim 10 wherein the cutting tool is coupled to the datum and position indicator.

12. The apparatus of claim 10 further comprising a guide lumen, the instrument tracking mechanism being adapted to detect passage of an instrument through the guide lumen and through the luminal wall opening formed by the cutting tool.

13. The apparatus of claim 12 wherein the guide lumen comprises a rigidizable guide tube.

14. The apparatus of claim 10 further comprising a luminal wall sterilizing mechanism.

15. The apparatus of claim 10 further comprising a instrument tracking monitor in communication with the tracking mechanism to receive instrument tracking information.

16. Apparatus for performing a transluminal procedure comprising:
a cutting tool;
a transluminal instrument; and
a datum and position indicator comprising a guide lumen, a luminal wall attachment mechanism and an instrument tracking mechanism adapted to detect passage of the instrument through a luminal wall opening formed by the cutting tool.

17. The apparatus of claim 16 wherein the guide lumen comprises a sheath and the transluminal instrument comprises a sheath piercing mechanism adapted to pierce the sheath.

18. The apparatus of claim 16 wherein the guide lumen comprises a rolled sheath adapted to unroll as the instrument advances through the guide lumen.

19. The apparatus of claim 16 further comprising an instrument control in communication with the instrument tracking mechanism to control articulation of the instrument.

20. A method for providing a sterile field during a transluminal procedure, comprising:
securing an elongated body to a wall of a lumen;
advancing a sterilization device through the elongated body to a position adjacent the lumen wall; and
sterilizing a target portion of the lumen wall with the sterilization device.

21. The method of claim 20 wherein the sterilizing step comprises spraying a sterile sealant onto the lumen wall.

22. The method of claim 20 wherein the sterilizing step comprises securing a patch against and completely covering the target portion of the lumen wall.

23. The method of claim 20 further comprising creating an opening through the lumen wall after the sterilizing step.

24. Apparatus for performing a transluminal procedure comprising:
an elongated body comprising a luminal wall attachment mechanism at a distal portion of the elongated body; and
a luminal wall sterilization device extending from a proximal portion of the elongated body to the distal portion of the elongated body.

25. The apparatus of claim 24 wherein the sterilization device comprises a sprayer and a sterile sealant source.

26. The apparatus of claim 24 wherein the sterilization device comprises a patch, the patch comprising a luminal wall attachment mechanism.

27. The apparatus of claim 24 further comprising a cutting tool extending from a proximal portion of the elongated body to the distal portion of the elongated body.

28. An apparatus for use in a transluminal procedure, comprising:
a housing having a guide lumen and a seal proximal to a distal end of the housing that extends across and completely seals the guide lumen;
a fixation element in the housing and adapted to secure the distal end of the housing to tissue; and
a channel extending through the side wall of the housing having an outlet in communication with the lumen distal of the seal.

29. The apparatus of claim 28 wherein the fixation element comprises a plurality of tines.

30. The apparatus of claim 28 wherein the fixation element comprises a shaft and a plurality of wires extending from the shaft.

31. The apparatus of claim 28 wherein the fixation element is adapted to engage with tissue by rotating less than one half of one revolution.

32. The apparatus of claim 28 further comprising at least one cutting blade distal to the seal.

33. The apparatus of claim 32 wherein the at least one cutting blade distal to the seal is disposed entirely within the sidewall of the housing.

34. The apparatus of claim 28 wherein the housing is a guide tube.

35. The apparatus of claim 34 wherein the guide tube is a semi-rigidizable guide tube.

36. A method for performing a transluminal procedure comprising:
securing a distal end of a housing to tissue, the housing comprising a guide lumen and a seal proximal to a distal end of the housing that extends across and completely seals the guide lumen; and
sterilizing a region within the guide lumen distal to the seal.

37. The method of claim 36 further comprising forming an opening in tissue distal to the seal after the sterilizing step.

38. The method of claim 37 further comprising advancing an instrument through the seal after the sterilizing step.
